(19) United States
(12) Patent Application Publication
(10) Pub. No.: US 2026/0132125 A1
(43) Pub. Date: May 14, 2026

(54) PESTICIDALLY ACTIVE FUSED BICYCLIC HETEROAROMATIC COMPOUNDS

(71) Applicant: SYNGENTA CROP PROTECTION AG, Basel (CH)

(72) Inventors: Jagadeesh Prathap KILARU, Corlim, Ilhas (IN); Simone BERARDOZZI, Stein (CH); André JEANGUENAT, Stein (CH); Michel MUEHLEBACH, Stein (CH); Thomas PITTERNA, Stein (CH); Matthias WEISS, Stein (CH); Christopher Charles SCARBOROUGH, Stein (CH); Andre STOLLER, Stein (CH)

(73) Assignee: SYNGENTA CROP PROTECTION AG, Basel (CH)

(21) Appl. No.: 18/877,877

(22) PCT Filed: Jun. 16, 2023
(86) PCT No.: PCT/EP2023/066278

§ 371 Date: Dec. 20, 2024

(30) Foreign Application Priority Data
Jun. 21, 2022 (IN) 202211035526
Dec. 20, 2022 (EP) 22215128.4
Dec. 27, 2022 (IN) 202211075969

(51) Int. Cl. C07D 403/14; A01N 43/60; A01N 43/653; A01N 43/78; A01N 43/84; A01N 47/02; A01N 53/00; A01P 5/00; A01P 7/02; A01P 7/04; A01P 9/00; C07D 409/14; C07D 413/14; C07D 417/14

(52) U.S. Cl. CPC C07D 403/14; A01N 43/60; A01N 43/653; A01N 43/78; A01N 43/84; A01N 47/02; A01N 53/00; A01P 5/00; A01P 7/02; A01P 7/04; A01P 9/00; C07D 409/14; C07D 413/14; C07D 417/14

(57) ABSTRACT

Compounds of formula (I), wherein the substituents are as defined in claim 1, and the agrochemically acceptable salts, stereoisomers, enantiomers, tautomers and N-oxides of those compounds, can be used as insecticides.

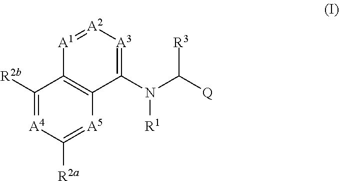

The present invention relates to pesticidally active, in particular insecticidally active quinazoline compounds, to processes for their preparation, to compositions comprising those compounds, and to their use for controlling animal pests, including arthropods and in particular insects or representatives of the order Acarina.

WO 2021/083936, WO 2021/148639 and WO 2021/177160 describe certain quinazoline, quinazolinone and quinoline compounds.

There have now been found further novel pesticidally active quinazoline compounds.

The present invention accordingly relates, in a first aspect, to a compound of the formula (I)

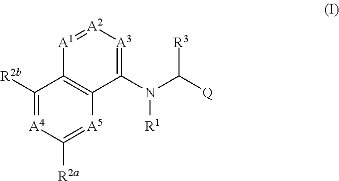

wherein:
A¹, A² and A³ are, independently from each other, N or CR^{Y}; or
A¹=A²-A³, taken together, are NR—C(═O)—N;
A⁴ and A⁵ are, independently from each other, N or CR^{YY};
Q is

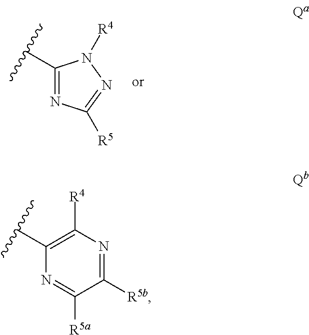

where the staggered line represents the connection of Q to the rest of compound of the formula (I);
R is hydrogen, C₁-C₃alkyl, C₁-C₃haloalkyl, C₁-C₃alkoxy, or C₁-C₃haloalkoxy;
R¹ is hydrogen, C₁-C₆alkyl, C₁-C₆cyanoalkyl, aminocarbonylC₁-C₆alkyl, hydroxycarbonylC₁-C₆alkyl, C₁-C₆nitroalkyl, trimethylsilaneC₁-C₆alkyl, C₁-C₃alkoxy-C₁-C₆alkyl, C₁-C₆haloalkyl, C₂-C₆alkenyl, C₂-C₆haloalkenyl, C₂-C₆alkynyl, C₂-C₆haloalkynyl, C₃-C₄cycloalkylC₁-C₂alkyl-, C₃-C₄cycloalkylC₁-C₂alkyl- wherein the C₃-C₄cycloalkyl group is substituted with 1 or 2 halogen atoms, oxetan-3-yl-CH₂—, C₁-C₆alkylcarbonyl, C₁-C₆alkoxycarbonyl, phenyloxycarbonyl, benzyloxycarbonyl, benzyl or benzyl substituted with 1 to 3 substituents independently selected from halogen, C₁-C₆alkoxy and C₁-C₆haloalkyl;
R^{2a} and R^{2b} are each independently selected from hydrogen, C₁-C₃alkyl, C₁-C₃haloalkyl, C₁-C₃haloalkylsulfanyl, C₁-C₃alkoxy, C₁-C₃haloalkoxy, halogen, NO₂, SF₅, CN, C(O)NH₂, C(O)OH, C(S)NH₂, C₃-C₆cycloalkyl, C₃-C₆cycloalkyl substituted with one to three substituents independently selected from R^{x}, C₃-C₆cycloalkylcarbonyl, phenyl, phenyl substituted with one to three substituents independently selected from R^{x}, heteroaryl, heteroaryl substituted with one to three substituents independently selected from R^{x}, OR⁶, piperidin-2-one-1-yl, piperidin-2-one-1-yl substituted with one to two substituents independently selected from R^{x}, pyridin-2-one-1-yl, pyridin-2-one-1-yl substituted with one to two substituents independently selected from R^{x}, azetidin-1-yl, azetidin-1-yl substituted with one to two substituents independently selected from R^{x}, pyrrolidin-1-yl, pyrrolidin-1-yl substituted with one to two substituents independently selected from R^{x}, C₃-C₆cycloalkylC₁-C₄alkyl, C₃-C₆cycloalkylC₁-C₄alkyl substituted with one to two substituents independently selected from R^{z}; C₃-C₆cycloalkylC₁-C₃alkoxy, C₃-C₆cycloalkylC₁-C₃alkoxy substituted with one to two substituents independently selected from R^{x}, C₁-C₅cyanoalkyl, C₁-C₅cyanoalkoxy, C₁-C₄alkylsulfanyl, C₁-C₄alkylsulfanyl substituted with one to three substituents independently selected from R^{x}, C₁-C₄alkylsulfonyl, C₁-C₄alkylsulfonyl substituted with one to three substituents independently selected from R^{x}, C₁-C₄alkylsulfinyl, and C₁-C₄alkylsulfinyl substituted with one to three substituents independently selected from R^{x};
R³ is C₁-C₃alkyl or C₁-C₃haloalkyl;
R⁴ and R^{4a} are independently pyrimidinyl, pyrazinyl, pyridazinyl or thiazolyl, each of which, independently of each other, is substituted with a single —C(O)NR¹⁰R¹¹; or
R^{4a} is N-linked pyrazolyl or N-linked triazolyl, each of which, independently of each other, is substituted with a single —C(O)NR¹⁰R¹¹; or
R⁴ and R^{4a} are 4-cyano-pyrimidin-6-yl;
R¹⁰ is hydrogen, hydroxy, C₁-C₃alkyl, C₁-C₃cyanoalkyl, C₁-C₃haloalkyl, C₁-C₃alkoxy, C₃-C₄cycloalkyl, C₃-C₄halocycloalkyl, cyanoC₃-C₄cycloalkyl, C₃-C₄cycloalkylC₁-C₃cyanoalkyl, oxetan-3-yl, thietan-3-yl, 3-methylthietan-3-yl, 1,1-dioxothietan-3-yl, or 3-methyl-1,1-dioxothietan-3-yl; and
R¹¹ is hydrogen, C₁-C₃alkyl or C₁-C₃haloalkyl; or
R¹⁰ and R¹¹, together with the nitrogen they are attached to, form a pyrrolidin-1-yl, piperidin-1-yl or 4-morpholinyl group;
R⁵ is hydrogen, C₁-C₃alkyl, C₁-C₃haloalkyl, C₃-C₄cycloalkyl, C₁-C₃alkoxy, C₃-C₄alkoxyC(O)—, (C₁-C₃alkoxy)₂CH—, halogen, CN, NH₂C(O), amino (i.e. NH₂), (C₁-C₃alkyl)amino, di(C₁-C₃alkyl)amino, hydroxy, C₃-C₄halocycloalkyl, C₃-C₄cyanocycloalkyl, C₂-C₆alkenyl, C₂-C₆haloalkenyl, C₂-C₆alkynyl, C₂-C₆haloalkynyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl, C₁-C₄alkylsulfonyl, C₁-C₃alkoxy-C₁-C₃alkyl, C₁-C₃alkoxy-C₁-C₃alkoxy-C₁-C₃alkyl, (C₁-C₃alkyl)sulfonylamino, (C₁-C₃alkyl)sulfonyl(C₁-C₃alkyl)amino, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), (C₁-C₃alkyl)C(O)(C₁-C₃alkyl)N, (C₁-C₃alkyl)C(O)NH, (C₁-C₃alkyl)C(O), (C₁-C₃alkoxy)C(O), HC(O), diphenylmethanimine, C₁-C₃haloalkoxy, phenyl, or a 5-membered heteroaromatic ring; or
R⁵ is phenyl substituted with one to three substituents selected from C₁-C₃alkyl, C₁-C₃haloalkyl, C₁-C₃alkoxy, C₃-C₄cycloalkyl, halogen, CN and hydroxyl; or
R⁵ is a 5-membered heteroaromatic ring substituted with one to three substituents selected from C₁-C₃alkyl, C₁-C₃haloalkyl, C₁-C₃alkoxy, C₃-C₄cycloalkyl, halogen, CN and hydroxyl;
R^{5a} and R^{5b} are, independently of each other, selected from hydrogen, halogen, CN, C₁-C₃alkyl, C₁-C₃haloalkyl, C₃-C₄cycloalkyl, C₁-C₃alkoxy, and C₁-C₃haloalkoxy;
R⁶ is phenyl, benzyl, heteroaryl, or C₃-C₆ cycloalkyl; or
R⁶ is phenyl, benzyl, heteroaryl, or C₃-C₆ cycloalkyl, each of which, independent of each other, is substituted with one to three substituents independently selected from R^{x};
R^{x} is independently selected from halogen, C₁-C₃alkyl, C₁-C₃haloalkyl, C₁-C₃alkoxy, C₁-C₃haloalkoxy, NO₂, SF₅, CN, C(O)NH₂, C(S)NH₂, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl and C₁-C₄alkylsulfonyl;
R^{Y} is selected from hydrogen, C₁-C₃ alkyl, C₁-C₃ haloalkyl, hydroxy, C₁-C₃ alkoxy, C₁-C₃ haloalkoxy, halogen, CN and cyclopropyl;
R^{YY} is selected from hydrogen, C₁-C₃ alkyl, C₁-C₃ haloalkyl, hydroxy, C₁-C₃ alkoxy, C₁-C₃ haloalkoxy, halogen, CN and cyclopropyl; and
R^{z} is selected from oxo, halogen, C₁-C₃ alkyl, C₁-C₃haloalkyl, C₁-C₃alkoxy, C₁-C₃haloalkoxy and CN;
and an agrochemically acceptable salt, stereoisomer, enantiomer, tautomer and N-oxide of the compound of formula (I).

Compounds of formula (I) which have at least one basic centre can form, for example, acid addition salts, for example with strong inorganic acids such as mineral acids, for example perchloric acid, sulfuric acid, nitric acid, nitrous acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds of formula (I) which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

In each case, the compounds of formula (I) according to the invention are in free form, in oxidized form as a N-oxide or in salt form, e.g. an agronomically usable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The compounds of formula (I) according to the invention also include hydrates which may be formed during the salt formation.

The term “C₁-Cₙalkyl” as used herein refers to a saturated straight-chain or branched hydrocarbon radical attached via any of the carbon atoms having 1 to n carbon atoms, for example, any one of the radicals methyl, ethyl, n-propyl, 1-methylbutyl, 2-methylbutyl, 3-methylbutyl, 2, 2-dimethylpropyl, 1-ethylpropyl, n-hexyl, n-pentyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl.

The term “C₁-Cₙhaloalkyl” as used herein refers to a straight-chain or branched saturated alkyl radical attached via any of the carbon atoms having 1 to n carbon atoms (as mentioned above), where some or all of the hydrogen atoms in these radicals may be replaced by fluorine, chlorine, bromine and/or iodine, i.e., for example, any one of chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl. According a term “C₁-C₂fluoroalkyl” would refer to a C₁-C₂alkyl radical which carries 1, 2, 3, 4, or 5 fluorine atoms, for example, any one of difluoromethyl, trifluoromethyl, 1-fluoroethyl, 2-fluoroethyl, 2,2-difluoroethyl, 2,2,2-trifluoroethyl, 1,1,2,2-tetrafluoroethyl or pentafluoroethyl.

The term “C₁-Cₙalkoxy” as used herein refers to a straight-chain or branched saturated alkyl radical having 1 to n carbon atoms (as mentioned above) which is attached via an oxygen atom, i.e., for example, any one of the radicals methoxy, ethoxy, n-propoxy, 1-methylethoxy, n-butoxy, 1-methylpropoxy, 2-methylpropoxy or 1,1-dimethylethoxy. The term “haloC₁-Cₙalkoxy” as used herein refers to a C₁-Cₙalkoxy radical where one or more hydrogen atoms on the alkyl radical is replaced by the same or different halo atom(s)—examples include trifluoromethoxy, 2-fluoroethoxy, 3-fluoropropoxy, 3,3,3-trifluoropropoxy, 4-chlorobutoxy.

The term “C₁-Cₙcyanoalkyl” as used herein refers to a straight chain or branched saturated C₁-Cₙalkyl radical having 1 to n carbon atoms (as mentioned above), where one of the hydrogen atoms in these radicals is replaced by a cyano group: for example, cyanomethyl, 2-cyanoethyl, 2-cyanopropyl, 3-cyanopropyl, 1-(cyanomethyl)-2-ethyl, 1-(methyl)-2-cyanoethyl, 4-cyanobutyl, and the like.

The term “C₃-Cₙcycloalkyl” as used herein refers to 3-n membered cycloalkyl groups such as cyclopropane, cyclobutane, cyclopentane and cyclohexane.

The term “cyanoC₃-C₄cycloalkyl” as used herein refers to cyclopropane or cyclobutane groups mono substituted by cyano.

The term “C₃-Cₙcycloalkylcarbonyl” as used herein refers to a 3-n membered cycloalkyl group attached to a carbonyl (C═O) group, which carbonyl group is connected to the rest of the molecule. Similarly the terms “C₁-Cₙalkylcarbony”, “C₁-Cₙalkoxycarbonyl”, “phenyloxycarbonyl” and “benzyloxycarbonyl” as used herein refers to an alkyl, alkoxy, phenyloxy and benzyloxy group attached to a carbonyl (C═O) group, which carbonyl group is connected to the rest of the molecule.

The term “C₃-C₄cycloalkyl-C₁-C₂alkyl” as used herein refers to 3 or 4 membered cycloalkyl group with either a methylene or ethylene group, which methylene or ethylene group is connected to the rest of the molecule. In the instance, the C₃-C₄cycloalkyl-C₁-C₂alkyl- group is substituted, the substituent(s) can be on the cycloalkyl group and/or on the alkyl group.

The term “C₃-C₆cycloalkylC₁-C₄haloalkoxy” as used herein refers to a 3 to 6 membered cycloalkyl group connected to a 1 to 4 membered haloalkoxy, which haloalkoxy group is connected to the rest of the molecule.

The term “aminocarbonylC₁-Cₙalkyl” as used herein refers to an alkyl radical where one of the hydrogen atoms in the radical is replaced by CONH2 group.

The term “hydroxycarbonylC₁-Cₙalkyl” as used herein refers to an alkyl radical where one of the hydrogen atoms in the radical is replaced by COOH group.

The term “C₁-Cₙalkylsulfanyl” as used herein refers to a C₁-Cₙalkyl moiety linked through a sulfur atom. Similarly, the term “C₁-Cₙhaloalkylthio” or “C₁-Cₙhaloalkylsulfanyl” as used herein refers to a C₁-Cₙhaloalkyl moiety linked through a sulfur atom. Similarly, the term “C₃-Cₙcycloalkylsulfanyl” refers to 3-n membered cycloalkyl moiety linked through a sulfur atom.

The term “C₁-Cₙalkylsulfinyl” as used herein refers to a C₁-Cₙalkyl moiety linked through the sulfur atom of the S(═O) group. Similarly, the term “C₁-Cₙhaloalkylsulfinyl “or “C₁-Cₙhaloalkylsulfinyl” as used herein refers to a C₁-Cₙhaloalkyl moiety linked through the sulfur atom of the S(═O) group. Similarly, the term “C₃-Cₙcycloalkylsulfinyl” refers to 3-n membered cycloalkyl moiety linked through the sulfur atom of the S(═O) group.

The term “C₁-Cₙalkylsulfonyl” as used herein refers to a C₁-Cₙalkyl moiety linked through the sulfur atom of the S(═O)₂ group. Similarly, the term “C₁-Cₙhaloalkylsulfonyl “or “C₁-Cₙhaloalkylsulfonyl” as used herein refers to a C₁-Cₙhaloalkyl moiety linked through the sulfur atom of the S(═O)₂ group. Similarly, the term “C₃-Cₙcycloalkylsulfonyl” refers to 3-n membered cycloalkyl moiety linked through the sulfur atom of the S(═O)₂ group

The term “trimethylsilaneC₁-Cₙalkyl” as used herein refers to an alkyl radical where one of the hydrogen atoms in the radical is replaced by a —Si(CH₃)₃ group.

The term “C₂-Cₙalkenyl” as used herein refers to a straight or branched alkenyl chain having from two to n carbon atoms and one or two double bonds, for example, ethenyl, prop-1-enyl, but-2-enyl.

The term “C₂-Cₙhaloalkenyl” as used herein refers to a C₂-Cₙalkenyl moiety substituted with one or more halo atoms which may be the same or different.

The term “C₂-Cₙalkynyl” as used herein refers to a straight or branched alkynyl chain having from two to n carbon atoms and one triple bond, for example, ethynyl, prop-2-ynyl, but-3-ynyl.

The term “C₂-Cₙhaloalkynyl” as used herein refers to a C₂-Cₙalkynyl moiety substituted with one or more halo atoms which may be the same or different.

Halogen or “halo” is generally fluorine, chlorine, bromine or iodine. This also applies, correspondingly, to halogen in combination with other meanings, such as haloalkyl

The term “heteroaryl” as used herein refers to a 5- or 6-membered aromatic monocyclic ring having 1 to 3 heteroatoms independently selected from N, O and S. Examples are heteroaryls J-1 to J-41 shown in Scheme A below. Preferred heteroaryl is pyridyl, pyrimidyl, and pyrazolyl.

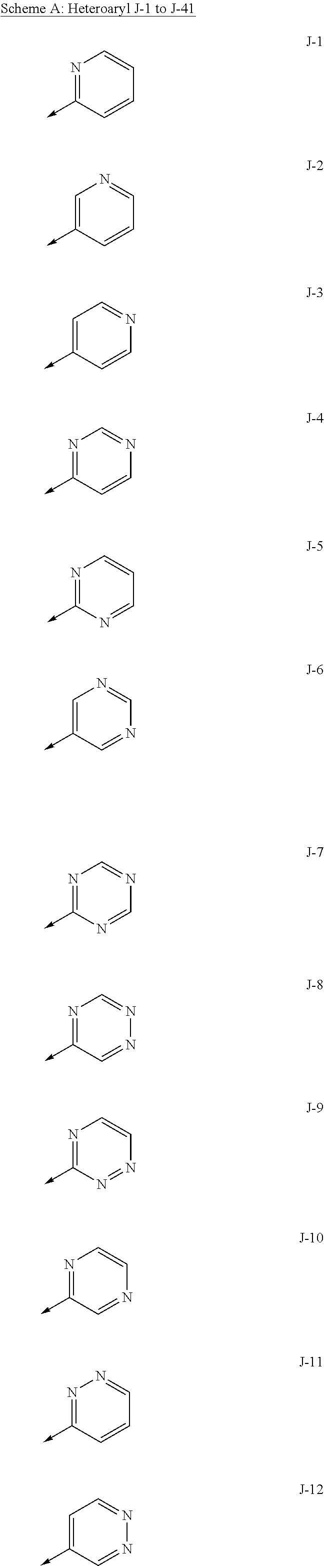

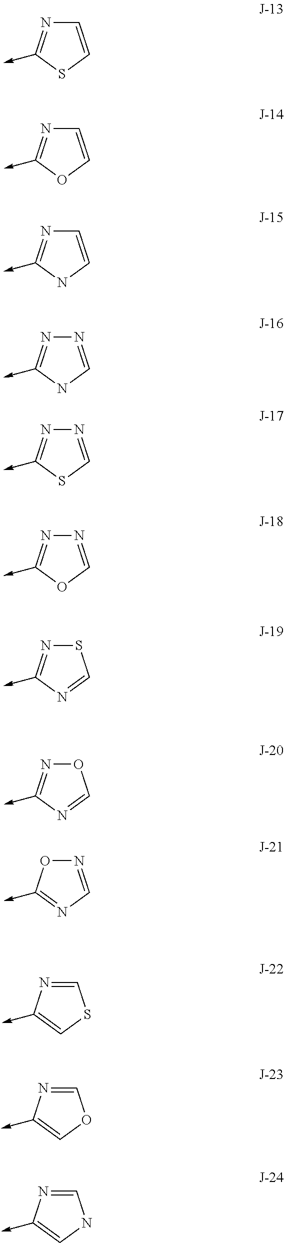

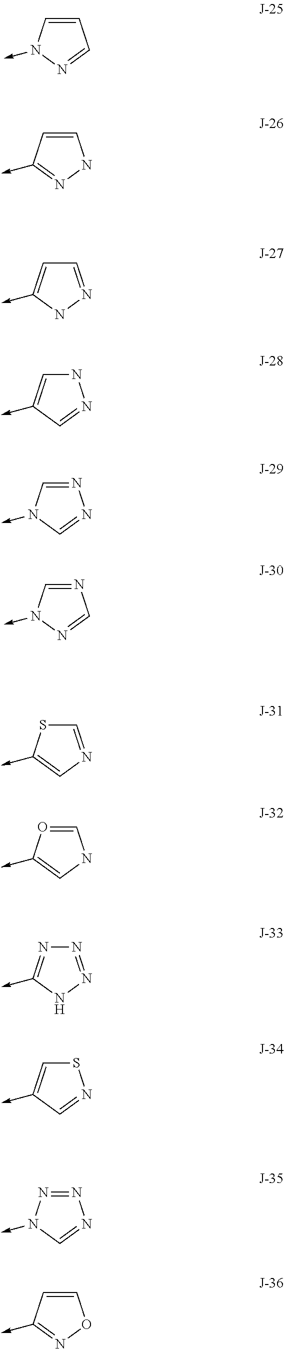

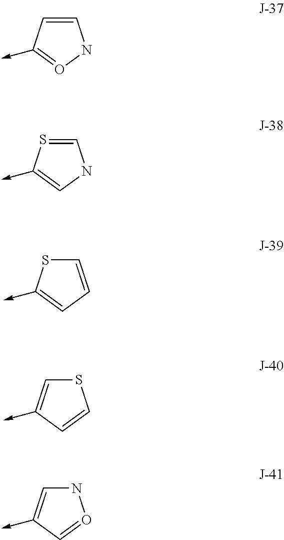

The pyridine, pyrimidine, pyrazine and pyridazine groups (unsubstituted or substituted) for R⁴ and R^{4a} are each connected via a carbon atom on the respective ring to the rest of the compound.

As used herein, the term “controlling” refers to reducing the number of pests, eliminating pests and/or preventing further pest damage such that damage to a plant or to a plant derived product is reduced.

The staggered line as used herein, for example, in Q^{a}-1 and Q^{b}-1, represent the point of connection/attachment to the rest of the compound.

As used herein, the term “pest” refers to insects, and molluscs that are found in agriculture, horticulture, forestry, the storage of products of vegetable origin (such as fruit, grain and timber); and those pests associated with the damage of man-made structures. The term pest encompasses all stages in the life cycle of the pest.

As used herein, the term “effective amount” refers to the amount of the compound, or a salt thereof, which, upon single or multiple applications provides the desired effect.

An effective amount is readily determined by the skilled person in the art, by the use of known techniques and by observing results obtained under analogous circumstances. In determining the effective amount a number of factors are considered including, but not limited to: the type of plant or derived product to be applied; the pest to be controlled & its lifecycle; the particular compound applied; the type of application; and other relevant circumstances.

As one of ordinary skill in the art will appreciate, compounds of formula (I) contain a stereogenic centre which is indicated with an asterisk in the structure below:

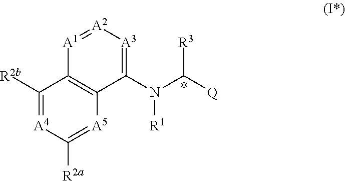

where R¹, R^{2a}, R^{2b}, R³, Q, A¹, A², A³, A⁴ and A⁵ are as defined in the first aspect.

The present invention contemplates both racemates and individual enantiomers. Compounds having preferred stereochemistry are set out below. Particularly preferred compounds of the present invention are compounds of formula (I′a):

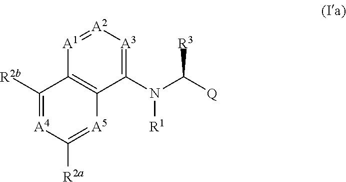

where R¹, R^{2a}, R^{2b}, R³, Q, A¹, A², A³, A⁴, and A⁵ are as defined in the first aspect, and stereoisomers, enantiomers, tautomers and N-oxides of the compounds of formula (I′a), and agrochemically acceptable salts thereof.

The term “optionally substituted” as used herein means that the group referenced is either unsubstituted or is substituted by a designated substituent, for example, “C₃-C₄cycloalkyl is optionally substituted with 1 or 2 halo atoms” means C₃-C₄cycloalkyl, C₃-C₄cycloalkyl substituted with 1 halo atom and C₃-C₄cycloalkyl substituted with 2 halo atoms.

Embodiments according to the invention are provided as set out below.

In an embodiment of each aspect of the invention, R is not hydrogen.
In an embodiment of each aspect of the invention,
A. A¹, A² and A³ are, independently from each other, N or CR^{Y}, with the proviso that no more than two out of the three are N; or
B. A¹ and A³ are N and A² is CR^{Y}; or
C. A¹, A² and A³ are, independently from each other, N or CH; or
D. A¹, A² and A³ are, independently from each other, N or CH, with the proviso that no more than two out of the three are N; or
E. A¹ is N, and A² and A³ are CH; or
F. A¹ and A² CH, and A³ is N; or
G. A¹ and A³ are N, and A² is CH; or
H. A¹=A²-A³, taken together, are NR—C(═O)—N; or
I. A¹=A²-A³, taken together, are NH—C(═O)—N; or
   In an embodiment of each aspect of the invention,
A. A⁴ is CR^{YY}, and A⁵ is N; or
B. A⁴ is CR^{YY}, and A⁵ is CH; or
C. A⁴ is CH, and A⁵ is N; or
D. A⁴ is N, and A⁵ is CH; or
E. A⁴ and A⁵ are both CH.
   In an embodiment of each aspect of the invention,
A. A¹ is N, A² and A³ are CH, and A⁴ and A⁵ are both CH; or
B. A¹ and A² are CH, A³ is N, and A⁴ and A⁵ are both CH.
C. A¹ and A³ are N, A² is CH, and A⁴ is CR and A⁵ is CH; or
D. A¹ and A³ are N, A² is CH, and A⁴ is CH and A⁵ is N; or
E. A¹ and A³ are N, A² is CH, and A⁴ is N and A⁵ is CH; or
F. A¹ and A³ are N, A² is CH, and A⁴ is N or CH and A⁵ is CH; or
G. A¹ and A³ are N, A² is CH, and A⁴ is CH and A⁵ is N or CH; or
H. A¹ and A³ are N, A² is CH, and A⁴ and A⁵ are both N; or
I. A¹ and A³ are N, A² is CH, and A⁴ and A⁵ are both CH; or
J. A¹=A²-A³, taken together, are NH—C(═O)—N and A⁴ and A⁵ are both CH.
   In an embodiment of each aspect of the invention where A¹=A²-A³, taken together, are NR—C(═O)—N, R is
A. hydrogen, methyl, ethyl, difluoroethyl or trifluoroethyl; or
B. hydrogen; methyl, ethyl, 2,2-difluoroethyl or 2,2,2,-trifluoroethyl or
C. hydrogen; or
D. methyl, or 2,2,2,-trifluoroethyl; or
E. methyl; or
F. hydrogen, or methyl.
   In preferred embodiments of each aspect of the invention, A¹ and A³ are nitrogen, A², A⁴ and A⁵ are CH.
   In an embodiment of each aspect of the invention, R¹ is
A. hydrogen, C₁-C₆alkyl, C₁-C₆cyanoalkyl, aminocarbonylC₁-C₆alkyl, hydroxycarbonylC₁-C₆alkyl, C₁-C₆nitroalkyl, trimethylsilaneC₁-C₆alkyl, C₁-C₃alkoxy-C₁-C₆alkyl, C₁-C₆haloalkyl, C₂-C₆alkenyl, C₂-C₆haloalkenyl, C₂-C₆alkynyl, C₂-C₆haloalkynyl, C₃-C₄cycloalkylC₁-C₂alkyl-, C₃-C₄cycloalkylC₁-C₂alkyl- wherein the C₃-C₄cycloalkyl group is substituted with 1 or 2 halogen atoms, oxetan-3-yl-CH₂—, C₁-C₃alkylcarbonyl, C₁-C₃alkoxycarbonyl, phenyloxycarbonyl, benzyloxycarbonyl, or benzyl; or
B. hydrogen, C₁-C₆alkyl, C₁-C₆cyanoalkyl, aminocarbonylC₁-C₆alkyl, hydroxycarbonylC₁-C₆alkyl, C₁-C₆nitroalkyl, trimethylsilaneC₁-C₆alkyl, C₁-C₃alkoxy-C₁-C₆alkyl, C₁-C₆haloalkyl, C₂-C₆alkenyl, C₂-C₆haloalkenyl, C₂-C₆alkynyl, C₂-C₆haloalkynyl, C₃-C₄cycloalkylC₁-C₂alkyl-, benzyloxycarbonyl, or benzyl; or
C. hydrogen, C₁-C₆alkyl, C₁-C₆cyanoalkyl, aminocarbonylC₁-C₆alkyl, hydroxycarbonylC₁-C₆alkyl, C₁-C₃alkoxy-C₁-C₆alkyl, C₁-C₆haloalkyl, C₂-C₆alkenyl, C₂-C₆haloalkenyl, C₂-C₆alkynyl, C₂-C₆haloalkynyl, C₃-C₄cycloalkylC₁-C₂alkyl-, benzyloxycarbonyl, or benzyl; or
D. hydrogen, C₁-C₆alkyl, C₁-C₆cyanoalkyl, C₁-C₃alkoxy-C₁-C₆alkyl, C₁-C₆haloalkyl, C₂-C₆alkenyl, C₂-C₆haloalkenyl, C₂-C₆alkynyl, C₂-C₆haloalkynyl, C₃-C₄cycloalkylC₁-C₂alkyl-, benzyloxycarbonyl, or benzyl; or
E. hydrogen, C₁-C₃alkyl, C₁-C₃cyanoalkyl, C₁-C₃alkoxy-C₁-C₃alkyl, C₁-C₃haloalkyl, C₂-C₄alkenyl, C₂-C₄haloalkenyl, C₂-C₄alkynyl, C₂-C₄haloalkynyl, C₃-C₄cycloalkylC₁-C₂alkyl-, benzyloxycarbonyl, or benzyl; or
F. hydrogen, C₁-C₃alkyl, C₁-C₃cyanoalkyl, C₁-C₃alkoxy-C₁-C₃alkyl, C₁-C₃haloalkyl, C₂-C₄alkenyl, C₂-C₄haloalkenyl, C₂-C₄alkynyl, C₂-C₄haloalkynyl, C₃-C₄cycloalkylC₁-C₂alkyl-, benzyloxycarbonyl, or benzyl; or
G. hydrogen, methyl, ethyl, cyanomethyl, methoxymethyl, cyclopropyl-methyl, allyl, propargyl, benzyloxycarbonyl, or benzyl; or
H. hydrogen, methyl, ethyl, allyl, propargyl or cyclopropyl-methyl; or
I. hydrogen, methyl, propargyl or cyclopropyl-methyl; or
J. hydrogen, methyl, or cyclopropyl-methyl; or
K. hydrogen, or methyl: or
L. hydrogen.
   In preferred embodiments of each aspect of the invention, R¹ is hydrogen, methyl or cyclopropyl-methyl, preferably, R¹ is hydrogen or methyl; more preferably, R¹ is hydrogen.
   In an embodiment of each aspect of the invention, R^{2a} is
A. hydrogen, halogen, C₁-C₃alkyl, C₁-C₃haloalkyl, C₁-C₃alkoxy, C₁-C₃haloalkoxy, CN, C₃-C₄cycloalkyl, C₃-C₆cycloalkylcarbonyl, phenyl, heteroaryl selected from J-1 and J-41, each of C₃-C₄cycloalkyl, phenyl or heteroaryl, independent of each other, is substituted with one to three substituents R^{x}, OR⁶, piperidin-2-one-1-yl, pyridin-2-one-1-yl, azetidin-1-yl optionally substituted with R^{x}, pyrrolidin-1-yl, C₃-C₆cycloalkylC₁-C₄alkyl substituted with one or two substituents R^{z}, C₃-C₆cycloalkylC₁-C₃alkoxy optionally substituted with R^{x}, C₁-C₅cyanoalkyl, C₁-C₅cyanoalkoxy, C₁-C₄alkylsulfanyl optionally substituted by one to three substituents R^{x}, C₁-C₄alkylsulfonyl optionally substituted by one to three substituents R^{x}, or C₁-C₄alkylsulfinyl optionally substituted by one to three substituents R^{x}; or
B. hydrogen, halogen, C₁-C₃alkyl, C₁-C₃haloalkyl, C₁-C₃alkoxy, C₁-C₃haloalkoxy, CN, C₃-C₄cycloalkyl, C₃-C₆cycloalkylcarbonyl, phenyl, pyrazolyl, each of C₃-C₄cycloalkyl, phenyl, pyrazolyl, independent of each other, is substituted with one to three substituents R^{x}, OR⁶, piperidin-2-one-1-yl, pyridin-2-one-1-yl, azetidin-1-yl optionally substituted with R^{x}, pyrrolidin-1-yl, C₃-C₆cycloalkylC₁-C₄alkyl optionally substituted with one or two substituents R^{z}, C₃-C₆cycloalkylC₁-C₃alkoxy optionally substituted with R^{x}, C₁-C₅cyanoalkyl, C₁-C₅cyanoalkoxy, C₁-C₄alkylsulfanyl optionally substituted by one to three substituents R^{x}, C₁-C₄alkylsulfonyl optionally substituted by one to three substituents R^{x}, or C₁-C₄alkylsulfinyl optionally substituted by one to three substituents R^{x}; or
C. hydrogen, halogen, C₁-C₃alkyl, C₁-C₃haloalkyl, C₁-C₃alkoxy, C₁-C₃haloalkoxy, CN, C₃-C₄cycloalkyl, C₃-C₆cycloalkylcarbonyl, phenyl or pyrazolyl, each of C₃-C₄cycloalkyl, phenyl, pyrazolyl, independent of each other, is substituted with one to two substituents R^{x}, OR⁶, azetidin-1-yl optionally substituted with R^{x}, C₃-C₆cycloalkylC₁-C₄alkyl optionally substituted with one or two substituents R^{z}, C₃-C₆cycloalkylC₁-C₃alkoxy optionally substituted with R^{x}, C₁-C₄alkylsulfanyl optionally substituted by one to three substituents R^{x}, C₁-C₄alkylsulfonyl optionally substituted by one to three substituents R^{x}, or C₁-C₄alkylsulfinyl optionally substituted by one to three substituents R^{x}; or
D. hydrogen, halogen, C₁-C₃alkyl, C₁-C₃haloalkyl, C₁-C₃alkoxy, C₁-C₃haloalkoxy, CN, C₃-C₄cycloalkyl, C₃-C₄cycloalkyl substituted with one to two substituents R^{x}, C₃-C₆cycloalkylcarbonyl, OR⁶, C₃-C₆cycloalkylC₁-C₄alkyl, C₃-C₆cycloalkylC₁-C₄alkyl substituted with one or two substituents R^{z}, C₁-C₄alkylsulfanyl, C₁-C₄alkylsulfanyl substituted by one to three substituents R^{x}, C₁-C₄alkylsulfonyl, C₁-C₄alkylsulfonyl substituted by one to three substituents R^{x}, C₁-C₄alkylsulfinyl, or C₁-C₄alkylsulfinyl substituted by one to three substituents R^{x}; or
E. hydrogen, halogen, C₁-C₃alkyl, C₁-C₃haloalkyl, C₁-C₃alkoxy, C₁-C₃haloalkoxy, CN, C₃-C₄cycloalkyl, C₃-C₄cycloalkyl substituted with one to two substituents independently selected from halogen, C₁-C₃alkyl and C₁-C₃haloalkyl, C₃-C₄cycloalkylcarbonyl, C₃-C₄cycloalkylmethyl, C₃-C₄cycloalkylmethyl substituted with one to two substituents independently selected from oxo, halogen, C₁-C₃alkyl, and C₁-C₃haloalkyl, C₁-C₂alkylsulfanyl substituted with one to three halogens or C₁-C₂alkylsulfonyl substituted with one to three halogens; or
F. hydrogen, halogen, C₁-C₃alkyl, C₁-C₃haloalkyl, C₁-C₃alkoxy, C₁-C₃haloalkoxy, cyclopropyl, cyclopropyl substituted with one to two substituents independently selected from halogen, methyl, and trifluoromethyl, cyclopropylcarbonyl, cyclopropylmethyl substituted with one to two substituents independently selected from oxo, halogen, and trifluoromethyl, or C₁-C₂alkylsulfanyl substituted with one to three halogens or C₁-C₂alkylsulfonyl substituted with one to three halogens; or
G. hydrogen, halogen, C₁-C₃alkyl, C₁-C₃haloalkyl, C₁-C₃haloalkylsulfanyl, C₁-C₃alkoxy, C₁-C₃haloalkoxy, CN, C₃-C₆cycloalkyl, C₃-C₆cycloalkyl substituted with one to three substituents independently selected from C₁-C₃alkyl, C₁-C₃haloalkyl, cyano, and halogen, cyclopropylcarbonyl, C₃-C₆cycloalkylC₁-C₄alkyl, C₃-C₆cycloalkylC₁-C₄alkyl substituted with one to five substituents independently selected from oxo, C₁-C₃alkyl, C₁-C₃haloalkyl, cyano, and halogen, C₁-C₅cyanoalkyl, C₁-C₄alkylsulfonyl, C₁-C₄haloalkylsulfonyl, C₁-C₄alkylsulfinyl, C₁-C₄haloalkylsulfinyl, C₃-C₆cycloalkylsulfanyl, C₃-C₆cycloalkylsulfinyl, or C₃-C₆cycloalkylsulfonyl; or
H. hydrogen, halogen, C₁-C₃alkyl, C₁-C₃haloalkyl, C₁-C₃haloalkylsulfanyl, C₁-C₃alkoxy, C₁-C₃haloalkoxy, CN, C₃-C₆cycloalkyl, C₃-C₆cycloalkyl substituted with one or two substituents independently selected from C₁-C₃haloalkyl, cyano, and halogen, C₃-C₄cycloalkylcarbonyl, C₃-C₆cycloalkylC₁-C₄alkyl, C₃-C₆cycloalkylC₁-C₄alkyl substituted with one to three substituents independently selected from oxo, C₁-C₃haloalkyl, cyano, and halogen, C₁-C₅cyanoalkyl, C₁-C₄alkylsulfonyl, C₁-C₄haloalkylsulfonyl, C₁-C₄alkylsulfinyl, C₁-C₄haloalkylsulfinyl, C₃-C₆cycloalkylsulfanyl, C₃-C₆cycloalkylsulfinyl, or C₃-C₆cycloalkylsulfonyl; or
I. hydrogen, halogen, C₁-C₃haloalkyl, C₁-C₃haloalkylsulfanyl, C₁-C₃haloalkoxy, C₃-C₆cycloalkyl, C₃-C₆cycloalkyl substituted with one or two substituents independently selected from C₁-C₃haloalkyl, cyano, and halogen, C₃-C₄cycloalkylcarbonyl, C₃-C₆cycloalkylC₁-C₄alkyl, C₃-C₆cycloalkylC₁-C₄alkyl substituted with one to three substituents independently selected from oxo, C₁-C₃haloalkyl, cyano, and halogen, C₁-C₅cyanoalkyl, C₁-C₄alkylsulfonyl, C₁-C₄haloalkylsulfonyl, C₁-C₄alkylsulfinyl, C₁-C₄haloalkylsulfinyl, C₃-C₆cycloalkylsulfanyl, C₃-C₆cycloalkylsulfinyl, or C₃-C₆cycloalkylsulfonyl; or
J. hydrogen, halogen, C₃-C₄cycloalkyl, C₃-C₄cycloalkylcarbonyl, C₃-C₄cycloalkyl-C₁-C₂alkyl optionally substituted with one to two substituents selected from oxo, halogen, C₁-C₃alkyl and C₁-C₃haloalkyl, C₁-C₃haloalkyl, C₁-C₃haloalkylsulfanyl, C₁-C₃haloalkysulfonyl, C₁-C₃alkoxy, C₁-C₃haloalkoxy, or CN; or
K. halogen, C₁-C₃haloalkyl, C₁-C₃haloalkylsulfanyl, C₁-C₃haloalkysulfonyl, or C₁-C₃haloalkoxy; or
L. halogen, C₁-C₂haloalkyl, C₁-C₂haloalkylsulfanyl, C₁-C₂haloalkysulfonyl, or C₁-C₂haloalkoxy; or
M. chlorine, fluorine, bromine, iodine, difluoromethyl, trifluoromethyl, trifluoromethylsulfanyl or trifluoromethylsulfonyl; or
N. fluorine, chlorine, bromine, iodine, trifluoromethylsulfanyl, trifluoromethylsulfonyl or trifluoromethyl; or
O. chlorine, bromine, iodine, trifluoromethylsulfonyl or trifluoromethyl; or
P. chlorine, bromine, iodine, methyl, trifluoromethylsulfonyl or trifluoromethyl; or
Q. chlorine, bromine, iodine, methyl, or trifluoromethyl; or
R. chlorine, bromine, iodine, or trifluoromethyl.
   In preferred embodiments of each aspect of the invention, R^{2a} is chlorine, bromine, iodine, or trifluoromethyl; preferably R^{2a} is chlorine or trifluoromethyl.
   In an embodiment of each aspect of the invention, R^{2b} is
A. hydrogen, halogen, C₁-C₃alkyl, C₁-C₃haloalkyl, C₃-C₄cycloalkyl, cyclopropylcarbonyl, C₃-C₆cycloalkylC₁-C₄alkyl optionally substituted with one or two substituents R^{z}, C₁-C₃alkoxy, C₁-C₃haloalkoxy, or CN, C₁-C₄alkylsulfanyl optionally substituted by one to three substituents R^{x}, C₁-C₄alkylsulfonyl optionally substituted by one to three substituents R^{x}, or C₁-C₄alkylsulfinyl optionally substituted by one to three substituents R^{x}; or
B. hydrogen, halogen, C₃-C₄cycloalkyl, cyclopropylcarbonyl, C₃-C₄cycloalkyl-C₁-C₂alkyl optionally substituted with one to two substituents selected from oxo, halogen, C₁-C₃alkyl and C₁-C₃haloalkyl, C₁-C₃haloalkyl, C₁-C₃haloalkysulfanyl, C₁-C₃haloalkysulfonyl, C₁-C₃alkoxy, C₁-C₃haloalkoxy, or CN; or
C. halogen, C₁-C₃haloalkyl, C₁-C₃haloalkylsulfanyl, C₁-C₃haloalkysulfonyl, or C₁-C₃haloalkoxy; or
D. halogen, C₁-C₂haloalkyl, C₁-C₂haloalkylsulfanyl, C₁-C₂haloalkysulfonyl, or C₁-C₂haloalkoxy; or
E. fluorine, chlorine, bromine, iodine, difluoromethyl, trifluoromethyl, trifluoromethylsulfanyl, trifluoromethylsulfonyl; or
F. fluorine, chlorine, bromine, iodine, trifluoromethylsulfanyl, trifluoromethylsulfonyl or trifluoromethyl; or
G. chlorine, bromine, iodine, trifluoromethylsulfonyl, or trifluoromethyl; or
H. chlorine, bromine, iodine, or trifluoromethyl; or
I. chlorine, or trifluoromethyl; or
J. chlorine, bromine, iodine, trifluoromethyl, difluoromethylsulfonyl, or trifluoromethylsulfonyl; or
K. difluoromethylsulfonyl, or trifluoromethylsulfonyl.
   In preferred embodiments of each aspect of the invention, R^{2b} is chlorine, bromine, iodine, trifluoromethyl, difluoromethylsulfonyl, or trifluoromethylsulfonyl; preferably R^{2b} is chlorine, bromine, iodine, or trifluoromethyl. In an embodiment of each aspect of the invention, R^{2b} is difluoromethylsulfonyl, or trifluoromethylsulfonyl.
   In an embodiment of each aspect of the invention,
A. R^{2a} and R^{2b} are independently selected from halogen, C₁-C₃haloalkyl, C₁-C₃haloalkysulfanyl, C₁-C₃haloalkysulfonyl, or C₁-C₃haloalkoxy; or
B. R^{2a} and R^{2b} are independently selected from fluorine, chlorine, bromine, iodine, trifluoromethylsulfanyl, trifluoromethylsulfonyl or trifluoromethyl; or
C. R^{2a} and R^{2b} are independently selected from chlorine, bromine, iodine, trifluoromethylsulfonyl or trifluoromethyl; or
D. R^{2a} is chlorine, bromine, iodine, methyl, trifluoromethylsulfonyl or trifluoromethyl; and R^{2b} is chlorine, bromine, iodine, trifluoromethylsulfonyl, or trifluoromethyl; or
E. R^{2a} is chlorine, bromine, iodine, methyl, or trifluoromethyl; and R^{2b} is chlorine, bromine, iodine, or trifluoromethyl; or
F. R^{2a} and R^{2b} are differently selected from chlorine, bromine, iodine, trifluoromethylsulfonyl or trifluoromethyl; or
G. R^{2a} and R^{2b} are differently selected from chlorine, bromine, iodine, or trifluoromethyl; or
H. R^{2a} is chlorine, bromine, iodine, or trifluoromethyl, and R^{2b} is chlorine, bromine, iodine, trifluoromethyl, difluoromethylsulfonyl, or trifluoromethylsulfonyl; or
I. R^{2a} is chlorine, or trifluoromethyl, and R^{2b} is chlorine, bromine, iodine, or trifluoromethyl.
   In preferred embodiments of each aspect of the invention, R^{2a} is chlorine, bromine, iodine, or trifluoromethyl and R^{2b} is chlorine, bromine, iodine, trifluoromethyl, difluoromethylsulfonyl, or trifluoromethylsulfonyl; preferably R^{2a} is chlorine, bromine, iodine, or trifluoromethyl, and R^{2b} is chlorine, bromine, iodine, or trifluoromethyl, or R^{2a} is chlorine or trifluoromethyl and R^{2b} is difluoromethylsulfonyl or trifluoromethylsulfonyl. More preferably, R^{2a} is chlorine or trifluoromethyl, and R^{2b} is chlorine, bromine, iodine, or trifluoromethyl.
   In an embodiment of each aspect of the invention, R³ is
A. C₁-C₃alkyl or C₁-C₃haloalkyl; or
B. methyl or trifluoromethyl; or
C. methyl.
   In preferred embodiments of each aspect of the invention, R³ is methyl.
   In an embodiment of each aspect of the invention, Q is
A. Q^{a}; or
B. Q^{b}.
   In an embodiment of each aspect of the invention, Q^{a} is
A. selected from Q^{a}-1 to Q^{a}-16; or
B. selected from Q^{a}-1, Q^{a}-6, Q^{a}-7, Q^{a}-10, and Q^{a}-15; or
C. Q^{a}-1 or Q^{a}-15;
D. Q^{a}-1

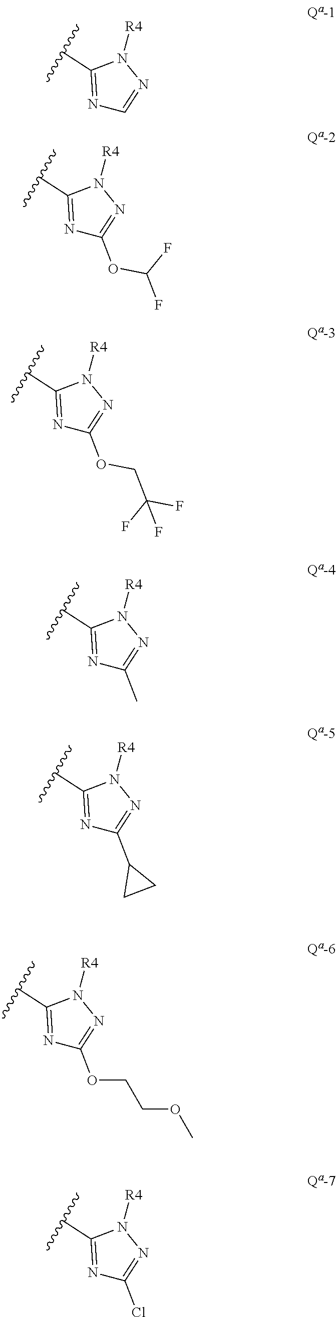

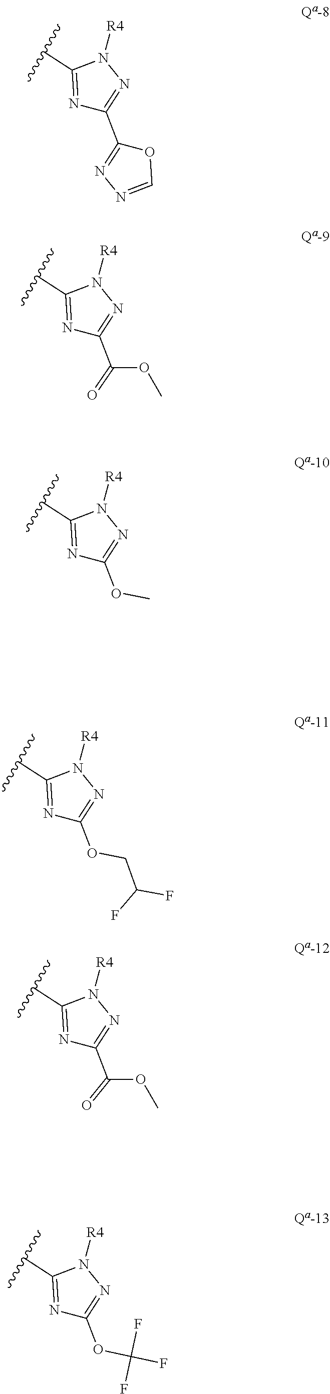

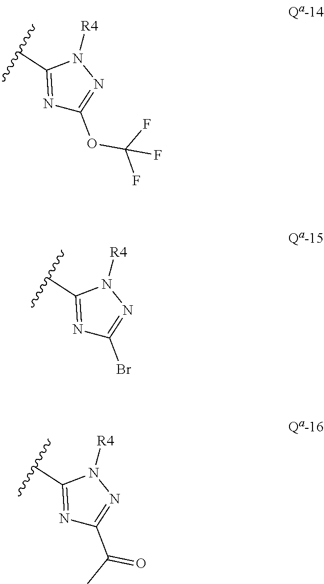

In an embodiment of each aspect of the invention, Q^{b} is
A. selected from Q^{b}-1 to Q^{b}-13; or
B. Q^{b}-1.

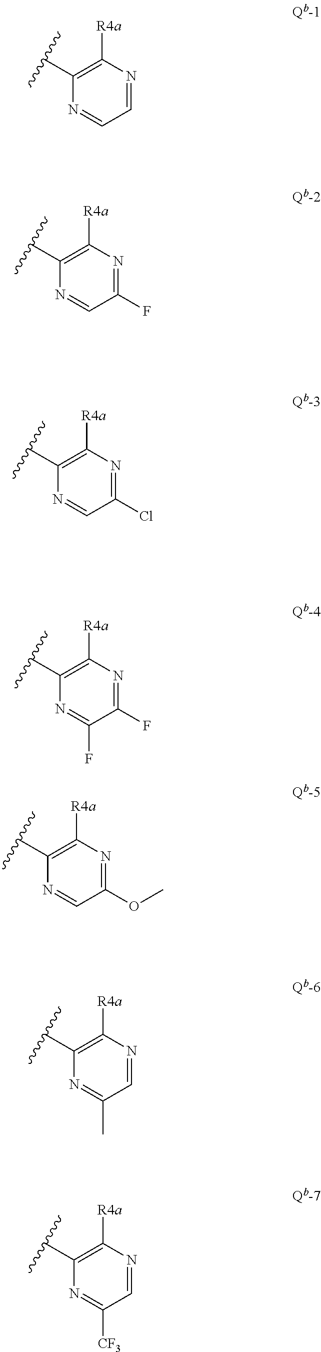

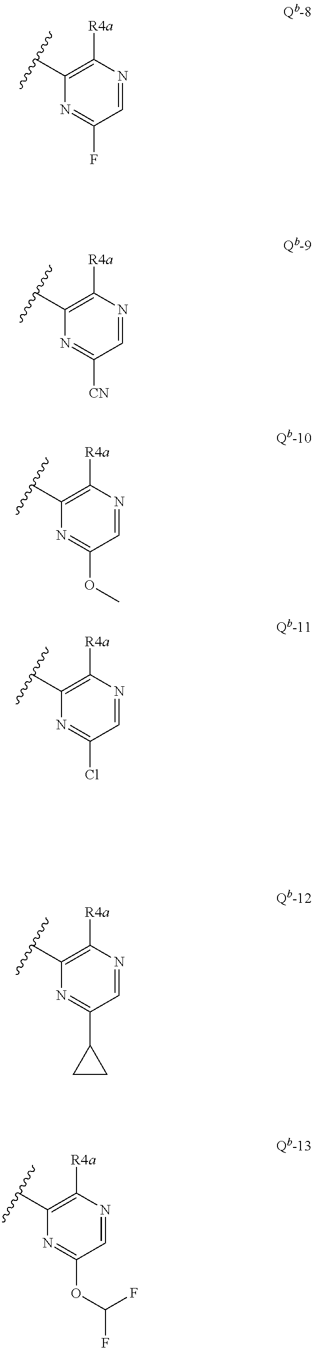

In a preferred embodiment of each aspect of the invention, Q is Q^{a}, and more preferably, Q is Q^{a}-1. In another preferred embodiment of each aspect of the invention, Q is Q^{b}, and more preferably Q is Q^{b}-1.
In preferred embodiments of the invention, Q is Q^{a}-1 or Q^{b}-1. More preferably, Q is Q^{a}-1 In an embodiment of each aspect of the invention, R⁴ is:
A. pyrimidin-4-yl, pyrimidin-2-yl, pyrazin-2-yl, pyridazin-3-yl, or thiazol-2-yl, each of which, independently of each other, is substituted with a single —C(O)NR¹⁰R¹¹, wherein R¹⁰ is hydrogen, C₁-C₃alkyl, C₁-C₃cyanoalkyl, C₁-C₃haloalkyl, C₃-C₄cycloalkyl, C₃-C₄halocycloalkyl, or cyanoC₃-C₄cycloalkyl; and R¹¹ is hydrogen, C₁-C₃alkyl, or C₁-C₃haloalkyl; or
B. pyrimidin-4-yl, pyrimidin-2-yl, pyrazin-2-yl, pyridazin-3-yl, or thiazol-2-yl, each of which, independently of each other, is substituted with a single —C(O)NR¹⁰R¹¹; wherein R¹⁰ is hydrogen, hydroxy, C₁-C₃alkyl, C₁-C₃cyanoalkyl, C₁-C₃haloalkyl, C₁-C₃alkoxy, C₃-C₄cycloalkyl, C₃-C₄halocycloalkyl, cyanoC₃-C₄cycloalkyl, C₃-C₄cycloalkylC₁-C₃cyanoalkyl, oxetan-3-yl, thietan-3-yl, 3-methylthietan-3-yl, 1,1-dioxothietan-3-yl, or 3-methyl-1,1-dioxothietan-3-yl; and R¹¹ is hydrogen, C₁-C₃alkyl or C₁-C₃haloalkyl; or R¹⁰ and R¹¹, together with the nitrogen they are attached to, form a pyrrolidin-1-yl, piperidin-1-yl or 4-morpholinyl group; or
C. selected from Q^{c}-1 to Q^{c}-6, and Q^{c}-10:

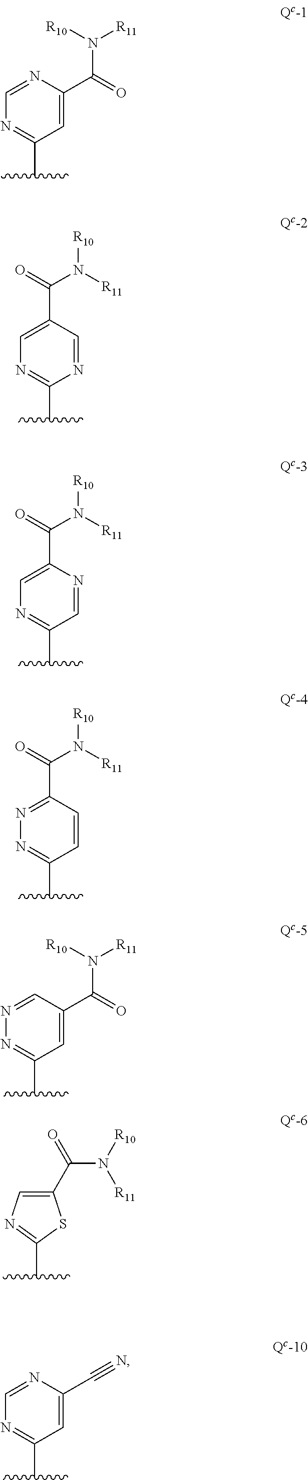

Q^{c}-10 or
D. selected from Q^{c}-1 to Q^{c}-6; or
E. Q^{c}-1, Q^{c}-3, Q^{c}-4; Q^{c}-6, or Q^{c}-10; or
F. Q^{c}-1, or Q^{c}-6; or
G. Q^{c}-1; or
H. Q^{c}-6; or
I. Q^{c}-10; or
J. Q^{c}-2; or
K. Q^{c}-3.
   and R¹⁰ and R¹¹ are as defined below.
   In an embodiment of each aspect of the invention, R^{4a}, is:
A. pyrimidin-4-yl, pyrimidin-2-yl, pyrazin-2-yl, pyridazin-3-yl, thiazol-2-yl, N-linked pyrazol-1-yl, or triazol-1-yl, each of which, independently of each other, is substituted with a single —C(O)NR¹⁰R¹¹; wherein R¹⁰ is hydrogen, C₁-C₃alkyl, C₁-C₃cyanoalkyl, C₁-C₃haloalkyl, C₃-C₄cycloalkyl, C₃-C₄halocycloalkyl, or cyanoC₃-C₄cycloalkyl; and R¹¹ is hydrogen, C₁-C₃alkyl, or C₁-C₃haloalkyl; or
B. pyrimidin-4-yl, pyrimidin-2-yl, pyrazin-2-yl, pyridazin-3-yl, or thiazol-2-yl, each of which, independently of each other, is substituted with a single —C(O)NR¹⁰R¹¹; wherein R¹⁰ is hydrogen, hydroxy, C₁-C₃alkyl, C₁-C₃cyanoalkyl, C₁-C₃haloalkyl, C₁-C₃alkoxy, C₃-C₄cycloalkyl, C₃-C₄halocycloalkyl, cyanoC₃-C₄cycloalkyl, C₃-C₄cycloalkylC₁-C₃cyanoalkyl, oxetan-3-yl, thietan-3-yl, 3-methylthietan-3-yl, 1,1-dioxothietan-3-yl, or 3-methyl-1,1-dioxothietan-3-yl; and R¹¹ is hydrogen, C₁-C₃alkyl or C₁-C₃haloalkyl; or R¹⁰ and R¹¹, together with the nitrogen they are attached to, form a pyrrolidin-1-yl, piperidin-1-yl or 4-morpholinyl group; or
C. pyrimidin-4-yl, pyrimidin-2-yl, pyrazin-2-yl, pyridazin-3-yl, or thiazol-2-yl, each of which, independently of each other, is substituted with a single —C(O)NR¹⁰R¹¹; wherein R¹⁰ is hydrogen, C₁-C₃alkyl, C₁-C₃cyanoalkyl, C₁-C₃haloalkyl, C₃-C₄cycloalkyl, C₃-C₄halocycloalkyl, or cyanoC₃-C₄cycloalkyl; and R¹¹ is hydrogen, C₁-C₃alkyl, or C₁-C₃haloalkyl; or
D. N-linked pyrazol-1-yl, or triazol-1-yl, each of which, independently of each other, is substituted with a single —C(O)NR¹⁰R¹¹; wherein R¹⁰ is hydrogen, C₁-C₃alkyl, C₁-C₃cyanoalkyl, C₁-C₃haloalkyl, C₃-C₄cycloalkyl, C₃-C₄halocycloalkyl, or cyanoC₃-C₄cycloalkyl; and R¹¹ is hydrogen, C₁-C₃alkyl, or C₁-C₃haloalkyl; or
E. selected from Q^{c}-1 to Q^{c}-8, and Q^{c}-10:

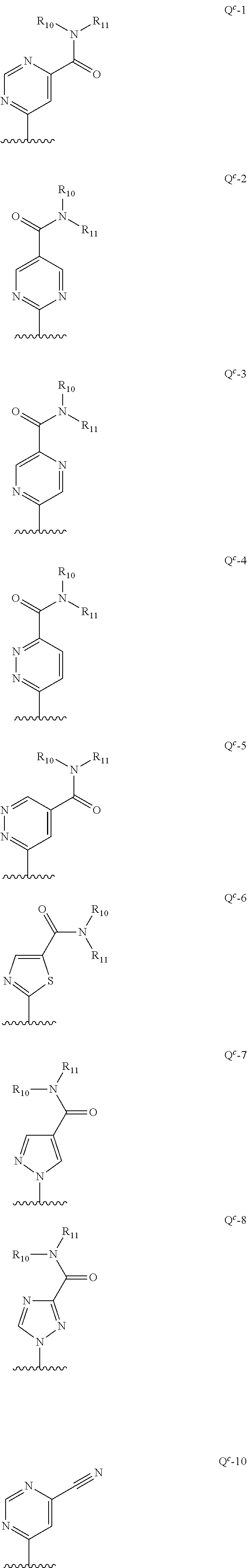

F. selected from Q^{c}-1 to Q^{c}-6, and Q^{c}-10; or
G. selected from Q^{c}-1 to Q^{c}-6; or
H. Q^{c}-1, Q^{c}-3, Q^{c}-4; Q^{c}-6, Q^{c}-7, Q^{c}-8, or Q^{c}-10; or
I. Q^{c}-1, Q^{c}-3, Q^{c}-4; Q^{c}-6, or Q^{c}-10; or
J. Q^{c}-1, Q^{c}-6, Q^{c}-7, Q^{c}-8, or Q^{c}-10; or
K. Q^{c}-1, Q^{c}-6, Q^{c}-7 or Q^{c}-8; or
L. Q^{c}-7, Q^{c}-8, or Q^{c}-10; or
M. Q^{c}-1, or Q^{c}-6; or
N. Q^{c}-7 or Q^{c}-8; or
O. Q^{c}-1; or
P. Q^{c}-6; or
Q. Q^{c}-7; or
R. Q^{c}-8; or
L. Q^{c}-10; or
M. Q^{c}-2; or
S. Q^{c}-3.
   and R¹⁰ and R¹¹ are as defined below.
   In an embodiment of each aspect of the invention, R⁴ and R⁴¹ are 4-cyano-pyrimidin-6-yl, which is depicted as Q^{c}-10 below:

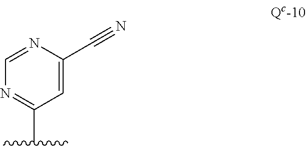 [SMILES: *c1cc(C#N)ncn1]

In an embodiment of each aspect of the invention, in connection with R⁴ and R^{4a}, R¹⁰ is
A. hydrogen, C₁-C₃alkyl, C₁-C₃cyanoalkyl, C₁-C₃haloalkyl, C₃-C₄cycloalkyl, C₃-C₄halocycloalkyl, or cyanoC₃-C₄cycloalkyl; or
B. hydrogen, C₁-C₃alkyl, C₁-C₃cyanoalkyl, C₁-C₃haloalkyl, C₃-C₄cycloalkyl, or cyanoC₃-C₄cycloalkyl; or
C. hydrogen, methyl, ethyl, cyanomethyl, difluoromethyl, trifluoromethyl, cyclopropyl, or 1-cyanocyclopropyl; or
D. hydrogen, methyl, ethyl, trifluoromethyl, cyclopropyl, or 1-cyanocyclopropyl; or
E. hydrogen, methyl, ethyl, cyclopropyl, or 1-cyanocyclopropyl; or
F. hydrogen, methyl, ethyl, or cyclopropyl; or
G. hydrogen, methyl, or ethyl; or
H. hydrogen, hydroxy, C₁-C₃alkyl, C₁-C₃cyanoalkyl, C₁-C₃haloalkyl, C₁-C₃alkoxy, C₃-C₄cycloalkyl, C₃-C₄halocycloalkyl, cyanoC₃-C₄cycloalkyl, C₃-C₄cycloalkylC₁-C₃cyanoalkyl, oxetan-3-yl, thietan-3-yl, 3-methylthietan-3-yl, 1,1-dioxothietan-3-yl, or 3-methyl-1,1-dioxothietan-3-yl; or
I. hydrogen, hydroxy, methyl, ethyl, trifluoromethyl, cyanoethyl, methoxy, ethoxy, cyclopropyl, 1-cyanocyclopropyl, 1-cyano-1-cyclopropyl-ethyl, oxetan-3-yl, thietan-3-yl, 3-methylthietan-3-yl, 1,1-dioxothietan-3-yl, or 3-methyl-1,1-dioxothietan-3-yl; or
J. hydrogen, hydroxy, methyl, ethyl, trifluoromethyl, cyanoethyl, methoxy, cyclopropyl, 1-cyanocyclopropyl, 1-cyano-1-cyclopropyl-ethyl, oxetan-3-yl, thietan-3-yl, or 1,1-dioxothietan-3-yl; or
K. hydrogen, hydroxy, methyl, ethyl, cyanomethyl, 2-cyanoethyl, 1-cyano-1-methyl-ethyl, methoxy, cyclopropyl, cyclopropylmethyl, 1-cyanocyclopropyl, 1-cyano-1-cyclopropyl-ethyl, oxetan-3-yl, thietan-3-yl, 3-methylthietan-3-yl, or 1,1-dioxothietan-3-yl.
   In an embodiment of each aspect of the invention, in connection with R⁴ and R^{4a}, R¹¹ is
A. hydrogen, C₁-C₃alkyl, or C₁-C₃haloalkyl; or
B. hydrogen, methyl, ethyl, difluoromethyl, or trifluoromethyl; or
C. hydrogen, methyl, or trifluoromethyl; or
D. hydrogen, methyl, or ethyl; or
E. hydrogen, or methyl.
   In an embodiment of each aspect of the invention, in connection with R⁴ and R^{4a}, R¹⁰ and R¹¹, together with the nitrogen they are attached to, form a pyrrolidin-1-yl, piperidin-1-yl or 4-morpholinyl group, such as a 4-morpholinyl group.
   Preferably, in an embodiment of each aspect of the invention, R⁴ and R^{4a} are independently Q^{c}-1, or Q^{c}-6; R¹⁰ is hydrogen, methyl, ethyl, cyclopropyl or 1-cyanocyclopropyl; and R¹¹ is hydrogen, methyl or ethyl. More preferably, in an embodiment of each aspect of the invention, R⁴ and R^{4a} are independently Q^{c}-1, or Q^{c}-6; R¹⁰ is hydrogen, methyl, ethyl or cyclopropyl; and R¹¹ is hydrogen, methyl or ethyl. Most preferably, in an embodiment of each aspect of the invention, R⁴ and R^{4a} are independently Q^{c}-1; R¹⁰ is hydrogen, methyl, or ethyl; and R¹¹ is hydrogen or methyl.
   In an embodiment of each aspect of the invention, Q is Q^{a}-1, R⁴ is Q^{c}-1, Q^{c}-2, Q^{c}-3, Q^{c}-4, Q^{c}-5, Q^{c}-6, or Q^{c}-10, such as Q^{c}-1 or Q^{c}-6, or such as Q^{c}-1, Q^{c}-3, Q^{c}-4, Q^{c}-6, or Q^{c}-10. Preferably, R⁴ is Q^{c}-1, Q^{c}-2, or Q^{c}-3. Most preferably, R⁴ is Q^{c}-1. In preferred embodiments, R¹⁰ is hydrogen, hydroxy, methyl, ethyl, cyanomethyl, 2-cyanoethyl, 1-cyano-1-methyl-ethyl, methoxy, cyclopropyl, cyclopropylmethyl, 1-cyanocyclopropyl, 1-cyano-1-cyclopropyl-ethyl, oxetan-3-yl, thietan-3-yl, 3-methylthietan-3-yl, or 1,1-dioxothietan-3-yl, and R¹¹ is hydrogen, methyl, or ethyl, preferably R¹¹ is hydrogen or methyl. In other preferred embodiments, R¹⁰ is hydrogen, methyl, ethyl, cyanomethyl, 2-cyanoethyl, 1-cyano-1-methyl-ethyl, cyclopropyl, cyclopropylmethyl, 1-cyanocyclopropyl, or 1-cyano-1-cyclopropyl-ethyl, and R¹¹ is hydrogen, methyl, or ethyl; preferably, R¹¹ is hydrogen or methyl. In other preferred embodiments, R¹⁰ is hydrogen, methyl, ethyl; and R¹¹ is hydrogen or methyl.
   In an embodiment of each aspect of the invention, Q is Q^{b}-1, R^{4a} is Q^{c}-1, and R¹⁰ and R¹¹ are both hydrogen.
   In an embodiment of each aspect of the invention, R⁵ is
A. hydrogen, C₁-C₃alkyl, C₁-C₃haloalkyl, C₃-C₄cycloalkyl, C₁-C₃alkoxy, halogen, C₁-C₃alkoxy-C₁-C₃alkyl, C₁-C₃alkoxy-C₁-C₃alkoxy-C₁-C₃alkyl, (C₁-C₃alkyl)C(O), (C₁-C₃alkoxy)C(O), HC(O), C₁-C₃haloalkoxy or a 5-membered heteroaromatic ring wherein the 5-membered heteroaromatic ring can be optionally substituted with one to three substituents selected from C₁-C₃alkyl, C₁-C₃haloalkyl, C₁-C₃alkoxy, C₃-C₄cycloalkyl, halogen, CN or hydroxy; or
B. hydrogen, C₁-C₃alkyl, C₁-C₃haloalkyl, C₃-C₄cycloalkyl, C₁-C₃alkoxy, halogen, C₁-C₃alkoxy-C₁-C₃alkyl, C₁-C₃alkoxy-C₁-C₃alkoxy-C₁-C₃alkyl, (C₁-C₃alkyl)C(O), (C₁-C₃alkoxy)C(O), HC(O) or C₁-C₃haloalkoxy; or
C. hydrogen, C₁-C₃alkyl, C₁-C₃haloalkyl, C₃-C₄cycloalkyl, C₁-C₃alkoxy, halogen, Cl, Br, C₁-C₃alkoxy-C₁-C₃alkyl, C₁-C₃alkoxy-C₁-C₃alkoxy-C₁-C₃alkyl, (C₁-C₃alkyl)C(O), (C₁-C₃alkoxy)C(O), or C₁-C₂haloalkoxy; or
D. hydrogen, C₁-C₃alkyl, C₁-C₃alkoxy, C₃-C₄cycloalkyl, C₁-C₃haloalkoxy, halogen, C₁-C₃alkoxy-C₁-C₃alkyl, C₁-C₃alkoxy-C₁-C₃alkoxy-C₁-C₃alkyl, (C₁-C₃alkyl)C(O), HC(O), or (C₁-C₃alkoxy)C(O); or
E. hydrogen, C₁-C₂alkyl, C₁-C₂alkoxy, C₃-C₄cycloalkyl, C₁-C₂haloalkoxy, halogen, C₁-C₂alkoxy-C₁-C₂alkyl, C₁-C₂alkoxy-C₁-C₂alkoxy-C₁-C₂alkyl, (C₁-C₂alkyl)C(O), HC(O), or (C₁-C₂alkoxy)C(O); or
F. hydrogen, methyl, trifluoromethoxy, methoxy, cyclopropyl, 2,2-difluroroethoxy, 2,2,2-trifluroroethoxy, difluoromethoxy, 2,2,2-trifluroroethyl, chloro, bromo, methoxyethoxy, methylcarbonyl, or methoxycarbonyl; or
G. hydrogen.
   In preferred embodiments of each aspect of the invention, R⁵ is hydrogen.
   In an embodiment of each aspect of the invention, R^{5a} is
A. hydrogen, halogen, CN, C₁-C₃alkyl, C₁-C₃haloalkyl, C₃-C₄cycloalkyl, C₁-C₃alkoxy or C₁-C₃haloalkoxy; or
B. hydrogen, halogen, CN, C₁-C₃alkyl, C₁-C₃haloalkyl, C₃-C₄cycloalkyl or C₁-C₃alkoxy; or
C. hydrogen, halogen, CN, C₁-C₃alkyl, C₁-C₃haloalkyl or C₁-C₃alkoxy; or
D. hydrogen, halogen, CN, C₁-C₃alkyl or C₁-C₃alkoxy; or
E. hydrogen or halogen; or
F. hydrogen.
   In an embodiment of each aspect of the invention, R^{5b} is
A. hydrogen, halogen, CN, C₁-C₃haloalkyl, C₃-C₄cycloalkyl, C₁-C₃alkoxy, or C₁-C₃haloalkoxy; or
B. hydrogen, halogen or C₁-C₃alkoxy; or
C. hydrogen.
   In preferred embodiments of each aspect of the invention, R^{5a} and R^{5b} are hydrogen.
   In an embodiment of each aspect of the invention, R⁶ is
A. phenyl, benzyl, heteroaryl, or C₃-C₆ cycloalkyl, each of which, independent of each other, is optionally substituted with one substituent selected from R^{x}; or
B. phenyl, benzyl, cyclopropyl or cyclopropyl substituted with one substituent selected from R^{x}.
   In an embodiment of each aspect of the invention, R^{x} is independently selected from
A. halogen, C₁-C₃haloalkyl, C₁-C₃alkoxy, C₁-C₃haloalkoxy or CN; or
B. F, C, Br, OCF₂H, OCH₃ or CN.
   In an embodiment of each aspect of the invention, R^{z} is independently selected from
A. oxo, halogen, C₁-C₃haloalkyl, C₁-C₃alkoxy, C₁-C₃haloalkoxy or CN; or
B. oxo, F, C, Br, OCF₂H, OCH₃ or CN.
   In an embodiment of each aspect of the invention, R^{Y} is independently selected from
A. hydrogen, C₁-C₃ alkyl, C₁-C₃ haloalkyl, C₁-C₃ alkoxy, C₁-C₃ haloalkoxy, halogen, CN and cyclopropyl; or
B. hydrogen, C₁-C₃ alkyl, C₁-C₃ haloalkyl, C₁-C₃ alkoxy, C₁-C₃ haloalkoxy, halogen, and cyclopropyl; or
C. hydrogen, C₁-C₃ alkyl, C₁-C₃ haloalkyl, and C₁-C₃ alkoxy; or
D. hydrogen, methyl, trifluoromethyl, and methoxy; or
E. hydrogen.
   In an embodiment of each aspect of the invention, R¹¹ is independently selected from
A. hydrogen, C₁-C₃ alkyl, C₁-C₃ haloalkyl, C₁-C₃ alkoxy, C₁-C₃ haloalkoxy, halogen, CN and cyclopropyl; or
B. hydrogen, C₁-C₃ alkyl, C₁-C₃ haloalkyl, C₁-C₃ alkoxy, C₁-C₃ haloalkoxy, halogen, and cyclopropyl; or
C. hydrogen, C₁-C₃ alkyl, C₁-C₃ haloalkyl, and C₁-C₃ alkoxy; or
D. hydrogen, methyl, trifluoromethyl, and methoxy; or
E. hydrogen.
   The present invention, accordingly, makes available a compound of formula (I) having the substituents R, R¹, R^{2a}, R^{2b}, R³, Q, A¹, A², A³, A⁴, and A⁵ as defined above in all combinations/each permutation. Accordingly, made available, for example, is a compound of formula (I) with A¹, A², and A³ being of the first aspect (i.e. A¹, A² and A³ are, independently from each other, N or CR^{Y}; and where R^{Y} is of embodiment D (i.e. R^{Y} is independently selected from hydrogen, methyl, trifluoromethyl, and methoxy); A⁴, and A⁵ being of the embodiment B (i.e. A⁴ is CR^{YY}, and A⁵ is CH where R^{YY} is of embodiment B (i.e. R^{Y} hydrogen, C₁-C₃ alkyl, C₁-C₃ haloalkyl, C₁-C₃ alkoxy, C₁-C₃ haloalkoxy, halogen, or cyclopropyl); R¹ being embodiment B (i.e. hydrogen, C₁-C₆alkyl, C₁-C₆cyanoalkyl, aminocarbonylC₁-C₆alkyl, hydroxycarbonylC₁-C₆alkyl, C₁-C₆nitroalkyl, trimethylsilaneC₁-C₆alkyl, C₁-C₃alkoxy-C₁-C₆alkyl, C₁-C₆haloalkyl, C₂-C₆alkenyl, C₂-C₆haloalkenyl, C₂-C₆alkynyl, C₂-C₆haloalkynyl, C₃-C₄cycloalkylC₁-C₂alkyl-, benzyloxycarbonyl, or benzyl); R^{2a} being an embodiment L (i.e. halogen, C₁-C₂haloalkyl, C₁-C₂haloalkylsulfanyl, C₁-C₂haloalkysulfonyl, or C₁-C₂haloalkoxy); R^{2b} being embodiment B (i.e. halogen, C₁-C₃haloalkyl, or C₁-C₃haloalkoxy); R³ being embodiment B (i.e. hydrogen, halogen, C₃-C₄cycloalkyl, cyclopropylcarbonyl, C₃-C₄cycloalkyl-C₁-C₂alkyl optionally substituted with one to two substituents selected from oxo, halogen, C₁-C₃alkyl and C₁-C₃haloalkyl, C₁-C₃haloalkyl, C₁-C₃haloalkysulfanyl, C₁-C₃haloalkysulfonyl, C₁-C₃alkoxy, C₁-C₃haloalkoxy, or CN); Q being embodiment A (i.e. Q is Q^{a}), wherein Q^{a} can be embodiment B (i.e. Q^{a} is selected from Q^{a}-1, Q^{a}-6, Q^{a}-7, Q^{a}-10, and Q^{a}-15); and R⁴ is embodiment D (i.e. Q^{c}-1, Q^{c}-2, Q^{c}-3, Q^{c}-4, Q^{c}-5, or Q^{c}-6; wherein R¹⁰ may be of embodiment F [i.e. hydrogen, methyl, ethyl, or cyclopropyl]; and R¹¹ may be of embodiment D [i.e. hydrogen, methyl, or ethyl]).
   In an embodiment of each aspect of the invention, the compound of formula (I) has as A¹, A² and A³, independently from each other, N or CR (wherein R^{Y} is hydrogen, methyl, trifluoromethyl, and methoxy); as A⁴ N or CH and A⁵ as CH; as R¹ hydrogen, methyl, propargyl or cyclopropyl-methyl; as R^{2a} hydrogen, halogen, C₁-C₃alkyl, C₁-C₃haloalkyl, C₁-C₃alkoxy, C₁-C₃haloalkoxy, cyclopropyl, cyclopropyl substituted with one to two substituents independently selected from halogen, methyl, and trifluoromethyl, cyclopropylcarbonyl, cyclopropylmethyl substituted with one to two substituents independently selected from oxo, halogen, and trifluoromethyl, or C₁-C₂alkylsulfanyl substituted with one to three halogens or C₁-C₂alkylsulfonyl substituted with one to three halogens; as R^{2b} hydrogen, halogen, C₃-C₄cycloalkyl, cyclopropylcarbonyl, C₃-C₄cycloalkyl-C₁-C₂alkyl optionally substituted with one to two substituents selected from oxo, halogen, C₁-C₃alkyl and C₁-C₃haloalkyl, C₁-C₃haloalkyl, C₁-C₃haloalkysulfanyl, C₁-C₃haloalkysulfonyl, C₁-C₃alkoxy, C₁-C₃haloalkoxy, or CN; as R³ methyl; and as Q selected from Q^{a}-1 to Q^{a}-16 and Q^{b}-1 to Q^{b}-13; as R⁴ (for Q^{a}-1 to Q^{a}-16), pyrimidin-4-yl, pyrimidin-2-yl, pyrazin-2-yl, pyridazin-3-yl, or thiazol-2-yl, each of which, independently of each other, is substituted with a single —C(O)NR¹⁰R¹¹; wherein R¹⁰ is hydrogen, C₁-C₃alkyl, C₁-C₃cyanoalkyl, C₁-C₃haloalkyl, C₃-C₄cycloalkyl, or cyanoC₃-C₄cycloalkyl; and R¹¹ is hydrogen, C₁-C₃alkyl, or C₁-C₃haloalkyl; or as R^{4a} (for Q^{b}-1 to Q^{b}-13), pyrimidin-4-yl, pyrimidin-2-yl, pyrazin-2-yl, pyridazin-3-yl, or thiazol-2-yl, each of which, independently of each other, is substituted with a single —C(O)NR¹⁰R¹¹; wherein R¹⁰ is hydrogen, C₁-C₃alkyl, C₁-C₃cyanoalkyl, C₁-C₃haloalkyl, C₃-C₄cycloalkyl, C₃-C₄halocycloalkyl, or cyanoC₃-C₄cycloalkyl; and R¹¹ is hydrogen, C₁-C₃alkyl, or C₁-C₃haloalkyl.
   In an embodiment of each aspect of the invention, the compound of formula (I) has as A¹=A²-A³ taken together NR—C(═O)—N, R is hydrogen, methyl or 2,2,2-trifluoroethyl; as A⁴ N or CH and A⁵ as CH; as R¹ hydrogen, methyl, propargyl or cyclopropyl-methyl; as R^{2a} hydrogen, halogen, C₁-C₃alkyl, C₁-C₃haloalkyl, C₁-C₃alkoxy, C₁-C₃haloalkoxy, cyclopropyl, cyclopropyl substituted with one to two substituents independently selected from halogen, methyl, and trifluoromethyl, cyclopropylcarbonyl, cyclopropylmethyl substituted with one to two substituents independently selected from oxo, halogen, and trifluoromethyl, or C₁-C₂alkylsulfanyl substituted with one to three halogens or C₁-C₂alkylsulfonyl substituted with one to three halogens; as R^{2b} hydrogen, halogen, C₃-C₄cycloalkyl, cyclopropylcarbonyl, C₃-C₄cycloalkyl-C₁-C₂alkyl optionally substituted with one to two substituents selected from oxo, halogen, C₁-C₃alkyl and C₁-C₃haloalkyl, C₁-C₃haloalkyl, C₁-C₃haloalkysulfanyl, C₁-C₃haloalkysulfonyl, C₁-C₃alkoxy, C₁-C₃haloalkoxy, or CN; as R³ methyl; and as Q selected from Q^{a}-1 to Q^{a}-16 and Q^{b}-1 to Q^{b}-13: as R⁴ (for Q^{a}-1 to Q^{a}-16):

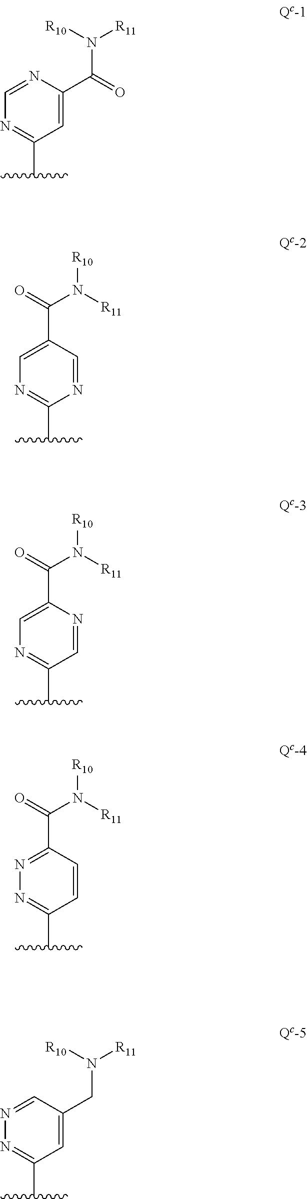

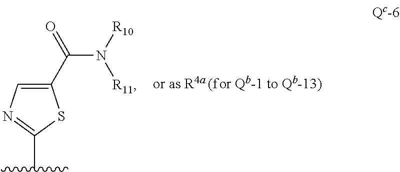

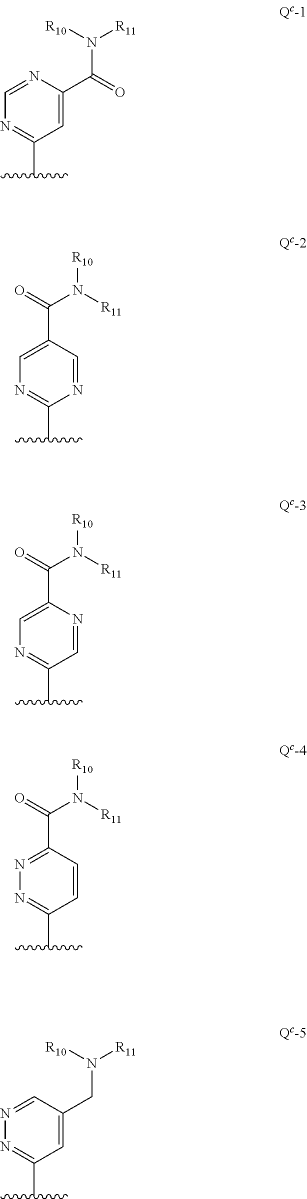

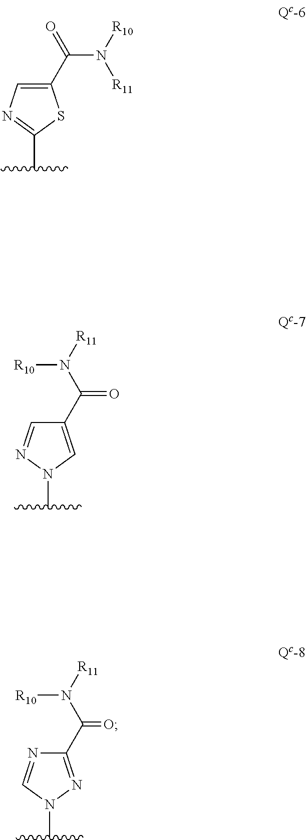

wherein R¹⁰ is hydrogen, C₁-C₃alkyl, C₁-C₃cyanoalkyl, C₁-C₃haloalkyl, C₃-C₄cycloalkyl, or cyanoC₃-C₄cycloalkyl; and R¹¹ is hydrogen, C₁-C₃alkyl, or C₁-C₃haloalkyl.
In an embodiment of each aspect of the invention, the compound of formula (I) has as A¹=A²-A³ taken together NR—C(═O)—N where R is methyl or 2,2,2-trifluoroethyl; as A⁴ N or CH and A⁵ as CH; as R¹ hydrogen, methyl, propargyl or cyclopropyl-methyl; as R^{2a} hydrogen, halogen, C₁-C₃alkyl, C₁-C₃haloalkyl, C₁-C₃alkoxy, C₁-C₃haloalkoxy, cyclopropyl, cyclopropyl substituted with one to two substituents independently selected from halogen, methyl, and trifluoromethyl, cyclopropylcarbonyl, cyclopropylmethyl substituted with one to two substituents independently selected from oxo, halogen, and trifluoromethyl, or C₁-C₂alkylsulfanyl substituted with one to three halogens or C₁-C₂alkylsulfonyl substituted with one to three halogens; as R^{2b} hydrogen, halogen, C₃-C₄cycloalkyl, cyclopropylcarbonyl, C₃-C₄cycloalkyl-C₁-C₂alkyl optionally substituted with one to two substituents selected from oxo, halogen, C₁-C₃alkyl and C₁-C₃haloalkyl, C₁-C₃haloalkyl, C₁-C₃haloalkysulfanyl, C₁-C₃haloalkysulfonyl, C₁-C₃alkoxy, C₁-C₃haloalkoxy, or CN; as R³ methyl; and as Q selected from Q^{a}-1 to Q^{a}-16 and Q^{b}-1 to Q^{b}-13; as R⁴ (for Q^{a}-1 to Q^{a}-16) pyrimidin-4-yl, pyrimidin-2-yl, pyrazin-2-yl, pyridazin-3-yl, or thiazol-2-yl, each of which, independently of each other, is substituted with a single —C(O)NR¹⁰R¹¹, wherein R¹⁰ is hydrogen, methyl, ethyl, cyanomethyl, difluoromethyl, trifluoromethyl, cyclopropyl, or 1-cyanocyclopropyl; and R¹¹ is hydrogen, methyl, ethyl, difluoromethyl, or trifluoromethyl; or as R^{4a} (for Q^{b}-1 to Q^{b}-13) pyrimidin-4-yl, pyrimidin-2-yl, pyrazin-2-yl, pyridazin-3-yl, thiazol-2-yl, N-linked pyrazol-1-yl, or triazol-1-yl, each of which, independently of each other, is substituted with a single —C(O)NR¹⁰R¹¹; wherein R¹⁰ is hydrogen, methyl, ethyl, cyanomethyl, difluoromethyl, trifluoromethyl, cyclopropyl, or 1-cyanocyclopropyl; and R¹¹ is hydrogen, methyl, ethyl, difluoromethyl, or trifluoromethyl.
In an embodiment of each aspect of the invention, the compound of formula (I) has as A¹, A² and A³, independently from each other, N or CR (wherein R^{Y} is hydrogen, methyl, trifluoromethyl, and methoxy); as A⁴ N or CH and A⁵ as CH; as R¹ hydrogen, methyl, propargyl or cyclopropyl-methyl; as R^{2a} hydrogen, halogen, C₁-C₃alkyl, C₁-C₃haloalkyl, C₁-C₃alkoxy, C₁-C₃haloalkoxy, cyclopropyl, cyclopropyl substituted with one to two substituents independently selected from halogen, methyl, and trifluoromethyl, cyclopropylcarbonyl, cyclopropylmethyl substituted with one to two substituents independently selected from oxo, halogen, and trifluoromethyl, or C₁-C₂alkylsulfanyl substituted with one to three halogens or C₁-C₂alkylsulfonyl substituted with one to three halogens; as R^{2b} hydrogen, halogen, C₃-C₄cycloalkyl, cyclopropylcarbonyl, C₃-C₄cycloalkyl-C₁-C₂alkyl optionally substituted with one to two substituents selected from oxo, halogen, C₁-C₃alkyl and C₁-C₃haloalkyl, C₁-C₃haloalkyl, C₁-C₃haloalkysulfanyl, C₁-C₃haloalkysulfonyl, C₁-C₃alkoxy, C₁-C₃haloalkoxy, or CN; as R³ methyl; and as Q selected from Q^{a}-1 and Q^{b}-1, and as R⁴, Q^{c}-1, Q^{c}-2, Q^{c}-3, Q^{c}-4, Q^{c}-5, or Q^{c}-6, or as R^{4a}, Q^{c}-1, Q^{c}-2, Q^{c}-3, Q^{c}-4, Q^{c}-5, Q^{c}-6, Q^{c}-7, or Q^{c}-8; wherein R¹⁰ is hydrogen, methyl, ethyl, trifluoromethyl, cyclopropyl, or 1-cyanocyclopropyl; and R¹¹ is hydrogen, methyl, ethyl, difluoromethyl, or trifluoromethyl; or as Q, Q-1 to Q-18 as shown in Table Z below.
In an embodiment of each aspect of the invention, the compound of formula (I) has as A¹, A² and A³, independently from each other, N or CH; as A⁴ and A⁵ each CH; as R¹ hydrogen, methyl, propargyl or cyclopropyl-methyl; as R^{2a} hydrogen, halogen, C₁-C₃alkyl, C₁-C₃haloalkyl, C₁-C₃alkoxy, C₁-C₃haloalkoxy, cyclopropyl, cyclopropyl substituted with one to two substituents independently selected from halogen, methyl, and trifluoromethyl, cyclopropylcarbonyl, cyclopropylmethyl substituted with one to two substituents independently selected from oxo, halogen, and trifluoromethyl, or C₁-C₂alkylsulfanyl substituted with one to three halogens or C₁-C₂alkylsulfonyl substituted with one to three halogens; as R^{2b} hydrogen, halogen, C₃-C₄cycloalkyl, cyclopropylcarbonyl, C₃-C₄cycloalkyl-C₁-C₂alkyl optionally substituted with one to two substituents selected from oxo, halogen, C₁-C₃alkyl and C₁-C₃haloalkyl, C₁-C₃haloalkyl, C₁-C₃haloalkysulfanyl, C₁-C₃haloalkysulfonyl, C₁-C₃alkoxy, C₁-C₃haloalkoxy, or CN; as R³ methyl; and as Q selected from Q^{a}-1 and Q^{b}-1; and as R⁴, Q^{c}-1, Q^{c}-2, Q^{c}-3, Q^{c}-4, Q^{c}-5, or Q^{c}-6; or as R^{4a}, Q^{c}-1, Q^{c}-2, Q^{c}-3, Q^{c}-4, Q^{c}-5, Q^{c}-6, Q^{c}-7, or Q^{c}-8; wherein R¹⁰ is hydrogen, methyl, ethyl, cyanomethyl, difluoromethyl, trifluoromethyl, cyclopropyl, or 1-cyanocyclopropyl; and R¹¹ is hydrogen, methyl, or ethyl; or as Q, Q-1 to Q-18 as shown in Table Z below.
In an embodiment of each aspect of the invention, the compound of formula (I) has as A¹, A² and A³, independently from each other, N or CH; as A⁴ and A⁵ each CH; as R¹ hydrogen, methyl, propargyl or cyclopropyl-methyl; as R^{2a} halogen, C₁-C₃haloalkyl, C₁-C₃haloalkoxy, cyclopropyl, cyclopropyl substituted with one to two substituents independently selected from halogen, methyl, and trifluoromethyl, cyclopropylcarbonyl, cyclopropylmethyl substituted with one to two substituents independently selected from oxo, halogen, and trifluoromethyl, or C₁-C₂alkylsulfanyl substituted with one to three halogens or C₁-C₂alkylsulfonyl substituted with one to three halogens; as R^{2b} halogen, C₃-C₄cycloalkyl, cyclopropylcarbonyl, C₃-C₄cycloalkyl-C₁-C₂alkyl optionally substituted with one to two substituents selected from oxo, halogen, C₁-C₃alkyl and C₁-C₃haloalkyl, C₁-C₃haloalkyl, C₁-C₃haloalkysulfanyl, C₁-C₃haloalkysulfonyl, C₁-C₃alkoxy, C₁-C₃haloalkoxy, or CN; as R³ methyl; and as Q selected from Q^{a}-1 and Q^{b}-1, and as R⁴, Q^{c}-1, or Q^{c}-6; or as R^{4a}, Q^{c}-1, Q^{c}-6, Q^{c}-7, or Q^{c}-8; wherein R¹⁰ is hydrogen, methyl, ethyl, or cyclopropyl; and R¹¹ is hydrogen, or methyl; or as Q, Q-1 to Q-18 as shown in Table Z below.
In an embodiment of each aspect of the invention, the compound of formula (I) has as A¹=A²-A³ taken together NR—C(═O)—N where R is methyl or 2,2,2-trifluoroethyl; as A⁴ N or CH and A⁵ as CH; as R¹ hydrogen, methyl, propargyl or cyclopropyl-methyl; as R^{2a} hydrogen, halogen, C₁-C₃alkyl, C₁-C₃haloalkyl, C₁-C₃alkoxy, C₁-C₃haloalkoxy, cyclopropyl, cyclopropyl substituted with one to two substituents independently selected from halogen, methyl, and trifluoromethyl, cyclopropylcarbonyl, cyclopropylmethyl substituted with one to two substituents independently selected from oxo, halogen, and trifluoromethyl, or C₁-C₂alkylsulfanyl substituted with one to three halogens or C₁-C₂alkylsulfonyl substituted with one to three halogens; as R^{2b} hydrogen, halogen, C₃-C₄cycloalkyl, cyclopropylcarbonyl, C₃-C₄cycloalkyl-C₁-C₂alkyl optionally substituted with one to two substituents selected from oxo, halogen, C₁-C₃alkyl and C₁-C₃haloalkyl, C₁-C₃haloalkyl, C₁-C₃haloalkysulfanyl, C₁-C₃haloalkysulfonyl, C₁-C₃alkoxy, C₁-C₃haloalkoxy, or CN; as R³ methyl; and as Q selected and as Q selected from Q^{a}-1 and Q^{b}-1, and as R⁴ or R^{4a}, Q^{c}-1, or Q^{c}-6, wherein R¹⁰ is hydrogen, methyl, or ethyl; and R¹¹ is hydrogen, or methyl; or as Q, Q-1 to Q-18 as shown in Table Z below.
In an embodiment of each aspect of the invention, the compound of formula (I) has as A¹, A² and A³, independently from each other, N or CH; as A⁴ and A⁵ each CH; as R¹ hydrogen, methyl, propargyl or cyclopropyl-methyl; as R^{2a} halogen, C₁-C₃haloalkyl, or C₁-C₂alkylsulfonyl substituted with one to three halogens; as R^{2b} halogen, or C₁-C₃haloalkyl; as R³ methyl; and as Q selected from Q^{a}-1 and Q^{b}-1, and as R⁴ or R^{4a}, Q^{c}-1, wherein R¹⁰ is hydrogen, methyl, or ethyl; and R¹¹ is hydrogen, or methyl.
In an embodiment of each aspect of the invention, the compound of formula (I), A¹ and A³ are N; A² is CH; as A⁴ and A⁵ each CH; R¹ is hydrogen, or methyl; R^{2a} is chlorine, bromine, iodine, methyl, or trifluoromethyl; R^{2b} is chlorine, bromine, iodine, or trifluoromethyl; R³ is methyl; Q is Q^{a}-1 or Q^{b}-1; R⁴ is Q^{c}-1, Q^{c}-3, Q^{c}-4; Q^{c}-6, or Q^{c}-10; R^{4a} is Q^{c}-1, Q^{c}-3, Q^{c}-4; Q^{c}-6, Q^{c}-7, Q^{c}-8, or Q^{c}-10; R¹⁰ is hydrogen, hydroxy, methyl, ethyl, trifluoromethyl, cyanoethyl, methoxy, cyclopropyl, 1-cyanocyclopropyl, 1-cyano-1-cyclopropyl-ethyl, oxetan-3-yl, thietan-3-yl, 1,1-dioxothietan-3-yl; and R¹¹ is hydrogen, or methyl; or R¹⁰ and R¹¹, together with the nitrogen they are attached to, are a pyrrolidin-1-yl, piperidin-1-yl or 4-morpholinyl.
In preferred embodiments of the invention, A¹ and A³ are nitrogen, A², A⁴ and A⁵ are CH; R¹ is hydrogen, methyl or cyclopropyl-methyl; R^{2a} is chlorine, bromine, iodine, or trifluoromethyl, and R^{2b} is chlorine, bromine, iodine, trifluoromethyl, difluoromethylsulfonyl, or trifluoromethylsulfonyl; R³ is methyl; Q is Q^{a}-1 or Q^{b}-1, preferably, Q is Q^{a}-1.
In preferred embodiments of the invention, A¹ and A³ are nitrogen, A², A⁴ and A⁵ are CH; R¹ is hydrogen, methyl or cyclopropyl-methyl; R^{2a} is chlorine, bromine, iodine, or trifluoromethyl, and R^{2b} is chlorine, bromine, iodine, trifluoromethyl, difluoromethylsulfonyl, or trifluoromethylsulfonyl; R³ is methyl; Q is Q^{a}-1; R⁴ is Q^{c}-1, Q^{c}-2, Q^{c}-3, Q^{c}-4, Q^{c}-5, Q^{c}-6, or Q^{c}-10, such as Q^{c}-1 or Q^{c}-6, or such as Q^{c}-1, Q^{c}-3, Q^{c}-4, Q°-6, or Q^{c}-10; R¹⁰ is hydrogen, hydroxy, methyl, ethyl, cyanomethyl, 2-cyanoethyl, 1-cyano-1-methyl-ethyl, methoxy, cyclopropyl, cyclopropylmethyl, 1-cyanocyclopropyl, 1-cyano-1-cyclopropyl-ethyl, oxetan-3-yl, thietan-3-yl, 3-methylthietan-3-yl, or 1,1-dioxothietan-3-yl, and R¹¹ is hydrogen, methyl, or ethyl, preferably R¹¹ is hydrogen or methyl. Preferably, R⁴ is Q^{c}-1, Q^{c}-2, or Q^{c}-3; most preferably, R⁴ is Q^{c}-1.
In other preferred embodiments of the invention, A¹ and A³ are nitrogen, A², A⁴ and A⁵ are CH; R¹ is hydrogen, methyl or cyclopropyl-methyl; R^{2a} is chlorine, bromine, iodine, or trifluoromethyl, and R^{2b} is chlorine, bromine, iodine, trifluoromethyl, difluoromethylsulfonyl, or trifluoromethylsulfonyl; R³ is methyl; Q is Q^{b}-1, R^{4a} is Q^{c}-1, and R¹⁰ and R¹¹ are both hydrogen. Preferably, R¹ is hydrogen or methyl; more preferably, R¹ is hydrogen. Preferably, R^{2a} is chlorine, bromine, iodine, or trifluoromethyl, and R^{2b} is chlorine, bromine, iodine, or trifluoromethyl, or R^{2a} is chlorine or trifluoromethyl and R^{2b} is difluoromethylsulfonyl or trifluoromethylsulfonyl. More preferably, R^{2a} is chlorine or trifluoromethyl, and R^{2b} is chlorine, bromine, iodine, or trifluoromethyl. Preferably, Q is Q^{a}-1, R¹⁰ is hydrogen, methyl, ethyl, cyanomethyl, 2-cyanoethyl, 1-cyano-1-methyl-ethyl, cyclopropyl, cyclopropylmethyl, 1-cyanocyclopropyl, or 1-cyano-1-cyclopropyl-ethyl, and R¹¹ is hydrogen, methyl, or ethyl; preferably, R¹¹ is hydrogen or methyl. In other preferred embodiments, R¹⁰ is hydrogen, methyl, ethyl; and R¹¹ is hydrogen or methyl.
In an embodiment, the compound of formula (I) is formula Iaa, Iab, Iac, Iad, or Iae (with asterisk indicating a stereogenic centre), wherein R¹, R^{2a}, R^{2b}, and R³, are as defined in the first aspect and Q₁ corresponds to Q as defined in the first aspect, each with the corresponding embodiments as described above.

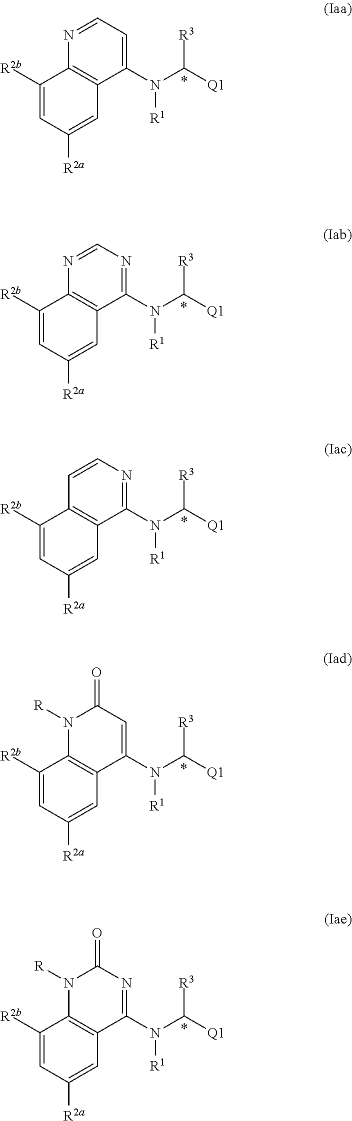

In an embodiment, compounds having preferred stereochemistry depicted in formula (I′a) would also be preferred for compounds of formulae Iaa, Iab and Iac. In a preferred embodiment, a compound of formulae I′ab and I′ae with the following stereochemistry is preferred:

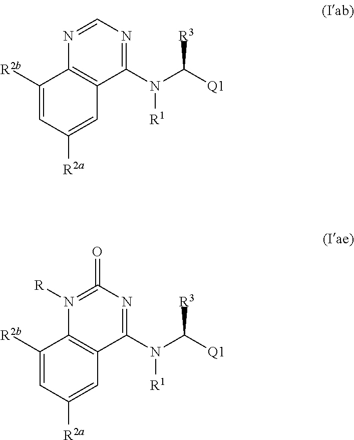

where R¹, R^{2a}, R^{2b}, R³, Q₁ (corresponding to Q in formula I) are as defined in the first aspect and R is C₁-C₃alkyl, C₁-C₃haloalkyl, C₁-C₃alkoxy, or C₁-C₃haloalkoxy, and stereoisomers, enantiomers, tautomers and N-oxides of the compounds of formula (I′ab), and agrochemically acceptable salts thereof.
In a second aspect, the present invention makes available a composition comprising a compound of formula (I) as defined in the first aspect, one or more auxiliaries and diluent, and optionally one or more other active ingredient.
In a third aspect, the present invention makes available a method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound as defined in the first aspect or a composition as defined in the second aspect. In a fourth aspect, the present invention makes available a method for the protection of plant propagation material from the attack by insects, acarines, nematodes or molluscs, which comprises treating the propagation material or the site, where the propagation material is planted, with an effective amount of a compound of formula (I) as defined in the first aspect or a composition as defined in the second aspect. In a fifth aspect, the present invention makes available a plant propagation material, such as a seed, comprising, or treated with or adhered thereto, a compound of formula (I) as defined in the first aspect or a composition as defined in the second aspect.
The present invention in a further aspect provides a method of controlling parasites in or on an animal in need thereof comprising administering an effective amount of a compound of the first aspect. The present invention further provides a method of controlling ectoparasites on an animal in need thereof comprising administering an effective amount of a compound of formula (I) as defined in the first aspect. The present invention further provides a method for preventing and/or treating diseases transmitted by ectoparasites comprising administering an effective amount of a compound of formula (I) as defined in the first aspect, to an animal in need thereof.
Compounds of formula (I) can be prepared by those skilled in the art following known methods. More specifically compounds of formulae I, and I′a, and intermediates therefor can be prepared as described below in the schemes and examples. Certain stereogenic centers have been left unspecified for the clarity and are not intended to limit the teaching of the schemes in any way.
The process according to the invention for preparing compounds of formula (I) is carried out by methods known to those skilled in the art.
Compounds of formula (I) can be made, for example, as shown in scheme 1.

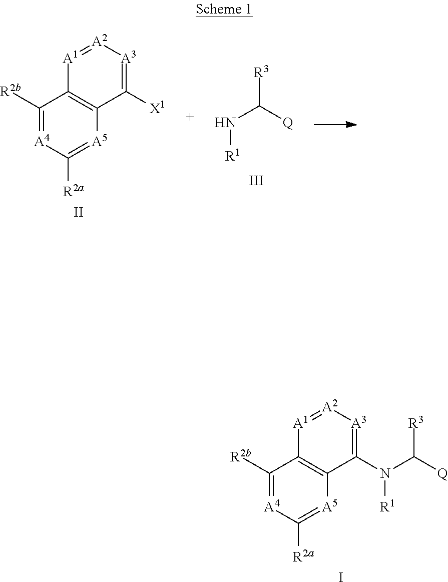

Reaction of a compound of the formula II, wherein A¹, A², A³, A⁴, A⁵, R^{2a} and R^{2b} are as defined above for compounds of the formula (I), and wherein X¹ is a leaving group, such as a halogen or sulfonate, for instance chloride, with a compound of formula III, or a salt thereof (such as a hydrohalide salt, preferably a hydrochloride or a hydrobromide salt, or a trifluoroacetic acid salt, or any other equivalent salt), wherein Q, R¹ and R³ have the same meaning as given above for compounds of the formula (I), gives a compound of the formula (I), wherein A¹, A², A³, A⁴, A⁵, R¹, R^{2a}, R^{2b}, R³ and Q have the same meaning as given above for compounds of the formula (I). The reaction can be conducted neat or in a solvent, preferably in a solvent, such as an organic solvent, for instance acetonitrile, tetrahydrofuran, 2-methyltetrahydrofuran, ethyl acetate, N,N-dimethylacetamide or N,N-dimethylformamide, in a temperature range of −100 to +300° C., preferably between ambient temperature and 200° C., with or without the presence of a catalyst, for instance a metal catalyst, such as a palladium complex, and with or without the addition of a base, such as an inorganic base, for instance sodium, potassium or cesium carbonate, or an organic base, such as, for example, triethylamine, diisopropylethylamine or pyridine.
Compounds of the formula II, wherein A¹, A², A³, A⁴, A⁵, R^{2a} and R^{2b} are as defined above for compounds of the formula (I), are either known, or can be prepared in analogy to descriptions found for example in WO 2021/083936 and WO 2021/177160, or they can be prepared by methods known to a person skilled in the art.

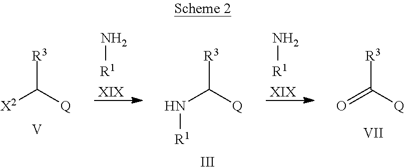

Compounds of formula III, or a salt thereof, wherein Q, R¹ and R³ have the same meaning as given above for compounds of the formula (I), can be made, for example, as shown in scheme 2. Treatment of a compound of the formula V, wherein R³ and Q have the same meaning as given above for compounds of the formula (I) and wherein X² is a leaving group, such as a halogen or sulfonate, for instance bromide, with an amine of the formula XIX, or a salt thereof, wherein R¹ has the same meaning as given above for compounds of the formula (I), gives compounds of the formula III, or a salt thereof, wherein Q, R¹ and R³ have the same meaning as given above for compounds of the formula (I). The reaction can be conducted neat or in a solvent, preferably in a solvent, such as an organic solvent, for instance acetonitrile, in a temperature range of −100 to +300° C., preferably between ambient temperature and 200° C., with or without the addition of a base, such as an inorganic base, for instance sodium, potassium or cesium carbonate, or an organic base, such as, for example, triethylamine, diisopropylethylamine or pyridine.
Alternatively, treatment of a compound of the formula VII, wherein R³ and Q have the same meaning as given above for compounds of the formula (I), with an amine of the formula XIX, or a salt thereof, wherein R¹ has the same meaning as given above for compounds of the formula (I), gives compounds of the formula III, or a salt thereof, wherein Q, R¹ and R³ have the same meaning as given above for compounds of the formula (I). This reaction is done in the presence of a reducing agent, such as for example hydrogen, or a hydride, such as sodium borohydride, with or without a catalyst, such as a hydrogenation catalyst, for example palladium on carbon, with or without the presence of an acid, such as acetic acid, or a Lewis acid, such as zinc bromide or titanium(IV) isopropoxide, in a solvent or without a solvent, such as, for instance, methanol. The reaction can be conducted in a temperature range of −100 to +300° C., preferably between ambient temperature and 200° C. Such methods, and the range of conditions to perform them, for the alkylation of amines and for the reductive alkylation of amines (e.g. in the presence of NaBH(OAc)₃ or NaBH₃CN, in a suitable solvent, preferably in acetic acid, at room temperature, analogous to WO2002/088073; or alternatively, by the use of a combination of Ti(i-OiPr)₄ and NaBH₄ as described in Synthesis 2003 (14), 2206) are well known to a person skilled in the art. The amines of formula XIX, or a salt thereof, wherein R¹ has the same meaning as given above for compounds of the formula (I), are either known, or they can be prepared by methods known to a person skilled in the art.

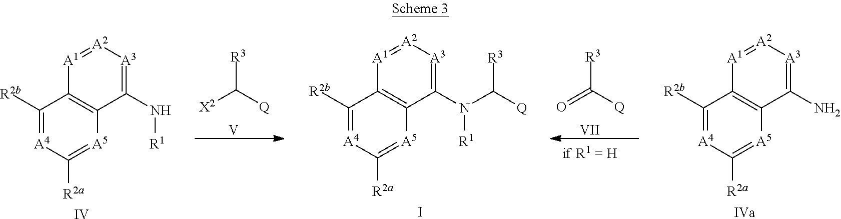

Alternatively, compounds of formula (I) can be made, for example, as shown in scheme 3. Reaction of an amine of the formula IV with a compound of the formula V, wherein X² is a leaving group, such as a halogen or sulfonate, for instance bromide, gives a compound of formula (I), wherein A¹, A², A³, A⁴, A⁵, R¹, R^{2a}, R^{2b}, R³ and Q have the same meaning as given above for compounds of the formula (I). The reaction can be conducted neat or in a solvent, preferably in a solvent, such as an organic solvent, for instance acetonitrile, in a temperature range of −100 to +300° C., preferably between ambient temperature and 200° C., with or without the addition of a base, such as an inorganic base, for instance potassium carbonate, or an organic base, such as, for example, triethylamine. Such methods for the alkylation of amines, and the range of conditions to perform them, are well known to a person skilled in the art.
Alternatively, reaction of an amine of the formula IVa with a compound of the formula VII gives a compound of the formula (I) wherein R¹ is H and A¹, A², A³, A⁴, A⁵, R^{2a}, R^{2b}, R³ and Q have the same meaning as given above for compounds of the formula (I). This reaction is done in the presence of a reducing agent, such as for example hydrogen, or a hydride, such as sodium borohydride, with or without a catalyst, such as a hydrogenation catalyst, for example palladium on carbon, with or without the presence of an acid, such as acetic acid, or a Lewis acid, such as zinc bromide, in a solvent or without a solvent, such as, for instance, methanol. The reaction can be conducted in a temperature range of −100 to +300° C., preferably between ambient temperature and 200° C.
Such methods for the reductive alkylation of amines, and the range of conditions to perform them, are well known to a person skilled in the art.

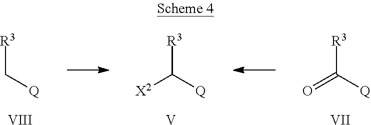

Compounds of formula V can be made, for example, as shown in scheme 4. Treatment of a compound of the formula VIII with a halogenating agent, such as chlorine or bromine or N-bromosuccinimide, for example, gives compound of the formula V, wherein the leaving group Q is a halogen, for instance chloride or bromide. This reaction is done with or without a solvent, preferably in a solvent, with or without an additive, such as a radical starter, such as, for example, benzoyl peroxide or azoisobutyronirile. The reaction can be done with or without exposure to visible light, or to UV light, and it can be conducted in a temperature range of −100 to +300° C., preferably between ambient temperature and 200° C.
Alternatively, a compound of the formula VII can be treated with a reducing agent, followed by reaction with a sulfonyl chloride, for instance methanesulfonyl chloride, to give a compound of the formula V, wherein the leaving group Q is a sulfonate, for instance a mesylate. This reaction can be done in a solvent, or without a solvent, in the presence of a base, such as an inorganic base, for instance potassium carbonate, or an organic base, such as an amine base, for instance trimethylamine, or without a base, and it can be conducted in a temperature range of −100 to +300° C., preferably between ambient temperature and 200° C. A suitable reducing agent could be, for example, hydrogen, or a hydride, such as sodium borohydride, with or without a catalyst, such as a hydrogenation catalyst, for example palladium on carbon, with or without the presence of an acid, such as acetic acid, or a Lewis acid, such as zinc bromide, in a solvent or without a solvent, such as, for instance, methanol. The reaction can be conducted in a temperature range of −100 to +300° C., preferably between ambient temperature and 200° C.
Such methods for the halogenation, the reduction of carbonyl compounds and the sulfonylation of alcohols, and the range of conditions to perform them, are well known to a person skilled in the art. The amines of formula VII and the compounds of formula VIII are either known, or they can be prepared by methods known to a person skilled in the art.

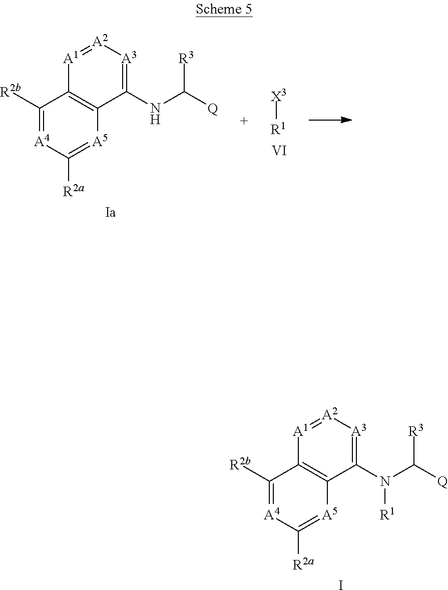

Alternatively, compounds of formula (I) wherein R¹ is different from hydrogen can be made, for example, as shown in scheme 5. A compound of the formula Ia, wherein A¹, A², A³, A⁴, A⁵, R^{2a}, R^{2b}, R³ and Q have the same meaning as given above for compounds of the formula (I), can be reacted with a compound of the formula VI, wherein R¹ has the same meaning as given above for compounds of the formula (I), except that R¹ is different from hydrogen, and wherein X³ is a leaving group, such as a halogen or sulfonate, for instance a chloride, bromide, iodide or mesylate, to give a compound of formula (I), wherein A¹, A², A³, A⁴, A⁵, R¹, R^{2a}, R^{2b}, R³ and Q have the same meaning as given above for compounds of the formula (I). This reaction can be conducted neat or in a solvent, preferably in a solvent, such as an organic solvent, for instance acetonitrile, N,N-dimethylformamide (DMF) or N,N-dimethylacetamide (DMA), or mixtures thereof, in a temperature range of −100 to +300° C., preferably between ambient temperature and 200° C., with or without the addition of a base, such as an inorganic base, for instance sodium, potassium or cesium carbonate, or an organic base, such as, for example, triethylamine, diisopropylethylamine or pyridine. Such methods for the alkylation of amines, and the range of conditions to perform them, are well known to a person skilled in the art, and are described for example in WO2021/083936.
Compounds of the formula (VI), wherein R¹ has the same meaning as given above for compounds of the formula (I), except that R¹ is different from hydrogen, and wherein X³ is a leaving group, such as a halogen or sulfonate, for instance a chloride, bromide, iodide or mesylate, are either known, or they can be prepared by methods known to a person skilled in the art.

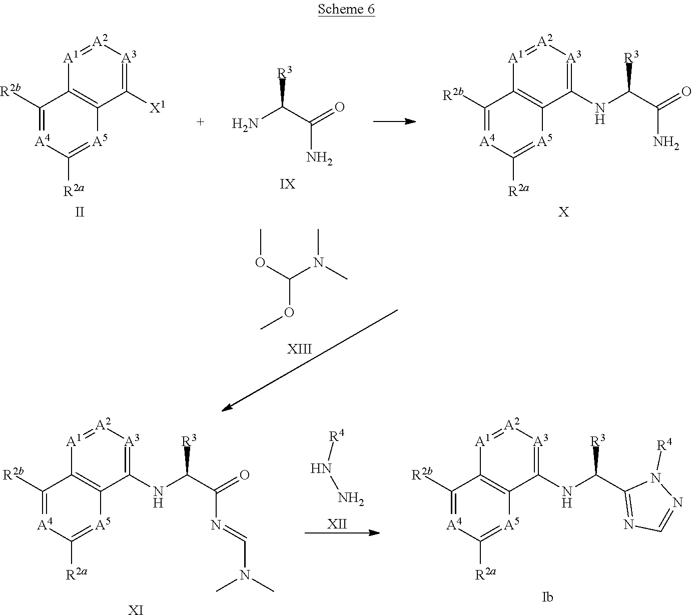

Compounds of formula Ib can be made, for example, as shown in scheme 6. Reaction of a compound of the formula II, wherein A¹, A², A³, A⁴, A⁵, R^{2a} and R^{2b} have the same meaning as given above for compounds of the formula (I), and wherein X¹ is a leaving group, such as a halogen or sulfonate, for instance chloride, with a compound of formula IX, or a salt thereof, wherein R³ has the same meaning as given above for compounds of the formula (I), gives a compound of the formula X, wherein A¹, A², A³, A⁴, A⁵, R^{2a}, R^{2b} and R³ have the same meaning as given above for compounds of the formula (I). The reaction can be conducted neat or in a solvent, preferably in a solvent, such as an organic solvent, for instance acetonitrile, tetrahydrofuran, 2-methyltetrahydrofuran, ethyl acetate, N,N-dimethylacetamide or N,N-dimethylformamide, in a temperature range of −100 to +300° C., preferably between ambient temperature and 200° C., with or without the presence of a catalyst, for instance a metal catalyst, such as a palladium complex, and with or without the addition of a base, such as an inorganic base, for instance sodium, potassium or cesium carbonate, or an organic base, such as, for example, triethylamine, diisopropylethylamine or pyridine.
Subsequent treatment of compound X, wherein A¹, A², A³, A⁴, A⁵, R^{2a}, R^{2b} and R³ have the same meaning as given above for compounds of the formula (I), with the known compound XIII (N,N-dimethylformamide dimethyl acetal, DMF-DMA) gives a compound of the formula XI, wherein A¹, A², A³, A⁴, A⁵, R^{2a}, R^{2b} and R³ have the same meaning as given above for compounds of the formula (I). This reaction can be conducted neat or in a solvent, preferably in a solvent, such as an organic solvent, for instance dichloromethane, tetrahydrofuran, 2-methyltetrahydrofuran or dioxane, in a temperature range of −100 to +300° C., preferably between ambient temperature and 100° C., or between ambient temperature and 50° C., without a base or in the presence of a base, such as an inorganic base, for instance sodium, potassium or cesium carbonate, or an organic base, such as, for example, triethylamine, diisopropylethylamine or pyridine.
Further reaction of compound XI, wherein A¹, A², A³, A⁴, A⁵, R^{2a}, R^{2b} and R³ have the same meaning as given above for compounds of the formula (I), with hydrazine compounds of formula XII or a tautomer thereof, or a salt thereof, wherein R⁴ has the same meaning as given above for compounds of the formula (I), gives the compound of formula Ib, wherein A¹, A², A³, A⁴, A⁵, R^{2a}, R^{2b}, R³ and R⁴ have the same meaning as given above for compounds of the formula (I). This reaction can be conducted neat or in a solvent, preferably in a solvent, such as an organic solvent, for instance 1,4-dioxane, or acetic acid, or a mixture of 1,4-dioxane and acetic acid, in a temperature range of −100 to +300° C., preferably between ambient temperature and 200° C., or between ambient temperature and 80° C. Within this sequence of transformations, the intermediate compounds of formula X and of formula XI can be used as crude products for the subsequent step, or they can be purified, for instance by chromatography, and used in purified form for the next transformation.
Compounds of the formula IX, or a salt thereof, wherein R³ has the same meaning as given above for compounds of the formula (I), are either known, or they can be prepared by methods known to a person skilled in the art. Hydrazine compounds of formula XII or a tautomer thereof, or a salt thereof, wherein R⁴ has the same meaning as given above for compounds of the formula (I), are either known, or they can be prepared by methods known to a person skilled in the art.
Compounds of the formula Ik

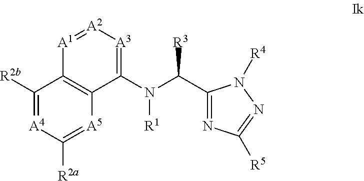

can be prepared by the reaction of an amine of the formula IIIf, or a salt thereof

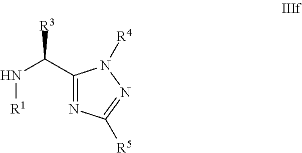

wherein R¹, R³, R⁴ and R⁵ are as described in formula (I), with a compound of the formula II

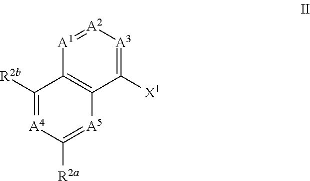

wherein A¹, A², A³, A⁴, A⁵, R^{2a} and R^{2b} are as described in formula (I) and X¹ is a leaving group, such as a halogen or a sulfonate, for instance chloride, under conditions already described in Scheme 1.

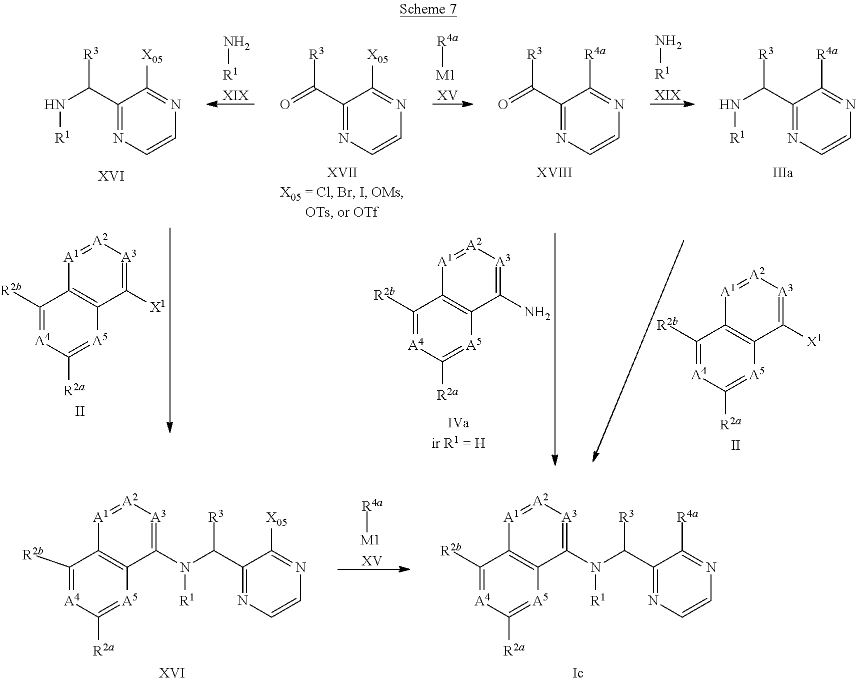

Compounds of formula Ic can be made, for example, as shown in scheme 7. Reaction of a compound of the formula XVII (wherein X₀₅ is a leaving group such as chlorine, bromine, iodine, arysulfonate, alkylsulfonate or trifluoromethanesulfonate) with an amine of the formula XIX gives compounds of the formula XVI. This reaction is done in the presence of a reducing agent, such as for example hydrogen, or a hydride, such as sodium borohydride, with or without a catalyst, such as a hydrogenation catalyst, for example palladium on carbon, with or without the presence of an acid, such as acetic acid, or a Lewis acid, such as zinc bromide, in a solvent or without a solvent, such as, for instance, methanol. The reaction can be conducted in a temperature range of −100 to +300° C., preferably between ambient temperature and 200° C. Such methods, and the range of conditions to perform them, for the reductive alkylation of amines are well known to a person skilled in the art.
Subsequent reaction of the intermediate of the formula XVI with a compound of the formula II gives a compound of the formula XIV. This reaction can be conducted neat or in a solvent, preferably in a solvent, such as an organic solvent, for instance acetonitrile, in a temperature range of −100 to +300° C., preferably between ambient temperature and 200° C., with or without the presence of a catalyst, for instance a metal catalyst, such as a palladium complex, and with or without the addition of a base, such as an inorganic base, for instance potassium carbonate, or an organic base, such as, for example, triethylamine.
Subsequently, the intermediate of the formula XIV is reacted with a compound of the formula XV to give the compound of formula Ic, wherein A¹, A², A³, A⁴, A⁵, R^{2a}, R^{2b}, R¹, R³ and R^{4a} have the same meaning as given above for compounds of the formula (I), and M1 in R^{4a}-M1 is a metal, such as for instance lithium, or —MgCl, or —ZnBr, or —B(OH)₂; or R^{4a}-M1 represents a boronate, such as a pinacol ester of a boronic acid, or a stannane such as R^{4a}—Sn(n-Bu)₃. Such transformations are known to a person skilled in the art as Suzuki-, Kumada-, Negishi- or Stille-coupling reactions, respectively. Such reactions are carried out in a temperature range of −100 to +300° C., preferably between ambient temperature and 200° C., in the presence of a catalyst, such as a metal catalyst, for instance a palladium catalyst, and a ligand, such as for example a phosphine ligand, or an N-heterocyclic carbene (NHC) ligand, or a phosphite ligand. The reaction can be done in the presence or absence of an additional metal catalyst, such as, for example, a copper salt, for instance CuI. The reaction is done with or without a base, which can be an inorganic base, such as potassium carbonate, or sodium hydroxide, or cesium carbonate, or an organic base, such as an amine base, for instance triethyl amine. This reaction is done with or without a solvent, preferentially in a solvent. Where the reaction mixture is heated, the reaction can be conducted under microwave irradiation or with conventional heating, such as heating the reaction vessel in an oil bath.
By an alternative route, compound XVII can be reacted with a compound of the formula XV to give intermediate XVIII. This reaction is done essentially under in the same range of conditions as described for the transformation of intermediate XIV to the compound of formula Ic.
Subsequently, the intermediate XVIII is reacted with amine IVa to give a compound of the formula Ic, wherein R¹ is hydrogen and A¹, A², A³, A⁴, A⁵, R^{2a}, R^{2b}, R³ and R^{4a} have the same meaning as given above for compounds of the formula (I). This reaction is done in the presence of a reducing agent, essentially under the same conditions as described above for the transformation of compound XVII to intermediate XVI.
By yet another alternative route, the intermediate compound of the formula XVIII can be reacted with an amine of the formula XIX to give the intermediate of the formula Illa. This reaction is done in the presence of a reducing agent, essentially under the same conditions as described above for the transformation of compound XVII to intermediate XVI.
Subsequently, the intermediate of the formula IIIa is reacted with a compound of the formula II to give the compound of the formula Ic, wherein A¹, A², A³, A⁴, A⁵, R^{2a}, R^{2b}, R¹, R³ and R^{4a} have the same meaning as given above for compounds of the formula (I). This reaction is done essentially under the same conditions as described above for the transformation of intermediate XVI to intermediate XIV. Within these different multistep sequences, the intermediate compounds of formulas XIV, XVI, XVIII and IIIa can be used as crude products for the respective subsequent step, or they can be purified, for instance by chromatography, and used in purified form for the next transformation. Compounds of the formula XVII are known, or they can be prepared by methods known to a person skilled in the art.
Compounds of the formula Id

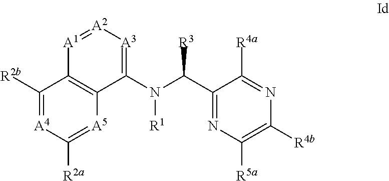

can be prepared by the reaction of an amine of the formula IIIb, or a salt thereof

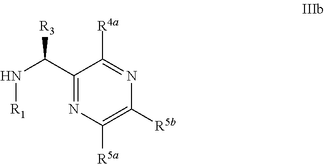

wherein R¹, R³, R^{4a}, R^{5a} and R^{5b} are as described in formula (I) with a compound of the formula II

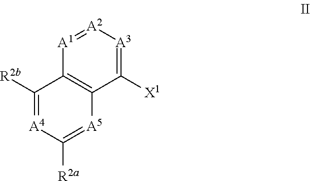

wherein A¹, A², A³, A⁴, A⁵, R^{2a} and R^{2b} are as described in formula (I) and X¹ is a leaving group, such as a halogen or a sulfonate, for instance chloride.
The chemistry is described in more detail in Scheme 8.

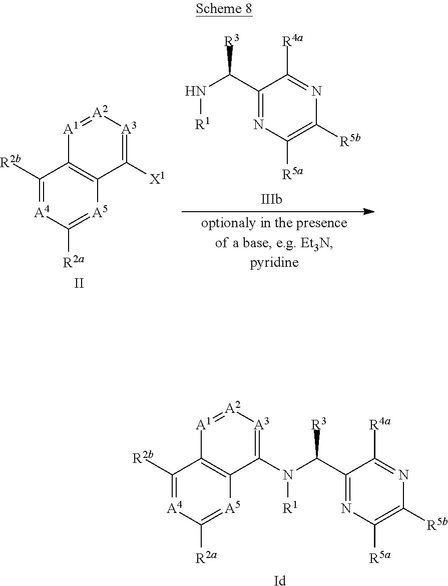

Reaction of a compound of the formula II, wherein X¹ is a leaving group, such as a halogen or sulfonate, for instance chloride, with a compound of formula IIIb gives a compound of the formula Id, wherein A¹, A², A³, A⁴, A⁵, R¹, R^{2a}, R^{2b}, R³, R^{4a}, R^{5a} and R^{5b} have the same meaning as given above for compounds of the formula (I). The reaction can be conducted neat or in a solvent, preferably in a solvent, such as an organic solvent, for instance acetonitrile, in a temperature range of −100 to +300° C., preferably between ambient temperature and 200° C., with or without the presence of a catalyst, for instance a metal catalyst, such as a palladium complex, and with or without the addition of a base, such as an inorganic base, for instance potassium carbonate, or an organic base, such as, for example, triethylamine.
The formation of compounds of formula IIIb is outlined in Scheme 9.

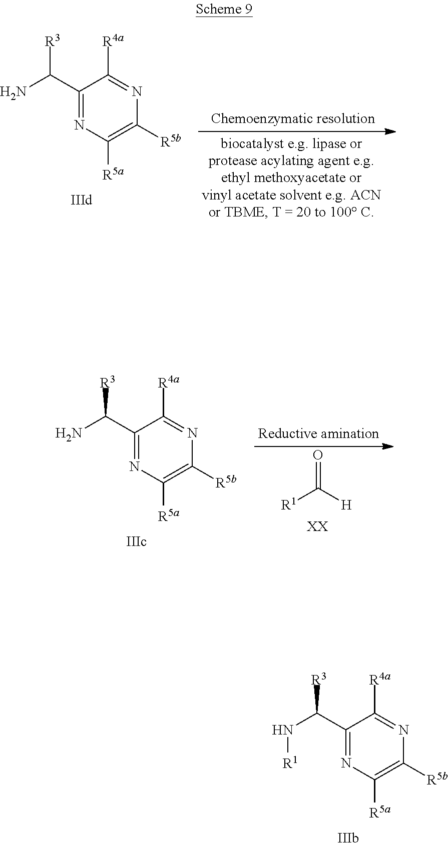

Compounds of formula IIIb can be prepared by treatment of compounds of formula IIIc, wherein R³, R^{4a}, R^{5a}, and R^{5b} are as described in formula (I), with compounds of formula XX wherein R¹ is as defined in formula (I), e.g. in the presence of NaBH(OAc)₃ or NaBH₃CN, in a suitable solvent, preferably in acetic acid at room temperature analogous to WO2002/088073, page 35. Alternatively, another reagent system for the reductive amination uses a combination of Ti(i-OiPr)₄ and NaBH₄ (see Synthesis 2003 (14), 2206).
Amines of formula IIIc may be obtained by biocatalyzed deracemization of amines of formula IIId. This may be done for instance using a lipase, e.g. Candida Antarctica lipase B or Pseudomonas fluorescens lipase, eventually in immobilized form (e.g. Novozym® 435) in presence of an acyl donor, e.g. ethyl methoxyacetate or vinyl acetate, in a suitable solvent such as acetonitrile or methyl tert-butyl ether at temperatures between 20° C. to 100° C. Such processes are described for instance in J. Org. Chem. 2007, 72, 6918-6923 or Adv. Synth. Catal. 2007, 349, 1481-1488. The expected stereochemical outcome of such enzymatic deracemization are known of those skilled in the art and are documented in the literature, for instance in J. Org. Chem. 1991, 56, 2656-2665 or J. Am. Chem. Soc. 2015, 137, 3996-4009.
In an alternative process, compounds of formula IIIc, or a salt thereof (such as a hydrohalide salt, preferably a hydrochloride or a hydrobromide salt, or a trifluoroacetic acid salt, or any other equivalent salt), can be obtained from compounds of the formula XXII, wherein R³, R^{4a}, R^{5a}, and R^{5b} are as described in formula (I), following the synthesis described in Scheme 10.

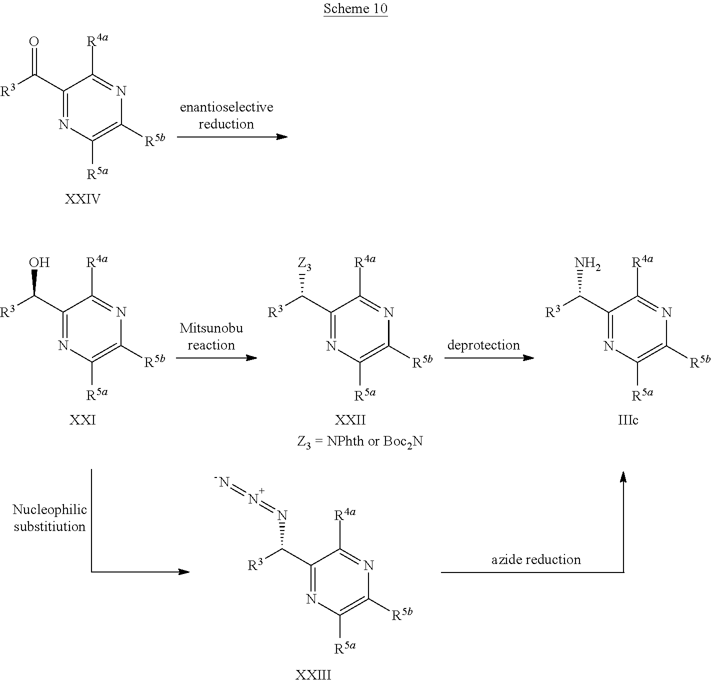

Amines of formula IIIc, or a salt thereof, may be obtained from intermediates of formula XXII, wherein R³, R^{4a}, R^{5a}, and R^{5b} are as described in formula (I) and Z₃ is —NPhth (N-phthalimide group) or —NBoc₂ (N-bis(tert-butyloxycarbonyl) group), typically by treatment with either hydrazine (preferably hydrazine hydrate or hydrazine monohydrate) in an alcohol solvent such as ethanol or isopropanol (Z₃ is —NPhth), or with an acid such as trifluoroacetic acid or hydrochloric acid in the presence of a suitable solvent such as dichloromethane, tetrahydrofuran or dioxane (Z₃ is —NBoc₂), under deprotection conditions known to a person skilled in the art, and described in the literature, such as for example in: Protective Groups in Organic Synthesis, 3rd Edition Theodora W. Green (The Rowland Institute for Science) and Peter G. M. Wuts (Pharmacia and Upjohn Company), John Wiley & Sons, Inc., New York, NY. 1999, ISBN 0-471-16019-9.
Such intermediates of formula XXII, wherein R³, R^{4a}, R^{5a}, and R^{5b} are as described in formula (I) and Z₃ is —NPhth (N-phthalimide group) or —NBoc₂ (N-bis(tert-butyloxycarbonyl) group), can be obtained from alcohols of formula XXI, wherein R³, R^{4a}, R^{5a}, and R^{5b} are as described in formula (I), by a Mitsunobu reaction, which involves treating alcohols of formula XXI with an azodicarboxylate, such as diethyl azodicarboxylate or diisopropyl azodicarboxylate in the presence of a phosphine, such as triphenylphosphine or tributylphosphine, and of an amine such as phthalimide (HNPhth) or bis(tert-butoxycarbonyl)amine(HNBoc₂). Mitsunobu reactions are known by those skilled in the art to proceed with inversion of the stereocenter, as described for instance in Chem. Rev. 2009, 109, 2551-2651. Alternatively, amines of formula IIIc may be obtained by reduction of azides of formula XXIII, wherein R³, R^{4a}, R^{5a}, and R^{5b} are as described in formula (I), by treatment with triphenylphosphine and water (Staudinger reaction) or by hydrogenation for example using a palladium catalyst in the presence of hydrogen. Azides of formula XXIII may be obtained by treatment of alcohols of formula XXI, wherein R³, R^{4a}, R^{5a}, and R^{5b} are as described in formula (I), with an azidation reagent such as diphenyl phosphoryl azide in a solvent such as toluene or THE in presence of a base such as DBU. Such processes are known by those skilled in the art to proceed with inversion of the stereocenter and are described in the literature for instance in Adv. Synth. Catal. 2018, 360, 2157-2165.
Alcohols of formula XXI may be obtained by enantioselective reduction of ketones of formula XXIV, wherein R³, R^{4a}, R^{5a}, and R^{5b} are as described in formula (I). Such reductions can be done using a catalyst, for instance a ruthenium or a rhodium catalyst with a chiral ligand such as RuCl[(R,R)-TsDPEN](mesitylene) or RuBF₄[(R,R)-TsDPEN](p-cymene) in the presence of a hydrogen donor system such as for example HCOOH/Et₃N or HCO₂NH₄. Such processes are described in the literature for instance in J. Org. Chem. 2017, 82, 5607.
Alternatively, compounds of formula IIIc may also be prepared as outlined in Scheme 11.

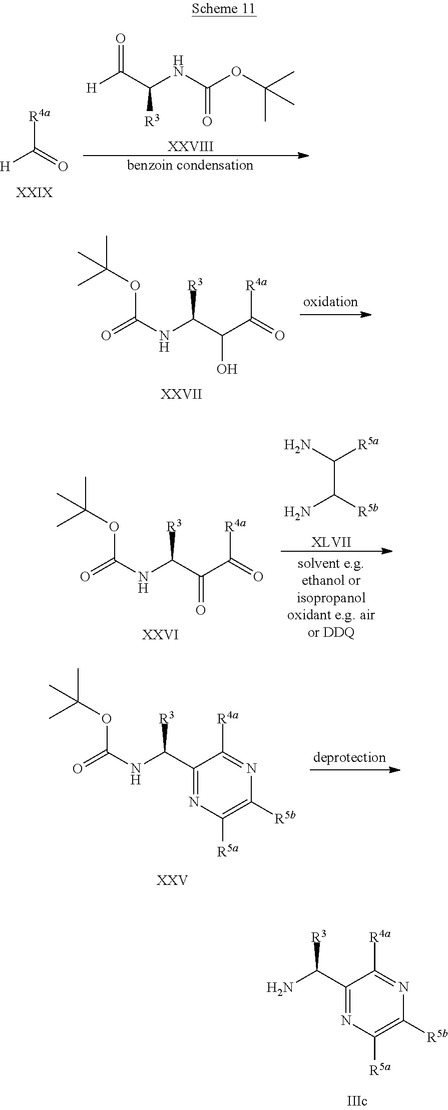

Amines of formula IIIc, or a salt thereof (such as a hydrohalide salt, preferably a hydrochloride or a hydrobromide salt, or a trifluoroacetic acid salt, or any other equivalent salt), can be prepared by deprotection of amines of formula XXV, wherein R³, R^{4a}, R^{5a}, and R^{5b} are as described in formula (I), for instance using an acid such as trifluoroacetic acid or hydrochloric acid, optionally in the presence of a suitable solvent such as dichloromethane, tetrahydrofuran or dioxane.
Amines of formula XXV can be obtained by condensation of diamines of formula XXVII, wherein R^{5a} and R^{5b} are as described in formula (I), on diketones of formula XXVI, wherein R³, and R^{4a} are as described in formula (I). This condensation can take place in the presence of a suitable solvent such as ethanol or isopropanol in presence of an oxidant such as air or DDQ.
Diketones of formula XXVI may be formed by oxidation of hydroxyketones of formula XXVII wherein R³, and R^{4a} are as described in formula (I). This oxidation can involve for instance SO₃-pyridine in presence of solvents such as dichloromethane or dimethyl sulfoxide DMSO, or mixtures thereof, and a base for instance triethylamine or alternatively sodium hypochlorite in presence of a catalyst such as TEMPO/Bu₄NHSO₄. Examples of such oxidations can be found in the literature, for instance in Synlett, 2014, 25, 596 or J. Am. Chem. Soc. 1990, 112, 5290-5313.
Hydroxyketones of formula XXVII may be synthesized by cross-benzoin condensation between aldehydes of formula XXIX, wherein R^{4a} is as described in formula (I), and aldehydes of formula XXVIII, wherein R³ is as described in formula (I).
Aldehydes of formula XXVIII are commercially available in chiral form, like for instance Boc-L-alaninal (CAS 79069-50-4) or tert-butyl N-[(1S)-1-(cyclopropylmethyl)-2-oxo-ethyl]carbamate (CAS 881902-36-9). Cross-benzoin condensations are done in the usual way by employing an organocatalyst such as a triazolium salt or a thiazolium salt in the presence of a base such as potassium tert-butoxide or N,N-isopropylethylamine in a suitable solvent such as DCM or THE at a temperature between −20° C. and the boiling point of the solvent. Examples of catalysts for such transformations have been described in the literature for instance in J. Am. Chem. Soc. 2014, 136, 7539-7542 or in Org. Lett. 2016, 18, 4518-4521.

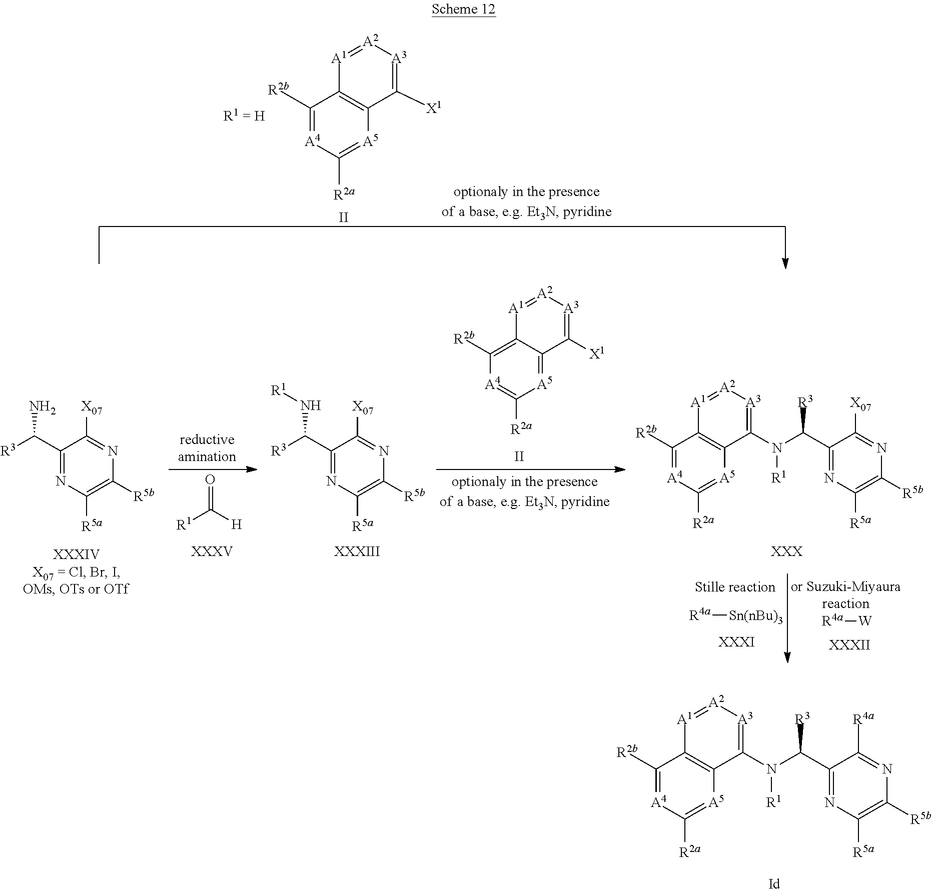

As shown in Scheme 12, compounds of formula Id can be alternatively prepared by reaction of compounds of formula XXX (wherein A¹, A², A³, A⁴, A⁵, R¹, R^{2a}, R^{2b}, R³, R^{5a}, and R^{5b} are as defined in formula (I) and X₀₇ is a leaving group like, for example, chlorine, bromine, iodine) with compounds of formula XXXI (Stille reaction; R^{4a} in XXXI is as defined in formula I) or compounds of formula XXXII (Suzuki-Miyaura reaction; R^{4a} in XXXII is as defined in formula (I) and W is a boronic acid B(OH)₂ group, or a corresponding boronate, such as a pinacol ester of said boronic acid) in the presence of a palladium catalyst as described in detail in Scheme 7.
Compounds of formula XXX can be prepared by coupling of amines of formula XXXIII and compounds of formula II, wherein A¹, A², A³, A⁴, A⁵, R^{2a}, R^{2b} and X¹ are described in Scheme 1, under the conditions described in detail in Scheme 1. Under the same conditions, if R¹=H, compounds of formula XXX may be obtained directly from compounds of formula XXXIV.
Compounds of formula XXXIII can be prepared by treatment of compounds of formula XXXIV, with compounds of formula XXXV (wherein R¹ is as defined in formula I), e.g. in the presence of NaBH(OAc)₃ or NaBH₃CN, in a suitable solvent, preferably in acetic acid at room temperature analogous to WO2002/088073, page 35. Alternatively, another reagent system for the reductive amination uses a combination of Ti(i-OiPr)₄ and NaBH₄ (see Synthesis 2003 (14), 2206).
Amines of formula XXXIV can be prepared by deracemization procedure method, which involves for example, a selective acylation of one enantiomer. Such an example is described more in details in Scheme 13.

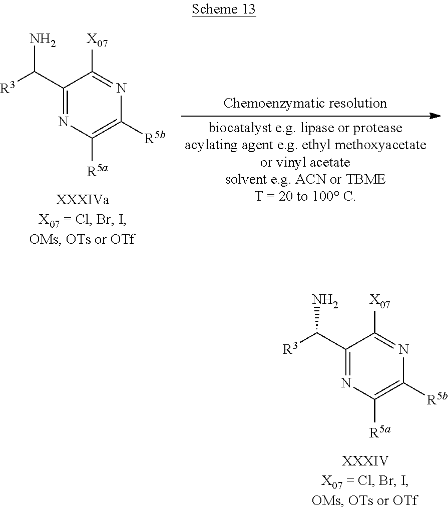

Amines of formula XXXIV may be obtained by biocatalyzed deracemization of amines of formula XXXIVa, wherein R³, R^{5a}, and R^{5b} are as in formula (I) and X₀₇ is a leaving group such as bromine, chlorine or iodine. This may be done for instance using a lipase, e.g. Candida Antarctica lipase B or Pseudomonas fluorescens lipase, eventually in immobilized form (e.g. Novozym® 435) in presence of an acyl donor, e.g. ethyl methoxyacetate or vinyl acetate, in a suitable solvent such as acetonitrile or methyl tert-butyl ether at temperatures between 20° C. to 100° C. Such processes are described for instance in J. Org. Chem. 2007, 72, 6918-6923 or Adv. Synth. Catal. 2007, 349, 1481-1488. The expected stereochemical outcome of such enzymatic deracemization are known of those skilled in the art and are documented in the literature, for instance in J. Org. Chem. 1991, 56, 2656-2665 or J. Am. Chem. Soc. 2015, 137, 3996-4009.
Alternatively, resolution of amines of formula XXXIVa to give amines of formula XXXIV may be achieved using a chiral auxiliary, as described in Scheme 14.

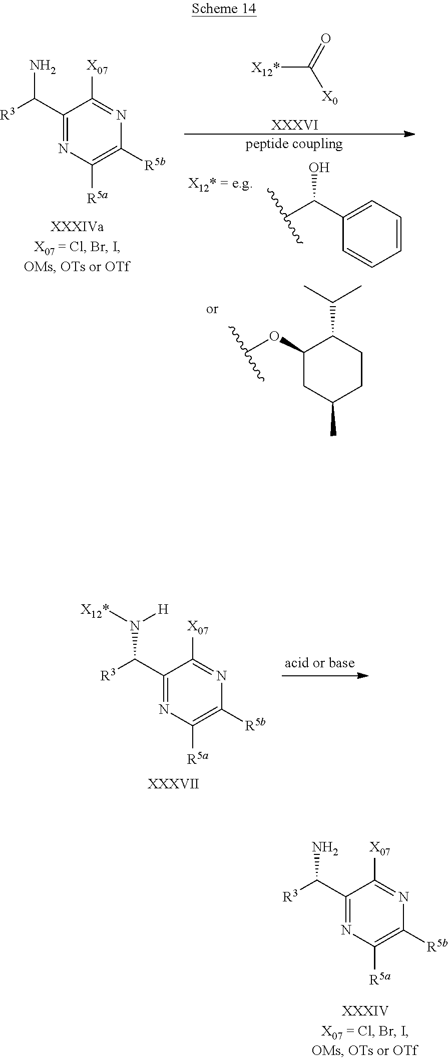

Amines of formula XXXIV can be prepared from intermediates of formula XXXVII, wherein R³, R^{5a}, and R^{5b} are as in compounds of the formula (I), X₀₇ is a leaving group such as bromine, chlorine or iodine, and X₁₂* is a chiral auxiliary, by treatment with acids such as HCl or bases such as NaOH. Chiral auxiliaries of formula XXXVI are for instance mandelic acid or (1R)-menthylchloroformate. Intermediates of formula XXXVII can be formed by coupling of a chiral auxiliary of formula XXXVI, wherein X₀ is a leaving group, such as chlorine, with amines of the formula XXXIVa following the conditions detailed in Scheme 1. Examples of such deracemization processes are reported in the literature, for instance in J. Org. Chem. 2007, 72, 485-493.
Alternatively, amines of formula XXXIV, or a salt thereof (such as a hydrohalide salt, preferably a hydrochloride or a hydrobromide salt, or a trifluoroacetic acid salt, or any other equivalent salt), can be formed as described in Scheme 15.

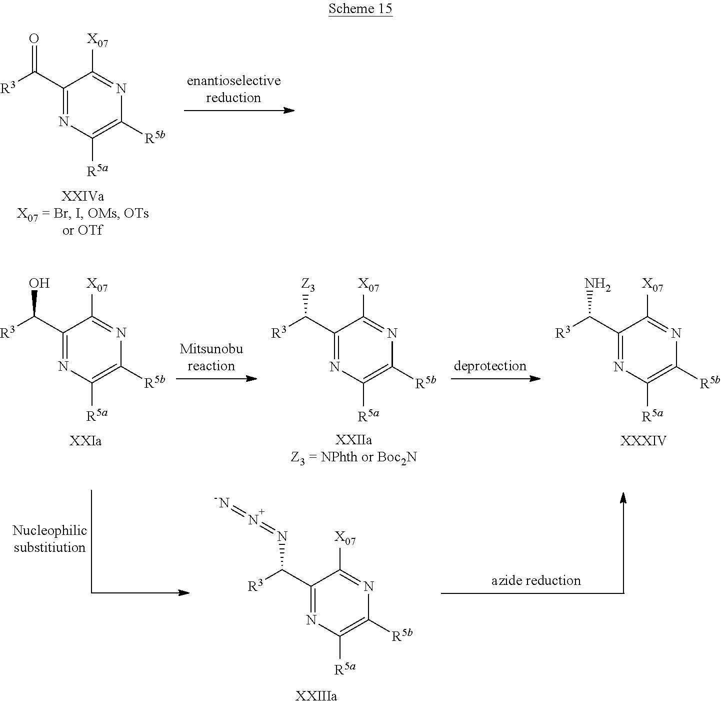

Amines of formula XXXIV, or a salt thereof, may be obtained from intermediates of formula XXIIa, wherein R³, R^{5a}, and R^{5b} are as described in formula (I), X₀₇ is a leaving group such as a halogen or sulfonate, for instance bromide, and Z₃ is —NPhth (N-phthalimide group) or —NBoc₂ (N-bis(tert-butyloxycarbonyl) group), typically by treatment with either hydrazine (preferably hydrazine hydrate or hydrazine monohydrate) in an alcohol solvent such as ethanol or isopropanol (Z₃ is —NPhth), or with an acid such as trifluoroacetic acid or hydrochloric acid in the presence of a suitable solvent such as dichloromethane, tetrahydrofuran or dioxane (Z₃ is —NBoc₂), under deprotection conditions known to a person skilled in the art, and described in the literature, such as for example in: Protective Groups in Organic Synthesis, 3rd Edition Theodora W. Green (The Rowland Institute for Science) and Peter G. M. Wuts (Pharmacia and Upjohn Company), John Wiley & Sons, Inc., New York, NY. 1999, ISBN 0-471-16019-9.
Such intermediates of formula XXIIa, wherein R³, R^{5a}, and R^{5b} are as described in formula (I), X₀₇ is a leaving group such as a halogen or sulfonate, for instance bromide, and Z₃ is —NPhth (N-phthalimide group) or —NBoc₂ (N-bis(tert-butyloxycarbonyl) group), can be obtained from alcohols of formula XXIa, wherein R³, R^{5a}, and R^{5b} are as described in formula (I) and X₀₇ is a leaving group, by a Mitsunobu reaction, which involves treating alcohols of formula XXIa with an azodicarboxylate, such as diethyl azodicarboxylate or diisopropyl azodicarboxylate, in the presence of a phosphine, such as triphenylphosphine or tributylphosphine, and of an amine such as phthalimide(HNPhth) or bis(tert-butoxycarbonyl)amine (HNBoc₂). Mitsunobu reactions are known by those skilled in the art to proceed with inversion of the stereocenter, as described for instance in Chem. Rev. 2009, 109, 2551-2651.
Alternatively, amines of formula XXXIV may be obtained by reduction of azides of formula XXIIIa, wherein R³, R^{5a}, and R^{5b} are as described in formula (I) and X₀₇ is a leaving group such as a halogen or sulfonate, for instance bromide, by treatment with triphenylphosphine and water (Staudinger reaction) or by hydrogenation for example using a palladium catalyst in the presence of hydrogen. Azides of formula XXIIIa may be obtained by treatment of alcohols of formula XXIa with an azidation reagent such as diphenyl phosphoryl azide in a solvent such as toluene or THE in presence of a base such as DBU. Such processes are known by those skilled in the art to proceed with inversion of the stereocenter and are described in the literature for instance in Adv. Synth. Catal. 2018, 360, 2157-2165.
Alcohols of formula XXIa may be obtained by enantioselective reduction of ketones of formula XXIVa, wherein R³, R^{5a}, and R^{5b} are as described in formula (I) and X₀₇ is a leaving group such as a halogen or sulfonate, for instance bromide. Such reductions can be done using catalysts, for instance a ruthenium or a rhodium catalyst with a chiral ligand such as RuCl[(R,R)-TsDPEN](mesitylene) or RuBF₄[(R,R)-TsDPEN](p-cymene) in the presence of a hydrogen donor system such as for example HCOOH/Et₃N or HCO₂NH₄. Such processes are described in the literature for instance in J. Org. Chem. 2017, 82, 5607.
Compounds of formula II

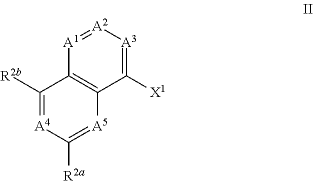

wherein X¹ is a leaving group, such as a halogen or sulfonate, for instance chloride, can be made, for example, as shown in Schemes 16-18.

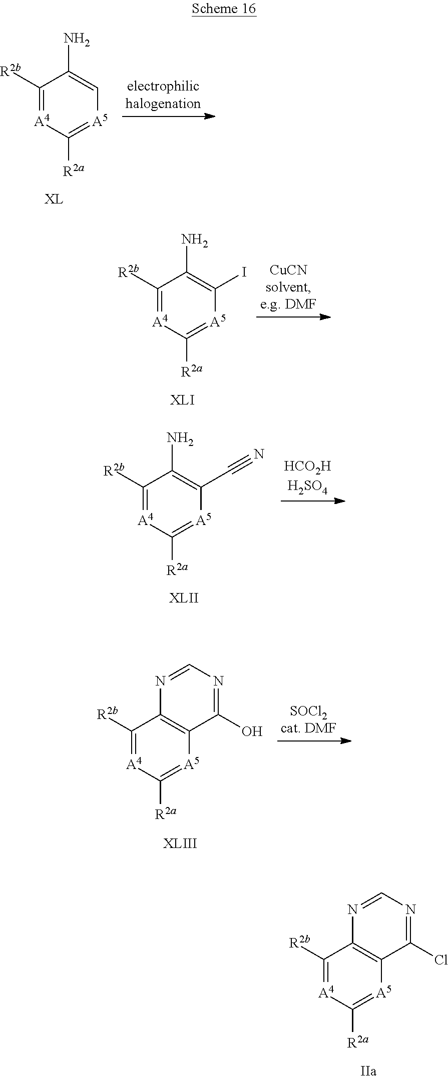

Compounds of formula Ila wherein R^{2a}, R^{2b}, A⁴ and A⁵ are as described in formula (I), can be prepared according to reaction scheme 16. Compounds of formula XLII are either known, or they can be prepared by methods known to a person skilled in the art. For example, a compound of formula XL is reacted with an electrophilic iodinating reagent, such as N-iodosuccinimide, in a solvent, such as hexafluoroisopropanol, as described for example in J. Org. Chem. 2018, 83, 930, to obtain compounds of formula XLI. Cyanation of compounds of formula XLI with copper(I) cyanide in a solvent, such as DMF, at temperatures such as 100° C., provides compounds of formula XLII (procedure analogous to WO2005/100298, page 44). Treatment of compounds of formula XLII with formic acid and sulfuric acid at temperatures between 8° and 100° C., provides compounds of formula XLIII (procedure analogous to WO2018/206539, page 80). Subsequent conversion of compounds of formula XLIII to compounds of formula Ila is accomplished via methods known to the person skilled in the art, for example with thionyl chloride in presence of catalytic N,N-dimethylformamide at reflux, analogous to WO2015/54572, page 263.
Compounds of formula XLIII, wherein R^{2a}, A⁴ and A⁵ are as described in formula (I) above, and in which R^{2b} is C₁-C₄alkylsulfonyl (more particularly methylsulfonyl) substituted with one to three substituents (more particularly two to three substituents) independently selected from R^{x}, wherein R^{x} is halogen (more particularly fluorine) can be represented by compounds of formula XLIIIa, wherein R^{2a}, A⁴ and A⁵ are as described in formula (I), and in which Gr is difluoromethyl or trifluoromethyl.

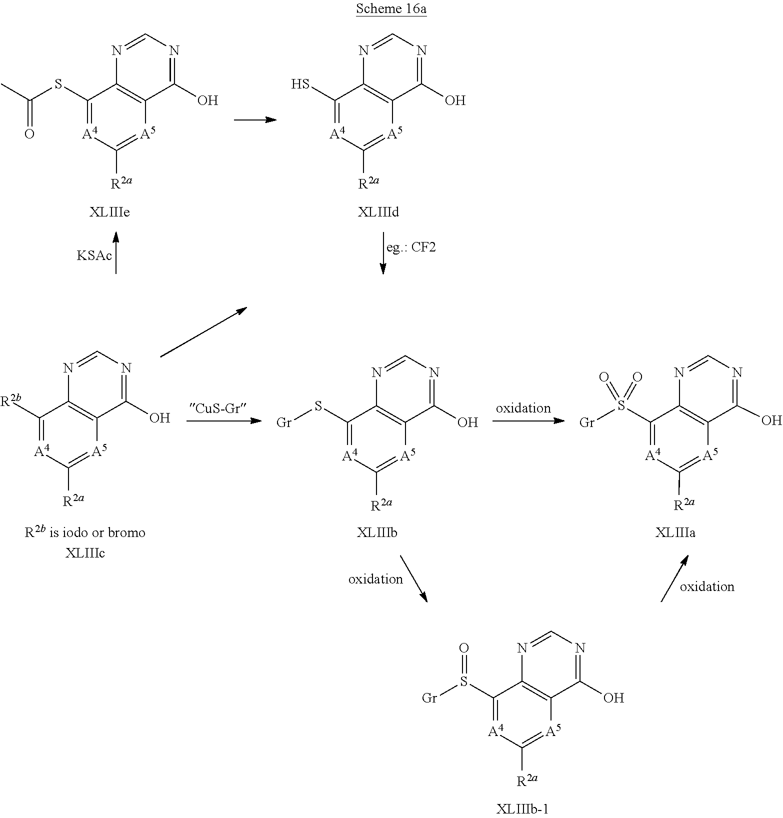

Such sulfone compounds of formula XLIIIa can be obtained (Scheme 16a) by means of an oxidation reaction of either the corresponding sulfide compounds of formula XLIIIb or the sulfoxide compounds of formula XLIIIb-1 involving reagents such as, for example, m-chloroperoxybenzoic acid (mCPBA), hydrogen peroxide, potassium peroxymonosulfate (Oxone®), sodium periodate, sodium hypochlorite or tert-butyl hypochlorite, amongst other oxidants, and optionally in the presence of a catalyst, such as ruthenium chloride, sodium tungstate or iron-, manganese-, cobalt- and vanadium-based catalysts.
This oxidation reaction is generally conducted in the presence of a solvent, such as for example aliphatic halogenated hydrocarbons (for instance dichloromethane, chloroform or carbon tetrachloride), esters (for instance ethyl acetate), alcohols (for instance methanol or ethanol), acetonitrile, acetic acid or water; or mixtures thereof. Similarly, sulfoxide compounds of formula XLIIIb-1 may be obtained via oxidation of the sulfide compounds of formula XLIIIb. The amount of the oxidant to be used in the reaction is generally 1 to 3 moles, preferably 1 to 1.2 moles, relative to 1 mole of the sulfide compounds XLIIIb to produce the sulfoxide compounds XLIIIb-1, and preferably 2 to 2.2 moles of oxidant, relative to 1 mole of the sulfide compounds XLIIIb to produce the sulfone compounds XLIIIa.
Compounds of the formula XLIIIb, wherein R^{2a}, A⁴ and A⁵ are as described in formula (I) above, and in which Gr is trifluoromethyl, can be prepared by reacting compounds of the formula XLIIIc, wherein R^{2a}, A⁴ and A⁵ are as described in formula (I) above, and in which R^{2b} is iodo or bromo, with a trifluoromethylthiolation reagent “CuS-Gr” such as copper(I) trifluoromethanethiolate (CAS 3872-23-9) or trifluoromethylthiolato(2,2-bipyridine)copper(I) (also known as (bpy)Cu(SCF3), CAS 1413732-47-4), in solvents such as acetonitrile, dioxane, N,N-dimethylformamide or N,N-dimethylacetamide, and at temperatures between 0 and 180° C., preferably between room temperature and 150° C. Such conditions are described in the literature, for example in J. Org. Chem. 1976, 41, 1644 or Synthesis 1975, 721 (CAS 3872-23-9) and Angew. Chem. Int. Ed. 2013, 52, 1548 or Tetrahedron, 2013, 69, 6046 (CAS 1413732-47-4).
Compounds of the formula XLIIIb, wherein R^{2a}, A⁴ and A⁵ are as described in formula (I) above, and in which Gr is difluoromethyl, may be obtained by treatment of compounds of the formula XLIIId, wherein R^{2a}, A⁴ and A⁵ are as described in formula (I) above, with a :CF2 carbene, generated for example from a difluoroacetate XcCF₂COONa or a difluoromethyl(phosphonate) XcCF₂P(O)(OEt)₂, wherein Xc can be chloro or bromo, in the presence of a base such as for example sodium or potassium carbonate, in an appropriate solvent like for example acetonitrile, N,N-dimethylformamide or N-methyl-2-pyrrolidone (NMP), at temperatures between room temperature and the boiling point of the reaction mixture. Such methods have been described in the literature, e.g. in Org. Lett. 15(19), 5036-5039; 2013, Tet 65(27), 5278-5283; 2009 or Chem. Commun. 53, 5706; 2017.
Alternatively, compounds of formula XLIIIb may be obtained by treatment of compounds of formula XLIIId with a difluoromethylsulfinate reagent, in the presence of an oxidant, as described for example in J. Fluor. Chem., 193, 113-117; 2017.
Compounds of the formula XLIIId, wherein R^{2a}, A⁴ and A⁵ are as described in formula (I) above, may be obtained by hydrolyzing compounds of the formula XLIIIe, wherein R^{2a}, A⁴ and A⁵ are as described in formula (I) above, via basic or acidic hydrolysis conditions known to a person skilled in the art (thioacetate hydrolysis, for example by treatment with an aqueous acid solution, such as aqueous HCl).
Compounds of the formula XLIIIe, wherein R^{2a}, A⁴ and A⁵ are as described in formula (I) above, may be obtained by treatment of compounds of the formula XLIIIc, wherein R^{2a}, A⁴ and A⁵ are as described in formula (I), and in which R^{2b} is iodo or bromo, with potassium thioacetate (KSAc) or sodium thioacetate under Ullmann-type cross-coupling copper-mediated reaction conditions, as described for example in J. Org. Chem. 21, 11464; 2017.
Alternatively, compounds of the formula XLIIId, wherein R^{2a}, A⁴ and A⁵ are as described in formula (I) above, may be obtained by treatment of compounds of the formula XLIIIc, wherein R^{2a}, A⁴ and A⁵ are as described in formula (I), and in which R^{2b} is iodo or bromo, with sodium or potassium sulfide, alternatively sodium or potassium hydrosulfide, optionally as a hydrate salt, in an appropriate solvent like for example N,N-dimethylformamide, N-methyl-2-pyrrolidone (NMP) or ethanol, at temperatures between room temperature and the boiling point of the solvent, as described for example in Bioorg. Med. Chem. Lett. 23(13), 3947-3953; 2013 or WO12/088190.
The chemistry shown above (Scheme 16a) for the transformation XLIIIc to XLIIId to XLIIIb can be performed with some additional protection/deprotection steps as shown in Scheme 16b.

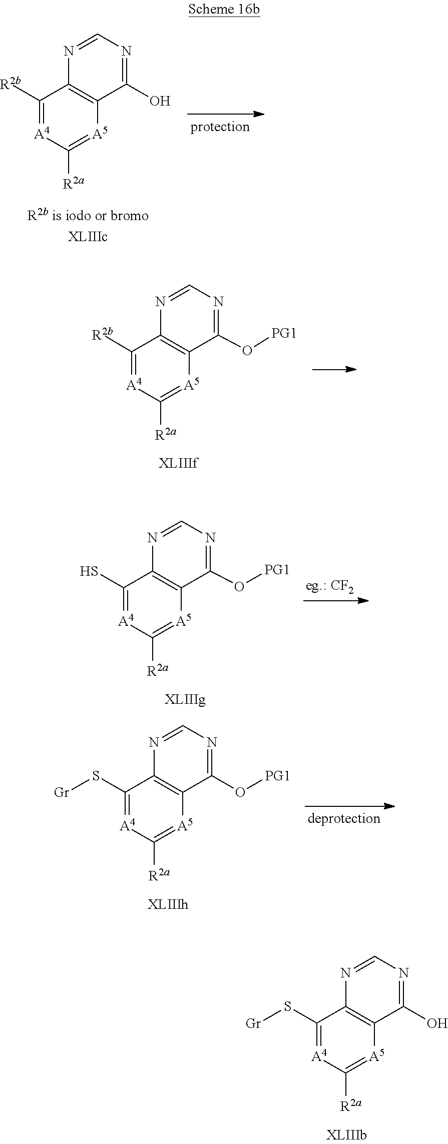

The chemistry and conditions for the transformation XLIIIf to XLIIIg to XLIIIh (Scheme 16b) is analogous to the descriptions above for the transformation XLIIIc to XLIIId to XLIIIb, wherein the definitions of the substituents Gr, R^{2a}, R^{2b}, A⁴ and A⁵ remains as detailed in Scheme 16a, and in which PG1 is a protective group, such as for example a tetrahydropyranyl (THP) or a tert-butyloxycarbonyl (Boc) group. Protection conditions (transformation XLIIIc to XLIIIf), respectively deprotection conditions (transformation XLIIIh to XLIIIb) are well known to a person skilled in the art, and described in the literature, such as for example in: Protective Groups in Organic Synthesis, 3rd Edition Theodora W. Green (The Rowland Institute for Science) and Peter G. M. Wuts (Pharmacia and Upjohn Company), John Wiley & Sons, Inc., New York, NY. 1999, ISBN 0-471-16019-9. For instance, THP protection of XLIIIc to generate XLIIIf, wherein PG1 is tetrahydropyranyl (THP), can be achieved by treatment with 3,4-dihydro-2H-pyran, in the presence of a Lewis acid such as trifluoroacetic acid, in an inert solvent such as dimethylsulfoxide, toluene or dioxane, and at temperatures between room temperature and the boiling point of the reaction mixture. Deprotection of XLIIIh, wherein PG1 is tetrahydropyranyl (THP), can be achieved by treatment with for instance p-methoxybenzenesulfonic acid or p-toluenesulfonic acid (PTSA), optionally as a hydrate salt, in an alcohol solvent such as methanol or ethanol, optionally in the presence of a co-solvent such as tetrahydrofuran, 2-methyltetrahydrofuran or dioxane, and at temperatures between 0 and 80° C., preferably around room temperature.
Alternatively, compounds of the formula XLIIIa, wherein R^{2a}, A⁴ and A⁵ are as described in formula (I), and in which Gr is difluoromethyl or trifluoromethyl,

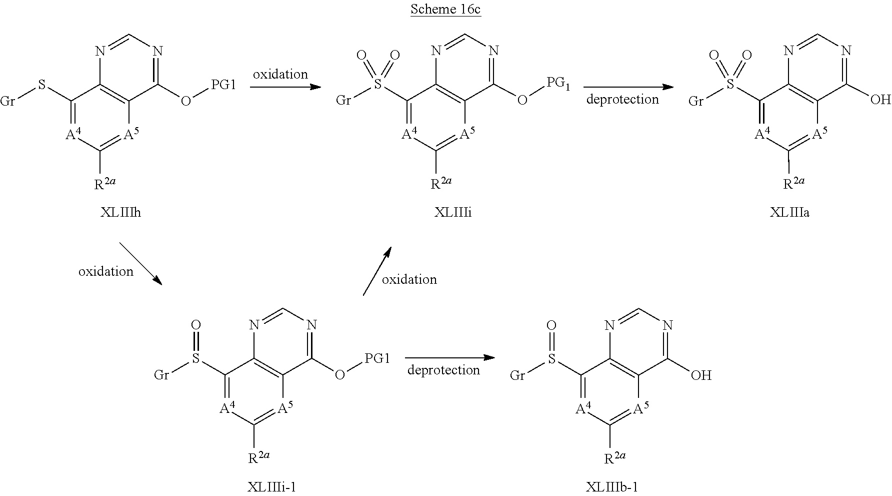

can be obtained (Scheme 16c) through deprotection of compounds of formula XLIIIi, wherein R^{2a}, A⁴ and A⁵ are as described in formula (I), and in which Gr is difluoromethyl or trifluoromethyl and PG1 is a protective group, such as for example a tetrahydropyranyl (THP) or a tert-butyloxycarbonyl (Boc) group, under conditions already detailed in Scheme 16b (transformation XLIIIh to XLIIIb). Similarly, compounds of the formula XLIIIb-1, wherein R^{2a}, A⁴ and A⁵ are as described in formula (I), and in which Gr is difluoromethyl or trifluoromethyl, may be obtained through deprotection of compounds of formula XLIIIi-1, wherein R^{2a}, A⁴ and A⁵ are as described in formula (I), and in which Gr is difluoromethyl or trifluoromethyl and PG1 is a protective group, such as for example a tetrahydropyranyl (THP) or a tert-butyloxycarbonyl (Boc) group.
Compounds of formula XLIIIi, wherein R^{2a}, A⁴ and A⁵ are as described in formula (I), and in which Gr is difluoromethyl or trifluoromethyl and PG1 is a protective group, such as for example a tetrahydropyranyl (THP) or a tert-butyloxycarbonyl (Boc) group, can be obtained by means of an oxidation reaction of either the corresponding sulfide compounds of formula XLIIIh or the sulfoxide compounds of formula XLIIIi-1, wherein R^{2a}, A⁴ and A⁵ are as described in formula (I), and in which Gr is difluoromethyl or trifluoromethyl and PG1 is a protective group, such as for example a tetrahydropyranyl (THP) or a tert-butyloxycarbonyl (Boc) group, under conditions already detailed in Scheme 16a (transformation XLIIIb to XLIIIa, respectively XLIIIb-1 to XLIIIa). In analogy, sulfoxide compounds of formula XLIIIi-1 may be obtained by oxidation of sulfide compounds of formula XLIIIh (transformation XLIIIb to XLIIIb-1 in Scheme 16a).

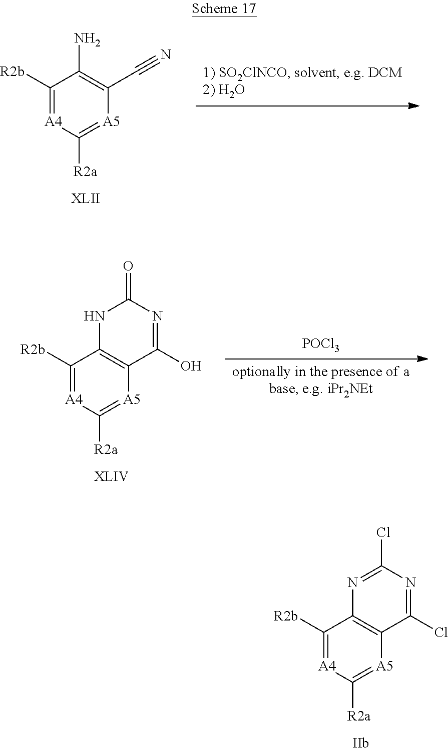

Compounds of formula IIb wherein R^{2a}, R^{2b}, A⁴ and A⁵ are as described in formula (I), can be prepared according to reaction scheme 17. A compound of formula XLII, prepared as in Scheme 16, is reacted with chlorosulfonyl isocyanate, then further reacted with water at reflux, as described for example in Synth. Commun. 1988, 18, 525, to provide intermediates of formula XLIV. Subsequent conversion of intermediates of formula XLIV to compounds of formula IIb is accomplished using chlorinating reagents, such as POCl₃, optionally in the presence of base, such as N,N-diisopropylethylamine. These chlorinating methods are well known to the person skilled in the art, and are described for example in WO2021/148639.

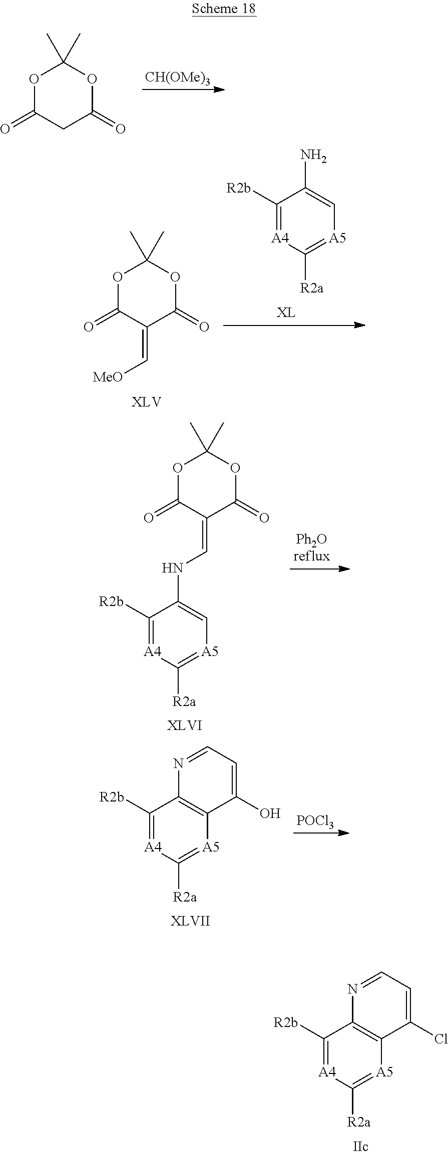

Compounds of formula IIc wherein R^{2a}, R^{2b}, A⁴ and A⁵ are as described in formula (I), can be prepared according to reaction scheme 18, analogous to procedures found in ChemCatChem 2017, 10, 965. Meldrum's acid is converted to compound XLV by refluxing in trimethyl orthoformate and further converted to compounds of formula XLVI in the same pot by addition of anilines of formula XL. Compounds of formula XLVI are refluxed in diphenyl ether to obtain 4-hydroxyquinolines of formula XLVII. Compounds of formula IIc are then obtained via chlorination of compounds of formula XLVII using chlorinating reagents, such as POCl₃, well known to the person skilled in the art.
Compounds of formula IVc

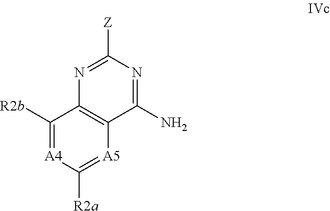

can be made, for example, as shown in Scheme 19.

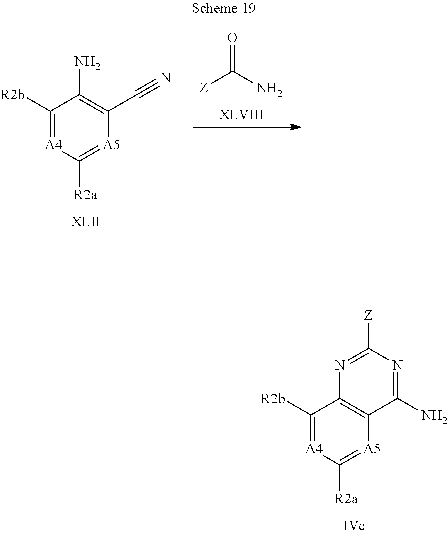

Compounds of formula IVc wherein Z is H, C₁-C₃ alkyl, cyclopropyl, CF₃, and wherein R^{2a}, R^{2b}, A⁴ and A⁵ are as described in formula (I), can be prepared according to reaction scheme 19. Compounds of formula XLII, prepared as in Scheme 16, are reacted in the presence of compounds of formula XLVIII at elevated temperatures, such as 180° C., as described for example in Eur. J. Med. Chem. 2017, 141, 446, to provide amines of formula IVc.
Alternatively, compounds of the formula Iab can be made, for example, as shown in Scheme 20.

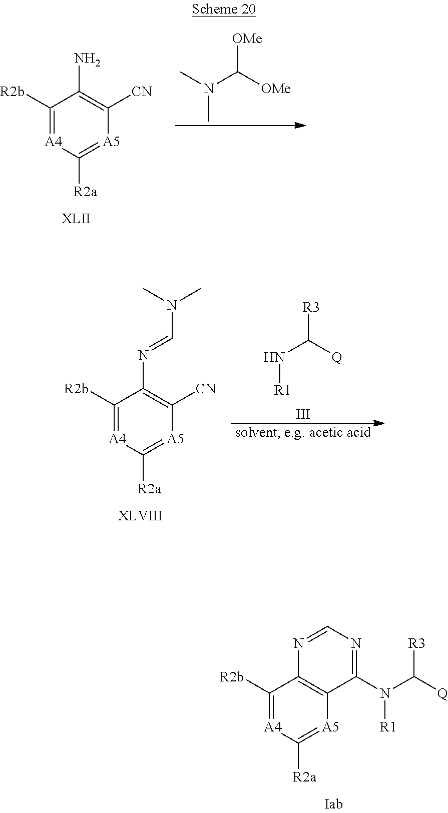

Compounds of formula Iab, wherein R¹, R^{2a}, R^{2b}, R³, A⁴, A⁵, Q is as described in formula (I), can be prepared according to scheme 20, analogous to the procedures in WO2010/093419, page 225. Compounds of formula XLII, prepared as in Scheme 16, are treated with N,N-dimethylformamide dimethyl acetal at elevated temperature, preferably 90° C., to provide formamidine products of formula XLVIII. Reaction with amines of formula III at elevated temperature, preferably 120° C., in a suitable solvent, preferably acetic acid, provides compounds of formula Iab.
Compounds of the formula If

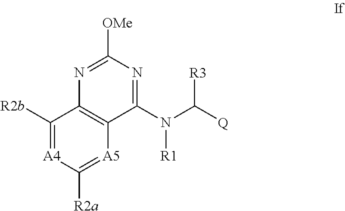

can be made, for example, as shown in Scheme 21.

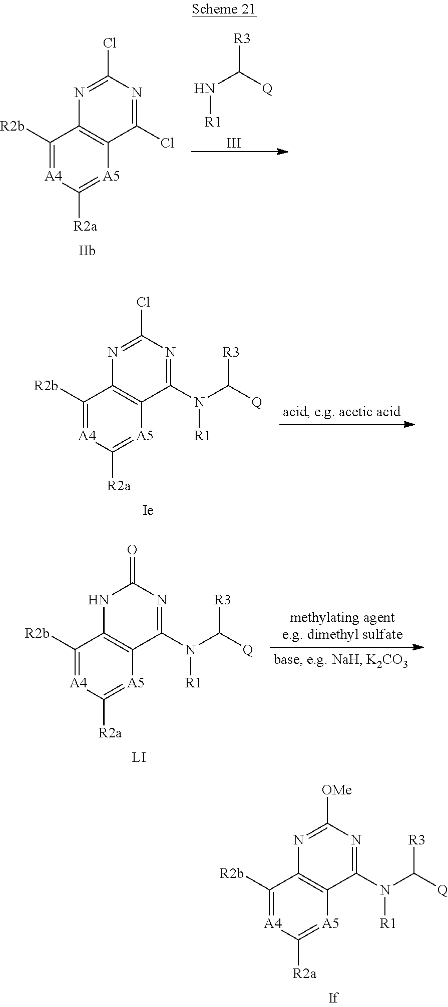

Compounds of formula If, wherein R¹, R^{2a}, R^{2b}, R³, A⁴, A⁵, Q is as described in formula (I), can be prepared according to Scheme 21. Reaction of compounds of formula IIb, prepared as in Scheme 17, wherein R^{2a}, R^{2b}, A⁴ and A⁵ are as described in formula (I), with amines of formula III, or a salt thereof (such as a hydrohalide salt, preferably a hydrochloride or a hydrobromide salt, or a trifluoroacetic acid salt, or any other equivalent salt), wherein R¹, R³ and Q are as described in formula (I), using procedures presented in Scheme 1, provides compounds of formula Ie. Treatment of compounds of formula Ie under acidic conditions, preferably with acetic acid, at elevated temperatures, preferably between 7° and 80° C., as described in Heterocycles 1996, 43, 2607, provides intermediates of formula LI. Methylation of compounds of formula LI to obtain compounds of formula If can be achieved using an electrophilic methyl sources, such as dimethyl sulfate or methyl iodide, in the presence of a base, such as potassium carbonate or sodium hydride, as well known to the person skilled in the art.
Compounds of the formula Ig

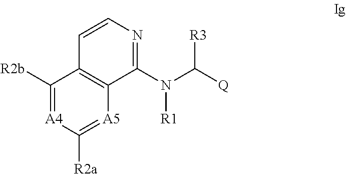

can be made, for example, as shown in Scheme 22.

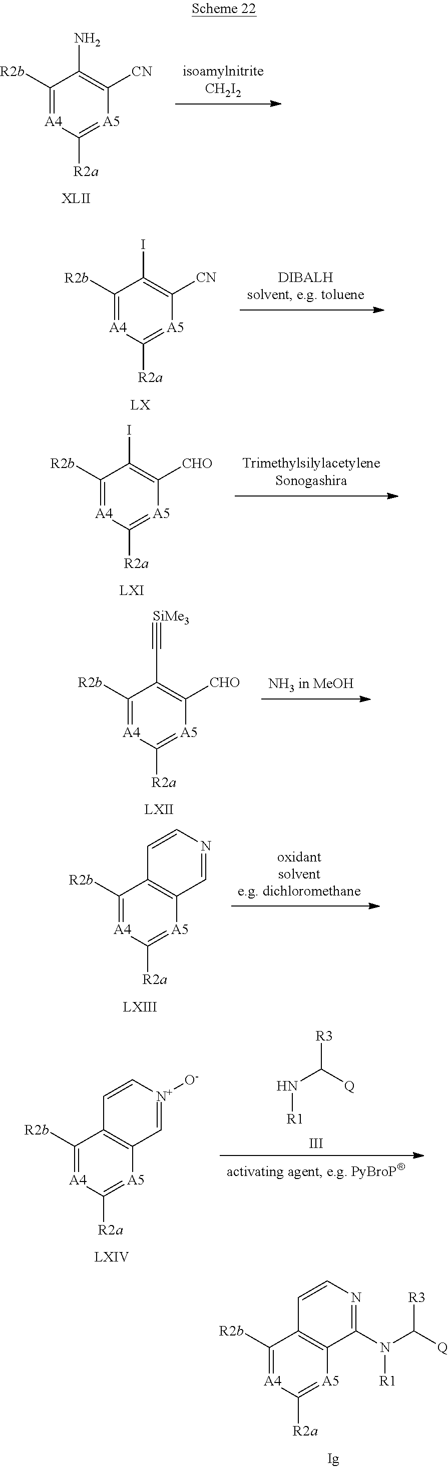

Compounds of formula Ig, wherein R¹, R^{2a}, R^{2b}, R³, A⁴, A⁵, Q is as described in formula (I), can be prepared according to Scheme 22. A compound of formula XLII, as prepared in Scheme 16, is treated with a diazotizing reagent, preferably isoamylnitrite, in diiodomethane solvent, at elevated temperature, preferably 80° C., as described in J. Org. Chem. 1990, 55, 2543, to provide intermediates of formula LX. Reduction of compounds of formula LX is achieved in the presence of a selective reductant, such as diisobutylaluminum hydride (DIBALH), in a solvent, such as toluene, at low temperatures, preferably −78° C., and gives a compound of formula LXI. Subsequent Sonogashira coupling in the presence of suitable palladium and copper catalysts, preferably bis(triphenylphosphine)palladium chloride and copper(I) iodide, with trimethylsilylacetylene, in a solvent, such as triethylamine, gives compounds of formula LXII. Cyclization with ammonia in methanol gives compounds of formula LXIII. Procedures are analogous to those described for example in Eur. J. Med. Chem. 2016, 118, 170. Treatment of compounds of formula LXIII with an oxidant, such as 3-chloro-benzenecarboperoxoic acid or hydrogen peroxide, in a solvent, preferably dichloromethane, gives N-oxides of formula LXIV. Such oxidations are well known to the person skilled in the art. Coupling of compounds of formula LXIV with amines of formula III in the presence of a suitable activator, such as bromotripyrrolidinophosphonium hexafluorophosphate (PyBroP®), potentially in the presence of base, such as N,N-diisopropylethylamine, analogous to procedure described in WO2016/123627, page 87, gives compounds of formula Ig.
Compounds of formula IId

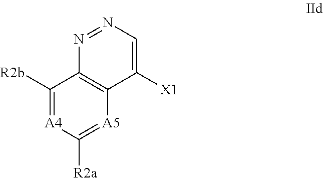

wherein X¹ is a leaving group, such as a halogen or sulfonate, for instance chloride, can be made, for example, as shown in Scheme 23.

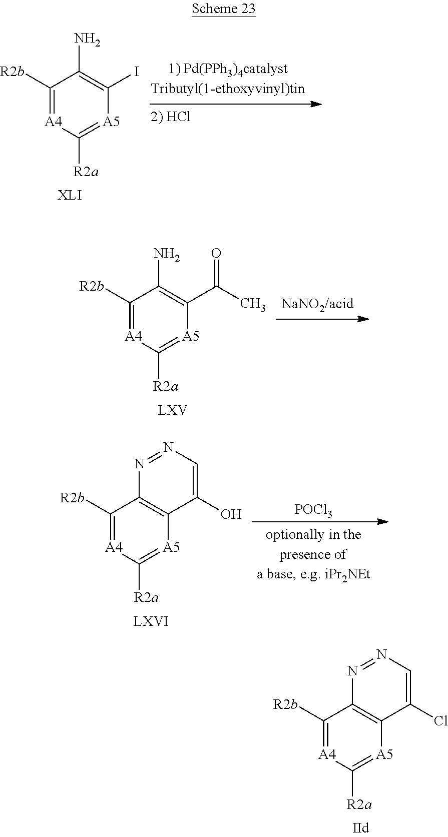

Compounds of formula IId, wherein R^{2a}, R^{2b}, A⁴, A⁵ are as described in formula (I), can be prepared according to Scheme 23. A compound of formula XLI, as prepared in Scheme 16, is treated with a palladium catalyst, preferably Pd(PPh₃)₄, along with tributyl(1-ethoxyvinyl)tin, at elevated temperature, preferably 105° C., as described in EP1782811, page 57, to provide intermediates of formula LXV. Treatment of compounds of formula LXV with aqueous sodium nitrite in the presence of acids, such as hydrochloric acid or a sulfuric acid/acetic acid mixture at low temperatures, preferably between 0 and 5° C., analogous to Bioorg. Med. Chem. Lett., 25, 919, gives compounds of formula LXVI. Compounds of formula IId are then obtained via chlorination of compounds of formula LXVI using chlorinating reagents, such as POCl₃, optionally in the presence of an amine base, such as N,N-diisopropylethylamine, well known to the person skilled in the art.
Compounds of formula IIe

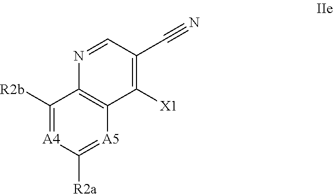

wherein X¹ is a leaving group, such as a halogen or sulfonate, for instance chloride, can be made, for example, as shown in Scheme 24.

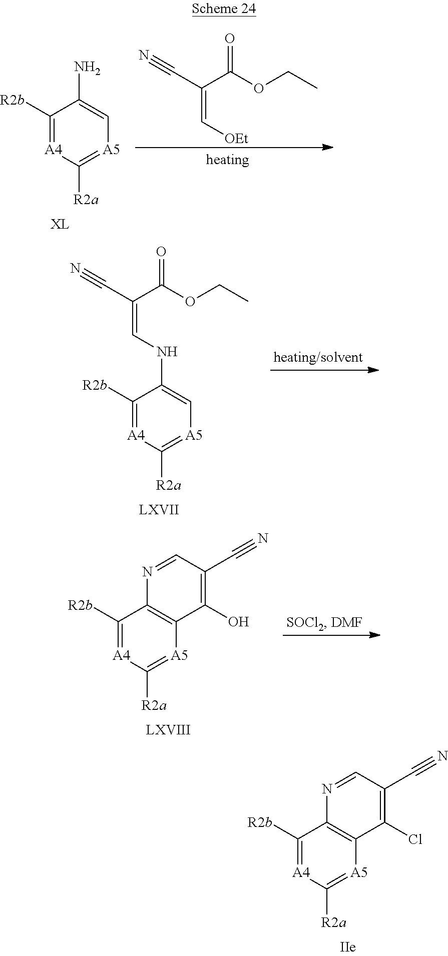

Compounds of formula IIe, wherein R^{2a}, R^{2b}, A⁴, A⁵ are as described in formula (I), can be prepared according to Scheme 24. A compound of formula XL is heated with ethyl 2-cyano-3-ethoxyacrylate at elevated temperature, preferably 140° C., as described in Tetrahedron Letters, 2015, 56, 5112, to provide intermediates of formula LXVII. Heating of compounds of formula LXVII at elevated temperatures, preferably between at 260° C., in a solvent, preferably diphenyl ether or diphenyl ether-biphenyl eutectic mixture (Dowtherm A®), gives compounds of formula LXVIII. Subsequent conversion to compounds of formula IIe is accomplished via according to methods known to the person skilled in the art, for example with thionyl chloride in presence of catalytic N,N-dimethylformamide at reflux, analogous to US2003/212276, page 15.
Compounds of the formula Ih

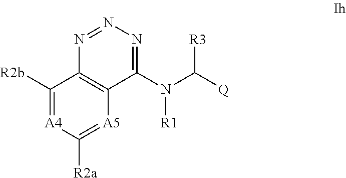

can be made, for example, as shown in Scheme 25.

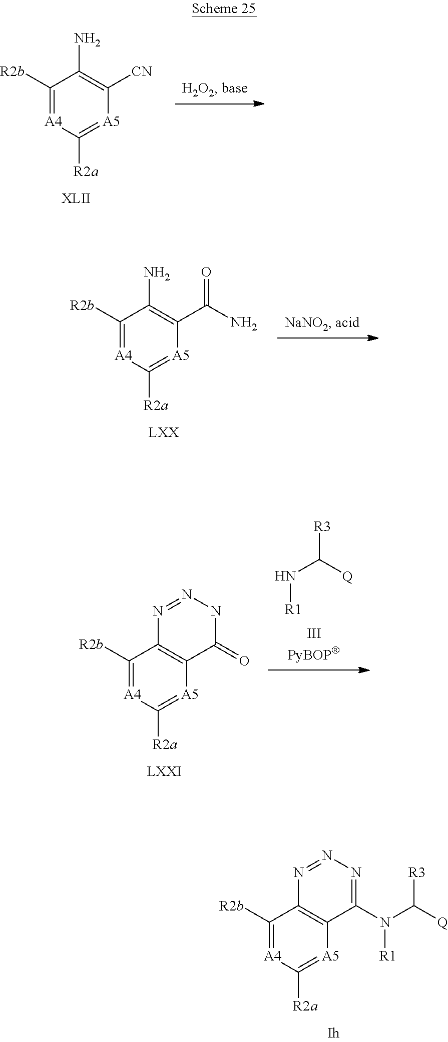

Compounds of formula Ih, wherein R¹, R^{2a}, R^{2b}, R³, A⁴, A⁵, Q is as described in formula (I), can be prepared according to Scheme 25. A compound of formula XLII, as prepared in Scheme 16, is treated with hydrogen peroxide, either as an aqueous solution or as an urea adduct, in methanol-water solvent, in the presence of base, preferably potassium carbonate, analogous to WO2011/4276, page 132, to provide intermediates of formula LXX. Treatment of compounds of formula LXX with aqueous sodium nitrite in the presence of acids, such as hydrochloric acid or a sulfuric acid/acetic acid mixture at low temperatures, preferably between 0 and 5° C., analogous to US2014/0275072, paragraph 133, gives compounds of formula LXXI. Coupling of compounds of formula LXXI with amines of formula III in the presence of a suitable activator, such as (benzotriazol-1-yloxy)tripyrrolidinophosphonium hexafluorophosphate (PyBoP®), in the presence of base, such as N,N-diisopropylethylamine, analogous to procedure described in WO2014/085528, page 55, gives compounds of formula Ih.
Compounds of the formula II

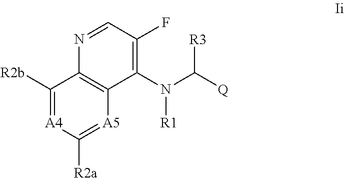

can be made, for example, as shown in Scheme 26.

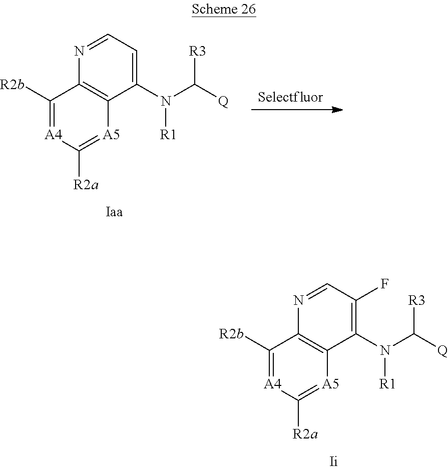

Compounds of formula Ii, wherein R¹, R^{2a}, R^{2b}, R³, A⁴, A⁵, Q is as described in formula (I), can be prepared according to Scheme 26. A compound of formula Iaa, as prepared in Scheme 1, 5 or 6, is treated with a fluorinating reagent, preferably 1-chloromethyl-4-fluoro-1,4-diazoniabicyclo[2.2.2]octane bis(tetrafluoroborate) (Selectfluor), in a solvent, preferably acetonitrile, analogous to WO2018/34917, page 91, to provide compounds of formula II.
Compounds of the formula Ij

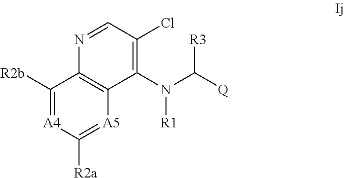

can be made, for example, as shown in Scheme 27.

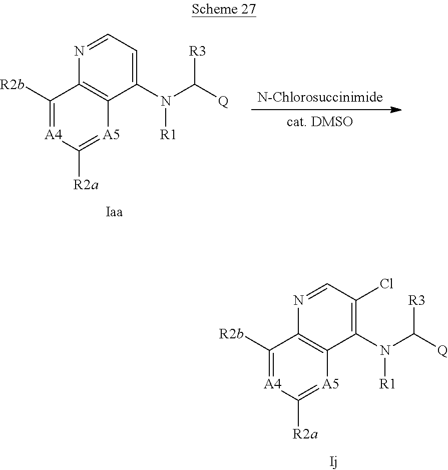

Compounds of formula Ij, wherein R¹, R^{2a}, R^{2b}, R³, A⁴, A⁵, Q is as described in formula (I), can be prepared according to Scheme 27. A compound of formula Iaa, as prepared in Scheme 1, 5 or 6, is treated with a chlorinating reagent, preferably N-chlorosuccinimide, in the presence of catalytic dimethyl sulfoxide (DMSO), in a solvent, preferably dichloromethane, analogous to Nature Catalysis, 2020, 3, 107, to provide compounds of formula Ij.
Certain compounds of formula III, or a salt thereof (such as a hydrohalide salt, preferably a hydrochloride or a hydrobromide salt, or any other equivalent salt), wherein R¹, R³ and Q have the same meaning as given above for compounds of the formula (I), are known in the literature. For example, compounds of the formula IIIa and IIIb, or a salt thereof as defined above, wherein R¹, R³, R^{4a}, R^{5a} and R^{5b} are as described in formula (I), particularly those compounds of the formula IIIa and IIIb, or a salt thereof as defined above, wherein R³ and R^{4a} are as described in formula (I) and in which R¹, R^{5a} and R^{5b} are hydrogen, can be prepared in analogy to descriptions found in WO 2021/069575.

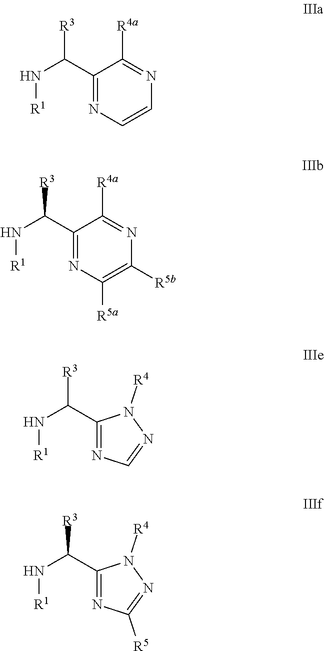

Similarly, compounds of the formula Ille and IIIf, or a salt thereof as defined above, wherein R¹, R³, R⁴ and R⁵ are as described in formula (I), particularly those compounds of the formula Ille and IIIf, or a salt thereof as defined above, wherein R³ and R⁴ are as described in formula (I) and in which R¹ is hydrogen and R⁵ is hydrogen, methyl or cyclopropyl, can be prepared in analogy to descriptions found for example in WO 2021/099303, WO 2021/105091, WO 2021/165195 and WO 2021/224323.

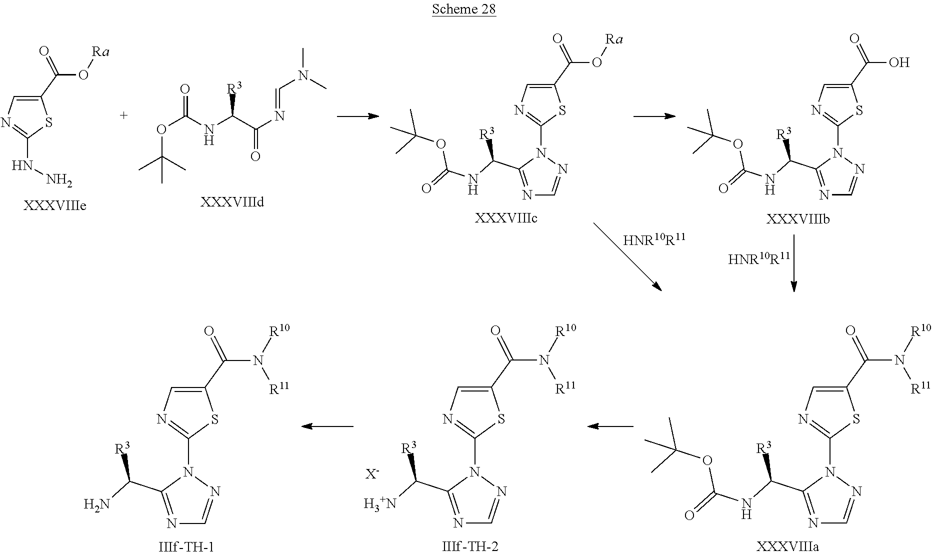

Compounds of the formula (IIIf-TH-1), a subset of compounds of formula (IIIf) wherein R¹ and R⁵ are hydrogen, R³ is as defined for compounds of the formula (I), and in which R⁴ is thiazolyl substituted with a single —C(O)NR¹⁰R¹¹, wherein R¹⁰ and R¹¹ are as defined for compounds of the formula (I), can be made (Scheme 28) from compounds of the formula (IIIf-TH-2), wherein R³, R¹⁰ and R¹¹ are as defined for compounds of the formula (I) and X⁻ is an anion, by treatment with a base, such as for example a hydroxide base or a carbonate base, for example sodium hydroxide or potassium carbonate, or an ion exchange resin. Such procedures are well known to a person skilled in the art, and known from the literature and text books. The anion X⁻ is the conjugate base of an acid, such as an inorganic acid, for instance hydrochloric acid, hydrobromic acid, hydrogen fluoride, hydrogen iodide, sulfuric acid, or the like, or of an organic acid, such as a carboxylic acid or a sulfonic acid, for instance trifluoroacetic acid, or methane sulfonic acid, or para-toluene sulfonic acid. A great number of such acids are known to a person skilled in the art.
Compounds of the formula (IIIf-TH-2), wherein R³, R¹⁰ and R¹¹ are as defined for compounds of the formula (I) and X⁻ is an anion, can be made from compounds of the formula (XXXVIIIa), wherein R³, R¹⁰ and R¹¹ are as defined for compounds of the formula (I), by treatment with an acid, such as the acids listed above. The reaction can be done neat or in a solvent, for instance an organic solvent, such as in methanol, tetrahydrofuran, dichloromethane or in dioxane, or in an inorganic solvent, such as in water, or in a mixture of such solvents. The reaction can be done in a temperature range between −100° C. and 200° C., more commonly between 0° C. and 150° C., such as, for example, at ambient temperature.
Compounds of the formula (XXXVIIIa), wherein R³, R¹⁰ and R¹¹ are as defined for compounds of the formula (I), can be made from compounds of the formula (XXXVIIIb), wherein R³ is as defined for compounds of the formula (I), by treatment with an amine compound of the formula HNR¹⁰R¹¹, or a salt thereof, wherein R¹⁰ and R¹¹ are as defined for compounds of the formula (I), under standard amide bond formation conditions known to a person skilled in the art. Typically therein, acid compounds of the formula (XXXVIIIb) will be activated to their corresponding acid chlorides with oxalyl chloride or thionyl chloride, in the presence of catalytic quantities of N,N-dimethylformamide (DMF), in inert solvents such as for instance dichloromethane (DCM) or tetrahydrofuran (THF), at temperatures between 0° C. to 100° C., preferably around 25° C. Such methods are known to those skilled in the art and described for example in Tetrahedron 2005, 61 (46), 10827-10852. Alternatively, it may be advantageous to carry out the reaction in the presence of a dehydration reagent, for instance a peptide coupling reagent, such as, for example, a carbodiimide, HATU (1-[bis(dimethylamino)-methylene]-1H-1,2,3-triazolo[4,5-b]pyridinium 3-oxide hexafluorophosphate, also known as Hexafluorophosphate Azabenzotriazole Tetramethyl Uronium) or propanephosphonic acid cyclic anhydride (T3P®). Such amidation reactions can be conducted neat or in a solvent, preferably in a solvent, such as an organic solvent, for instance acetonitrile, tetrahydrofuran, 2-methyltetrahydrofuran, ethyl acetate, N,N-dimethylacetamide or N,N-dimethylformamide, in a temperature range of −100 to +300° C., preferably between ambient temperature and 200° C., with or without the presence of a catalyst, for instance an acylation catalyst, such as 4-dimethylaminopyridine (DMAP), and with or without the addition of a base, such as an inorganic base, for instance sodium, potassium or cesium carbonate, or an organic base, such as, for example, triethylamine, diisopropylethylamine or pyridine. In the particular amidation reaction featuring amine compound of the formula HNR¹⁰R¹¹ wherein R¹⁰=R¹¹=hydrogen (i.e. HNR¹⁰R¹¹ is ammonia), it may be advantageous to use ammonia surrogates such as ammonium salts (for instance ammonium hydroxide or ammonium chloride) or silica gel-supported ammonium salts (for instance silica gel-supported ammonium chloride NH₄Cl/SiO₂ as described in Tetrahedron Letters 2005, 46, 6879-6882).
Compounds of the formula (XXXVIIIb), wherein R³ is as defined for compounds of the formula (I), can be prepared by saponification of the compounds of the formula (XXXVIIIc), wherein R³ is as defined for compounds of the formula (I), and in which Ra is C₁-C₆alkyl, under conditions known to a person skilled in the art (using for example conditions such as: aqueous sodium, potassium or lithium hydroxide in methanol, ethanol, tetrahydrofuran or dioxane at room temperature, or up to refluxing conditions).
Alternatively, compounds of the formula (XXXVIIIa), wherein R³, R¹⁰ and R¹¹ are as defined for compounds of the formula (I), can be made directly from compounds of the formula (XXXVIIIc), wherein R³ is as defined for compounds of the formula (I), and in which Ra is C₁-C₆alkyl, by reaction with amine compounds of the formula HNR¹⁰R¹¹, or a salt thereof, wherein R¹⁰ and R¹¹ are as defined for compounds of the formula (I), optionally in the presence of a base, such as an inorganic base, for instance sodium, potassium or cesium carbonate, or an organic base, such as, for example, triethylamine, diisopropylethylamine or pyridine, in a solvent, such as an organic solvent, for instance methanol, acetonitrile, tetrahydrofuran, 2-methyltetrahydrofuran, ethyl acetate, dimethyl sulfoxide, N,N-dimethylacetamide or N,N-dimethylformamide, in a temperature range of −100 to +300° C., preferably between ambient temperature and 100° C., optionally in the microwave.
Compounds of the formula (XXXVIIIc), wherein R³ is as defined for compounds of the formula (I), and in which Ra is C₁-C₆alkyl, can be prepared by reaction of compounds of the formula (XXXVIIIe), or a tautomer thereof, or a salt thereof, wherein Ra is C₁-C₆alkyl, with a compound of the formula (XXXVIIId), wherein R³ is as defined for compounds of the formula (I). The reaction can be done neat, or in a solvent, for instance an organic solvent, or in a mixture of solvents, such as in dioxane and acetic acid as a solvent. The reaction can be performed in the presence or in the absence of a drying agent, such as for example in the presence of molecular sieves, at a temperature between −100° C. and 200° C., more commonly between 0° C. and 150° C., such as, for example, at 80° C.
Compounds of the formula (XXXVIIId), wherein R³ is as defined for compounds of the formula (I), are known, for instance from WO2021/083936 or WO2021/165195, or they can be made in analogy to descriptions found therein. Compounds of the formula (XXXVIIIe), or a tautomer thereof, or a salt thereof, wherein Ra is C₁-C₆alkyl, are known or even commercially available, or they can be made by known methods.

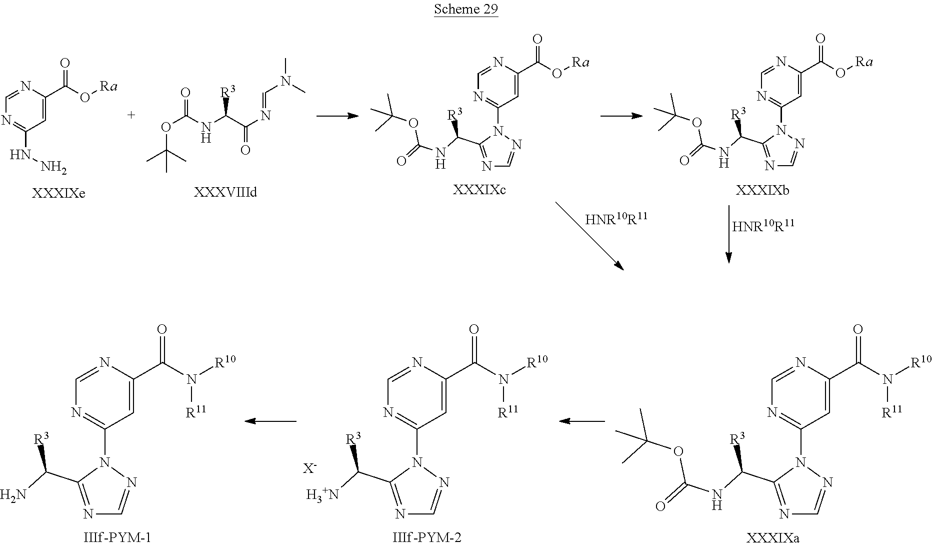

Similarly, compounds of the formula (IIIf-PYM-1), a subset of compounds of formula (IIIf) wherein R¹ and R⁵ are hydrogen, R³ is as defined for compounds of the formula (I), and in which R⁴ is pyrimidinyl substituted with a single —C(O)NR¹⁰R¹¹, wherein R¹⁰ and R¹¹ are as defined for compounds of the formula (I), or a salt thereof (IIIf-PYM-2), in which X⁻ is an anion as defined above in Scheme 28, can be made (Scheme 29) from compounds of the formula (XXXIXe), or a tautomer thereof, or a salt thereof, wherein Ra is C₁-C₆alkyl, and compounds of the formula (XXXVIIId), wherein R³ is as defined for compounds of the formula (I), following the chemistry and conditions described above in Scheme 28.
Compounds of the formula (XXXIXe), or a tautomer thereof, or a salt thereof, wherein Ra is C₁-C₆alkyl, are known or even commercially available, or they can be made by known methods.

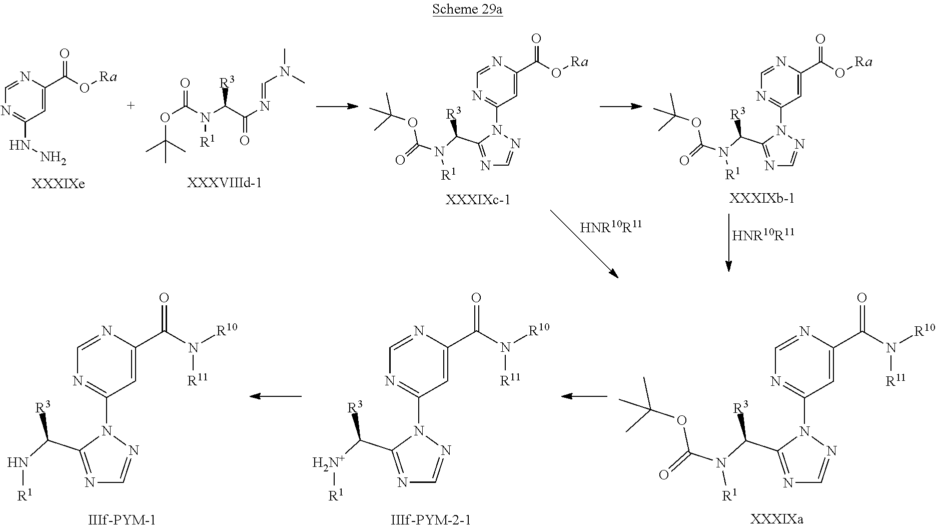

Similarly, compounds of the formula (IIIf-PYM-1-1), a subset of compounds of formula (IIIf) wherein R⁵ is hydrogen, R¹ and R³ are as defined for compounds of the formula (I), and in which R⁴ is pyrimidinyl substituted with a single —C(O)NR¹⁰R¹¹, wherein R¹⁰ and R¹¹ are as defined for compounds of the formula (I), or a salt thereof (IIIf-PYM-2-1), in which X⁻ is an anion as defined above in Scheme 28, can be made (Scheme 29a) from compounds of the formula (XXXIXe), or a tautomer thereof, or a salt thereof, wherein Ra is C₁-C₆alkyl, and compounds of the formula (XXXVIIId-1), wherein R¹ and R³ are as defined for compounds of the formula (I), following the chemistry and conditions described above in Schemes 28 and 29.
Compounds of the formula (XXXVIIId-1), wherein R¹ and R³ are as defined for compounds of the formula (I), are either known or they can be made in analogy to descriptions found for instance in WO2021/083936 or WO2021/165195.

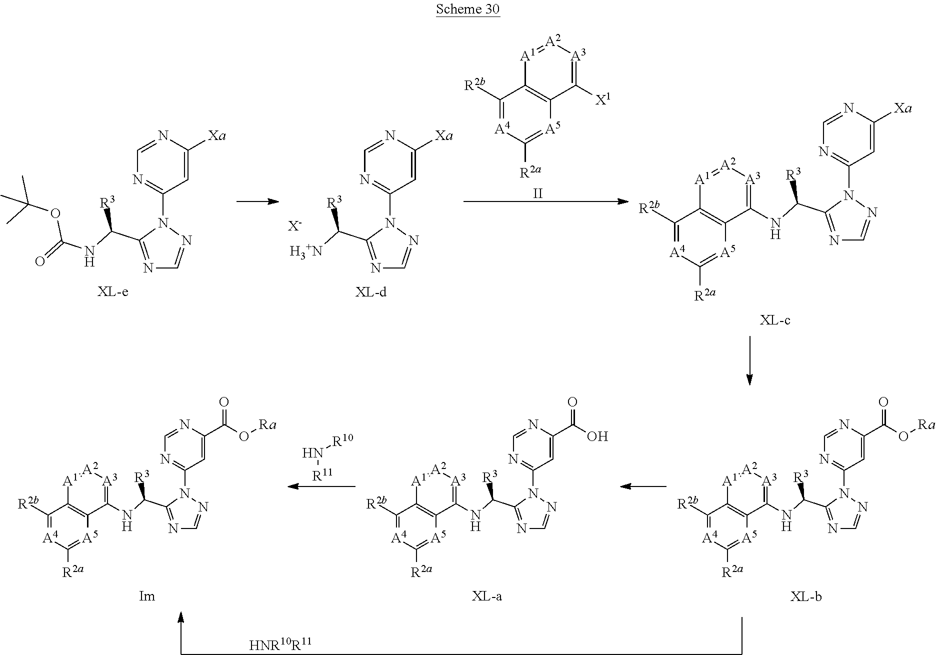

Compounds of the formula (Im), a subset of compounds of formula (I), wherein R^{2a}, R^{2b}, R³, A¹, A², A³, A⁴, A⁵, R¹⁰ and R¹¹ are as defined for compounds of the formula (I), can be prepared (Scheme 30) from compounds of the formula (XL-a), wherein R^{2a}, R^{2b}, R³, A¹, A², A³, A⁴ and A⁵ are as defined for compounds of the formula (I), by treatment with an amine compound of the formula HNR¹⁰R¹¹, or a salt thereof, wherein R¹⁰ and R¹¹ are as defined for compounds of the formula (I), under standard amide bond formation conditions known to a person skilled in the art. Typically therein, acid compounds of the formula (XL-a) are activated to their corresponding acid chlorides with oxalyl chloride or thionyl chloride, in the presence of catalytic quantities of N,N-dimethylformamide (DMF), in inert solvents such as for instance dichloromethane (DCM) or tetrahydrofuran (THF), at temperatures between 0° C. to 100° C., preferably around 25° C. Such methods are known to those skilled in the art and described for example in Tetrahedron 2005, 61 (46), 10827-10852. Alternatively, it may be advantageous to carry out the reaction in the presence of a dehydration reagent, for instance a peptide coupling reagent, such as, for example, a carbodiimide, HATU (1-[bis(dimethylamino)-methylene]-1H-1,2,3-triazolo[4,5-b]pyridinium 3-oxide hexafluorophosphate, also known as Hexafluorophosphate Azabenzotriazole Tetramethyl Uronium) or propanephosphonic acid cyclic anhydride (T3P®). Such amidation reactions can be conducted neat or in a solvent, preferably in a solvent, such as an organic solvent, for instance acetonitrile, tetrahydrofuran, 2-methyltetrahydrofuran, ethyl acetate, N,N-dimethylacetamide or N,N-dimethylformamide, in a temperature range of −100 to +300° C., preferably between ambient temperature and 200° C., with or without the presence of a catalyst, for instance an acylation catalyst, such as 4-dimethylaminopyridine (DMAP), and with or without the addition of a base, such as an inorganic base, for instance sodium, potassium or cesium carbonate, or an organic base, such as, for example, triethylamine, diisopropylethylamine or pyridine. In the particular amidation reaction featuring amine compound of the formula HNR¹⁰R¹¹ wherein R¹⁰=R¹¹=hydrogen (i.e. HNR¹⁰R¹¹ is ammonia), it may be advantageous to use ammonia surrogates such as ammonium salts (for instance ammonium hydroxide or ammonium chloride) or silica gel-supported ammonium salts (for instance silica gel-supported ammonium chloride NH₄Cl/SiO₂ as described in Tetrahedron Letters 2005, 46, 6879-6882).
Compounds of the formula (XL-a), wherein R^{2a}, R^{2b}, R³, A¹, A², A³, A⁴ and A⁵ are as defined for compounds of the formula (I), can be prepared by saponification of the compounds of the formula (XL-b), wherein R^{2a}, R^{2b}, R³, A¹, A², A³, A⁴ and A⁵ are as defined for compounds of the formula (I), and in which Ra is C₁-C₆alkyl or benzyl, under conditions known to a person skilled in the art (using for example conditions such as: aqueous sodium, potassium or lithium hydroxide in methanol, ethanol, tetrahydrofuran or dioxane at room temperature, or up to refluxing conditions).
Alternatively, compounds of the formula (Im), wherein R^{2a}, R^{2b}, R³, A¹, A², A³, A⁴, A⁵, R¹⁰ and R¹¹ are as defined for compounds of the formula (I), can be made directly from compounds of the formula (XL-b), wherein R^{2a}, R^{2b}, R³, A¹, A², A³, A⁴ and A⁵ are as defined for compounds of the formula (I), and in which Ra is C₁-C₆alkyl or benzyl, by reaction with amine compounds of the formula HNR¹⁰R¹¹, or a salt thereof, wherein R¹⁰ and R¹¹ are as defined for compounds of the formula (I), optionally in the presence of a base, such as an inorganic base, for instance sodium, potassium or cesium carbonate, or an organic base, such as, for example, triethylamine, diisopropylethylamine or pyridine, in a solvent, such as an organic solvent, for instance methanol, acetonitrile, tetrahydrofuran, 2-methyltetrahydrofuran, ethyl acetate, dimethyl sulfoxide, N,N-dimethylacetamide or N,N-dimethylformamide, in a temperature range of −100 to +300° C., preferably between ambient temperature and 100° C., optionally in the microwave.
Compounds of the formula (XL-b), wherein R^{2a}, R^{2b}, R³, A¹, A², A³, A⁴ and A⁵ are as defined for compounds of the formula (I), and in which Ra is C₁-C₆alkyl or benzyl, can be prepared by means of a carbonylation reaction on compounds of the formula (XL-c), wherein R^{2a}, R^{2b}, R³, A¹, A², A³, A⁴ and A⁵ are as defined for compounds of the formula (I), and in which Xa is a halogen, preferably Br, Cl or I (even more preferably Cl), in the presence of an alcohol RaOH, wherein Ra is C₁-C₆alkyl or benzyl. Typically in such a carbonylation reaction, the compounds of formula (XL-c) are reacted with carbon monoxide CO (usually under pressure, for example in the range of 5 to 200 bar), in the presence of a metal catalyst such as a palladium catalyst (for example: palladium(II) acetate, or [1,1′-bis(diphenylphosphino)ferrocene]dichloropalladium(II) PdCl₂(dppf), optionally in the presence of a phosphine ligand, preferably in the presence of a base such as triethylamine, diisopropylethylamine or pyridine, in an alcohol RaOH solvent (optionally in presence of an organic co-solvent), and at temperatures ranging between 0 and 250° C., preferably between room temperature and 200° C.
Compounds of the formula (XL-c), wherein R^{2a}, R^{2b}, R³, A¹, A², A³, A⁴ and A⁵ are as defined for compounds of the formula (I), and in which Xa is a halogen, preferably Br, Cl or I (even more preferably Cl), can be prepared by reacting compounds of the formula II, wherein R^{2a}, R^{2b}, A¹, A², A³, A⁴ and A⁵ are as defined for compounds of the formula (I), and wherein X¹ is a leaving group, such as a halogen or sulfonate, for instance chloride, with compounds of the formula (XL-d), or a free base thereof, wherein R³ is as defined for compounds of the formula (I), and in which Xa is a halogen, preferably Br, Cl or I (even more preferably C₁) and X⁻ is an anion as defined in Scheme 28, under analogous conditions already described above in Scheme 1.
Compounds of the formula (XL-d), or a free base thereof, wherein R³ is as defined for compounds of the formula (I), and in which Xa is a halogen, preferably Br, Cl or I (even more preferably Cl) and X⁻ is an anion as defined in Scheme 28, can be prepared from compounds of the formula (XL-e), wherein R³ is as defined for compounds of the formula (I), and in which Xa is a halogen, preferably Br, Cl or I (even more preferably Cl), by treatment with an acid under analogous conditions already described above in Scheme 28 (transformation XXXVIIIa into IIIf-TH-2).
Certain compounds of the formula (XL-e), wherein R³ is as defined for compounds of the formula (I), and in which Xa is a halogen, preferably Br, Cl or I (even more preferably Cl), are known for example from WO 2021/224323 or WO 2021/165195, or they can be made by known methods.

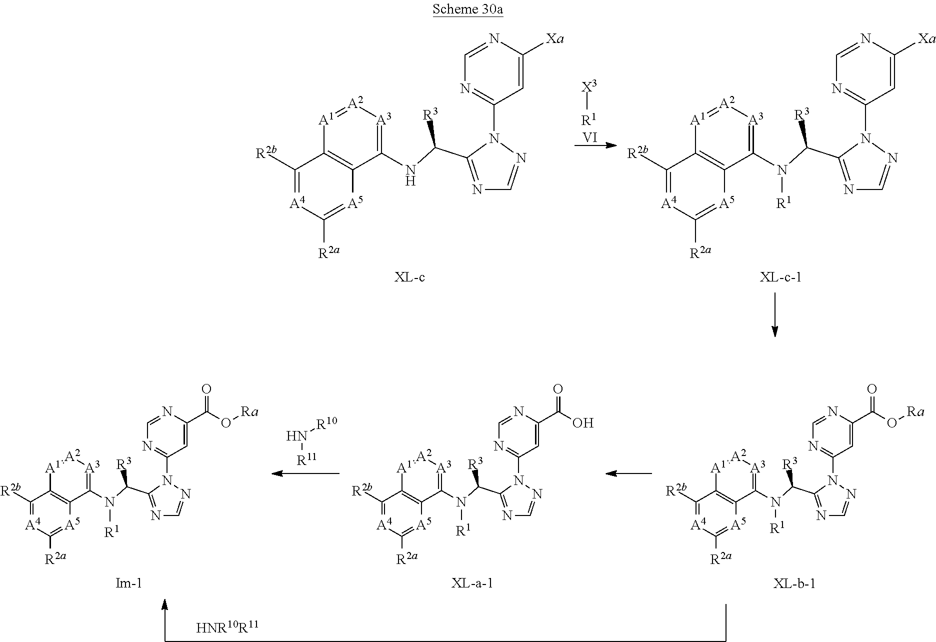

Similarly, compounds of the formula (Im-1), a subset of compounds of formula (I), wherein R¹, R^{2a}, R^{2b}, R³, A¹, A², A³, A⁴, A⁵, R¹⁰ and R¹¹ are as defined for compounds of the formula (I), can be prepared (Scheme 30a) from compounds of the formula (XL-c-1), wherein R¹, R^{2a}, R^{2b}, R³, A¹, A², A³, A⁴ and A⁵ are as defined for compounds of the formula (I), and in which Xa is a halogen, preferably Br, Cl or I (even more preferably Cl), following the chemistry and conditions described above in Scheme 30.
Compounds of the formula (XL-c-1), wherein R¹, R^{2a}, R^{2b}, R³, A¹, A², A³, A⁴ and A⁵ are as defined for compounds of the formula (I), and in which Xa is a halogen, preferably Br, Cl or I (even more preferably Cl), can be prepared by reacting compounds of the formula (XL-c), wherein R^{2a}, R^{2b}, R³, A¹, A², A³, A⁴ and A⁵ are as defined for compounds of the formula (I), and in which Xa is a halogen, preferably Br, Cl or I (even more preferably Cl), with compounds of the formula R¹—X³ (VI), wherein R¹ has the same meaning as given above for compounds of the formula (I), except that R¹ is different from hydrogen, and wherein X³ is a leaving group, such as a halogen or sulfonate, for instance a chloride, bromide, iodide or mesylate, under conditions already described above in Scheme 5.

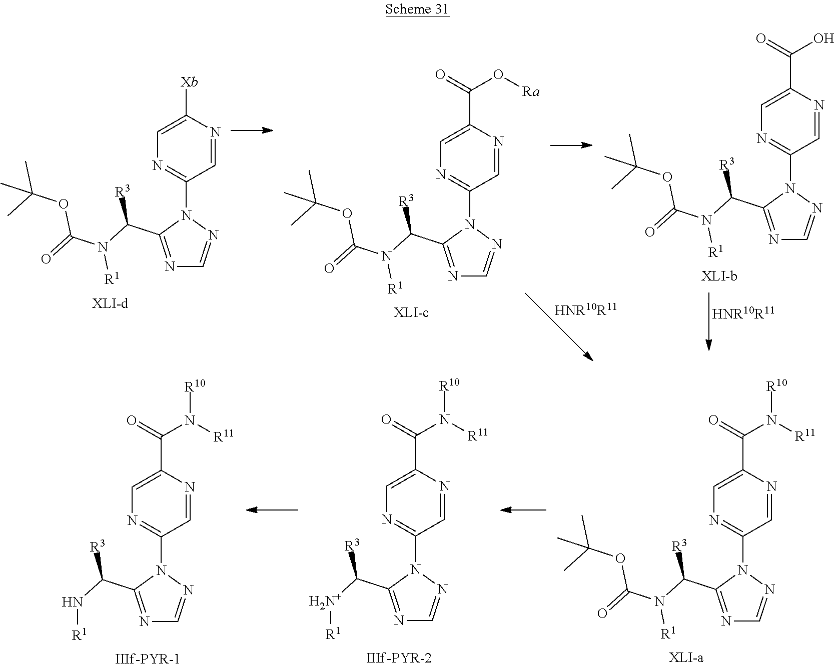

Compounds of the formula (IIIf-PYR-1), a subset of compounds of formula (IIIf) wherein R⁵ is hydrogen, R¹ and R³ are as defined for compounds of the formula (I), and in which R⁴ is pyrazinyl substituted with a single —C(O)NR¹⁰R¹¹, wherein R¹⁰ and R¹¹ are as defined for compounds of the formula (I), or a salt thereof (IIIf-PYR-2), in which X⁻ is an anion as defined above in Scheme 28, can be made (Scheme 31) from compounds of the formula (XLI-c), wherein R¹ and R³ are as defined for compounds of the formula (I), and in which Ra is C₁-C₆alkyl or benzyl, following the chemistry and conditions described above in Schemes 28, 29 and 29a.
Compounds of the formula (XLI-c), wherein R¹ and R³ are as defined for compounds of the formula (I), and in which Ra is C₁-C₆alkyl or benzyl, can be prepared by means of a carbonylation reaction on compounds of the formula (XLI-d), wherein R¹ and R³ are as defined for compounds of the formula (I), and in which Xb is a halogen, preferably Br, Cl or I (even more preferably Br or Cl), in the presence of an alcohol RaOH, wherein Ra is C₁-C₆alkyl or benzyl. Typically in such a carbonylation reaction, the compounds of formula (XLI-d) are reacted with carbon monoxide CO (usually under pressure, for example in the range of 5 to 200 bar), in the presence of a metal catalyst such as a palladium catalyst (for example: palladium(II) acetate, or [1,1′-bis(diphenylphosphino)ferrocene]dichloropalladium(II) PdCl₂(dppf)), optionally in the presence of a phosphine ligand, preferably in the presence of a base such as triethylamine, diisopropylethylamine or pyridine, in an alcohol RaOH solvent (optionally in presence of an organic co-solvent), and at temperatures ranging between 0 and 250° C., preferably between room temperature and 200° C.

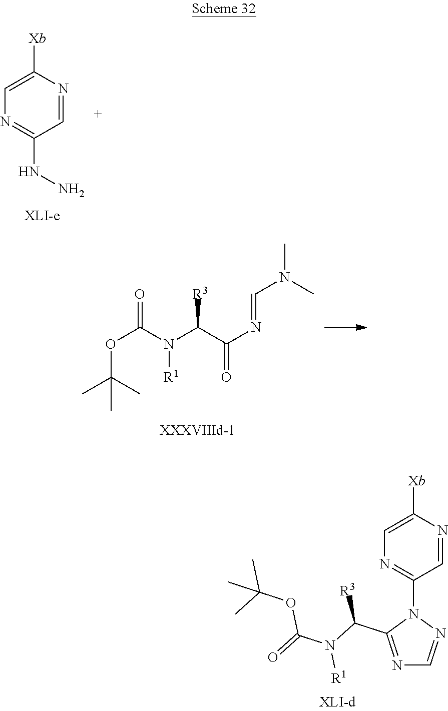

Compounds of the formula (XLI-d), wherein R¹ and R³ are as defined for compounds of the formula (I), and in which Xb is a halogen, preferably Br, Cl or I (even more preferably Br or Cl), can be prepared (Scheme 32) by reaction of compounds of the formula (XLI-e), or a tautomer thereof, or a salt thereof, wherein Xb is a halogen, preferably Br, Cl or I (even more preferably Br or Cl), with a compound of the formula (XXXVIIId-1), wherein R¹ and R³ are as defined for compounds of the formula (I). The reaction can be done neat, or in a solvent, for instance an organic solvent, such as dioxane or acetic acid, or a mixture thereof. The reaction can be performed in the presence or in the absence of a drying agent, such as for example in the presence of molecular sieves, at a temperature between −100° C. and 200° C., more commonly between 0° C. and 150° C., such as, for example, at 80° C.
Compounds of the formula (XLI-e), or a tautomer thereof, or a salt thereof, wherein Xb is a halogen, preferably Br, Cl or I (even more preferably Br or Cl), are known or even commercially available, or they can be made by known methods.

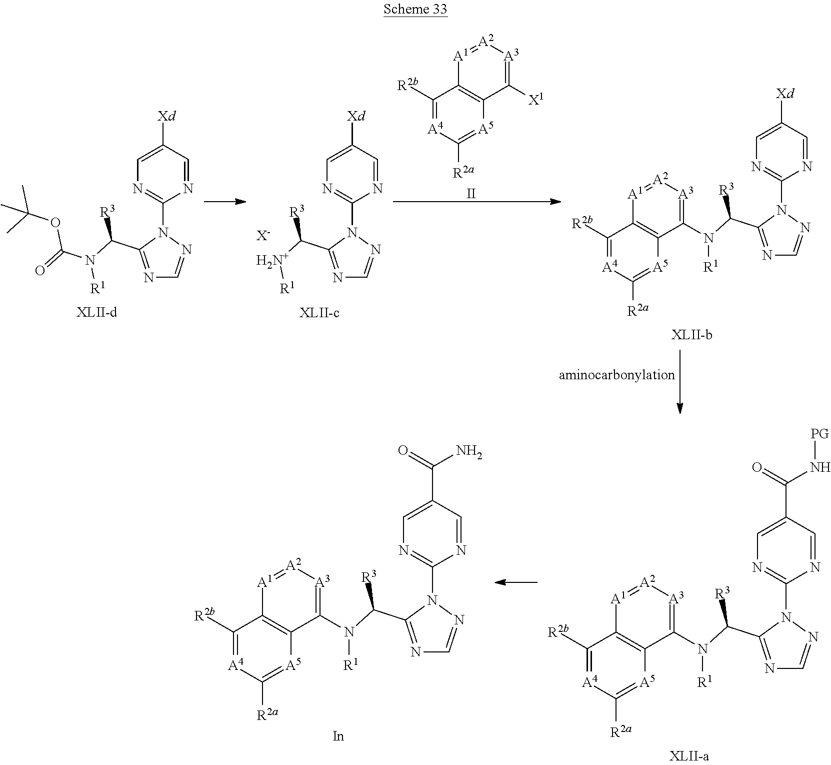

Compounds of the formula (In), a subset of compounds of formula (I), wherein R^{2a}, R^{2b}, R¹, R³, A¹, A², A³, A⁴ and A⁵ are as defined for compounds of the formula (I), can be prepared (Scheme 33) by deprotecting compounds of the formula (XLII-a), wherein R^{2a}, R^{2b}, R¹, R³, A¹, A², A³, A⁴ and A⁵ are as defined for compounds of the formula (I), and in which PG is a benzyl, a para-methoxybenzyl, a 3,4-dimethoxybenzyl or a 2,4-dimethoxybenzyl group. Such deprotection reactions are known to a person skilled in the art, and described in the literature, such as for example in: Protective Groups in Organic Synthesis, 3rd Edition Theodora W. Green (The Rowland Institute for Science) and Peter G. M. Wuts (Pharmacia and Upjohn Company), John Wiley & Sons, Inc., New York, NY. 1999, ISBN 0-471-16019-9. Benzylic deprotection may be achieved via catalytic hydrogenolysis, treatment with acids (such as for example trifluoroacetic) or under oxidative conditions by treatment with ceric ammonium nitrate (CAN) or 2,3-dichloro-5,6-dicyano-1,4-benzoquinone (DDQ). In particular, reaction of compounds of the formula (XLII-a), wherein PG is a methoxybenzyl group, with aqueous ceric ammonium nitrate, in a solvent such as acetonitrile and at temperatures between 0 to 100° C., preferably around room temperature, can generate compounds of the formula (In).
Compounds of the formula (XLII-a), wherein R^{2a}, R^{2b}, R¹, R³, A¹, A², A³, A⁴ and A⁵ are as defined for compounds of the formula (I), and in which PG is a benzyl, a para-methoxybenzyl, a 3,4-dimethoxybenzyl or a 2,4-dimethoxybenzyl group, can be prepared by means of an aminocarbonylation reaction of compounds of the formula (XLII-b), wherein R^{2a}, R^{2b}, R¹, R³, A¹, A², A³, A⁴ and A⁵ are as defined for compounds of the formula (I), and in which Xd is a halogen, preferably Br, C₁ or I (even more preferably Br), in the presence of benzylamine, para-methoxybenzylamine, 3,4-dimethoxybenzylamine or 2,4-dimethoxybenzylamine. Typically in such an aminocarbonylation reaction, the compounds of formula (XLII-b) are reacted with carbon monoxide CO (usually under pressure, for example in the range of 5 to 200 bar), in the presence of a metal catalyst such as a palladium catalyst (for example: palladium(II) acetate, bis(benzonitrile)palladium(II) chloride or [1,1′-bis(diphenylphosphino)ferrocene]-dichloropalladium(II) PdCl₂(dppf), optionally in the presence of a phosphine ligand (such as Xantphos), in the presence of above benzylamines, preferably in the presence of a base such as triethylamine, diisopropylethylamine or pyridine, in an inert solvent (such as toluene), and at temperatures ranging between 0 and 250° C., preferably between room temperature and 200° C.
Compounds of the formula (XLII-b), wherein R^{2a}, R^{2b}, R¹, R³, A¹, A², A³, A⁴ and A⁵ are as defined for compounds of the formula (I), and in which Xd is a halogen, preferably Br, Cl or I (even more preferably Br), can be prepared by reacting compounds of the formula II, wherein R^{2a}, R^{2b}, A¹, A², A³, A⁴ and A⁵ are as defined for compounds of the formula (I), and wherein X¹ is a leaving group, such as a halogen or sulfonate, for instance chloride, with compounds of the formula (XLII-c), or a free base thereof, wherein R¹ and R³ are as defined for compounds of the formula (I), and in which Xd is a halogen, preferably Br, Cl or I (even more preferably Br), and X⁻ is an anion as defined in Scheme 28, under analogous conditions already described above in Scheme 1.
Compounds of the formula (XLII-c), or a free base thereof, wherein R¹ and R³ are as defined for compounds of the formula (I), and in which Xd is a halogen, preferably Br, Cl or I (even more preferably Br), and X⁻ is an anion as defined in Scheme 28, can be prepared from compounds of the formula (XLII-d), wherein R¹ and R³ are as defined for compounds of the formula (I), and in which Xd is a halogen, preferably Br, Cl or I (even more preferably Br), by treatment with an acid under analogous conditions already described above in Scheme 28 (transformation XXXVIIIa into IIIf-TH-2).
Compounds of the formula (XLII-d), wherein R¹ and R³ are as defined for compounds of the formula (I), and in which Xd is a halogen, preferably Br, Cl or I (even more preferably Br), can be prepared by following descriptions herein above in Scheme 32 or in analogy to conditions found for example in WO 21/083936.
Depending on the procedure or the reaction conditions, the reactants can be reacted in the presence of a base. Examples of suitable bases are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylammonium hydroxide and 1,8-diazabicyclo[5.4.0]undec-7-ene (DBU).
The reactants can be reacted with each other as such, i.e. without adding a solvent or diluent. In most cases, however, it is advantageous to add an inert solvent or diluent or a mixture of these. If the reaction is carried out in the presence of a base, bases which are employed in excess, such as triethylamine, pyridine, N-methylmorpholine or N,N-diethylaniline, may also act as solvents or diluents.
The reactions are advantageously carried out in a temperature range from approximately −80° C. to approximately +140° C., preferably from approximately −30° C. to approximately +100° C., in many cases in the range between ambient temperature and approximately +80° C.
Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.
Salts of compounds of formula (I) can be prepared in a manner known per se. Thus, for example, acid addition salts of compounds of formula (I) are obtained by treatment with a suitable acid or a suitable ion exchanger reagent and salts with bases are obtained by treatment with a suitable base or with a suitable ion exchanger reagent.
Salts of compounds of formula (I) can be converted in the customary manner into the free compounds I, acid addition salts, for example, by treatment with a suitable basic compound or with a suitable ion exchanger reagent and salts with bases, for example, by treatment with a suitable acid or with a suitable ion exchanger reagent.
Salts of compounds of formula (I) can be converted in a manner known per se into other salts of compounds of formula (I), acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.
Depending on the procedure or the reaction conditions, the compounds of formula (I), which have salt-forming properties can be obtained in free form or in the form of salts.
The compounds of formula (I) and, where appropriate, the tautomers thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and hereinbelow, even when stereochemical details are not mentioned specifically in each case.
Diastereomer mixtures or racemate mixtures of compounds of formula (I), in free form or in salt form, which can be obtained depending on which starting materials and procedures have been chosen can be separated in a known manner into the pure diastereomers or racemates on the basis of the physicochemical differences of the components, for example by fractional crystallization, distillation and/or chromatography.
Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.
Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.
N-oxides can be prepared by reacting a compound of the formula (I) with a suitable oxidizing agent, for example the H₂O₂/urea adduct in the presence of an acid anhydride, e.g. trifluoroacetic anhydride. Such oxidations are known from the literature, for example from J. Med. Chem., 32 (12), 2561-73, 1989 or WO 2000/15615.
It is advantageous to isolate or synthesize in each case the biologically more effective isomer, for example enantiomer or diastereomer, or isomer mixture, for example enantiomer mixture or diastereomer mixture, if the individual components have a different biological activity.
The compounds of formula (I) and, where appropriate, the tautomers thereof, in each case in free form or in salt form, can, if appropriate, also be obtained in the form of hydrates and/or include other solvents, for example those which may have been used for the crystallization of compounds which are present in solid form.
The compounds of formula (I) according to the following Tables A-1 to A-71 can be prepared according to the methods described above. The examples which follow are intended to illustrate the invention and show preferred compounds of formula (I), in the form of a compound of formula I-A.

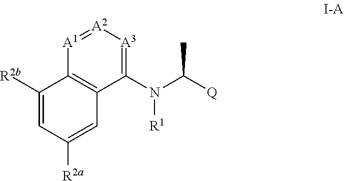

Table A-1 provides 38 compounds A-1.001 to A-1.038 of formula I-A wherein A¹ is N, A² is CH, A³ is N, R¹ is H, R^{2a} is CF₃, R^{2b} is CF₃ and Q is as defined in table Z. For example, compound A-13.003 is

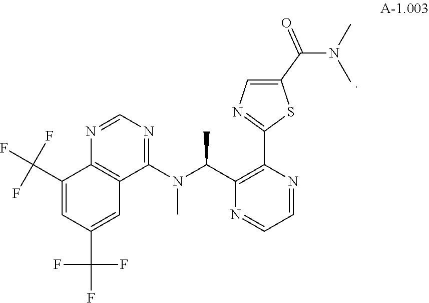 [SMILES: C[C@@H](c1nccnc1-c1ncc(C(=O)N(C)C)s1)N(C)c1ncnc2c(C(F)(F)F)cc(C(F)(F)F)cc12]

Table A-2 provides 38 compounds A-2.001 to A-2.038 of formula I-A wherein A¹ is N, A² is CH, A³ is N, R¹ is H, R^{2a} is CF₃, R^{2b} is Cl and Q is as defined in table Z.
Table A-3 provides 38 compounds A-3.001 to A-3.038 of formula I-A wherein A¹ is N, A² is CH, A³ is N, R¹ is H, R^{2a} is CF₃, R^{2b} is Br and Q is as defined in table Z.
Table A-4 provides 38 compounds A-4.001 to A-4.038 of formula I-A wherein A¹ is N, A² is CH, A³ is N, R¹ is H, R^{2a} is Cl, R^{2b} is CF₃ and Q is as defined in table Z.
Table A-5 provides 38 compounds A-5.001 to A-5.038 of formula I-A wherein A¹ is N, A² is CH, A³ is N, R¹ is H, R^{2a} is Cl, R^{2b} is Cl and Q is as defined in table Z.
Table A-6 provides 38 compounds A-6.001 to A-6.038 of formula I-A wherein A¹ is N, A² is CH, A³ is N, R¹ is H, R^{2a} is Cl, R^{2b} is Br and Q is as defined in table Z.
Table A-7 provides 38 compounds A-7.001 to A-7.038 of formula I-A wherein A¹ is N, A² is CH, A³ is N, R¹ is H, R^{2a} is Br, R^{2b} is CF₃ and Q is as defined in table Z.
Table A-8 provides 38 compounds A-8.001 to A-8.038 of formula I-A wherein A¹ is N, A² is CH, A³ is N, R¹ is H, R^{2a} is Br, R^{2b} is C and Q is as defined in table Z.
Table A-9 provides 38 compounds A-9.001 to A-9.038 of formula I-A wherein A¹ is N, A² is CH, A³ is N, R¹ is H, R^{2a} is Br, R^{2b} is Br and Q is as defined in table Z.
Table A-10 provides 38 compounds A-10.001 to A-10.038 of formula I-A wherein A¹ is N, A² is CH, A³ is N, R¹ is H, R^{2a} is SO₂—CF₃, R^{2b} is CF₃ and Q is as defined in table Z.
Table A-11 provides 38 compounds A-11.001 to A-11.038 of formula I-A wherein A¹ is N, A² is CH, A³ is N, R¹ is H, R^{2a} is SO₂—CF₃, R^{2b} is C and Q is as defined in table Z.
Table A-12 provides 38 compounds A-12.001 to A-12.038 of formula I-A wherein A¹ is N, A² is CH, A³ is N, R¹ is H, R^{2a} is SO₂—CF₃, R^{2b} is Br and Q is as defined in table Z.
Table A-13 provides 38 compounds A-13.001 to A-13.038 of formula I-A wherein A¹ is N, A² is CH, A³ is N, R¹ is CH₃, R^{2a} is CF₃, R^{2b} is CF₃ and Q is as defined in table Z.
Table A-14 provides 38 compounds A-14.001 to A-14.038 of formula I-A wherein A¹ is N, A² is CH, A³ is N, R¹ is CH₃, R^{2a} is CF₃, R^{2b} is Cl and Q is as defined in table Z.
Table A-15 provides 38 compounds A-15.001 to A-15.038 of formula I-A wherein A¹ is N, A² is CH, A³ is N, R¹ is CH₃, R^{2a} is CF₃, R^{2b} is Br and Q is as defined in table Z.
Table A-16 provides 38 compounds A-16.001 to A-16.038 of formula I-A wherein A¹ is N, A² is CH, A³ is N, R¹ is CH₃, R^{2a} is Cl, R^{2b} is CF₃ and Q is as defined in table Z.
Table A-17 provides 38 compounds A-17.001 to A-17.038 of formula I-A wherein A¹ is N, A² is CH, A³ is N, R¹ is CH₃, R^{2a} is Cl, R^{2b} is Cl and Q is as defined in table Z.
Table A-18 provides 38 compounds A-18.001 to A-18.038 of formula I-A wherein A¹ is N, A² is CH, A³ is N, R¹ is CH₃, R^{2a} is Cl, R^{2b} is Br and Q is as defined in table Z.
Table A-19 provides 38 compounds A-19.001 to A-19.038 of formula I-A wherein A¹ is N, A² is CH, A³ is N, R¹ is CH₃, R^{2a} is Br, R^{2b} is CF₃ and Q is as defined in table Z.
Table A-20 provides 38 compounds A-20.001 to A-20.038 of formula I-A wherein A¹ is N, A² is CH, A³ is N, R¹ is CH₃, R^{2a} is Br, R^{2b} is Cl and Q is as defined in table Z.
Table A-21 provides 38 compounds A-21.001 to A-21.038 of formula I-A wherein A¹ is N, A² is CH, A³ is N, R¹ is CH₃, R^{2a} is Br, R^{2b} is Br and Q is as defined in table Z.
Table A-22 provides 38 compounds A-22.001 to A-22.038 of formula I-A wherein A¹ is N, A² is CH, A³ is N, R¹ is CH₃, R^{2a} is SO₂—CF₃, R^{2b} is CF₃ and Q is as defined in table Z.
Table A-23 provides 38 compounds A-23.001 to A-23.038 of formula I-A wherein A¹ is N, A² is CH, A³ is N, R¹ is CH₃, R^{2a} is SO₂—CF₃, R^{2b} is C and Q is as defined in table Z.
Table A-24 provides 38 compounds A-24.001 to A-24.038 of formula I-A wherein A¹ is N, A² is CH, A³ is N, R¹ is CH₃, R^{2a} is SO₂—CF₃, R^{2b} is Br and Q is as defined in table Z.
Table A-25 provides 38 compounds A-25.001 to A-25.038 of formula I-A wherein A¹ is N, A² is CH, A³ is N, R¹ is CH₂-cyclopropyl, R^{2a} is CF₃, R^{2b} is CF₃ and Q is as defined in table Z.
Table A-26 provides 38 compounds A-26.001 to A-26.038 of formula I-A wherein A¹ is N, A² is CH, A³ is N, R¹ is CH₂-cyclopropyl, R^{2a} is CF₃, R^{2b} is C and Q is as defined in table Z.
Table A-27 provides 38 compounds A-27.001 to A-27.038 of formula I-A wherein A¹ is N, A² is CH, A³ is N, R¹ is CH₂-cyclopropyl, R^{2a} is CF₃, R^{2b} is Br and Q is as defined in table Z.
Table A-28 provides 38 compounds A-28.001 to A-28.038 of formula I-A wherein A¹ is N, A² is CH, A³ is N, R¹ is CH₂-cyclopropyl, R^{2a} is Cl, R^{2b} is CF₃ and Q is as defined in table Z.
Table A-29 provides 38 compounds A-29.001 to A-29.038 of formula I-A wherein A¹ is N, A² is CH, A³ is N, R¹ is CH₂-cyclopropyl, R^{2a} is Cl, R^{2b} is C and Q is as defined in table Z.
Table A-30 provides 38 compounds A-30.001 to A-30.038 of formula I-A wherein A¹ is N, A² is CH, A³ is N, R¹ is CH₂-cyclopropyl, R^{2a} is Cl, R^{2b} is Br and Q is as defined in table Z.
Table A-31 provides 38 compounds A-31.001 to A-31.038 of formula I-A wherein A¹ is N, A² is CH, A³ is N, R¹ is CH₂-cyclopropyl, R^{2a} is Br, R^{2b} is CF₃ and Q is as defined in table Z.
Table A-32 provides 38 compounds A-32.001 to A-32.038 of formula I-A wherein A¹ is N, A² is CH, A³ is N, R¹ is CH₂-cyclopropyl, R^{2a} is Br, R^{2b} is Cl and Q is as defined in table Z.
Table A-33 provides 38 compounds A-33.001 to A-33.038 of formula I-A wherein A¹ is N, A² is CH, A³ is N, R¹ is CH₂-cyclopropyl, R^{2a} is Br, R^{2b} is Br and Q is as defined in table Z.
Table A-34 provides 38 compounds A-34.001 to A-34.038 of formula I-A wherein A¹ is N, A² is CH, A³ is N, R¹ is CH₂-cyclopropyl, R^{2a} is SO₂—CF₃, R^{2b} is CF₃ and Q is as defined in table Z.
Table A-35 provides 38 compounds A-35.001 to A-35.038 of formula I-A wherein A¹ is N, A² is CH, A³ is N, R¹ is CH₂-cyclopropyl, R^{2a} is SO₂—CF₃, R^{2b} is Cl and Q is as defined in table Z.
Table A-36 provides 38 compounds A-36.001 to A-36.038 of formula I-A wherein A¹ is N, A² is CH, A³ is N, R¹ is CH₂-cyclopropyl, R^{2a} is SO₂—CF₃, R^{2b} is Br and Q is as defined in table Z.
Table A-37 provides 38 compounds A-37.001 to A-37.038 of formula I-A wherein A¹ is N, A² is CH, A³ is N, R¹ is H, R^{2a} is CF₃, R^{2b} is SO₂—CF₃ and Q is as defined in table Z.
Table A-38 provides 38 compounds A-38.001 to A-38.038 of formula I-A wherein A¹ is N, A² is CH, A³ is N, R¹ is H, R^{2a} is Cl, R^{2b} is SO₂—CF₃ and Q is as defined in table Z.
Table A-39 provides 38 compounds A-39.001 to A-39.038 of formula I-A wherein A¹ is N, A² is CH, A³ is N, R¹ is H, R^{2a} is Br, R^{2b} is SO₂—CF₃ and Q is as defined in table Z.
Table A-40 provides 38 compounds A-40.001 to A-40.038 of formula I-A wherein A¹ is N, A² is CH, A³ is N, R¹ is H, R^{2a} is Cl, R^{2b} is I and Q is as defined in table Z.
Table A-41 provides 38 compounds A-41.001 to A-41.038 of formula I-A wherein A¹ is N, A² is CH, A³ is N, R¹ is H, R^{2a} is I, R^{2b} is C and Q is as defined in table Z.
Table A-42 provides 38 compounds A-42.001 to A-42.038 of formula I-A wherein A¹ is N, A² is CH, A³ is N, R¹ is H, R^{2a} is Br, R^{2b} is I and Q is as defined in table Z.
Table A-43 provides 38 compounds A-43.001 to A-43.038 of formula I-A wherein A¹ is N, A² is CH, A³ is N, R¹ is H, R^{2a} is I, R^{2b} is Br and Q is as defined in table Z.
Table A-44 provides 38 compounds A-44.001 to A-44.038 of formula I-A wherein A¹ is N, A² is CH, A³ is N, R¹ is H, R^{2a} is CF₃, R^{2b} is I and Q is as defined in table Z.
Table A-45 provides 38 compounds A-45.001 to A-45.038 of formula I-A wherein A¹ is N, A² is CH, A³ is N, R¹ is H, R^{2a} is I, R^{2b} is CF₃ and Q is as defined in table Z.
Table A-46 provides 38 compounds A-46.001 to A-46.038 of formula I-A wherein A¹ is N, A² is CH, A³ is N, R¹ is CH₃, R^{2a} is CF₃, R^{2b} is SO₂—CF₃ and Q is as defined in table Z.
Table A-47 provides 38 compounds A-47.001 to A-47.038 of formula I-A wherein A¹ is N, A² is CH, A³ is N, R¹ is CH₃, R^{2a} is Cl, R^{2b} is SO₂—CF₃ and Q is as defined in table Z.
Table A-48 provides 38 compounds A-48.001 to A-48.038 of formula I-A wherein A¹ is N, A² is CH, A³ is N, R¹ is CH₃, R^{2a} is Br, R^{2b} is SO₂—CF₃ and Q is as defined in table Z.
Table A-49 provides 38 compounds A-49.001 to A-49.038 of formula I-A wherein A¹ is N, A² is CH, A³ is N, R¹ is CH₃, R^{2a} is Cl, R^{2b} is I and Q is as defined in table Z.
Table A-50 provides 38 compounds A-50.001 to A-50.038 of formula I-A wherein A¹ is N, A² is CH, A³ is N, R¹ is CH₃, R^{2a} is I, R^{2b} is C and Q is as defined in table Z.
Table A-51 provides 38 compounds A-51.001 to A-51.038 of formula I-A wherein A¹ is N, A² is CH, A³ is N, R¹ is CH₃, R^{2a} is Br, R^{2b} is I and Q is as defined in table Z.
Table A-52 provides 38 compounds A-52.001 to A-52.038 of formula I-A wherein A¹ is N, A² is CH, A³ is N, R¹ is CH₃, R^{2a} is I, R^{2b} is Br and Q is as defined in table Z.
Table A-53 provides 38 compounds A-53.001 to A-53.038 of formula I-A wherein A¹ is N, A² is CH, A³ is N, R¹ is CH₃, R^{2a} is CF₃, R^{2b} is I and Q is as defined in table Z.
Table A-54 provides 38 compounds A-54.001 to A-54.038 of formula I-A wherein A¹ is N, A² is CH, A³ is N, R¹ is CH₃, R^{2a} is 1, R^{2b} is CF₃ and Q is as defined in table Z.
Table A-55 provides 38 compounds A-55.001 to A-55.038 of formula I-A wherein A¹ is N, A² is CH, A³ is N, R¹ is CH₂-cyclopropyl, R^{2a} is CF₃, R^{2b} is SO₂—CF₃ and Q is as defined in table Z.
Table A-56 provides 38 compounds A-56.001 to A-56.038 of formula I-A wherein A¹ is N, A² is CH, A³ is N, R¹ is CH₂-cyclopropyl, R^{2a} is Cl, R^{2b} is SO₂—CF₃ and Q is as defined in table Z.
Table A-57 provides 38 compounds A-57.001 to A-57.038 of formula I-A wherein A¹ is N, A² is CH, A³ is N, R¹ is CH₂-cyclopropyl, R^{2a} is Br, R^{2b} is SO₂—CF₃ and Q is as defined in table Z.
Table A-58 provides 38 compounds A-58.001 to A-58.038 of formula I-A wherein A¹ is N, A² is CH, A³ is N, R¹ is CH₂-cyclopropyl, R^{2a} is Cl, R^{2b} is I and Q is as defined in table Z.
Table A-59 provides 38 compounds A-59.001 to A-59.038 of formula I-A wherein A¹ is N, A² is CH, A³ is N, R¹ is CH₂-cyclopropyl, R^{2a} is I, R^{2b} is C and Q is as defined in table Z.
Table A-60 provides 38 compounds A-60.001 to A-60.038 of formula I-A wherein A¹ is N, A² is CH, A³ is N, R¹ is CH₂-cyclopropyl, R^{2a} is Br, R^{2b} is I and Q is as defined in table Z.
Table A-61 provides 38 compounds A-61.001 to A-61.038 of formula I-A wherein A¹ is N, A² is CH, A³ is N, R¹ is CH₂-cyclopropyl, R^{2a} is I, R^{2b} is Br and Q is as defined in table Z.
Table A-62 provides 38 compounds A-62.001 to A-62.038 of formula I-A wherein A¹ is N, A² is CH, A³ is N, R¹ is CH₂-cyclopropyl, R^{2a} is CF₃, R^{2b} is I and Q is as defined in table Z.
Table A-63 provides 38 compounds A-63.001 to A-63.038 of formula I-A wherein A¹ is N, A² is CH, A³ is N, R¹ is CH₂-cyclopropyl, R^{2a} is I, R^{2b} is CF₃ and Q is as defined in table Z.
Table A-64 provides 38 compounds A-64.001 to A-64.038 of formula I-A wherein A¹ is N, A² is CH, A³ is N, R¹ is H, R^{2a} is SO₂—CHF₂, R^{2b} is C and Q is as defined in table Z.
Table A-65 provides 38 compounds A-65.001 to A-65.038 of formula I-A wherein A¹ is N, A² is CH, A³ is N, R¹ is H, R^{2a} is SO₂—CHF₂, R^{2b} is Br and Q is as defined in table Z.
Table A-66 provides 38 compounds A-66.001 to A-66.038 of formula I-A wherein A¹ is N, A² is CH, A³ is N, R¹ is CH₃, R^{2a} is SO₂—CHF₂, R^{2b} is C and Q is as defined in table Z.
Table A-67 provides 38 compounds A-67.001 to A-67.038 of formula I-A wherein A¹ is N, A² is CH, A³ is N, R¹ is CH₃, R^{2a} is SO₂—CHF₂, R^{2b} is Br and Q is as defined in table Z.
Table A-68 provides 38 compounds A-68.001 to A-68.038 of formula I-A wherein A¹ is N, A² is CH, A³ is N, R¹ is H, R^{2a} is Cl, R^{2b} is SO₂—CHF₂ and Q is as defined in table Z.
Table A-69 provides 38 compounds A-69.001 to A-69.038 of formula I-A wherein A¹ is N, A² is CH, A³ is N, R¹ is H, R^{2a} is Br, R^{2b} is SO₂—CHF₂ and Q is as defined in table Z.
Table A-70 provides 38 compounds A-70.001 to A-70.038 of formula I-A wherein A¹ is N, A² is CH, A³ is N, R¹ is CH₃, R^{2a} is C, R^{2b} is SO₂—CHF₂ and Q is as defined in table Z.
Table A-71 provides 38 compounds A-71.001 to A-71.038 of formula I-A wherein A¹ is N, A² is CH, A³ is N, R¹ is CH₃, R^{2a} is Br, R^{2b} is SO₂—CHF₂ and Q is as defined in table Z.

TABLE Z
Substituent definitions of Q

| Index | Q |
|---|---|
| 1 | 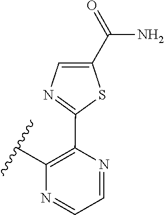 [SMILES: *c1nccnc1-c1ncc(C(N)=O)s1] |
| 2 | 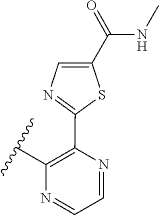 [SMILES: *c1nccnc1-c1ncc(C(=O)NC)s1] |
| 3 | 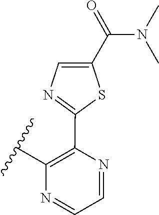 [SMILES: *c1nccnc1-c1ncc(C(=O)N(C)C)s1] |
| 4 | 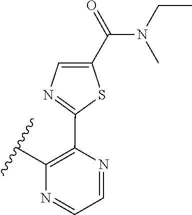 [SMILES: *c1nccnc1-c1ncc(C(=O)N(C)CC)s1] |
| 5 | 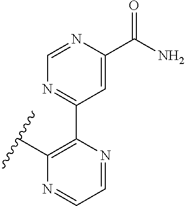 [SMILES: *c1nccnc1-c1cc(C(N)=O)ncn1] |
| 6 | 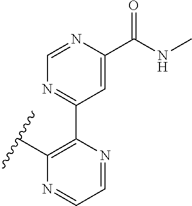 [SMILES: *c1nccnc1-c1cc(C(=O)NC)ncn1] |
| 7 | 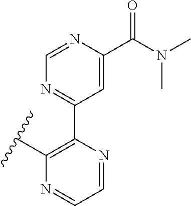 [SMILES: *c1nccnc1-c1cc(C(=O)N(C)C)ncn1] |
| 8 | 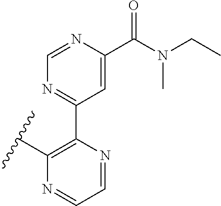 [SMILES: *c1nccnc1-c1cc(C(=O)N(C)CC)ncn1] |
| 9 | 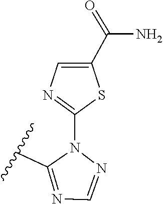 [SMILES: *c1ncnn1-c1ncc(C(N)=O)s1] |
| 10 | 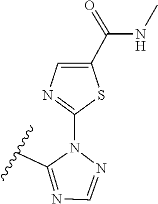 [SMILES: *c1ncnn1-c1ncc(C(=O)NC)s1] |
| 11 | 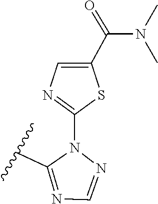 [SMILES: *c1ncnn1-c1ncc(C(=O)N(C)C)s1] |
| 12 | 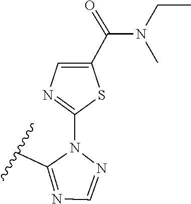 [SMILES: *c1ncnn1-c1ncc(C(=O)N(C)CC)s1] |
| 13 | 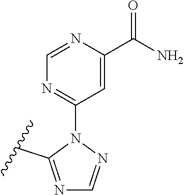 [SMILES: *c1ncnn1-c1cc(C(N)=O)ncn1] |
| 14 | 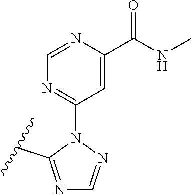 [SMILES: *c1ncnn1-c1cc(C(=O)NC)ncn1] |
| 15 | 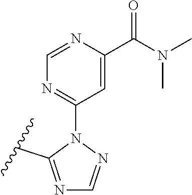 [SMILES: *c1ncnn1-c1cc(C(=O)N(C)C)ncn1] |
| 16 | 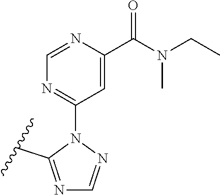 [SMILES: *c1ncnn1-c1cc(C(=O)N(C)CC)ncn1] |
| 17 | 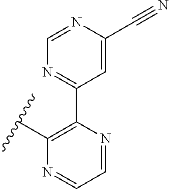 [SMILES: *c1nccnc1-c1cc(C#N)ncn1] |
| 18 | 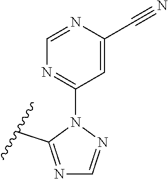 [SMILES: *c1ncnn1-c1cc(C#N)ncn1] |
| 19 | 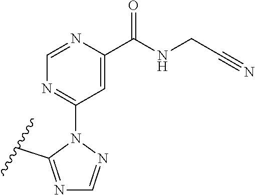 [SMILES: *c1ncnn1-c1cc(C(=O)NCC#N)ncn1] |
| 20 | 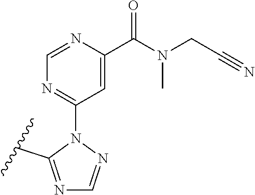 [SMILES: *c1ncnn1-c1cc(C(=O)N(C)CC#N)ncn1] |
| 21 | 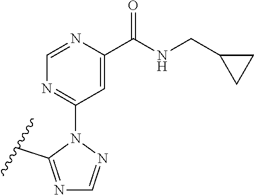 [SMILES: *c1ncnn1-c1cc(C(=O)NCC2CC2)ncn1] |
| 22 | 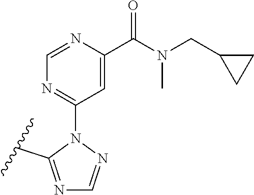 [SMILES: *c1ncnn1-c1cc(C(=O)N(C)CC2CC2)ncn1] |
| 23 | 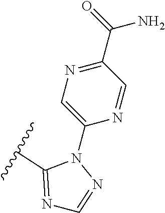 [SMILES: *c1ncnn1-c1cnc(C(N)=O)cn1] |
| 24 | 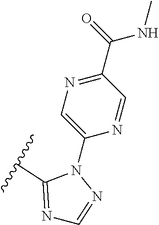 [SMILES: *c1ncnn1-c1cnc(C(=O)NC)cn1] |
| 25 | 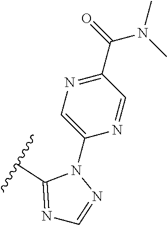 [SMILES: *c1ncnn1-c1cnc(C(=O)N(C)C)cn1] |
| 26 | 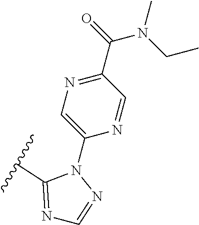 [SMILES: *c1ncnn1-c1cnc(C(=O)N(C)CC)cn1] |
| 27 | 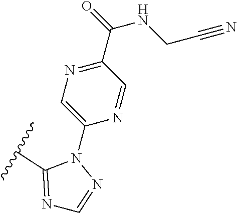 [SMILES: *c1ncnn1-c1cnc(C(=O)NCC#N)cn1] |
| 28 | 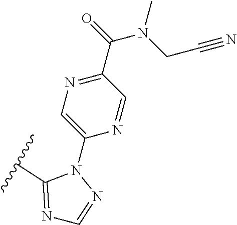 [SMILES: *c1ncnn1-c1cnc(C(=O)N(C)CC#N)cn1] |
| 29 | 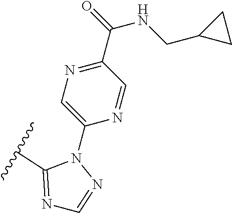 [SMILES: *c1ncnn1-c1cnc(C(=O)NCC2CC2)cn1] |
| 30 | 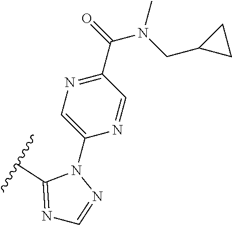 [SMILES: *c1ncnn1-c1cnc(C(=O)N(C)CC2CC2)cn1] |
| 31 | 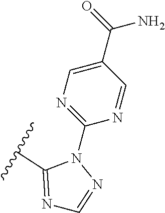 [SMILES: *c1ncnn1-c1ncc(C(N)=O)cn1] |
| 32 | 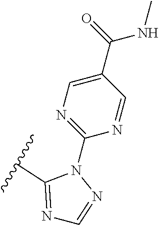 [SMILES: *c1ncnn1-c1ncc(C(=O)NC)cn1] |
| 33 | 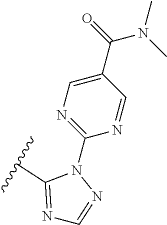 [SMILES: *c1ncnn1-c1ncc(C(=O)N(C)C)cn1] |
| 34 | 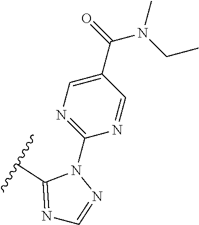 [SMILES: *c1ncnn1-c1ncc(C(=O)N(C)CC)cn1] |
| 35 | 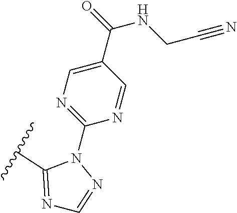 [SMILES: *c1ncnn1-c1ncc(C(=O)NCC#N)cn1] |
| 36 | 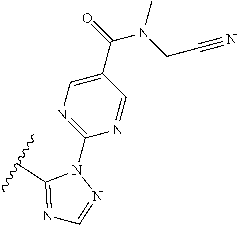 [SMILES: *c1ncnn1-c1ncc(C(=O)N(C)CC#N)cn1] |
| 37 | 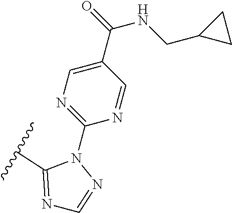 [SMILES: *c1ncnn1-c1ncc(C(=O)NCC2CC2)cn1] |
| 38 | 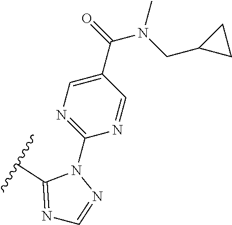 [SMILES: *c1ncnn1-c1ncc(C(=O)N(C)CC2CC2)cn1] |

Also made available are certain intermediate compounds of formulae II(i), III(i), IV(i), V(i), VII(i), XI(i), and XIV(i), or other intermediate compounds shown in Schemes 1 to 33, some of which are novel. For example:
A compound of formula II(i), wherein (i) X¹ is Cl and A¹, A², A³, A⁴, A⁵, R^{2a} and R^{2b} are as defined in any one of Tables A-1 to A-71; or wherein (ii) X¹ is Br and A¹, A², A³, A⁴, A⁵, R^{2a} and R^{2b} are as defined in any one of Tables A-1 to A-71.

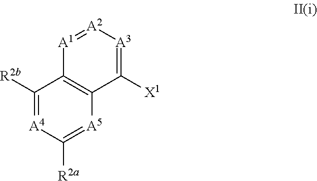

A compound of formula III(i), wherein (i) R¹ is H and Q is as defined in table Z; or wherein (ii) R¹ is CH₃ and Q is as defined in table Z; or wherein (iii) R¹ is CH₂-cyclopropyl and Q is as defined in table Z.

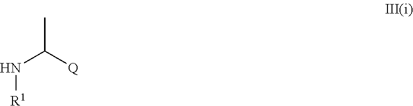

A compound of formula IV(i), wherein A¹, A², A³, A⁴, A⁵, R^{2a} and R^{2b} are as defined in any one of Tables A-1 to A-71.

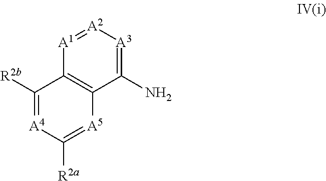

A compound of formula V(i), wherein (i) X² is Cl and Q is as defined in table Z; or wherein (ii) X² is Br and Q is as defined in table Z; or wherein (iii) X² is I and Q is as defined in table Z.

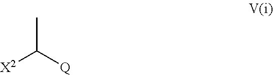

A compound of formula VII(i), wherein Q is as defined in table Z.

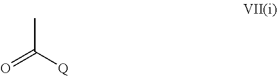

A compound of formula XI(i), wherein A¹, A², A³, A⁴, A⁵, R¹, R^{2a} and R^{2b} are as defined in any one of Tables A-1 to A-71.

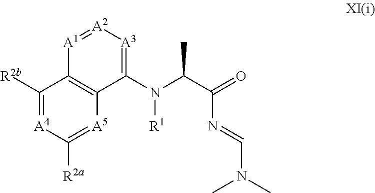

A compound of formula XIV(i), wherein A¹, A², A³, A⁴, A⁵, R¹, R^{2a} and R^{2b} are as defined in any one of Tables A-1 to A-71.

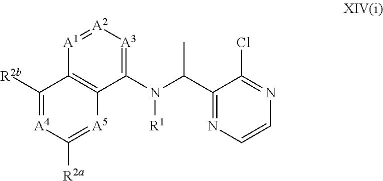

A compound of formula XL-Q^{ac}-a, XL-Q^{ac}-b, or XL-Q^{ac}-c:

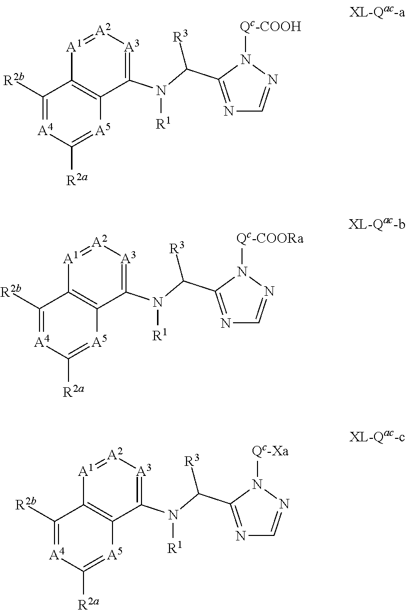

wherein in each case, as applicable, A¹, A², A³, A⁴, A⁵, R^{2a}, and R^{2b} are as defined in any one of Tables A-1 to A-71, R¹ is hydrogen or methyl, R³ is methyl, Ra is benzyl or C₁-C₆alkyl, preferably Ra is methyl, and Xa is a halogen, such as Br, Cl or I, preferably C₁, and wherein Q^{c}-COOH, Q^{c}-COORa and Q^{c}-Xa are as defined in Table Z1:

TABLE Z1
Substituent definitions of Q^{c}-a, Q^{c}-b and Q^{c}-c

| Index | Q^{c1} | Q^{c2} | Q^{c3} | Q^{c4} | Q^{c5} | Q^{c6} |
|---|---|---|---|---|---|---|
| Q^{c}-COOH | 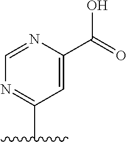 [SMILES: *c1cc(C(=O)O)ncn1] | 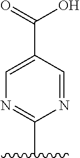 [SMILES: *c1ncc(C(=O)O)cn1] | 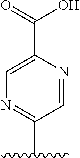 [SMILES: *c1cnc(C(=O)O)cn1] | 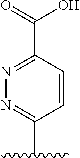 [SMILES: *c1ccc(C(=O)O)nn1] | 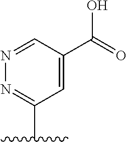 [SMILES: *c1cc(C(=O)O)cnn1] | 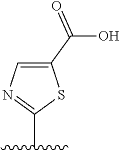 [SMILES: *c1ncc(C(=O)O)s1] |
| | Q^{c1}-a | Q^{c2}-a | Q^{c3}-a | Q^{c4}-a | Q^{c5}-a | Q^{c6}-a |
| Q^{c}-COORa | 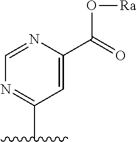 | 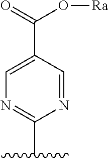 | 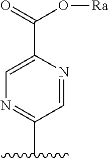 | 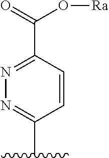 | 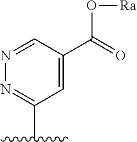 | 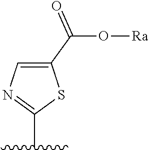 |
| | Q^{c1}-b | Q^{c2}-b | Q^{c3}-b | Q^{c4}-b | Q^{c5}-b | Q^{c6}-b |
| Q^{c}-Xa | 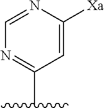 Q^{c1}-c | 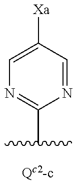 | 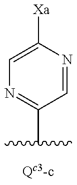 | 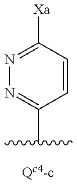 | 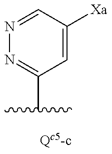 | 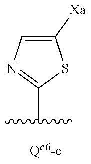 |

Particularly considered are compounds of formula

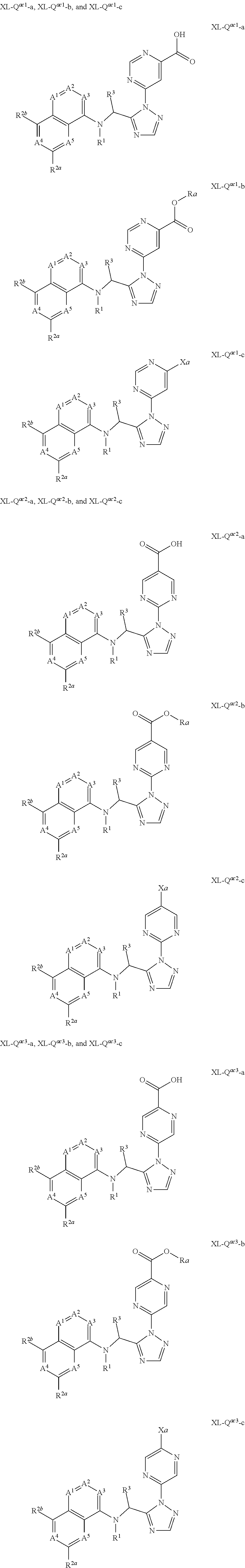

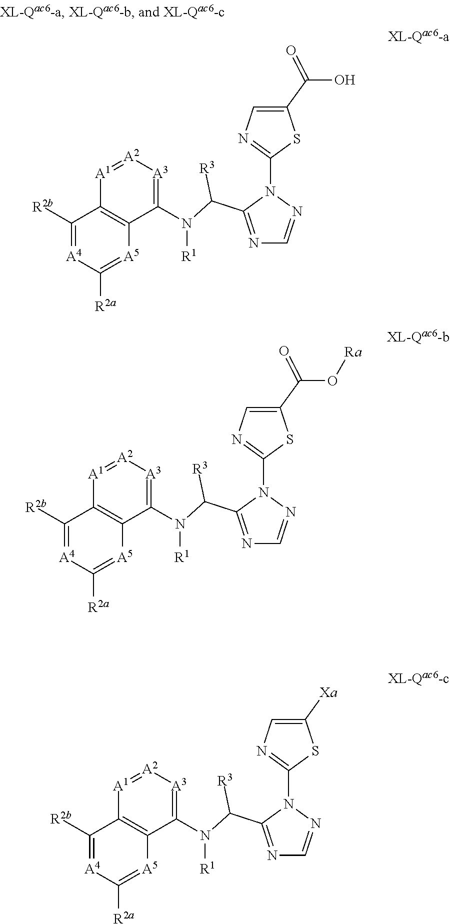

wherein in each case, as applicable, A¹, A², A³, A⁴, A⁵, R^{2a}, and R^{2b} are as defined in any one of Tables A-1 to A-71, R¹ is hydrogen or methyl, R³ is methyl, Ra is benzyl or C₁-C₆alkyl, preferably Ra is methyl, and Xa is a halogen, such as Br, Cl or I, preferably Cl.
A compound of formula XL-Q^{b}c-a, XL-Q^{b}c-b, or XL-Q^{b}c-c:

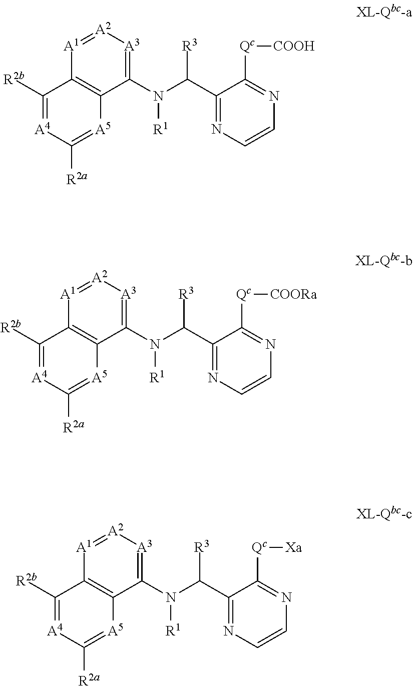

wherein in each case, as applicable, A¹, A², A³, A⁴, A⁵, R^{2a}, and R^{2b} are as defined in any one of Tables A-1 to A-71, R¹ is hydrogen or methyl, R³ is methyl, Ra is benzyl or C₁-C₆alkyl, preferably Ra is methyl, and Xa is a halogen, such as Br, Cl or I, preferably C₁, and wherein Q^{c}-COOH, Q^{c}-COORa and Q^{c}-Xa are Q^{c1}-a, Q^{c1}-b and Q^{c1}-c respectively, as defined in Table Z1 above.
A compound of formula XLIIIa(i), XLIIIb(i), XLIIIb-1(i), XLIIId(i), or XLIIIe(i):

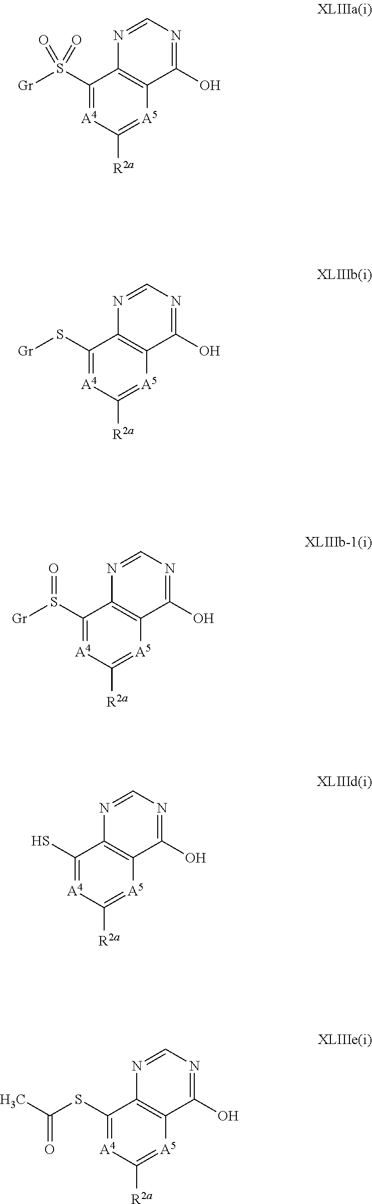

wherein A⁴, A⁵, and R^{2a} are as defined in any one of Tables A-1 to A-71, and wherein Gr is difluoromethyl or trifluoromethyl.
In further aspect, the present invention accordingly makes available compounds of formulae II(i), III(i), IV(i), V(i), VII(i), XI(i), and XIV(i), wherein in each case, as applicable, A¹, A², A³, A⁴, A⁵, R¹, R^{2a} and R^{2b} and Q is as defined for formula (I) in the first aspect; and in respect of formula II(i), X¹ is a halogen, preferably chloro or bromo. Furthermore, the corresponding embodiments illustrated for formula (I) also apply to the compounds of formulae II(i), III(i), IV(i), V(i), VII(i), XI(i), and XIV(i).
In further aspect, the present invention accordingly makes available compounds of formulae XL-Q^{ac}-a, XL-Q^{ac}-b, XL-Q^{ac}-c, XL-Q^{b}c-a, XL-Q^{b}c-b, and XL-Q^{b}c-c, wherein in each case, as applicable, A¹, A², A³, A⁴, A⁵, R¹, R^{2a} and R^{2b} and Q are as defined for formula (I) in the first aspect; and in respect of compounds of formulae XL-Q^{ac}-b, XL-Q^{ac}-c, XL-Q^{bc}-b, and XL-Q^{bc}-c, Ra is benzyl or C₁-C₆alkyl, preferably Ra is methyl, and Xa is a halogen, such as Br, Cl or I, preferably C₁. Furthermore, the corresponding embodiments illustrated for formula (I) also apply to the compounds of formulae XL-Q^{a}C-a, XL-Q^{ac}-b, XL-Q^{ac}-c, XL-Q^{bc}-a, XL-Q^{bc}-b, and XL-Q^{bc}-c.
In further aspect, the present invention accordingly makes available compounds of formulae XLIIIa(i), XLIIIb(i), XLIIIb-1(i), XLIIId(i), or XLIIIe(i), wherein in each case, as applicable, A⁴, A⁵, and R^{2a} is as defined for formula (I) in the first aspect; and Gr is difluoromethyl or trifluoromethyl. Furthermore, the corresponding embodiments illustrated for formula (I) also apply to the compounds of formulae XLIIIa(i), XLIIIb(i), XLIIIb-1(i), XLIIId(i), or XLIIIe(i).
The compounds of formula (I) according to the invention are preventively and/or curatively valuable active ingredients in the field of pest control, even at low rates of application, which have a very favorable biocidal spectrum and are well tolerated by warm-blooded species, fish and plants. The active ingredients according to the invention act against all or individual developmental stages of normally sensitive, but also resistant, animal pests, such as insects or representatives of the order Acarina. The insecticidal or acaricidal activity of the active ingredients according to the invention can manifest itself directly, i.e. in destruction of the pests, which takes place either immediately or only after some time has elapsed, for example during ecdysis, or indirectly, for example in a reduced oviposition and/or hatching rate.
Examples of the above mentioned animal pests are:
from the order Acarina, for example,
Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;
from the order Anoplura, for example,
Haematopinus spp., Linognathus spp., Pediculus spp., Pemphigus spp. and Phylloxera spp.; from the order Coleoptera, for example,
Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemlineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.;
from the order Diptera, for example,
Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;
from the order Hemiptera, for example,
Acanthocoris scabrator, Acrosternum spp, Adelphocoris lineolatus, Aleurodes spp., Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euchistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens; Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris;
from the order Hymenoptera, for example,
Acromyrmex, Arge spp, Atta spp., Cephus spp., Diprion spp., Diprionidae, Gilpinia polytoma, Hoplocampa spp., Lasius spp., Monomorium pharaonis, Neodiprion spp., Pogonomyrmex spp, Slenopsis invicta, Solenopsis spp. and Vespa spp.;
from the order Isoptera, for example,
Coptotermes spp, Corniternes cumulans, Incisitermes spp, Macrotermes spp, Mastotermes spp, Microtermes spp, Reticulitermes spp.; Solenopsis geminate
from the order Lepidoptera, for example,
Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Elasmopalpus lignosellus, Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Grapholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypiella, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;
from the order Mallophaga, for example,
Damalinea spp. and Trichodectes spp.;
from the order Orthoptera, for example,
Blatta spp., Blattella spp., Gryllotalpa spp., Leucophaea maderae, Locusta spp., Neocurtilla hexadactyla, Periplaneta spp., Scapteriscus spp, and Schistocerca spp.;
from the order Psocoptera, for example,
Liposcelis spp.;
from the order Siphonaptera, for example,
Ceratophyllus spp., Ctenocephalides spp. and Xenopsylla cheopis;
from the order Thysanoptera, for example,
Calliothrips phaseoli, Frankliniella spp., Heliothrips spp, Hercinothrips spp., Parthenothrips spp, Scirtothrips aurantii, Sericothrips variabilis, Taeniothrips spp., Thrips spp;
from the order Thysanura, for example, Lepisma saccharina.
   In a further aspect, the invention may also relate to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.
   The compounds of the invention may also have activity against the molluscs. Examples of which include, for example, Ampullariidae; Arion (A. ater, A. circumscriptus, A. hortensis, A. rufus); Bradybaenidae (Bradybaena fruticum); Cepaea (C. hortensis, C. Nemoralis); Ochlodina; Deroceras (D. agrestis, D. empiricorum, D. laeve, D. reticulatum); Discus (D. rotundatus); Euomphalia; Galba (G. trunculata); Helicelia (H. itala, H. obvia); Helicidae Helicigona arbustorum); Helicodiscus; Helix (H. aperta); Limax (L. cinereoniger, L. flavus, L. marginatus, L. maximus, L. tenellus); Lymnaea; Milax (M. gagates, M. marginatus, M. sowerbyi); Opeas; Pomacea (P. canaticulata); Vallonia and Zanitoides.
   The active ingredients according to the invention can be used for controlling, i.e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.
   Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.
   The compositions and/or methods of the present invention may be also used on any ornamental and/or vegetable crops, including flowers, shrubs, broad-leaved trees and evergreens.
   For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperflorens, B. tubereux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime), Coreopsis spp., Crassula coccinea, Cuphea ignea, Dahlia spp., Delphinium spp., Dicentra spectabilis, Dorotheantus spp., Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. Walleriana), Iresines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Bellis spp., Pelargonium spp. (P. peltatum, P. Zonale), Viola spp. (pansy), Petunia spp., Phlox spp., Plecthranthus spp., Poinsettia spp., Parthenocissus spp. (P. quinquefolia, P. tricuspidata), Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.
   For example the invention may be used on any of the following vegetable species: Allium spp. (A. sativum, A. cepa, A. oschaninii, A. Porrum, A. ascalonicum, A. fistulosum), Anthriscus cerefolium, Apium graveolus, Asparagus officinalis, Beta vulgarus, Brassica spp. (B. Oleracea, B. Pekinensis, B. rapa), Capsicum annuum, Cicer arietinum, Cichorium endivia, Cichorum spp. (C. intybus, C. endivia), Citrillus lanatus, Cucumis spp. (C. sativus, C. melo), Cucurbita spp. (C. pepo, C. maxima), Cyanara spp. (C. scolymus, C. cardunculus), Daucus carota, Foeniculum vulgare, Hypericum spp., Lactuca sativa, Lycopersicon spp. (L. esculentum, L. lycopersicum), Mentha spp., Ocimum basilicum, Petroselinum crispum, Phaseolus spp. (P. vulgaris, P. coccineus), Pisum sativum, Raphanus sativus, Rheum rhaponticum, Rosemarinus spp., Salvia spp., Scorzonera hispanica, Solanum melongena, Spinacea oleracea, Valerianella spp. (V. locusta, V. eriocarpa) and Vicia faba.
   Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.
   The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).
   The compounds of formula (I) are particularly suitable for control of
a pest of the order Hemiptera, for example, one or more of the species Bemisia tabaci, Aphis craccivora, Myzus persicae, Rhopalosiphum Padi, Nilaparvata lugens, and Euschistus heros (preferably in vegetables, soybeans, and sugarcane);
a pest of the order Lepidoptera, for example, one or more of the species Spodoptera littoralis, Spodoptera frugiperda, Plutella xylostella, Cnaphalocrocis medinalis, Cydia pomonella, Chrysodeixis includes, Chilo suppressalis, Elasmopalpus lignosellus, Pseudoplusia includens, and Tuta absoluta (preferably in vegetables and corn);
a pest of the order Thysanoptera, such as the family Thripidae, for example, one or more of Thrips tabaci and Frankliniella occidentalis (preferably in vegetables); and
soil pests (such as of the order Coleoptera), for example, the species Diabrotica balteata, Agriotes spp. and Leptinotarsa decemlineata (preferably in vegetables and corn).
   The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.
   Toxins that can be expressed by such transgenic plants include, for example, insecticidal proteins, for example insecticidal proteins from Bacillus cereus or Bacillus popilliae; or insecticidal proteins from Bacillus thuringiensis, such as 6-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.
   In the context of the present invention there are to be understood by 6-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).
   Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.
   The processes for the preparation of such transgenic plants are generally known to the person skilled in the art and are described, for example, in the publications mentioned above. Cryl-type deoxyribonucleic acids and their preparation are known, for example, from WO 95/34656, EP-A-0 367 474, EP-A-0 401 979 and WO 90/13651.
   The toxin contained in the transgenic plants imparts to the plants tolerance to harmful insects. Such insects can occur in any taxonomic group of insects, but are especially commonly found in the beetles (Coleoptera), two-winged insects (Diptera) and moths (Lepidoptera).
   Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1 Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a CrylAb toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.
   Further examples of such transgenic crops are:
   1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated CrylAb toxin. Bt11 maize also transgenically expresses the enzyme PAT to achieve tolerance to the herbicide glufosinate ammonium.
   2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a CrylAb toxin. Bt176 maize also transgenically expresses the enzyme PAT to achieve tolerance to the herbicide glufosinate ammonium.
   3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
   4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
   5. IPC 531 Cotton from Monsanto Europe S.A. 270-272 Avenue de Tervuren, B-1150 Brussels, Belgium, registration number C/ES/96/02.
   6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
   7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a CrylAb toxin obtained from Bacillus thuringiensis subsp. kurstaki which brings about tolerance to certain Lepidoptera, include the European corn borer.
   Transgenic crops of insect-resistant plants are also described in BATS (Zentrum für Biosicherheit und Nachhaltigkeit, Zentrum BATS, Clarastrasse 13, 4058 Basel, Switzerland) Report 2003, (http://bats.ch).
   The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.
   Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.
   Crops also include those that have enhanced resistance to nematodes, such as the soybean cyst nematode.
   Crops that are tolerance to abiotic stress include those that have enhanced tolerance to drought, high salt, high temperature, chill, frost, or light radiation, for example through expression of NF—YB or other proteins known in the art.
   Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defense (so-called “plant disease resistance genes”, as described in WO 03/000906).
   Further areas of use of the compositions according to the invention are the protection of stored goods and store rooms and the protection of raw materials, such as wood, textiles, floor coverings or buildings, and also in the hygiene sector, especially the protection of humans, domestic animals and productive livestock against pests of the mentioned type.
   The present invention provides a compound of the first aspect for use in therapy. The present invention provides a compound of the first aspect, for use in controlling parasites in or on an animal. The present invention further provides a compound of the first aspect, for use in controlling ectoparasites on an animal. The present invention further provides a compound of the first aspect, for use in preventing and/or treating diseases transmitted by ectoparasites.
   The present invention provides the use of a compound of the first aspect, for the manufacture of a medicament for controlling parasites in or on an animal. The present invention further provides the use of a compound of the first aspect, for the manufacture of a medicament for controlling ectoparasites on an animal. The present invention further provides the use of a compound of the first aspect, for the manufacture of a medicament for preventing and/or treating diseases transmitted by ectoparasites.
   The present invention provides the use of a compound of the first aspect, in controlling parasites in or on an animal. The present invention further provides the use of a compound of the first aspect, in controlling ectoparasites on an animal.
   The term “controlling” when used in context of parasites in or on an animal refers to reducing the number of pests or parasites, eliminating pests or parasites and/or preventing further pest or parasite infestation.
   The term “treating” when used in context of parasites in or on an animal refers to restraining, slowing, stopping or reversing the progression or severity of an existing symptom or disease.
   The term “preventing” when used in context of parasites in or on an animal refers to the avoidance of a symptom or disease developing in the animal.
   The term “animal” when used in context of parasites in or on an animal may refer to a mammal and a non-mammal, such as a bird or fish. In the case of a mammal, it may be a human or non-human mammal. Non-human mammals include, but are not limited to, livestock animals and companion animals. Livestock animals include, but are not limited to, cattle, camelids, pigs, sheep, goats and horses. Companion animals include, but are not limited to, dogs, cats and rabbits.
   A “parasite” is a pest which lives in or on the host animal and benefits by deriving nutrients at the host animal's expense. An “endoparasite” is a parasite which lives in the host animal. An “ectoparasite” is a parasite which lives on the host animal. Ectoparasites include, but are not limited to, acari, insects and crustaceans (e.g. sea lice). The Acari (or Acarina) sub-class comprises ticks and mites. Ticks include, but are not limited to, members of the following genera: Rhipicaphalus, for example, Rhipicaphalus (Boophilus) microplus and Rhipicephalus sanguineus; Amblyomrna; Dermacentor; Haemaphysalis; Hyalomma; Ixodes; Rhipicentor; Margaropus; Argas; Otobius; and Ornithodoros. Mites include, but are not limited to, members of the following genera: Chorioptes, for example Chorioptes bovis; Psoroptes, for example Psoroptes ovis; Cheyletiella; Dermanyssus; for example Dermanyssus gallinae; Ortnithonyssus; Demodex, for example Demodex canis; Sarcoptes, for example Sarcoptes scabiei; and Psorergates. Insects include, but are not limited to, members of the orders: Siphonaptera, Diptera, Phthiraptera, Lepidoptera, Coleoptera and Homoptera. Members of the Siphonaptera order include, but are not limited to, Ctenocephalides felis and Ctenocephatides canis. Members of the Diptera order include, but are not limited to, Musca spp.; bot fly, for example Gasterophilus intestinalis and Oestrus ovis; biting flies; horse flies, for example Haematopota spp. and Tabunus spp.; Haematobia, for example Haematobia irritans; Stomoxys; Lucilia; midges; and mosquitoes. Members of the Phthiraptera class include, but are not limited to, blood sucking lice and chewing lice, for example Bovicola Ovis and Bovicola Bovis.
   The term “effective amount” when used in context of parasites in or on an animal refers to the amount or dose of the compound of the invention, or a salt thereof, which, upon single or multiple dose administration to the animal, provides the desired effect in or on the animal. The effective amount can be readily determined by the attending diagnostician, as one skilled in the art, by the use of known techniques and by observing results obtained under analogous circumstances. In determining the effective amount a number of factors are considered by the attending diagnostician, including, but not limited to: the species of mammal; its size, age, and general health; the parasite to be controlled and the degree of infestation; the specific disease or disorder involved; the degree of involvement or the severity of the disease or disorder; the response of the individual; the particular compound administered; the mode of administration; the bioavailability characteristics of the preparation administered; the dose regimen selected; the use of concomitant medication; and other relevant circumstances.
   The compounds of the invention may be administered to the animal by any route which has the desired effect including, but not limited to topically, orally, parenterally and subcutaneously. Topical administration is preferred. Formulations suitable for topical administration include, for example, solutions, emulsions and suspensions and may take the form of a pour-on, spot-on, spray-on, spray race or dip. In the alternative, the compounds of the invention may be administered by means of an ear tag or collar.
   Salt forms of the compounds of the invention include both pharmaceutically acceptable salts and veterinary acceptable salts, which can be different to agrochemically acceptable salts. Pharmaceutically and veterinary acceptable salts and common methodology for preparing them are well known in the art. See, for example, Gould, P. L., “Salt selection for basic drugs”, International Journal of Pharmaceutics, 33: 201-217 (1986); Bastin, R. J., et al. “Salt Selection and Optimization Procedures for Pharmaceutical New Chemical Entities”, Organic Process Research and Development, 4: 427-435 (2000); and Berge, S. M., et al., “Pharmaceutical Salts”, Journal of Pharmaceutical Sciences, 66: 1-19, (1977). One skilled in the art of synthesis will appreciate that the compounds of the invention are readily converted to and may be isolated as a salt, such as a hydrochloride salt, using techniques and conditions well known to one of ordinary skill in the art. In addition, one skilled in the art of synthesis will appreciate that the compounds of the invention are readily converted to and may be isolated as the corresponding free base from the corresponding salt.
   The present invention also provides a method for controlling pests (such as mosquitoes and other disease vectors; see also http://www.who.int/malaria/vector_control/irs/en/). In one embodiment, the method for controlling pests comprises applying the compositions of the invention to the target pests, to their locus or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which can be used in the manufacture of) netting, clothing, bedding, curtains and tents.
   In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on a substrate such as a fabric material in the form of (or which can be used in the manufacture of) netting, clothing, bedding, curtains and tents.
   Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO 2008/151984, WO 2003/034823, U.S. Pat. No. 5,631,072, WO 2005/64072, WO2006/128870, EP 1724392, WO 2005113886 or WO 2007/090739.
   Further areas of use of the compositions according to the invention are the field of tree injection/trunk treatment for all ornamental trees as well all sort of fruit and nut trees.
   In the field of tree injection/trunk treatment, the compounds according to the present invention are especially suitable against wood-boring insects from the order Lepidoptera as mentioned above and from the order Coleoptera, especially against woodborers listed in the following tables A and B:

TABLE A
Examples of exotic woodborers of economic importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE B
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus vittaticolllis | Apple, Pear, Cranberry, Serviceberry, Hawthorn |
| | Chrysobothris femorata | Apple, Apricot, Beech, Boxelder, Cherry, Chestnut, |
| | | Currant, Elm, Hawthorn, Hackberry, Hickory, |
| | | Horsechestnut, Linden, Maple, Mountain-ash, Oak, |
| | | Pecan, Pear, Peach, Persimmon, Plum, Poplar, |
| | | Quince, Redbud, Serviceberry, Sycamore, Walnut, |
| | | Willow |
| | Texania campestris | Basswood, Beech, Maple, Oak, Sycamore, Willow, |
| | | Yellow-poplar |
| Cerambycidae | Goes pulverulentus | Beech, Elm, Nuttall, Willow, Black oak, Cherrybark |
| | | oak, Water oak, Sycamore |
| | Goes tigrinus | Oak |
| | Neoclytus acuminatus | Ash, Hickory, Oak, Walnut, Birch, Beech, Maple, |
| | | Eastern hophornbeam, Dogwood, Persimmon, |
| | | Redbud, Holly, Hackberry, Black locust, |
| | | Honeylocust, Yellow-poplar, Chestnut, Osage- |
| | | orange, Sassafras, Lilac, Mountain-mahogany, |
| | | Pear, Cherry, Plum, Peach, Apple, Elm, |
| | | Basswood, Sweetgum |
| | Neoptychodes trilineatus | Fig, Alder, Mulberry, Willow, Netleaf hackberry |
| | Oberea ocellata | Sumac, Apple, Peach, Plum, Pear, Currant, |
| | | Blackberry |
| | Oberea tripunctata | Dogwood, Viburnum, Elm, Sourwood, Blueberry, |
| | | Rhododendron, Azalea, Laurel, Poplar, Willow, |
| | | Mulberry |
| | Oncideres cingulata | Hickory, Pecan, Persimmon, Elm, Sourwood, |
| | | Basswood, Honeylocust, Dogwood, Eucalyptus, |
| | | Oak, Hackberry, Maple, Fruit trees |
| | Saperda calcarata | Poplar |
| | Strophiona nitens | Chestnut, Oak, Hickory, Walnut, Beech, Maple |
| Scolytidae | Corthylus columbianus | Maple, Oak, Yellow-poplar, Beech, Boxelder, |
| | | Sycamore, Birch, Basswood, Chestnut, Elm |
| | Dendroctonus frontalis | Pine |
| | Dryocoetes betulae | Birch, Sweetgum, Wild cherry, Beech, Pear |
| | Monarthrum fasciatum | Oak, Maple, Birch, Chestnut, Sweetgum, |
| | | Blackgum, Poplar, Hickory, Mimosa, Apple, Peach, |
| | | Pine |
| | Phloeotribus liminaris | Peach, Cherry, Plum, Black cherry, Elm, Mulberry, |
| | | Mountain-ash |
| | Pseudopityophthorus | Oak, American beech, Black cherry, Chickasaw |
| | pruinosus | plum, Chestnut, Maple, Hickory, Hornbeam, |
| | | Hophornbeam |
| Sesiidae | Paranthrene simulans | Oak, American chestnut |
| | Sannina uroceriformis | Persimmon |
| | Synanthedon exitiosa | Peach, Plum, Nectarine, Cherry, Apricot, Almond, |
| | | Black cherry |
| | Synanthedon pictipes | Peach, Plum, Cherry, Beach, Black Cherry |
| | Synanthedon rubrofascia | Tupelo |
| | Synanthedon scitula | Dogwood, Pecan, Hickory, Oak, Chestnut, Beech, |
| | | Birch, Black cherry, Elm, Mountain-ash, Viburnum, |
| | | Willow, Apple, Loquat, Ninebark, Bayberry |
| | Vitacea polistiformis | Grape |

The present invention may be also used to control any insect pests that may be present in turfgrass, including for example beetles, caterpillars, fire ants, ground pearls, millipedes, sow bugs, mites, mole crickets, scales, mealybugs, ticks, spittlebugs, southern chinch bugs and white grubs. The present invention may be used to control insect pests at various stages of their life cycle, including eggs, larvae, nymphs and adults.
In particular, the present invention may be used to control insect pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass ataenius, A. spretulus), Maladera spp. (e.g. Asiatic garden beetle, M. castanea) and Tomarus spp.), ground pearls (Margarodes spp.), mole crickets (tawny, southern, and short-winged; Scapteriscus spp., Gryllotalpa africana) and leatherjackets (European crane fly, Tipula spp.).
The present invention may also be used to control insect pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).
The present invention may also be used to control insect pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs.
The present invention may also be used to control other pests of turfgrass such as red imported fire ants (Solenopsis invicta) that create ant mounds in turf.
In the hygiene sector, the compositions according to the invention are active against ectoparasites such as hard ticks, soft ticks, mange mites, harvest mites, flies (biting and licking), parasitic fly larvae, lice, hair lice, bird lice and fleas.
Examples of such parasites are:
Of the order Anoplurida: Haematopinus spp., Linognathus spp., Pediculus spp. and Phtirus spp., Solenopotes spp.
Of the order Mallophagida: Trimenopon spp., Menopon spp., Trinoton spp., Bovicola spp., Werneckiella spp., Lepikentron spp., Damalina spp., Trichodectes spp. and Felicola spp.
Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.
Of the order Siphonapterida, for example Pulex spp., Ctenocephalides spp., Xenopsylla spp., Ceratophyllus spp.
Of the order Heteropterida, for example Cimex spp., Triatoma spp., Rhodnius spp., Panstrongylus spp.
Of the order Blattarida, for example Blatta orientalis, Periplaneta americana, Blattela germanica and Supella spp.
Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.
Of the orders Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.
The compositions according to the invention are also suitable for protecting against insect infestation in the case of materials such as wood, textiles, plastics, adhesives, glues, paints, paper and card, leather, floor coverings and buildings.
The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec. and Dinoderus minutus, and also hymenopterans such as Sirex juvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.
The compounds of formulae I, and I′a, or salts thereof, are especially suitable for controlling one or more pests selected from the family: Noctuidae, Plutellidae, Chrysomelidae, Thripidae, Pentatomidae, Tortricidae, Delphacidae, Aphididae, Noctuidae, Crambidae, Meloidogynidae, and Heteroderidae. In a preferred embodiment of each aspect, a compound TX (where the abbreviation “TX” means “one compound selected from the compounds defined in Tables A-1 to A-71, and compounds P1 to P85 (i.e. compounds P1 to P5, P6 to P17, P18 to P41, and P42 to P85) of Table P controls one or more of pests selected from the family: Noctuidae, Plutellidae, Chrysomelidae, Thripidae, Pentatomidae, Tortricidae, Delphacidae, Aphididae, Noctuidae, Crambidae, Meloidogynidae, and Heteroderidae.
The compounds of formulae I, and I′a, or salts thereof, are especially suitable for controlling one or more of pests selected from the genus: Spodoptera spp, Plutella spp, Frankliniella spp, Thrips spp, Euschistus spp, Cydia spp, Nilaparvata spp, Myzus spp, Aphis spp, Diabrotica spp, Rhopalosiphum spp, Pseudoplusia spp and Chilo spp. In a preferred embodiment of each aspect, a compound TX (where the abbreviation “TX” means “one compound selected from the compounds defined in Tables A-1 to A-71, and compounds P1 to P85 (i.e. compounds P1 to P5, P6 to P17, P18 to P41, and P42 to P85) of Table P controls one or more of pests selected from the genus: Spodoptera spp, Plutella spp, Frankliniella spp, Thrips spp, Euschistus spp, Cydia spp, Nilaparvata spp, Myzus spp, Aphis spp, Diabrotica spp, Rhopalosiphum spp, Pseudoplusia spp and Chilo spp.
The compounds of formulae I, and I′a, or salts thereof, are especially suitable for controlling one or more of Spodoptera littoralis, Plutella xylostella, Frankliniella occidentalis, Thrips tabaci, Euschistus heros, Cydia pomonella, Nilaparvata lugens, Myzus persicae, Chrysodeixis includens, Aphis craccivora, Diabrotica balteata, Rhopalosiphum padi, and Chilo suppressalis.
In a preferred embodiment of each aspect, a compound TX (where the abbreviation “TX” means “one compound selected from the compounds defined in Tables A-1 to A-71, and compounds P1 to P85 (i.e. compounds P1 to P5, P6 to P17, P18 to P41, and P42 to P85) of Table P controls one or more of Spodoptera littoralis, Plutella xylostella, Frankliniella occidentalis, Thrips tabaci, Euschistus heros, Cydia pomonella, Nilaparvata lugens, Myzus persicae, Chrysodeixis includens, Aphis craccivora, Diabrotica balteata, Rhopalosiphum Padia, and Chilo Suppressalis, such as Spodoptera littoralis+TX, Plutella xylostella+TX; Frankliniella occidentalis+TX, Thrips tabaci+TX, Euschistus heros+TX, Cydia pomonella+TX, Nilaparvata lugens+TX, Myzus persicae+TX, Chrysodeixis includens+TX, Aphis craccivora+TX, Diabrotica balteata+TX, Rhopalosiphum Padi+TX, and Chilo suppressalis+TX.
In an embodiment, of each aspect, one compound from Tables A-1 to A-71, and compounds P1 to P85 (i.e. compounds P1 to P5, P6 to P17, P18 to P41, and P42 to P85) of Table P is suitable for controlling Spodoptera littoralis, Plutella xylostella, Frankliniella occidentalis, Thrips tabaci, Euschistus heros, Cydia pomonella, Nilaparvata lugens, Myzus persicae, Chrysodeixis includens, Aphis craccivora, Diabrotica balteata, Rhopalosiphum Padia, and Chilo Suppressalis in cotton, vegetable, maize, cereal, rice and soya crops.
In an embodiment, one compound from Tables A-1 to A-71, and compounds P1 to P85 (i.e. compounds P1 to P5, P6 to P17, P18 to P41, and P42 to P85) of Table P is suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).
Compounds according to the invention may possess any number of benefits including, inter alia, advantageous levels of biological activity for protecting plants against insects or superior properties for use as agrochemical active ingredients (for example, greater biological activity, an advantageous spectrum of activity, an increased safety profile (against non-target organisms above and below ground (such as fish, birds and bees), improved physico-chemical properties, or increased biodegradability). In particular, it has been surprisingly found that certain compounds of formula (I) may show an advantageous safety profile with respect to non-target arthropods, in particular pollinators such as honey bees, solitary bees, and bumble bees. Most particularly, Apis mellifera.
The compounds according to the invention can be used as pesticidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.
The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.
The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.
The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.
Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.
A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood New Jersey (1981).
Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.
The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.
The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.
The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 I/ha, especially from 10 to 1000 I/ha.
Preferred formulations can have the following compositions (weight %):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85% The following Examples further illustrate, but do not limit, the invention.

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether (7-8 mol of | — | 2% | — |
| ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The combination is thoroughly mixed with the adjuvants and the mixture is thoroughly ground in a suitable mill, affording powders that can be used directly for seed treatment.

Emulsifiable concentrate

| | |
|---|---|
| active ingredients | 10% |
| octylphenol polyethylene glycol ether (4-5 mol of ethylene oxide) | 3% |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any require dilution, which can be use in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

| Extruder granules | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

| Coated granules | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

### Suspension Concentrate

| | |
|---|---|
| active ingredients | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Flowable Concentrate for Seed Treatment

| | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one (in the form of a | 0.5% |
| 20% solution in water) | |
| monoazo-pigment calcium salt | 5% |
| Silicone oil (in the form of a 75% emulsion in water) | 0.2% |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.
Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.
The activity of the compositions according to the invention can be broadened considerably, and adapted to prevailing circumstances, by adding other insecticidally, acaricidally and/or fungicidally active ingredients. The mixtures of the compounds of formula (I) with other insecticidally, acaricidally and/or fungicidally active ingredients may also have further surprising advantages which can also be described, in a wider sense, as synergistic activity. For example, better tolerance by plants, reduced phytotoxicity, insects can be controlled in their different development stages or better behaviour during their production, for example during grinding or mixing, during their storage or during their use.
Suitable additions to active ingredients here are, for example, representatives of the following classes of active ingredients: organophosphorus compounds, nitrophenol derivatives, thioureas, juvenile hormones, formamidines, benzophenone derivatives, ureas, pyrrole derivatives, carbamates, pyrethroids, chlorinated hydrocarbons, acylureas, pyridylmethyleneamino derivatives, macrolides, neonicotinoids and Bacillus thuringiensis preparations.

### PREPARATORY EXAMPLES

“Mp” means melting point in ° C. Free radicals represent methyl groups. 1H NMR measurements were recorded on a Brucker 400 MHz spectrometer, chemical shifts are given in ppm relevant to a TMS standard. Spectra measured in deuterated solvents as indicated. Either one of the LCMS methods below was used to characterize the compounds. The characteristic LCMS values obtained for each compound were the retention time (“Rt”, recorded in minutes) and the measured molecular ion (M+H)⁺ or (M−H)⁻.

### Method 1:

Spectra were recorded on a Mass Spectrometer from Waters Corporation (SQD, SQDII or QDA Single quadrupole mass spectrometer) equipped with an electrospray source (Polarity: positive and negative ions), Capillary: 0.8-3.00 kV, Cone: 5-30 V, Source Temperature: 120-150° C., Desolvation Temperature: 350-600° C., Cone Gas Flow: 50-150 I/h, Desolvation Gas Flow: 650-1000 I/h, Mass range: 100 to 900 Da and an Acquity UPLC from Waters Corporation: Binary pump, heated column compartment, diode-array detector and ELSD. Column: Waters UPLC HSS T3, 1.8 μm, 30×2.1 mm, Temp: 60° C., DAD Wavelength range (nm): 210 to 400, Runtime: 1.5 min; Solvents: A=water+5% MeOH+0.05% HCOOH, B=Acetonitrile+0.05% HCOOH; Flow (ml/min) 0.85, Gradient: 10% B isocratic for 0.2 min, then 10-100% B in 1.0 min, 100% B isocratic for 0.2 min, 100-10% B in 0.05 min, 10% B isocratic for 0.05 min.

### Method 2:

Spectra were recorded on a Mass Spectrometer from Waters (SQD, SQDII Single quadrupole mass spectrometer) equipped with an electrospray source (Polarity: positive and negative ions, Capillary: 3.00 kV, Cone range: 41 V, Extractor: 2.00 V, Source Temperature: 150° C., Desolvation Temperature: 5000° C., Cone Gas Flow: 50 l/h, Desolvation Gas Flow: 1000 l/h, Mass range: 110 to 800 Da) and an Acquity UPLC from Waters: Binary pump, heated column compartment, diode-array detector and ELSD detector. Column: Waters UPLC HSS T3 C18, 1.8 μm, 30×2.1 mm, Temp: 40° C., PDA Wavelength range (nm): 200 to 400, Solvent Gradient: A=water+5% Acetonitrile+0.1% HCOOH, B=Acetonitrile+0.05% HCOOH, gradient: 10-100% B in 1.3 min; Flow (ml/min) 0.6.

### Method 3:

Spectra were recorded on a Mass Spectrometer from Waters (SQD2 or QDA Single quadrupole mass spectrometer) equipped with an electrospray source (Polarity: Positive and Negative Polarity Switch), Capillary: 0.8-3.00 kV, Cone range: 25 Source Temperature: 120-150° C., Desolvation Temperature: 500-600° C., Cone Gas Flow: 50 L/h, Desolvation Gas Flow: 1000 L/h, Mass range: 110 to 850 Da) and an Acquity UPLC from Waters: Quaternary solvent manager, heated column compartment, diode-array detector. Column: Acquity UPLC HSS T3 C18, 1.8 μm, 30×2.1 mm, Temp: 40° C., DAD Wavelength range (nm): 200 to 400, Solvent Gradient: A=water+5% Acetonitrile+0.1% HCOOH, B=Acetonitrile+0.05% HCOOH: gradient: 0 min 10% B; 0-0.2 min 10-50% B; 0.2-0.6 min 50-100% B; 0.6-1.3 min 100% B; 1.3-1.4 min 100-10% B; 1.4-1.6 min 10% B; Flow (mL/min) 0.6.

### Method 4:

Spectra were recorded on a Mass Spectrometer from Waters (SQD2 or QDA Single quadrupole mass spectrometer) equipped with an electrospray source (Polarity: Positive and Negative Polarity Switch), Capillary: 0.8-3.00 kV, Cone range: 25 Source Temperature: 120-150° C., Desolvation Temperature: 500-600° C., Cone Gas Flow: 50 L/h, Desolvation Gas Flow: 1000 L/h, Mass range: 110 to 850 Da) and an Acquity UPLC from Waters: Quaternary solvent manager, heated column compartment, diode-array detector. Column: Acquity UPLC HSS T3 C18, 1.8 μm, 30×2.1 mm, Temp: 40° C., DAD Wavelength range (nm): 200 to 400, Solvent Gradient: A=water+5% Acetonitrile+0.1% HCOOH, B=Acetonitrile+0.05% HCOOH: gradient: 0 min 10% B; 0.0-0.5 min 10% B; 0.5-2 min 100% B; 2-3 min 100% B; 3-3.5 min 10% B; 3.5-4 min 10% B; Flow (mL/min) 0.6.

### Method 5:

Spectra were recorded on a ACQUITY Mass Spectrometer from Waters Corporations (QDa or SQDII Single quadrupole mass spectrometer) equipped with an electrospray source (Polarity: positive or negative ions, Capillary: 3.0 kV, Cone: 30V, Extractor: 3.00 V, Source Temperature: 150° C., Desolvation Temperature: 400° C., Cone Gas Flow: 60 L/hr, Desolvation Gas Flow: 700 L/hr, Mass range: 140 to 800 Da) and an ACQUITY UPLC from Waters Corporations with solvent degasser, binary pump, heated column compartment and diode-array detector. Column: Waters UPLC HSS T3, 1.8 μm, 30×2.1 mm, Temp: 60° C., DAD Wavelength range (nm): 210 to 400, Solvent Gradient: A=Water/Methanol 9:1+0.1% formic acid, B=Acetonitrile+0.1% formic acid, gradient: 0-100% B in 3.0 min; Flow (ml/min) 0.75.

### Example E1: Preparation of 2-[5-[(1S)-1-[[6-chloro-8-(trifluoromethyl)quinazolin-4-yl]amino]ethyl]-1,2,4-triazol-1-yl]-N-ethyl-N-methyl-thiazole-5-carboxamide (compound P6)

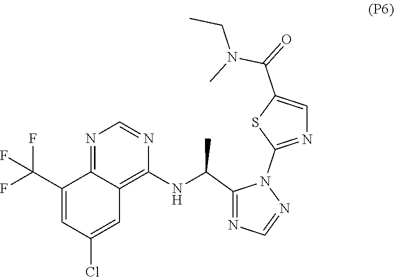 [SMILES: CCN(C)C(=O)c1cnc(-n2ncnc2[C@H](C)Nc2ncnc3c(C(F)(F)F)cc(Cl)cc23)s1]

### Step 1: Preparation of methyl 2-[5-[(1S)-1-(tert-butoxycarbonylamino)ethyl]-1,2,4-triazol-1-yl]thiazole-5-carboxylate (I-1)

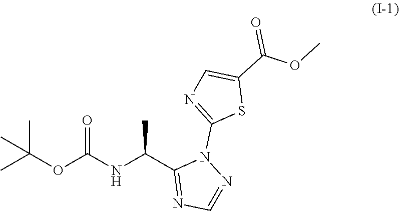 [SMILES: COC(=O)c1cnc(-n2ncnc2[C@H](C)NC(=O)OC(C)(C)C)s1]

To a solution of tert-butyl N-[(1S)-2-[(E)-dimethylaminomethyleneamino]-1-methyl-2-oxo-ethyl]carbamate (CAS 2641011-39-2, prepared as described for example in WO21/083936) (5.2 g, 21 mmol) in 1,4-dioxane (31 mL) and acetic acid (31 mL) was added methyl 2-hydrazinothiazole-5-carboxylate (CAS 2648006-04-4) (3.7 g, 21 mmol). The reaction mixture was heated at 50° C. for 16 hours, then cooled to RT and diluted with EtOAc and water. The phases were separated, the aqueous layer extracted with EtOAc, the combined organic layers washed with water and brine, dried over sodium sulfate, filtered and concentrated under reduced pressure. The residue was purified by combiflash (gradient ethyl acetate in cyclohexane) to afford methyl 2-[5-[(1S)-1-(tert-butoxycarbonylamino)ethyl]-1,2,4-triazol-1-yl]thiazole-5-carboxylate. LCMS (method 2): retention time 1.11 min, m/z 298 [M+H-tBu]⁺.

### Step 2: Preparation of 2-[5-[(1S)-1-(tert-butoxycarbonylamino)ethyl]-1,2,4-triazol-1-yl]thiazole-5-carboxylic acid (I-2)

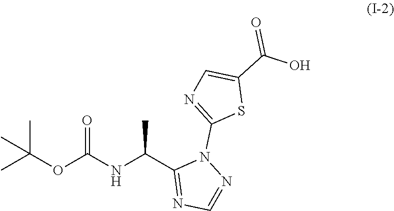 [SMILES: C[C@H](NC(=O)OC(C)(C)C)c1ncnn1-c1ncc(C(=O)O)s1]

A mixture of methyl 2-[5-[(1S)-1-(tert-butoxycarbonylamino)ethyl]-1,2,4-triazol-1-yl]thiazole-5-carboxylate (I-1) (3.7 g, 10 mmol) and lithium hydroxide (0.78 g, 31 mmol) in tetrahydrofuran (37 mL) and water (3.7 mL) was stirred at room temperature for 16 hours, then diluted with EtOAc, water and a 5% aqueous monosodium phosphate (NaH₂PO₄) solution. The phases were separated and the aqueous layer washed with EtOAc, cooled by addition of crushed ice, then acidified using a 10% aqueous HCl solution. The formed precipitate was filtered, the solid washed with water and dried. The solid was further washed with pentane and dried in vacuo to afford 2-[5-[(1S)-1-(tert-butoxycarbonylamino)ethyl]-1,2,4-triazol-1-yl]thiazole-5-carboxylic acid as an off-white solid. This material was used without further purification for the next step.
LCMS (method 2): retention time 1.03 min, m/z 284 [M+H-tBu]⁺.

### Step 3: Preparation of tert-butyl N-[(1S)-1-[2-[5-[ethyl(methyl)carbamoyl]thiazol-2-yl]-1,2,4-triazol-3-yl]ethyl]carbamate (I-3)

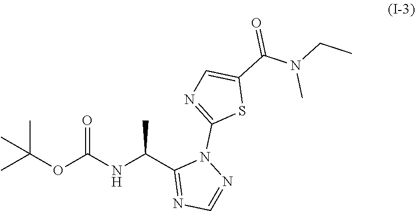 [SMILES: CCN(C)C(=O)c1cnc(-n2ncnc2[C@H](C)NC(=O)OC(C)(C)C)s1]

To a suspension of 2-[5-[(1S)-1-(tert-butoxycarbonylamino)ethyl]-1,2,4-triazol-1-yl]thiazole-5-carboxylic acid (I-2) (0.5 g, 1.43 mmol) in EtOAc (11.8 mL) were added ethylmethylamine (0.19 mL, 2.21 mmol), N,N-diisopropylethylamine (0.76 mL, 4.42 mmol), followed by propanephosphonic acid cyclic anhydride (T3P®, 50 wt. % in ethyl acetate, 2.63 mL, 4.42 mmol). The reaction mixture was stirred at room temperature overnight. After dilution with water, the product was extracted with EtOAc (3×), the combined organic layers washed with water and brine, dried over sodium sulfate, filtered and concentrated under reduced pressure. The residue was purified by combiflash (gradient ethyl acetate in cyclohexane) to afford tert-butyl N-[(1S)-1-[2-[5-[ethyl(methyl)carbamoyl]thiazol-2-yl]-1,2,4-triazol-3-yl]ethyl]carbamate. LCMS (method 2): retention time 1.09 min, m/z 381 [M+H]⁺.

### Step 4: Preparation of [(1S)-1-[2-[5-[ethyl(methyl)carbamoyl]thiazol-2-yl]-1,2,4-triazol-3-yl]ethyl]ammonium chloride (I-4)

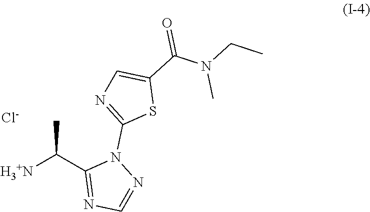

To a solution of tert-butyl N-[(1S)-1-[2-[5-[ethyl(methyl)carbamoyl]thiazol-2-yl]-1,2,4-triazol-3-yl]ethyl]carbamate (I-3) (0.43 g, 1.13 mmol) in 1,4-dioxane (3 mL) was added hydrochloric acid (4.0 M in 1,4-dioxane, 3 mL, 12 mmol). The reaction mixture was stirred at room temperature for 5 hours, then concentrated in vacuo to dryness. The residue was triturated with TBME, the formed solid filtered and dried to afford [(1S)-1-[2-[5-[ethyl(methyl)carbamoyl]thiazol-2-yl]-1,2,4-triazol-3-yl]ethyl]ammonium chloride as a white solid.
LCMS (method 2): retention time 0.17 min. m/z 281 [M+H]⁺ of the corresponding free base.

### Step 5: Preparation of 2-[5-[(1S)-1-[[6-chloro-8-(trifluoromethyl)quinazolin-4-yl]amino]ethyl]-1,2,4-triazol-1-yl]-N-ethyl-N-methyl-thiazole-5-carboxamide (compound P6)

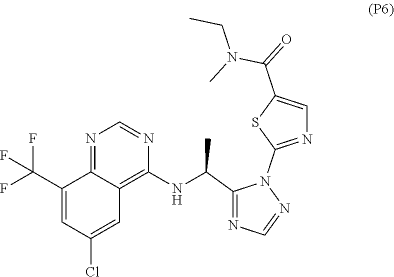 [SMILES: CCN(C)C(=O)c1cnc(-n2ncnc2[C@H](C)Nc2ncnc3c(C(F)(F)F)cc(Cl)cc23)s1]

A stirred solution of 4,6-dichloro-8-(trifluoromethyl)quinazoline (prepared as described in WO 2021/083936) (155 mg, 90%, 0.52 mmol), [(1S)-1-[2-[5-[ethyl(methyl)carbamoyl]thiazol-2-yl]-1,2,4-triazol-3-yl]ethyl]ammonium chloride (I-4) (150 mg, 0.47 mmol) and triethylamine (241 mg, 2.37 mmol) in tetrahydrofuran (3 mL) was heated in the microwave at 100° C. for 1 hour. The reaction mixture was evaporated under reduced pressure and the residue purified by combiflash (gradient ethyl acetate in cyclohexane) to afford 2-[5-[(1S)-1-[[6-chloro-8-(trifluoromethyl)quinazolin-4-yl]amino]ethyl]-1,2,4-triazol-1-yl]-N-ethyl-N-methyl-thiazole-5-carboxamide (compound P6) as a white solid.
LCMS (method 3): retention time 1.09 min, m/z 511/513 [M+H]⁺.

### Example E2: Preparation of 6-[5-[(1S)-1-[[6-chloro-8-(trifluoromethyl)quinazolin-4-yl]amino]ethyl]-1,2,4-triazol-1-yl]pyrimidine-4-carboxamide (compound P5)

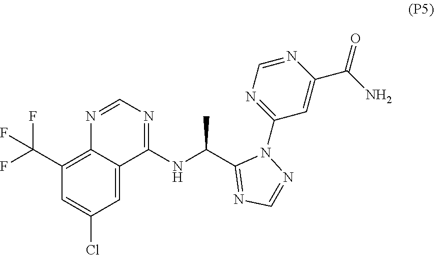 [SMILES: C[C@H](Nc1ncnc2c(C(F)(F)F)cc(Cl)cc12)c1ncnn1-c1cc(C(N)=O)ncn1]

A solution of 4,6-dichloro-8-(trifluoromethyl)quinazoline (prepared as described in WO 2021/083936) (150 mg, 0.562 mmol), 6-[5-[(1S)-1-aminoethyl]-1,2,4-triazol-1-yl]pyrimidine-4-carboxamide hydrochloride (prepared as described in WO 2021/165195) (181.8 mg, 0.674 mmol) and cesium carbonate (549.1 mg, 1.685 mmol) in acetonitrile (3 mL) was heated at 80° C. for 1 hour. The reaction mixture was cooled to room temperature, diluted with water, the formed precipitate filtered, washed with water followed by pentane, and dried. The residue was purified by combiflash (ethyl acetate in cyclohexane) to afford 6-[5-[(1S)-1-[[6-chloro-8-(trifluoromethyl)quinazolin-4-yl]amino]ethyl]-1,2,4-triazol-1-yl]pyrimidine-4-carboxamide (compound P5) as an off-white solid (85 mg).
LCMS (method 2): retention time 1.07 min, m/z 464/466 [M+H]⁺. ¹H NMR (400 MHz, DMSO-d₆) δ ppm 9.30 (s, 1H), 9.14 (br d, 1H), 8.97 (d, 1H), 8.46 (br s, 1H), 8.40 (s, 2H), 8.20-8.30 (m, 2H), 8.13 (br s, 1H), 6.56 (m, 1H), 1.76 (d, 3H).

### Example E3: Preparation of 6-[5-[(1S)-1-[(6,8-dibromoquinazolin-4-yl)-methyl-amino]ethyl]-1,2,4-triazol-1-yl]-N-methyl-pyrimidine-4-carboxamide (compound P11)

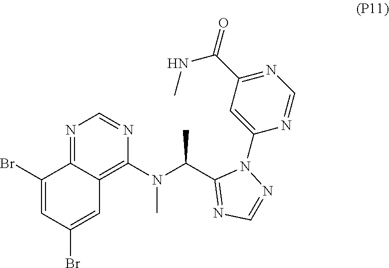 [SMILES: CNC(=O)c1cc(-n2ncnc2[C@H](C)N(C)c2ncnc3c(Br)cc(Br)cc23)ncn1]

To a stirred solution of 6-[5-[(1S)-1-[(6,8-dibromoquinazolin-4-yl)amino]ethyl]-1,2,4-triazol-1-yl]-N-methyl-pyrimidine-4-carboxamide (compound P12) (0.14 g, 0.262 mmol) in acetonitrile (2.8 mL) was added cesium carbonate (0.27 g, 0.787 mmol) and the mixture was heated to 90° C. Iodomethane (0.392 g, 2.62 mmol) was added at 90° C. and the reaction mixture stirred at this temperature for 1 hour. After cooling to room temperature, water was added and the product extracted with EtOAc. The combined organic layers were washed with brine, dried over sodium sulfate, filtered and concentrated under reduced pressure. The residue was purified by combiflash (ethyl acetate in cyclohexane) to afford 6-[5-[(1S)-1-[(6,8-dibromoquinazolin-4-yl)-methyl-amino]ethyl]-1,2,4-triazol-1-yl]-N-methyl-pyrimidine-4-carboxamide (P11) as a solid.
LCMS (method 3): retention time 1.00 min, m/z 546/548/550 [M+H]⁺. ¹H NMR (400 MHz, DMSO-d₆) δ ppm 9.06 (q, 1H), 8.57 (d, 1H), 8.53 (s, 1H), 8.40 (s, 1H), 8.37 (d, 1H), 8.34 (d, 1H), 7.99 (d, 1H), 6.79 (q, 1H), 3.23 (s, 3H), 2.84 (d, 3H), 1.83 (d, 3H).

### Example E4: Preparation of 6-[5-[(1S)-1-[[6-chloro-8-(trifluoromethyl)quinazolin-4-yl]amino]ethyl]-1,2,4-triazol-1-yl]-N-(1-cyanocyclopropyl)pyrimidine-4-carboxamide (compound P22)

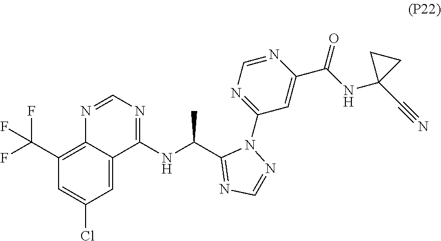 [SMILES: C[C@H](Nc1ncnc2c(C(F)(F)F)cc(Cl)cc12)c1ncnn1-c1cc(C(=O)NC2(C#N)CC2)ncn1]

### Step 1: Preparation of [(1S)-1-[2-(6-chloropyrimidin-4-yl)-1,2,4-triazol-3-yl]ethyl]ammonium chloride (I-17)

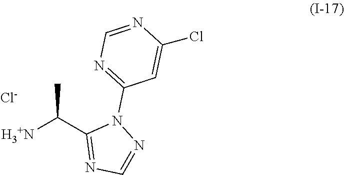

To a solution of tert-butyl N-[(1S)-1-[2-(6-chloropyrimidin-4-yl)-1,2,4-triazol-3-yl]ethyl]carbamate (CAS 2694010-23-4, prepared as described for example in WO21/165195) (20.0 g, 61.58 mmol) in 1,4-dioxane (200 mL) was added a hydrochloric acid solution (4M in dioxane) (77.0 mL, 308 mmol) at room temperature. The reaction mixture was stirred at room temperature for 25 hours, the formed precipitate isolated by filtration, and the solid dried in vacuo to afford [(1S)-1-[2-(6-chloropyrimidin-4-yl)-1,2,4-triazol-3-yl]ethyl]ammonium chloride (I-17) as a white solid.
LCMS (method 3): retention time 0.20 min, m/z 225/227 [M+H]⁺ of the corresponding free base.

### Step 2: Preparation of 6-chloro-N-[(1S)-1-[2-(6-chloropyrimidin-4-yl)-1,2,4-triazol-3-yl]ethyl]-8-(trifluoromethyl)quinazolin-4-amine (I-18)

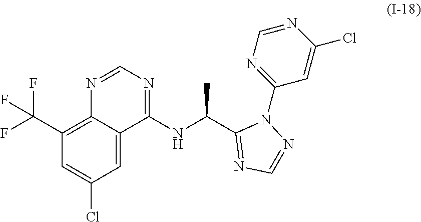 [SMILES: C[C@H](Nc1ncnc2c(C(F)(F)F)cc(Cl)cc12)c1ncnn1-c1cc(Cl)ncn1]

To a mixture of 4,6-dichloro-8-(trifluoromethyl)quinazoline (prepared as described in WO 21/083936) (9.0 g, 33.70 mmol) and [(1S)-1-[2-(6-chloropyrimidin-4-yl)-1,2,4-triazol-3-yl]ethyl]ammonium chloride (1-17) (9.24 g, 35.39 mmol) in tetrahydrofuran (90 mL) was added triethylamine (10.34 g, 14.2 mL, 101.11 mmol) at room temperature. The reaction mixture was stirred at 70° C. for 1 hour, then diluted with water (1000 mL), the formed precipitate isolated by filtration, the solid washed with water and dried in vacuo to afford 6-chloro-N-[(1S)-1-[2-(6-chloropyrimidin-4-yl)-1,2,4-triazol-3-yl]ethyl]-8-(trifluoromethyl)quinazolin-4-amine (I-18) as a pale yellowish solid.
LCMS (method 3): retention time 1.33 min, m/z 455/457 [M+H]⁺.

### Step 3: Preparation of methyl 6-[5-[(1S)-1-[[6-chloro-8-(trifluoromethyl)quinazolin-4-yl]amino]ethyl]-1,2,4-triazol-1-yl]pyrimidine-4-carboxylate (I-19)

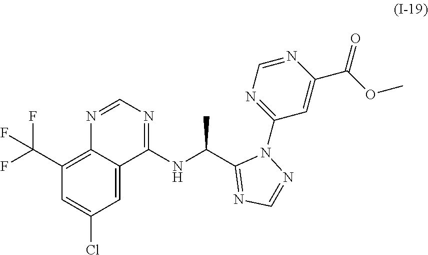 [SMILES: COC(=O)c1cc(-n2ncnc2[C@H](C)Nc2ncnc3c(C(F)(F)F)cc(Cl)cc23)ncn1]

A pressure vessel was charged with 6-chloro-N-[(1S)-1-[2-(6-chloropyrimidin-4-yl)-1,2,4-triazol-3-yl]ethyl]-8-(trifluoromethyl)quinazolin-4-amine (I-18) (3.8 g, 5.84 mmol), [1,1′-bis(diphenylphosphino) ferrocene]dichloropalladium(II) (PdCl₂(dppf, 0.487 g, 0.584 mmol), triethylamine (0.597 g, 0.823 mL, 5.84 mmol) and methanol (76 mL). The vessel was flushed with nitrogen, then with carbon monoxide. The reaction mixture was heated to 80° C. under a 15 bar carbon monoxide pressure for 6 hours. After cooling to room temperature, the pressure was carefully released and the vessel flushed with nitrogen. The mixture was filtered through a celite bed, the filter cake washed with EtOAc and the filtrate concentrated under reduced pressure. The residue was purified by combiflash (ethyl acetate in cyclohexane) to afford methyl 6-[5-[(1S)-1-[[6-chloro-8-(trifluoromethyl)quinazolin-4-yl]amino]ethyl]-1,2,4-triazol-1-yl]pyrimidine-4-carboxylate (I-19) as a white solid.
LCMS (method 3): retention time 1.17 min, m/z 479/481 [M+H]⁺.
Similarly, methyl 6-[5-[(1S)-1-[[6-chloro-8-(trifluoromethyl)quinazolin-4-yl]-methyl-amino]ethyl]-1,2,4-triazol-1-yl]pyrimidine-4-carboxylate (I-22) can be obtained from intermediate (I-21) with above protocol. LCMS (method 3): retention time 1.21 min, m/z 493/495 [M+H]⁺.

### Step 4: Preparation of 6-[5-[(1S)-1-[[6-chloro-8-(trifluoromethyl)quinazolin-4-yl]amino]ethyl]-1,2,4-triazol-1-yl]pyrimidine-4-carboxylic acid (I-20)

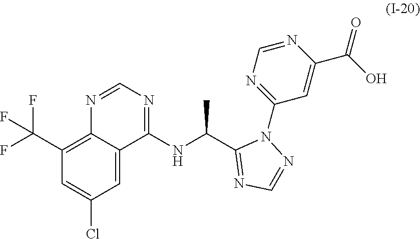 [SMILES: C[C@H](Nc1ncnc2c(C(F)(F)F)cc(Cl)cc12)c1ncnn1-c1cc(C(=O)O)ncn1]

To a solution of methyl 6-[5-[(1S)-1-[[6-chloro-8-(trifluoromethyl)quinazolin-4-yl]amino]ethyl]-1,2,4-triazol-1-yl]pyrimidine-4-carboxylate (I-19) (2.2 g, 4.60 mmol) in methanol (44 mL) and water (44 mL) was added sodium hydroxide (0.2205 g, 5.51 mmol) at room temperature. The reaction mixture was stirred at room temperature for 20 hours, diluted with water (20 mL) and extracted once with TBME (50 mL). The aqueous layer was acidified with citric acid, stirred for 10 minutes, the formed precipitate isolated by filtration, and the solid washed with water and dried in vacuo to afford 6-[5-[(1S)-1-[[6-chloro-8-(trifluoromethyl)quinazolin-4-yl]amino]ethyl]-1,2,4-triazol-1-yl]pyrimidine-4-carboxylic acid (I-20) as s white solid. LCMS (method 3): retention time 1.06 min, m/z 465/467 [M+H]⁺.
Similarly, 6-[5-[(1S)-1-[[6-chloro-8-(trifluoromethyl)quinazolin-4-yl]-methyl-amino]ethyl]-1,2,4-triazol-1-yl]pyrimidine-4-carboxylic acid (I-23) can be obtained from intermediate (I-22) with above protocol.
LCMS (method 3): retention time 1.12 min, m/z 479/481 [M+H]⁺.

### Step 5: Preparation of 6-[5-[(1S)-1-[[6-chloro-8-(trifluoromethyl)quinazolin-4-yl]amino]ethyl]-1,2,4-triazol-1-yl]-N-(1-cyanocyclopropyl)pyrimidine-4-carboxamide (compound P22)

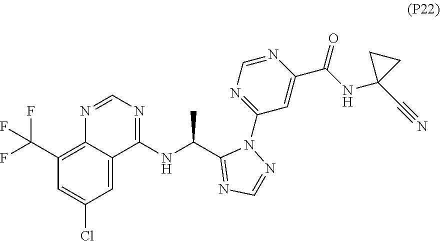 [SMILES: C[C@H](Nc1ncnc2c(C(F)(F)F)cc(Cl)cc12)c1ncnn1-c1cc(C(=O)NC2(C#N)CC2)ncn1]

To a solution of 6-[5-[(1S)-1-[[6-chloro-8-(trifluoromethyl)quinazolin-4-yl]amino]ethyl]-1,2,4-triazol-1-yl]pyrimidine-4-carboxylic acid (I-20) (0.15 g, 0.323 mmol) and (1-cyanocyclopropyl)ammonium chloride (0.153 g, 1.291 mmol) in acetonitrile (3 mL) were added 1-propanphosphonic acid cyclic anhydride (50 mass % in EtOAc, 0.5763 mL, 0.968 mmol), followed by triethylamine (0.164 g, 0.226 mL, 1.614 mmol) at room temperature. The reaction mixture was stirred at room temperature for 25 hours, then diluted with an aqueous saturated NaHCO₃ solution and the product extracted twice with EtOAc. The combined organic layers were washed with brine, dried over sodium sulfate, filtered and concentrated in vacuo. The residue was purified by combiflash (ethyl acetate in cyclohexane) to afford 6-[5-[(1S)-1-[[6-chloro-8-(trifluoromethyl)quinazolin-4-yl]amino]ethyl]-1,2,4-triazol-1-yl]-N-(1-cyanocyclopropyl)pyrimidine-4-carboxamide (compound P22) as a white solid.
LCMS (method 3): retention time 1.22 min, m/z 529/531 [M+H]⁺. ¹H NMR (400 MHz, CDCl₃) δ ppm: 9.26 (d, 1H), 8.84 (d, 1H), 8.59 (s, 1H), 8.44 (s, 1H), 8.15 (s, 1H), 8.01 (d, 1H), 7.86-7.94 (m, 2H), 6.70 (quint, 1H), 1.84 (d, 3H), 1.71-1.78 (m, 2H), 1.39-1.44 (m, 2H).

### Example E5: Preparation of 6-[5-[(1S)-1-[[6-chloro-8-(trifluoromethyl)quinazolin-4-yl]amino]ethyl]-1,2,4-triazol-1-yl]-N-(thietan-3-yl)pyrimidine-4-carboxamide (compound P25)

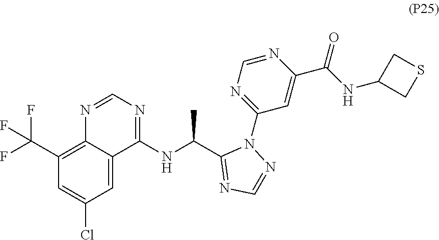 [SMILES: C[C@H](Nc1ncnc2c(C(F)(F)F)cc(Cl)cc12)c1ncnn1-c1cc(C(=O)NC2CSC2)ncn1]

Obtained in analogy from 6-[5-[(1S)-1-[[6-chloro-8-(trifluoromethyl)quinazolin-4-yl]amino]ethyl]-1,2,4-triazol-1-yl]pyrimidine-4-carboxylic acid (I-20) (0.30 g, 0.645 mmol), thietan-3-ylammonium chloride (0.162 g, 1.290 mmol), 1-propanphosphonic acid cyclic anhydride (50 mass % in EtOAc, 0.5763 mL, 0.968 mmol) and triethylamine (0.144 g, 0.199 mL, 1.420 mmol) in acetonitrile (6 mL) according to procedure Example E4, step 5. After stirring at room temperature for 25 hours, the reaction mixture was diluted with an aqueous NaHCO₃ solution, stirring continued for 20 minutes, the formed precipitate isolated by filtration, and the solid washed with water and dried in vacuo to afford 6-[5-[(1S)-1-[[6-chloro-8-(trifluoromethyl)quinazolin-4-yl]amino]ethyl]-1,2,4-triazol-1-yl]-N-(thietan-3-yl)pyrimidine-4-carboxamide (compound P25) as a white solid.
LCMS (method 3): retention time 1.28 min, m/z 536/538 [M+H]⁺. ¹H NMR (400 MHz, DMSO-d₆) δ ppm: 9.83 (d, 1H), 9.33 (s, 1H), 9.16 (d, 1H), 8.96 (d, 1H), 8.40 (s, 1H), 8.38 (d, 1H), 8.28 (s, 1H), 8.23 (d, 1H), 6.55 (quint, 1H), 5.23-5.36 (m, 1H), 3.71 (m, 2H), 3.15-3.28 (m, 2H), 1.76 (d, 3H).
Similarly, 6-[5-[(1S)-1-[[6-chloro-8-(trifluoromethyl)quinazolin-4-yl]amino]ethyl]-1,2,4-triazol-1-yl]-N-cyclopropyl-pyrimidine-4-carboxamide (compound P23) can be obtained with above protocol when replacing thietan-3-ylammonium chloride with cyclopropanamine.
LCMS (method 3): retention time 1.24 min, m/z 504/506 [M+H]⁺.

### Example E6: Preparation of 6-[5-[(1S)-1-[[6-chloro-8-(trifluoromethyl)quinazolin-4-yl]amino]ethyl]-1,2,4-triazol-1-yl]-N-(1,1-dioxothietan-3-yl)pyrimidine-4-carboxamide (compound P24)

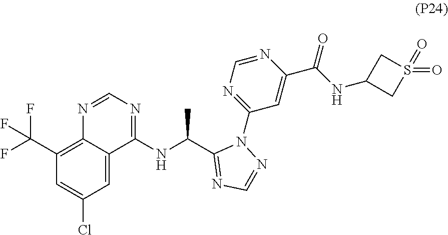 [SMILES: C[C@H](Nc1ncnc2c(C(F)(F)F)cc(Cl)cc12)c1ncnn1-c1cc(C(=O)NC2CS(=O)(=O)C2)ncn1]

To a solution of 6-[5-[(1S)-1-[[6-chloro-8-(trifluoromethyl)quinazolin-4-yl]amino]ethyl]-1,2,4-triazol-1-yl]-N-(thietan-3-yl)pyrimidine-4-carboxamide (compound P25) (0.140 g, 0.261 mmol) in a mixture of acetonitrile (1.4 mL), carbon tetrachloride (1.4 mL) and water (2.8 mL) were added ruthenium(III) chloride (5.42 mg, 0.026 mmol), followed by sodium periodate (0.118 g, 0.549 mmol) at room temperature. The reaction mixture was stirred at room temperature for 1 hour, then quenched with an aqueous saturated Na₂S₂O₅ solution and the product extracted twice with EtOAc. The combined organic layers were washed with brine, dried over sodium sulfate, filtered and concentrated in vacuo. The residue was purified by combiflash (ethyl acetate in cyclohexane) to afford 6-[5-[(1S)-1-[[6-chloro-8-(trifluoromethyl)quinazolin-4-yl]amino]ethyl]-1,2,4-triazol-1-yl]-N-(1,1-dioxothietan-3-yl)pyrimidine-4-carboxamide (compound P24) as a white solid.
LCMS (method 3): retention time 1.20 min, m/z 568/570 [M+H]⁺. ¹H NMR (400 MHz, DMSO-d₆) δ ppm: 9.90 (d, 1H), 9.36 (d, 1H), 9.16 (d, 1H), 8.97 (d, 1H), 8.37-8.41 (m, 2H), 8.28 (s, 1H), 8.24 (d, 1H), 6.56 (quint, 1H), 4.47-4.71 (m, 5H), 1.76 (d, 3H).

### Example E7: Preparation of 6-[5-[(1S)-1-[[6-chloro-8-(trifluoromethyl)quinazolin-4-yl]amino]ethyl]-1,2,4-triazol-1-yl]-N-methoxy-pyrimidine-4-carboxamide (compound P28)

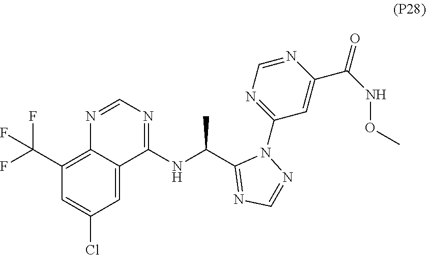 [SMILES: CONC(=O)c1cc(-n2ncnc2[C@H](C)Nc2ncnc3c(C(F)(F)F)cc(Cl)cc23)ncn1]

Obtained in analogy from 6-[5-[(1S)-1-[[6-chloro-8-(trifluoromethyl)quinazolin-4-yl]amino]ethyl]-1,2,4-triazol-1-yl]pyrimidine-4-carboxylic acid (I-20) (0.15 g, 0.323 mmol), methoxyammonium chloride (0.135 g, 1.614 mmol), 1-propanphosphonic acid cyclic anhydride (50 mass % in EtOAc, 0.2882 mL, 0.484 mmol) and triethylamine (0.197 g, 0.271 mL, 1.936 mmol) in acetonitrile (3 mL) according to procedure Example E4, step 5. After stirring at room temperature for 25 hours, the reaction mixture was diluted with an aqueous NaHCO₃ solution, stirring continued for 20 minutes, the formed precipitate isolated by filtration, and the solid washed with water and dried in vacuo to afford 6-[5-[(1S)-1-[[6-chloro-8-(trifluoromethyl)quinazolin-4-yl]amino]ethyl]-1,2,4-triazol-1-yl]-N-methoxy-pyrimidine-4-carboxamide (compound P28) as a white solid.
LCMS (method 3): retention time 1.16 min, m/z 494/496 [M+H]⁺. ¹H NMR (DMSO-d₆) δ ppm: 12.53 (s, 1H), 9.28 (s, 1H), 9.16 (d, 1H), 8.97 (d, 1H), 8.40 (s, 1H), 8.37 (s, 1H), 8.28 (s, 1H), 8.24 (d, 1H), 6.55 (quint, 1H), 3.75 (s, 3H), 1.76 (d, 3H).

### Example E8: Preparation of 6-[5-[(1S)-1-[[6-chloro-8-(trifluoromethyl)quinazolin-4-yl]amino]ethyl]-1,2,4-triazol-1-yl]-N-ethyl-N-methyl-pyrimidine-4-carboxamide (compound P31)

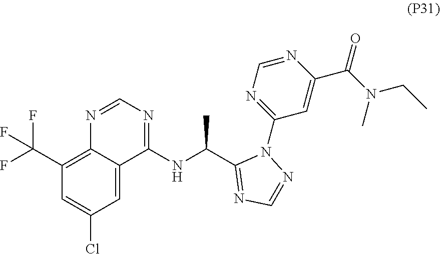 [SMILES: CCN(C)C(=O)c1cc(-n2ncnc2[C@H](C)Nc2ncnc3c(C(F)(F)F)cc(Cl)cc23)ncn1]

Obtained in analogy from 6-[5-[(1S)-1-[[6-chloro-8-(trifluoromethyl)quinazolin-4-yl]amino]ethyl]-1,2,4-triazol-1-yl]pyrimidine-4-carboxylic acid (I-20) (0.15 g, 0.323 mmol), N-methylethanamine (0.095 g, 1.614 mmol) and 1-propanphosphonic acid cyclic anhydride (50 mass % in EtOAc, 0.5763 mL, 0.968 mmol) in acetonitrile (3 mL) according to procedure Example E4, step 5 (in which triethylamine is omitted here). After stirring at room temperature for 24 hours, the reaction mixture was diluted with water, stirring continued for 20 minutes, the formed precipitate isolated by filtration, and the solid washed with water and dried in vacuo to afford 6-[5-[(1S)-1-[[6-chloro-8-(trifluoromethyl)quinazolin-4-yl]amino]ethyl]-1,2,4-triazol-1-yl]-N-ethyl-N-methyl-pyrimidine-4-carboxamide (compound P31) as a white solid. LCMS (method 3): retention time 1.23 min. m/z 506/508 [M+H]⁺.

¹H NMR (400 MHz, d₆-DMSO) δ ppm: 9.24 (dd, 2H), 9.16 (br d, 2H), 8.98 (s, 2H), 8.40 (s, 2H), 8.26 (s, 2H), 8.24 (d, 2H), 8.04 (d, 2H), 6.52 (quint, 2H), 3.52 (q, 2H), 3.28 (q, 2H), 3.02 (s, 3H), 2.94 (s, 3H), 1.78 (d, 6H), 1.18 (t, 3H), 1.14 (t, 3H) as a mixture of 2 rotamers.

Similarly, [6-[5-[(1S)-1-[[6-chloro-8-(trifluoromethyl)quinazolin-4-yl]amino]ethyl]-1,2,4-triazol-1-yl]pyrimidin-4-yl]-morpholino-methanone (compound P32) can be obtained with above protocol when replacing N-methylethanamine with morpholine. The crude product obtained after aqueous workup was purified by reversed phase column chromatography (C₁₈ column, H₂O:ACN eluent).
LCMS (method 3): retention time 1.20 min, m/z 534/536 [M+H]⁺.

### Example E9: Preparation of 6-[5-[(1S)-1-[[6-chloro-8-(trifluoromethyl)quinazolin-4-yl]amino]ethyl]-1,2,4-triazol-1-yl]-N-(cyanomethyl)pyrimidine-4-carboxamide (compound P38)

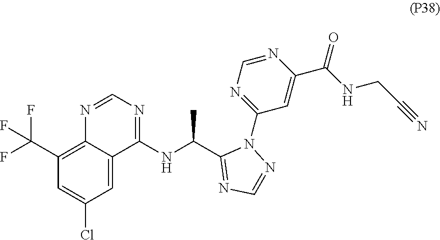 [SMILES: C[C@H](Nc1ncnc2c(C(F)(F)F)cc(Cl)cc12)c1ncnn1-c1cc(C(=O)NCC#N)ncn1]

Obtained in analogy from 6-[5-[(1S)-1-[[6-chloro-8-(trifluoromethyl)quinazolin-4-yl]amino]ethyl]-1,2,4-triazol-1-yl]pyrimidine-4-carboxylic acid (I-20) (0.15 g, 0.323 mmol), cyanomethylammonium chloride (0.119 g, 1.291 mmol), 1-propanphosphonic acid cyclic anhydride (50 mass % in EtOAc, 0.5763 mL, 0.968 mmol) and triethylamine (0.164 g, 0.226 mL, 1.614 mmol) in acetonitrile (3 mL) according to procedure Example E4, step 5. Purification by combiflash afforded 6-[5-[(1S)-1-[[6-chloro-8-(trifluoromethyl)quinazolin-4-yl]amino]ethyl]-1,2,4-triazol-1-yl]-N-(cyanomethyl)pyrimidine-4-carboxamide (compound P38) as a white solid.
LCMS (method 3): retention time 1.18 min, m/z 503/505 [M+H]⁺. ¹H NMR (400 MHz, CDCl₃) δ ppm: 9.31 (s, 1H), 8.90 (s, 1H), 8.77 (br d, 1H), 8.40-8.52 (m, 2H), 8.24 (s, 1H), 7.94 (s, 1H), 7.72 (s, 1H), 6.67 (quint, 1H), 4.38-4.60 (m, 2H), 1.89 (d, 3H).

### Example E10: Preparation of 6-[5-[(1S)-1-[(8-chloro-6-iodo-quinazolin-4-yl)amino]ethyl]-1,2,4-triazol-1-yl]pyrimidine-4-carbonitrile (compound P18)

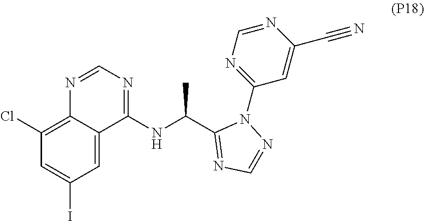 [SMILES: C[C@H](Nc1ncnc2c(Cl)cc(I)cc12)c1ncnn1-c1cc(C#N)ncn1]

A solution of 4,8-dichloro-6-iodo-quinazoline (CAS 100948-96-7, prepared in analogy to descriptions found for example in WO 2021/083936) (100 mg, 0.277 mmol, 90 mass %), [(1S)-1-[2-(6-cyanopyrimidin-4-yl)-1,2,4-triazol-3-yl]ethyl]ammonium 2,2,2-trifluoroacetate (I-13, CAS 2694010-00-7 prepared as described in WO 2021/165195) (101.3 mg, 0.277 mmol, 90 mass %) and triethylamine (0.194 mL, 0.141 g, 1.385 mmol) in tetrahydrofuran (2 mL) was heated in the microwave at 100° C. for 1 hour. The reaction mixture was cooled to room temperature and evaporated in vacuo. The residue was purified by combiflash (ethyl acetate in cyclohexane) and the obtained product washed with TBME, filtered and dried under high vacuum to afford 6-[5-[(1S)-1-[(8-chloro-6-iodo-quinazolin-4-yl)amino]ethyl]-1,2,4-triazol-1-yl]pyrimidine-4-carbonitrile (compound P18) as a white solid. LCMS (method 3): retention time 1.13 min, m/z 504/506 [M+H]⁺. ¹H NMR (400 MHz, DMSO-d₆) δ ppm: 1.73 (d, 3H), 6.47 (quint, 1H), 8.27 (d, 1H), 8.30 (s, 1H), 8.36 (s, 1H), 8.61 (d, 1H), 8.90 (d, 1H), 9.05 (d, 1H), 9.36 (d, 1H).

### Example E11: Preparation of 6-[5-[(1S)-1-[(6-chloro-8-iodo-quinazolin-4-yl)-methyl-amino]ethyl]-1,2,4-triazol-1-yl]pyrimidine-4-carboxamide (compound P47)

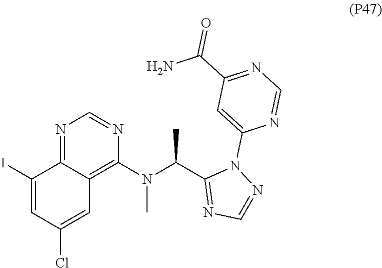 [SMILES: C[C@@H](c1ncnn1-c1cc(C(N)=O)ncn1)N(C)c1ncnc2c(I)cc(Cl)cc12]

To a mixture of 4,6-dichloro-8-iodoquinazoline (CAS 100949-33-5) (0.1 g, 0.307 mmol) and [(1S)-1-[2-(6-carbamoylpyrimidin-4-yl)-1,2,4-triazol-3-yl]ethyl]-methyl-ammonium chloride (I-29) (0.096 g, 0.338 mmol) in ACN (2 mL) was added triethylamine (0.156 g, 1.53 mmol) and the mixture was heated to 90° C. for 1 hour. After cooling to room temperature, ice cold water was added and the product extracted with EtOAc. The combined organic layers were dried over sodium sulfate, filtered and concentrated under reduced pressure. The residue was purified by silica gel column chromatography (ethyl acetate in cyclohexane) to afford 6-[5-[(1S)-1-[(6-chloro-8-iodo-quinazolin-4-yl)-methyl-amino]ethyl]-1,2,4-triazol-1-yl]pyrimidine-4-carboxamide (P47) as a solid. LCMS (method 3): retention time 1.11 min, m/z 536/538 [M+H]⁺. ¹H NMR (400 MHz, DMSO-d₆) δ ppm 8.55 (s, 1H), 8.49 (s, 1H), 8.35-8.44 (m, 4H), 8.08 (br s, 1H), 7.89 (d, 1H), 6.79 (q, 1H), 3.23 (s, 3H), 1.83 (d, 3H).

### Example E12: Preparation of 5-[5-[(1S)-1-[[6-chloro-8-(trifluoromethyl)quinazolin-4-yl]amino]ethyl]-1,2,4-triazol-1-yl]pyrazine-2-carboxamide (compound P21)

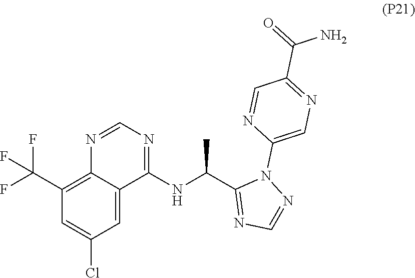 [SMILES: C[C@H](Nc1ncnc2c(C(F)(F)F)cc(Cl)cc12)c1ncnn1-c1cnc(C(N)=O)cn1]

To a mixture of 4,6-dichloro-8-(trifluoromethyl)quinazoline (prepared as described in WO 21/083936) (100 mg, 0.374 mmol) and [(1S)-1-[2-(5-carbamoylpyrazin-2-yl)-1,2,4-triazol-3-yl]ethyl]ammonium chloride (I-16) (0.411 mmol) were added ACN (2 mL) and triethylamine (1.87 mmol). The reaction mixture was stirred at 80° C. for 1 hour, then evaporated under reduced pressure. The residue was purified by silica gel column chromatography (ethyl acetate in cyclohexane) to afford 5-[5-[(1S)-1-[[6-chloro-8-(trifluoromethyl)quinazolin-4-yl]amino]ethyl]-1,2,4-triazol-1-yl]pyrazine-2-carboxamide (P21) as a pale yellow solid. LCMS (method 4): retention time 1.74 min, m/z 464/466 [M+H]⁺.

### Example E13: Preparation of 6-[5-[(1S)-1-[[6-chloro-8-(trifluoromethyl)quinazolin-4-yl]amino]ethyl]-1,2,4-triazol-1-yl]-N-cyclopropyl-N-methyl-pyrimidine-4-carboxamide (compound P63)

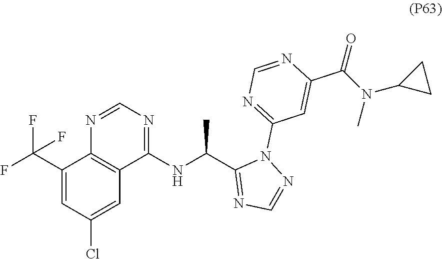 [SMILES: C[C@H](Nc1ncnc2c(C(F)(F)F)cc(Cl)cc12)c1ncnn1-c1cc(C(=O)N(C)C2CC2)ncn1]

To a mixture of 6-[5-[(1S)-1-[[6-chloro-8-(trifluoromethyl)quinazolin-4-yl]amino]ethyl]-1,2,4-triazol-1-yl]pyrimidine-4-carboxylic acid (I-20) (200 mg, 0.43 mmol) and cyclopropyl(methyl)ammonium chloride (4.3 mmol) in acetonitrile (4 mL) was added HATU (0.245 mmol) at room temperature, followed by triethylamine (1.29 mmol) at 0-5° C. The reaction mixture was stirred at room temperature for 14 hours and further at 80° C. for 11 hours, then diluted with water and an aqueous saturated sodium bicarbonate solution. The product was extracted with EtOAc, the combined organic layers were dried over sodium sulfate, filtered and concentrated in vacuo. The residue was purified by reversed phase combiflash using a C₁₈ column (acetonitrile in water) to afford 6-[5-[(1S)-1-[[6-chloro-8-(trifluoro-methyl)quinazolin-4-yl]amino]ethyl]-1,2,4-triazol-1-yl]-N-cyclopropyl-N-methyl-pyrimidine-4-carboxamide (P63) as white solid. LCMS (method 3): retention time 1.11 min, m/z 518/520 [M+H]⁺. ¹H NMR (DMSO-d₆) δ ppm: 9.22 (s, 1H), 9.15 (br d, 1H), 8.95 (m, 1H), 8.39 (s, 1H), 8.25 (s, 1H), 8.22 (m, 1H), 8.10 (s, 1H), 6.50 (quint, 1H), 3.04 (s, 3H), 2.80-2.97 (m, 1H), 1.78 (br d, 3H), 0.40-0.59 (m, 4H).

### Example E14: Preparation of 6-[5-[(1S)-1-[[6-chloro-8-(trifluoromethylsulfonyl)quinazolin-4-yl]amino]ethyl]-1,2,4-triazol-1-ylpyrimidine-4-carboxamide (compound P59)

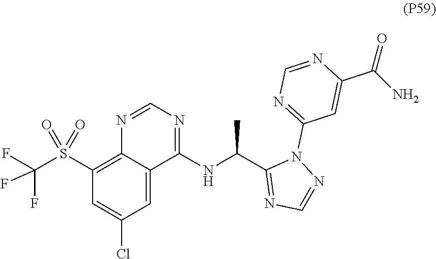 [SMILES: C[C@H](Nc1ncnc2c(S(=O)(=O)C(F)(F)F)cc(Cl)cc12)c1ncnn1-c1cc(C(N)=O)ncn1]

### Step 1: Preparation of 6-chloro-8-(trifluoromethylsulfanyl)quinazolin-4-ol

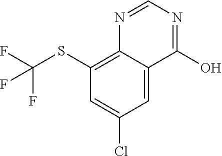 [SMILES: Oc1ncnc2c(SC(F)(F)F)cc(Cl)cc12]

To a solution of 6-chloro-8-iodo-quinazolin-4-ol (CAS 101581-08-2) (4 g, 13.05 mmol) in degassed N,N-dimethylacetamide (40 mL) was added copper(I) trifluoromethanethiolate (4.94 g, 30.02 mmol) at room temperature. The reaction mixture was heated at 90° C. for 48 hours. After cooling, the mixture was diluted with aqueous 2N HCl (100 mL) and water (300 mL). The precipitated solid was filtered, the solid re-dissolved in ethyl acetate and the solution passed though a celite bed. The filtrate was dried over sodium sulfate, filtered and concentrated in vacuo. The residue was purified by combiflash (ethyl acetate in cyclohexane) to afford 6-chloro-8-(trifluoromethylsulfanyl)quinazolin-4-ol as a pale brown solid. LCMS (method 3): retention time 1.11 min, m/z 279/281 [M−H]⁻.

¹H NMR (DMSO-d₆) δ: 12.79 (br s, 1H), 8.31 (s, 1H), 8.20 (s, 1H), 8.13 (s, 1H).

### Step 2: Preparation of 6-chloro-8-(trifluoromethylsulfonyl)quinazolin-4-ol (I-45)

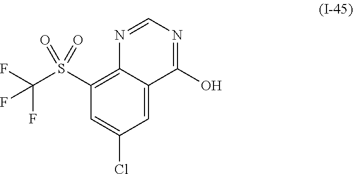 [SMILES: O=S(=O)(c1cc(Cl)cc2c(O)ncnc12)C(F)(F)F]

To a solution of 6-chloro-8-(trifluoromethylsulfanyl)quinazolin-4-ol (prepared as described above) (0.65 g, 2.32 mmol) in acetonitrile (6.5 mL), carbon tetrachloride (6.5 mL) and water (13 mL) was added ruthenium(III) chloride (0.048 g, 0.23 mmol), followed by sodium periodate (1.05 g, 4.86 mmol) at room temperature. After addition, the reaction mixture was stirred at room temperature for 2 hours, then diluted with an aqueous solution of sodium metabisulfite and the product extracted twice with EtOAc. The combined organic layers were washed with brine, dried over sodium sulfate, filtered and concentrated in vacuo. The residue was purified by combiflash (ethyl acetate in cyclohexane) to afford 6-chloro-8-(trifluoromethylsulfonyl)quinazolin-4-ol as white solid. LCMS (method 3): retention time 1.08 min, m/z 313/315 [M+H]⁺. ¹H NMR (DMSO-d₆) δ: 13.05 (br s, 1H), 8.60 (d, 1H), 8.53 (d, 1H), 8.43 (s, 1H).

### Step 3: Preparation of 4,6-dichloro-8-(trifluoromethylsulfonyl)quinazoline

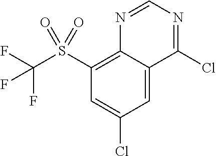 [SMILES: O=S(=O)(c1cc(Cl)cc2c(Cl)ncnc12)C(F)(F)F]

A mixture of 6-chloro-8-(trifluoromethylsulfonyl)quinazolin-4-ol (I-45, prepared as described above) (0.43 g, 1.37 mmol), thionyl chloride (117.9 mmol) and N,N-dimethylformamide (0.068 mmol) was heated at 100° C. for 8 hours. The reaction mixture was concentrated under reduced pressure to afford crude 4,6-dichloro-8-(trifluoromethylsulfonyl)quinazoline (460 mg) as pale yellow solid, which was used in the next step immediately. ¹H NMR (CDCl₃) δ: 9.30 (s, 1H), 8.81 (d, 1H), 8.72 (d, 1H).
Similarly, 4,6-dichloro-8-(difluoromethylsulfonyl)quinazoline can be obtained from 6-chloro-8-(difluoromethylsulfonyl)quinazolin-4-ol (I-44) with above protocol.

¹H NMR (400 MHz, DMSO-d₆) δ ppm 8.58 (s, 1H), 8.07 (d, 1H), 7.96 (d, 1H), 7.89 (t, 1H).

### Step 4: Preparation of 6-[5-[(1S)-1-[[6-chloro-8-(trifluoromethylsulfonyl)quinazolin-4-yl]amino]ethyl]-1,2,4-triazol-1-yl]pyrimidine-4-carboxamide (compound P59)

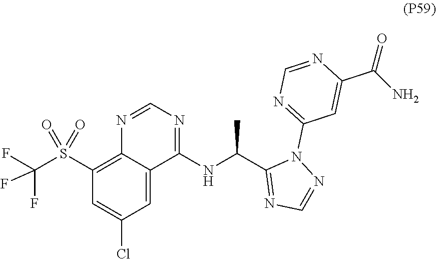 [SMILES: C[C@H](Nc1ncnc2c(S(=O)(=O)C(F)(F)F)cc(Cl)cc12)c1ncnn1-c1cc(C(N)=O)ncn1]

To a mixture of 4,6-dichloro-8-(trifluoromethylsulfonyl)quinazoline (prepared as described above) (0.07 g, 0.21 mmol) and [(1S)-1-[2-(6-carbamoylpyrimidin-4-yl)-1,2,4-triazol-3-yl]ethyl]ammonium chloride (I-12) (0.085 g, 0.32 mmol) in tetrahydrofuran (2 mL) was added triethylamine (0.63 mmol) at room temperature. The reaction mixture was heated at 70° C. for 1 hour, cooled to room temperature and diluted with water (20 mL). The precipitated solid was isolated by filtration and washed with water, then dried in vacuo. This crude material was purified by reversed phase column chromatography using a C18 (40-60 μm) column (acetonitrile in water) to afford 6-[5-[(1S)-1-[[6-chloro-8-(trifluoromethyl-sulfonyl)quinazolin-4-yl]amino]ethyl]-1,2,4-triazol-1-yl]pyrimidine-4-carboxamide (P59) as white solid.
LCMS (method 3): retention time 1.11 min, m/z 528/530 [M+H]⁺. ¹H NMR (acetonitrile-d₃) δ: 9.12 (s, 1H), 8.55 (s, 1H), 8.53 (s, 1H), 8.36 (s, 2H), 8.01 (s, 1H), 7.98 (br d, 1H), 7.74 (br s, 1H), 6.59 (quint, 1H), 6.47 (br s, 1H), 1.76 (d, 3H).
Similarly, 6-[5-[(1S)-1-[[6-chloro-8-(difluoromethylsulfonyl)quinazolin-4-yl]amino]ethyl]-1,2,4-triazol-1-yl]pyrimidine-4-carboxamide (compound P84) can be obtained from 4,6-dichloro-8-(difluoromethylsulfonyl)quinazoline (1.0 equiv.) and [(1S)-1-[2-(6-carbamoylpyrimidin-4-yl)-1,2,4-triazol-3-yl]ethyl]ammonium chloride (I-12, 1.2 equiv.) in the presence of potassium carbonate (3.0 equiv.) in acetonitrile. The mixture is stirred at 0° C. to room temperature for 2 hours. Following standard workup, the crude material is purified by combiflash (ethyl acetate in cyclohexane), then by reversed phase column chromatography (acetonitrile in water) to afford the desired compound P84. ¹H NMR (400 MHz, DMSO-d₆) δ ppm 9.40 (d, 1H), 9.29 (s, 1H), 9.14 (d, 1H), 8.46 (br s, 1H), 8.42 (m, 2H), 8.40 (s, 1H), 8.28 (s, 1H), 8.13 (br s, 1H), 7.67 (t, 1H), 6.57 (quint, 1H), 1.77 (d, 3H).

### Example E15: Preparation of 6-[3-[1-[(6-chloro-8-iodo-quinazolin-4-yl)amino]ethyl]pyrazin-2-yl]pyrimidine-4-carboxamide (compound P58)

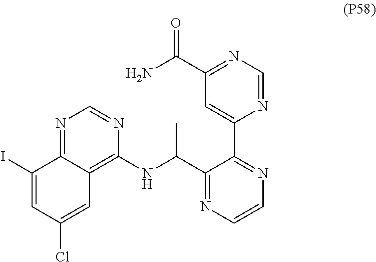 [SMILES: CC(Nc1ncnc2c(I)cc(Cl)cc12)c1nccnc1-c1cc(C(N)=O)ncn1]

### Step 1: Preparation of 2-(1-methylprop-2-ynyl)isoindoline-1,3-dione

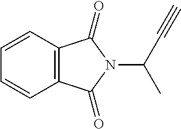

To a mixture of triphenylphosphane (37.4 g, 135.6 mmol) and phthalimide (18 g, 116.250 mmol) in anhydrous THE (100 mL) was added 3-butyn-2-ol (7 g, 96.8 mmol). The reaction mixture was cooled to 0° C. and diisopropyl azodicarboxylate (116.2 mmol) was added slowly at 0-5° C. After stirring at room temperature for 16 h, the reaction mixture was quenched with an aqueous ammonium chloride solution, the product extracted with ethyl acetate, the combined organic layers dried over sodium sulfate, filtered and concentrated in vacuo. The residue was purified by combiflash (ethyl acetate in cyclohexane) to afford 2-(1-methylprop-2-ynyl)isoindoline-1,3-dione as a white solid. ¹H NMR (400 MHz, DMSO-d₆) δ ppm 7.84-7.92 (m, 4H), 5.11 (dd, 1H), 3.33-3.37 (m, 1H), 1.62 (d, 3H).

### Step 2: Preparation of 6-[3-(1,3-dioxoisoindolin-2-yl)but-1-ynyl]pyrimidine-4-carboxamide

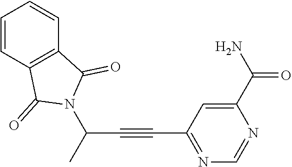 [SMILES: CC(C#Cc1cc(C(N)=O)ncn1)N1C(=O)c2ccccc2C1=O]

A mixture of 6-chloropyrimidine-4-carboxamide (4.95 g, 31.4 mmol), cesium carbonate (18.2 g, 94.3 mmol) and XPhos (3.77 mmol) in ACN (49.5 mL) was flushed with nitrogen for 15 minutes. Then 2-(1-methylprop-2-ynyl)isoindoline-1,3-dione (prepared as described above) (8.14 g, 40.8 mmol), bis(acetonitrile)dichloropalladium(II) (1.57 mmol) and copper(I)iodide (3.14 mmol) were added and the reaction mixture was stirred at 100° C. for 2 hours. The mixture was diluted with water and the product extracted twice with ethyl acetate. The combined organic layers were washed with water and brine, dried over sodium sulfate, filtered and evaporated under reduced pressure. The residue was purified by combiflash (ethyl acetate in cyclohexane) to afford 6-[3-(1,3-dioxoisoindolin-2-yl)but-1-ynyl]pyrimidine-4-carboxamide as a solid. LCMS (method 3): retention time 1.01 min, m/z 321 [M+H]⁺.

### Step 3: Preparation of 6-[3-(1,3-dioxoisoindolin-2-yl)-2-oxo-butyl]pyrimidine-4-carboxamide

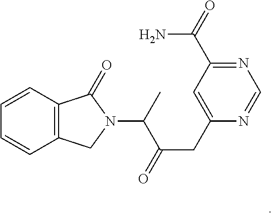 [SMILES: CC(C(=O)Cc1cc(C(N)=O)ncn1)N1Cc2ccccc2C1=O ; "."]

To 6-[3-(1,3-dioxoisoindolin-2-yl)but-1-ynyl]pyrimidine-4-carboxamide (prepared as described above) (300 mg, 0.936 mmol) was added sulfuric acid (3 mL) at 0-5° C. slowly. The reaction mixture was stirred room temperature for 6 hours, then carefully poured into cold water. The precipitated solid was isolated by filtration and dried in vacuo to afford 6-[3-(1,3-dioxoisoindolin-2-yl)-2-oxo-butyl]pyrimidine-4-carboxamide. LCMS (method 3): retention time 1.00 min, m/z 339 [M+H]⁺.

### Step 4: Preparation of 6-[3-(1,3-dioxoisoindolin-2-yl)-2-oxo-butanoyl]pyrimidine-4-carboxamide

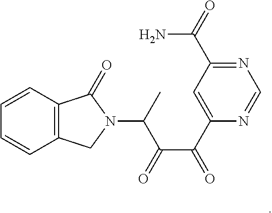 [SMILES: CC(C(=O)C(=O)c1cc(C(N)=O)ncn1)N1Cc2ccccc2C1=O ; "."]

To 6-[3-(1,3-dioxoisoindolin-2-yl)-2-oxo-butyl]pyrimidine-4-carboxamide (prepared as described above) (1.6 g, 4.7 mmol) in tetrahydrofuran (32 mL) was added selenium dioxide (1.1 g, 9.5 mmol) at room temperature. The reaction mixture was stirred at 65° C. for 16 hours, cooled to room temperature and filtered through a celite bed. The filter cake was washed with EtOAc and the filtrate concentrated in vacuo to afford crude 6-[3-(1,3-dioxoisoindolin-2-yl)-2-oxo-butanoyl]pyrimidine-4-carboxamide, which was used directly into the next step.

### Step 5: Preparation of 6-[3-(1-aminoethyl)pyrazin-2-yl]pyrimidine-4-carboxamide

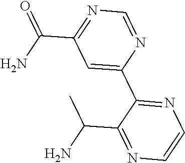 [SMILES: CC(N)c1nccnc1-c1cc(C(N)=O)ncn1]

To a mixture of crude 6-[3-(1,3-dioxoisoindolin-2-yl)-2-oxo-butanoyl]pyrimidine-4-carboxamide (prepared as described above) (1.7 g) in ethanol (17 mL) at 0° C. was added a solution of ethane-1,2-diamine (2.9 g, 48 mmol, 3.3 mL) in ethanol (17 mL) dropwise at 0° C. After addition, the reaction mixture was stirred at room temperature for 24 h, then diluted with water and the product extracted with 30% ACN in ethylacetate. The combined organic layers were washed with water, dried over sodium sulfate, filtered and concentrated under reduced pressure to afford crude 6-[3-(1-aminoethyl)pyrazin-2-yl]pyrimidine-4-carboxamide, which was used directly into the next step. LCMS (method 3): retention time 0.17 min, m/z 245 [M+H]⁺.

### Step 6: Preparation of 6-[3-[1-[(6-chloro-8-iodo-quinazolin-4-yl)amino]ethyl]pyrazin-2-yl]pyrimidine-4-carboxamide (compound P58)

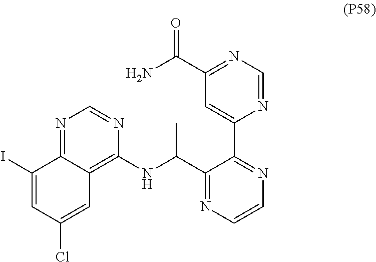 [SMILES: CC(Nc1ncnc2c(I)cc(Cl)cc12)c1nccnc1-c1cc(C(N)=O)ncn1]

To a mixture of 4,6-dichloro-8-iodoquinazoline (CAS 100949-33-5) (0.088 g, 0.27 mmol) and crude 6-[3-(1-aminoethyl)pyrazin-2-yl]pyrimidine-4-carboxamide (prepared as described above) (0.07 g, 0.286 mmol) in ACN (1.4 mL) was added triethylamine (0.573 mmol). The reaction mixture was heated at 90° C. for 1 hour, cooled to room temperature and quenched over ice cold water. The product was extracted with ethyl acetate several times, the combined organic layers were dried over sodium sulfate, filtered and concentrated in vacuo. The residue was purified by combiflash (ethyl acetate in cyclohexane) to afford 6-[3-[1-[(6-chloro-8-iodo-quinazolin-4-yl)amino]ethyl]pyrazin-2-yl]pyrimidine-4-carboxamide (P58) as a solid. LCMS (method 3): retention time 1.09 min, m/z 533/535 [M+H]⁺. ¹H NMR (400 MHz, DMSO-d₆) δ ppm 9.46 (s, 1H), 8.88 (br d, 1H), 8.77 (d, 1H), 8.72 (d, 1H), 8.64 (d, 1H), 8.57 (s, 1H), 8.46 (s, 1H), 8.35 (d, 1H), 8.02-8.11 (m, 2H), 6.15 (quint, 1H), 1.77 (d, 3H).

### Example E16: Preparation of 2-[5-[(1S)-1-[[6-chloro-8-(trifluoromethyl)quinazolin-4-yl]amino]ethyl]-1,2,4-triazol-1-ylpyrimidine-5-carboxamide (compound P68)

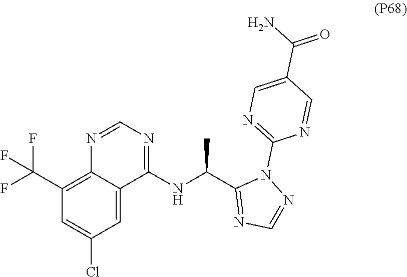 [SMILES: C[C@H](Nc1ncnc2c(C(F)(F)F)cc(Cl)cc12)c1ncnn1-c1ncc(C(N)=O)cn1]

### Step 1: Preparation of N-[(1 S)-1-[2-(5-bromopyrimidin-2-yl)-1,2,4-triazol-3-yl]ethyl]-6-chloro-8-(trifluoromethyl)quinazolin-4-amine

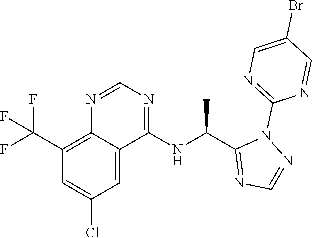 [SMILES: C[C@H](Nc1ncnc2c(C(F)(F)F)cc(Cl)cc12)c1ncnn1-c1ncc(Br)cn1]

To a mixture of 4,6-dichloro-8-(trifluoromethyl)quinazoline (prepared as described in WO 21/083936) (4.04 g, 15.1 mmol) and [(1S)-1-[2-(5-bromopyrimidin-2-yl)-1,2,4-triazol-3-yl]ethyl]ammonium chloride (prepared in analogy according to procedures found for example in WO 21/083936) (5.09 g, 16.6 mmol) in ACN (80.8 mL) was added triethylamine (4.62 g, 45.4 mmol, 5.32 mL) at room temperature. The reaction mixture was stirred at 90° C. for 1 hour, cooled to room temperature, then diluted with ice cold water (50 mL) and the product extracted with EtOAc (5×60 mL). The combined organic layers were washed with brine, dried over sodium sulfate, filtered and concentrated in vacuo. The residue was purified by combiflash (ethyl acetate in cyclohexane) to afford N-[(1S)-1-[2-(5-bromopyrimidin-2-yl)-1,2,4-triazol-3-yl]ethyl]-6-chloro-8-(trifluoromethyl)quinazolin-4-amine as a solid.
LCMS (method 3): retention time 1.14 min, m/z 499/501/503 [M+H]⁺.

### Step 2: Preparation of 2-[5-[(1 S)-1-[[6-chloro-8-(trifluoromethyl)quinazolin-4-yl]amino]ethyl]-1,2,4-triazol-1-yl]-N-[(4-methoxyphenyl)methyl]pyrimidine-5-carboxamide

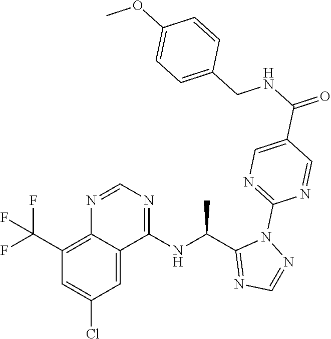 [SMILES: COc1ccc(CNC(=O)c2cnc(-n3ncnc3[C@H](C)Nc3ncnc4c(C(F)(F)F)cc(Cl)cc34)nc2)cc1]

A reactor vessel was charged with N-[(1S)-1-[2-(5-bromopyrimidin-2-yl)-1,2,4-triazol-3-yl]ethyl]-6-chloro-8-(trifluoromethyl)quinazolin-4-amine (prepared as described above) (700 mg, 1.41 mmol), 4-methoxybenzylamine (0.211 g, 1.54 mmol), bis(benzonitrile)palladium(II) chloride (0.1 equiv., 0.141 mmol), 4,5-bis(diphenylphosphino)-9,9-dimethylxanthene (Xantphos, 0.141 mmol), toluene (7 mL) and triethylamine (2.1 mmol). The vessel was flushed with nitrogen, then with carbon monoxide. The reaction mixture was heated to 80° C. under an 8 bar carbon monoxide pressure for 3 hours. After cooling to room temperature, the pressure was carefully released and the vessel flushed with nitrogen. The mixture was filtered through a celite bed, the filter cake washed with EtOAc and the filtrate concentrated under reduced pressure. The residue was purified by column chromatography (ethyl acetate in cyclohexane) to afford 2-[5-[(1S)-1-[[6-chloro-8-(trifluoromethyl)quinazolin-4-yl]amino]ethyl]-1,2,4-triazol-1-yl]-N-[(4-methoxyphenyl)methyl]-pyrimidine-5-carboxamide as a solid. LCMS (method 3): retention time 1.10 min, m/z 584/586 [M+H]⁺.

¹H NMR (400 MHz, DMSO-d₆) δ ppm 9.41 (t, 1H), 9.33 (s, 2H), 9.11 (d, 1H), 8.91 (d, 1H), 8.28 (s, 1H), 8.20 (d, 1H), 8.18 (s, 1H), 7.31 (d, 2H), 6.92 (d, 2H), 6.32 (quint, 1H), 4.47 (d, 2H), 3.74 (s, 3H), 1.77 (d, 3H).

### Step 3: Preparation of 2-[5-[(1S)-1-[[6-chloro-8-(trifluoromethyl)quinazolin-4-yl]amino]ethyl]-1,2,4-triazol-1-yl]pyrimidine-5-carboxamide (compound P68)

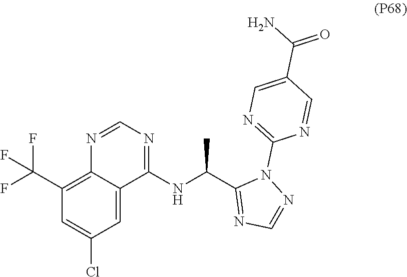 [SMILES: C[C@H](Nc1ncnc2c(C(F)(F)F)cc(Cl)cc12)c1ncnn1-c1ncc(C(N)=O)cn1]

To a solution of 2-[5-[(1S)-1-[[6-chloro-8-(trifluoromethyl)quinazolin-4-yl]amino]ethyl]-1,2,4-triazol-1-yl]-N-[(4-methoxyphenyl)methyl]pyrimidine-5-carboxamide (prepared as described above) (0.15 g, 0.256 mmol) in acetonitrile (3.75 mL) was added a solution of cerium(IV) ammonium nitrate (0.283 g, 0.513 mmol) in water (1 mL). The reaction mixture was stirred at room temperature for 16 hours, then diluted with an aqueous saturated sodium bicarbonate solution. The product was extracted several times with ethyl acetate, the combined organic layers washed with brine, dried over sodium sulfate, filtered and concentrated in vacuo. The residue was purified by combiflash (ethyl acetate in cyclohexane) to afford 2-[5-[(1S)-1-[[6-chloro-8-(trifluoromethyl)quinazolin-4-yl]amino]ethyl]-1,2,4-triazol-1-yl]pyrimidine-5-carboxamide (P68) as a solid. LCMS (method 3): retention time 0.99 min, m/z 464/466 [M+H]⁺.

### Example E17: Preparation of 6-[5-[(1S)-1-[[6-chloro-8-(trifluoromethyl)quinazolin-4-yl]-methyl-amino]ethyl]-1,2,4-triazol-1-yl]-N-(2-cyanoethyl)pyrimidine-4-carboxamide (compound P53)

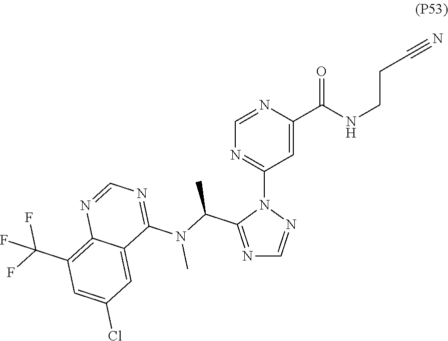 [SMILES: C[C@@H](c1ncnn1-c1cc(C(=O)NCCC#N)ncn1)N(C)c1ncnc2c(C(F)(F)F)cc(Cl)cc12]

A solution of methyl 6-[5-[(1S)-1-[[6-chloro-8-(trifluoromethyl)quinazolin-4-yl]-methyl-amino]ethyl]-1,2,4-triazol-1-yl]pyrimidine-4-carboxylate (I-22) (0.15 g, 0.304 mmol) and 3-aminopropanenitrile (0.913 mmol) in methanol (1.5 mL) was stirred at room temperature for 18 hours. The reaction mixture was diluted with water (15 mL) and the formed solid isolated by filtration. This crude solid material was purified by combiflash (ethyl acetate in cyclohexane) to afford 6-[5-[(1S)-1-[[6-chloro-8-(trifluoromethyl)quinazolin-4-yl]-methyl-amino]ethyl]-1,2,4-triazol-1-yl]-N-(2-cyanoethyl)pyrimidine-4-carboxamide (P53) as a white solid. LCMS (method 3): retention time 1.13 min, m/z 531/533 [M+H]⁺. ¹H NMR (CDCl₃) δ: 8.71 (d, 1H), 8.64 (d, 1H), 8.52 (s, 1H), 8.35 (br t, 1H), 8.07 (s, 1H), 8.04 (s, 2H), 6.91 (quint, 1H), 3.81 (m, 2H), 3.45 (s, 3H), 2.79 (t, 2H), 1.96 (d, 3H).

TABLE P
Examples of compounds of formula (I)

| | | | | [M + | | |
|---|---|---|---|---|---|---|
| | | | | H]⁺ | | |
| En- | | | RT | (meas- | Meth- | |
| try | IUPAC name | STRUCTURE | (min) | ured) | od | MP |
| P1 | 6-[5-[(1S)-1-[[6-iodo-8- (trifluoromethyl)quina- zolin- 4-yl]amino]ethyl]-1,2,4- triazol-1-yl]pyrimidine-4- carboxamide | 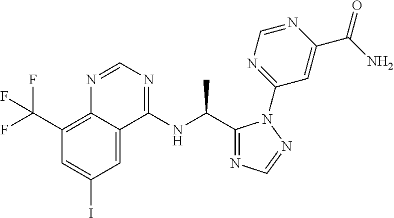 [SMILES: C[C@H](Nc1ncnc2c(C(F)(F)F)cc(I)cc12)c1ncnn1-c1cc(C(N)=O)ncn1] | 1.10 | 556 | 2 | 246- 248 |
| P2 | 6-[5-[(1S)-1-[(8-chloro-6- iodo-quinazolin-4- yl)amino]ethyl]-1,2,4- triazol-1-yl]pyrimidine-4- carboxamide | 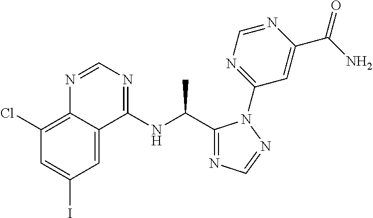 [SMILES: C[C@H](Nc1ncnc2c(Cl)cc(I)cc12)c1ncnn1-c1cc(C(N)=O)ncn1] | 0.96 | 522/524 | 2 | — |
| P3 | 6-[5-[(1S)-1-[[6-chloro-8- (trifluoromethyl)quina- zolin- 4-yl]amino]ethyl]-1,2,4- triazol-1-yl]-N-methyl- pyrimidine-4- carboxamide | 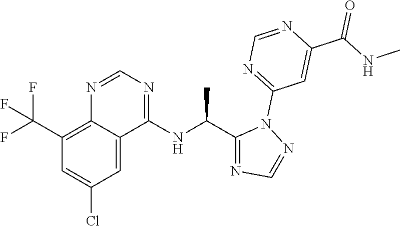 [SMILES: CNC(=O)c1cc(-n2ncnc2[C@H](C)Nc2ncnc3c(C(F)(F)F)cc(Cl)cc23)ncn1] | 1.05 | 478/480 | 2 | 228- 230 |
| P4 | 6-[5-[(1S)-1-[(8-chloro-6- iodo-quinazolin-4- yl)amino]ethyl]-1,2,4- triazol-1-yl]-N-methyl- pyrimidine-4- carboxamide | 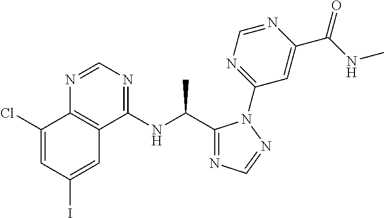 [SMILES: CNC(=O)c1cc(-n2ncnc2[C@H](C)Nc2ncnc3c(Cl)cc(I)cc23)ncn1] | 0.99 | 536/538 | 2 | 190- 192 |
| P5 | 6-[5-[(1S)-1-[[6-chloro-8- (trifluoromethyl)quina- zolin- 4-yl]amino]ethyl]-1,2,4- triazol-1-yl]pyrimidine-4- carboxamide | 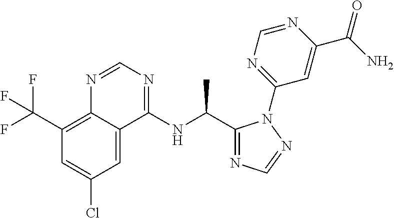 [SMILES: C[C@H](Nc1ncnc2c(C(F)(F)F)cc(Cl)cc12)c1ncnn1-c1cc(C(N)=O)ncn1] | 1.07 | 464/466 | 2 | 231- 233 |
| P6 | 2-[5-[(1S)-1-[[6-chloro-8- (trifluoromethyl)quina- zolin- 4-yl]amino]ethyl]-1,2,4- triazol-1-yl]-N-ethyl-N- methyl-thiazole-5- carboxamide | 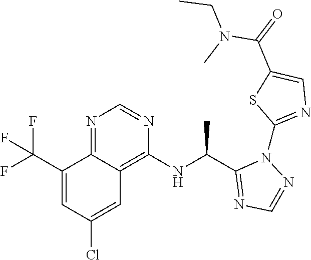 [SMILES: CCN(C)C(=O)c1cnc(-n2ncnc2[C@H](C)Nc2ncnc3c(C(F)(F)F)cc(Cl)cc23)s1] | 1.09 | 511/513 | 3 | 164- 166 |
| P7 | 2-[5-[(1S)-1-[[6-chloro-8- (trifluoromethyl)quina- zolin- 4-yl]amino]ethyl]-1,2,4- triazol-1-yl]-N-methyl- thiazole-5-carboxamide | 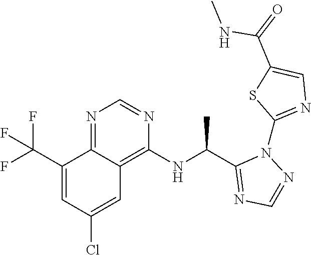 [SMILES: CNC(=O)c1cnc(-n2ncnc2[C@H](C)Nc2ncnc3c(C(F)(F)F)cc(Cl)cc23)s1] | 0.99 | 483/485 | 3 | 174- 176 |
| P8 | 2-[5-[(1S)-1-[6-chloro-8- (trifluoromethyl)quina- zolin- 4-yl]amino]ethyl]-1,2,4- triazol-1-yl]thiazole-5- carboxamide | 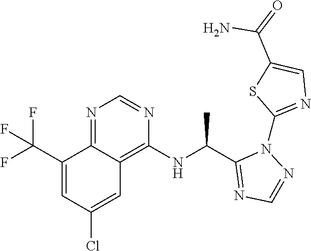 [SMILES: C[C@H](Nc1ncnc2c(C(F)(F)F)cc(Cl)cc12)c1ncnn1-c1ncc(C(N)=O)s1] | 0.95 | 469/471 | 3 | 200- 202 |
| P9 | 6-[5-[(1S)-1-[[6-chloro-8- (trifluoromethyl)quina- zolin-4-yl]-methyl- amino]ethyl]-1,2,4- triazol-1-yl]-N-methyl- pyrimidine-4- carboxamide | 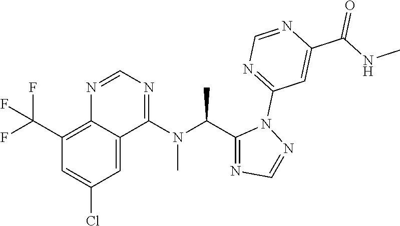 [SMILES: CNC(=O)c1cc(-n2ncnc2[C@H](C)N(C)c2ncnc3c(C(F)(F)F)cc(Cl)cc23)ncn1] | 1.06 | 492/494 | 3 | 113- 115 |
| P10 | 6-[5-[(1S)-1-[(6,8- dibromoquinazolin-4- yl)amino]ethyl]-1,2,4- triazol-1-yl]pyrimidine-4- carboxamide | 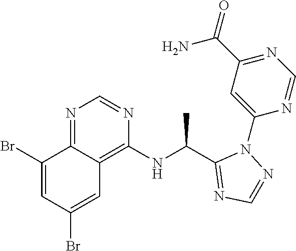 [SMILES: C[C@H](Nc1ncnc2c(Br)cc(Br)cc12)c1ncnn1-c1cc(C(N)=O)ncn1] | 1.47 | 518/520/ 522 | 4 | 199- 201 |
| P11 | 6-[5-[(1S)-1-[(6,8- dibromoquinazolin-4-yl)- methyl-amino]ethyl]- 1,2,4-triazol-1-yl]-N- methyl-pyrimidine-4- carboxamide | 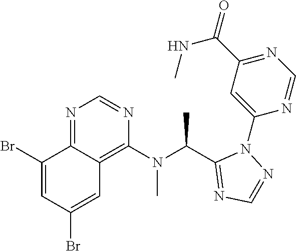 [SMILES: CNC(=O)c1cc(-n2ncnc2[C@H](C)N(C)c2ncnc3c(Br)cc(Br)cc23)ncn1] | 1.00 | 546/548/ 550 | 3 | 153- 155 |
| P12 | 6-[5-[(1S)-1-[(6,8- dibromoquinazolin-4- yl)amino]ethyl]-1,2,4- triazol-1-yl]-N-methyl- pyrimidine-4- carboxamide | 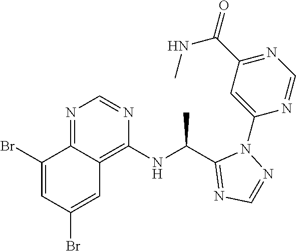 [SMILES: CNC(=O)c1cc(-n2ncnc2[C@H](C)Nc2ncnc3c(Br)cc(Br)cc23)ncn1] | 0.88 | 532/534/ 536 | 3 | 230- 232 |
| P13 | 6-[5-[(1S)-1-[(6-chloro- 8-iodo-quinazolin-4- yl)amino]ethyl]-1,2,4- triazol-1-yl]pyrimidine- 4-carboxamide | 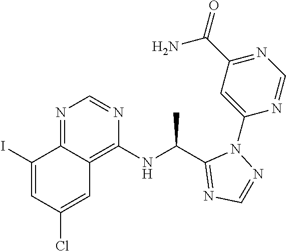 [SMILES: C[C@H](Nc1ncnc2c(I)cc(Cl)cc12)c1ncnn1-c1cc(C(N)=O)ncn1] | 1.07 | 522/524 | 3 | — |
| P14 | 6-[5-[(1S)-1-[(6-chloro-8- iodo-quinazolin-4- yl)amino]ethyl]-1,2,4- triazol-1-yl]-N-methyl- pyrimidine-4- carboxamide | 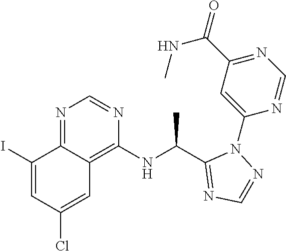 [SMILES: CNC(=O)c1cc(-n2ncnc2[C@H](C)Nc2ncnc3c(I)cc(Cl)cc23)ncn1] | 1.09 | 536/538 | 3 | 179- 181 |
| P15 | 6-[5-[(1S)-1-[[8-chloro-6- (trifluoromethyl)quina- zolin-4-yl]-methyl- amino]ethyl]-1,2,4- triazol-1-yl]-N-methyl- pyrimidine-4- carboxamide | 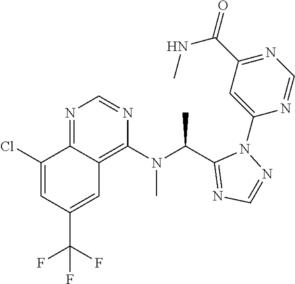 [SMILES: CNC(=O)c1cc(-n2ncnc2[C@H](C)N(C)c2ncnc3c(Cl)cc(C(F)(F)F)cc23)ncn1] | 1.05 | 492/494 | 3 | 130- 132 |
| P16 | 6-[5-[(1S)-1-[[8-chloro-6- (trifluoromethyl)quina- zolin- 4-yl]amino]ethyl]-1,2,4- triazol-1-yl]-N-methyl- pyrimidine-4- carboxamide | 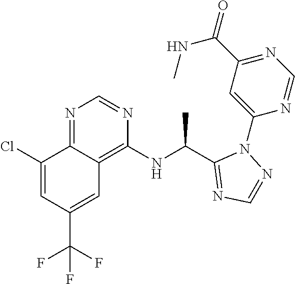 [SMILES: CNC(=O)c1cc(-n2ncnc2[C@H](C)Nc2ncnc3c(Cl)cc(C(F)(F)F)cc23)ncn1] | 1.68 | 478/480 | 4 | 163- 165 |
| P17 | 6-[5-[(1S)-1-[[8-chloro-6- (trifluoromethyl)quina- zolin- 4-yl]amino]ethyl]-1,2,4- triazol-1-yl]pyrimidine-4- carboxamide | 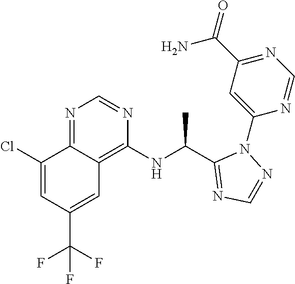 [SMILES: C[C@H](Nc1ncnc2c(Cl)cc(C(F)(F)F)cc12)c1ncnn1-c1cc(C(N)=O)ncn1] | 1.03 | 464/466 | 3 | 175- 177 |
| P18 | 6-[5-[(1S)-1-[(8-chloro-6- iodo-quinazolin-4- yl)amino]ethyl]-1,2,4- triazol-1-yl]pyrimidine-4- carbonitrile | 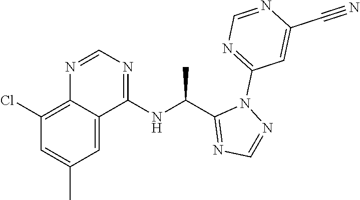 [SMILES: Cc1cc(Cl)c2ncnc(N[C@@H](C)c3ncnn3-c3cc(C#N)ncn3)c2c1] | 1.13 | 504/506 | 3 | — |
| P19 | 6-[5-[(1S)-1-[[6-chloro-8- (trifluoromethyl)quina- zolin- 4-yl]amino]ethyl]-1,2,4- triazol-1-yl]pyrimidine-4- carbonitrile | 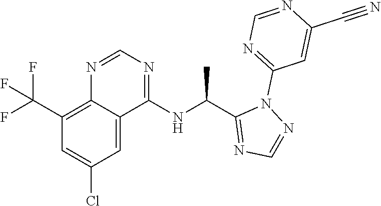 [SMILES: C[C@H](Nc1ncnc2c(C(F)(F)F)cc(Cl)cc12)c1ncnn1-c1cc(C#N)ncn1] | 1.14 | 446/448 | 3 | 250- 252 |
| P20 | 6-[5-[(1S)-1-[[6-chloro-8- (trifluoromethyl)quina- zolin- 4-yl]amino]ethyl]-1,2,4- triazol-1-yl]pyridazine-3- carboxamide | 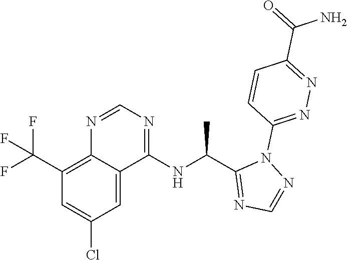 [SMILES: C[C@H](Nc1ncnc2c(C(F)(F)F)cc(Cl)cc12)c1ncnn1-c1ccc(C(N)=O)nn1] | 1.84 | 464/466 | 4 | 250- 251 |
| P21 | 5-[5-[(1S)-1-[[6-chloro-8- (trifluoromethyl)quina- zolin- 4-yl]amino]ethyl]-1,2,4- triazol-1-yl]pyrazine-2- carboxamide | 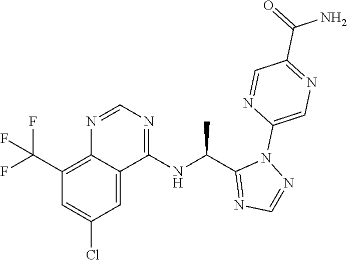 [SMILES: C[C@H](Nc1ncnc2c(C(F)(F)F)cc(Cl)cc12)c1ncnn1-c1cnc(C(N)=O)cn1] | 1.74 | 464/466 | 4 | 280- 280 |
| P22 | 6-[5-[(1S)-1-[6-chloro-8- (trifluoromethyl)quina- zolin- 4-yl]amino]ethyl]-1,2,4- triazol-1-yl]-N-(1- cyanocyclopropyl)pyrimi- dine-4-carboxamide | 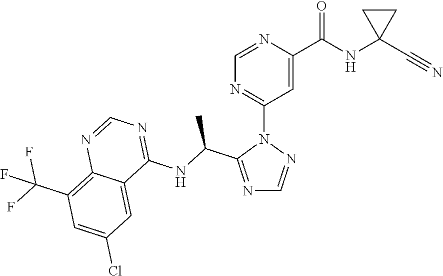 [SMILES: C[C@H](Nc1ncnc2c(C(F)(F)F)cc(Cl)cc12)c1ncnn1-c1cc(C(=O)NC2(C#N)CC2)ncn1] | 1.22 | 529/531 | 3 | 173- 175 |
| P23 | 6-[5-[(1S)-1-[[6-chloro-8- (trifluoromethyl)quina- zolin- 4-yl]amino]ethyl]-1,2,4- triazol-1-yl]-N- cyclopropyl-pyrimidine-4- carboxamide | 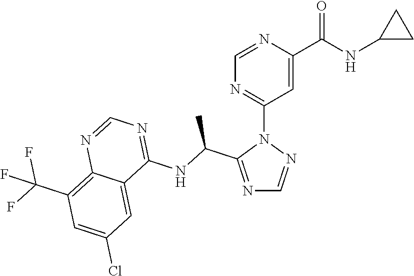 [SMILES: C[C@H](Nc1ncnc2c(C(F)(F)F)cc(Cl)cc12)c1ncnn1-c1cc(C(=O)NC2CC2)ncn1] | 1.24 | 504/506 | 3 | 156- 158 |
| P24 | 6-[5-[(1S)-1-[[6-chloro-8- (trifluoromethyl)quina- zolin- 4-yl]amino]ethyl]-1,2,4- triazol-1-yl]-N-(1,1- dioxothietan-3- yl)pyrimidine-4- carboxamide | 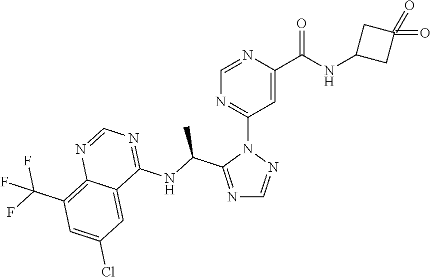 | 1.20 | 568/570 | 3 | 180- 182 |
| P25 | 6-[5-[(1S)-1-[[6-chloro-8- (trifluoromethyl)quina- zolin- 4-yl]amino]ethyl]-1,2,4- triazol-1-yl]-N-(thietan-3- yl)pyrimidine-4- carboxamide | 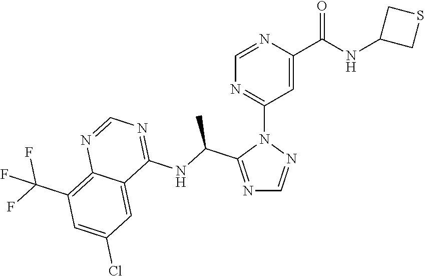 [SMILES: C[C@H](Nc1ncnc2c(C(F)(F)F)cc(Cl)cc12)c1ncnn1-c1cc(C(=O)NC2CSC2)ncn1] | 1.28 | 536/538 | 3 | 159- 160 |
| P26 | 6-[5-[(1S)-1-[[6-chloro-8- (trifluoromethyl)quina- zolin- 4-yl]amino]ethyl]-1,2,4- triazol-1-yl]pyrimidine-4- carbohydroxamic acid | 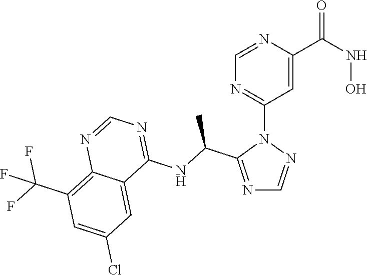 [SMILES: C[C@H](Nc1ncnc2c(C(F)(F)F)cc(Cl)cc12)c1ncnn1-c1cc(C(=O)NO)ncn1] | 1.14 | 480/482 | 3 | 195- 197 |
| P27 | 6-[5-[(1S)-1-[[6-chloro-8- (trifluoromethyl)quina- zolin- 4-yl]amino]ethyl]-1,2,4- triazol-1-yl]-N-methoxy- N-methyl-pyrimidine-4- carboxamide | 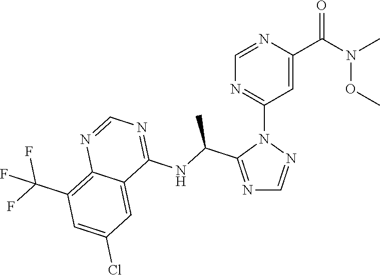 [SMILES: CON(C)C(=O)c1cc(-n2ncnc2[C@H](C)Nc2ncnc3c(C(F)(F)F)cc(Cl)cc23)ncn1] | 1.22 | 508/510 | 3 | 143- 145 |
| P28 | 6-[5-[(1S)-1-[[6-chloro-8- (trifluoromethyl)quina- zolin- 4-yl]amino]ethyl]-1,2,4- triazol-1-yl]-N-methoxy- pyrimidine-4- carboxamide | 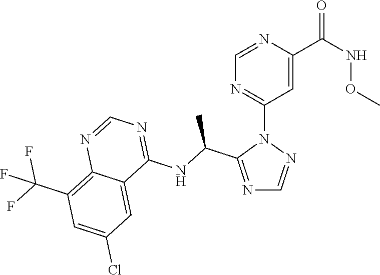 [SMILES: CONC(=O)c1cc(-n2ncnc2[C@H](C)Nc2ncnc3c(C(F)(F)F)cc(Cl)cc23)ncn1] | 1.16 | 494/496 | 3 | 204- 206 |
| P29 | 2-[5-[(1S)-1-[(6-chloro-8- iodo-quinazolin-4- yl)amino]ethyl]-1,2,4- triazol-1-yl]thiazole-5- carboxamide | 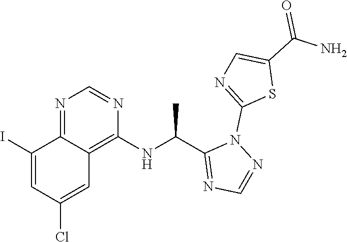 [SMILES: C[C@H](Nc1ncnc2c(I)cc(Cl)cc12)c1ncnn1-c1ncc(C(N)=O)s1] | 1.06 | 527/529 | 3 | 220- 222 |
| P30 | 6-[5-[(1S)-1-[[6-chloro-8- (trifluoromethyl)quina- zolin- 4-yl]amino]ethyl]-1,2,4- triazol-1-yl]-N,N- dimethyl-pyrimidine-4- carboxamide | 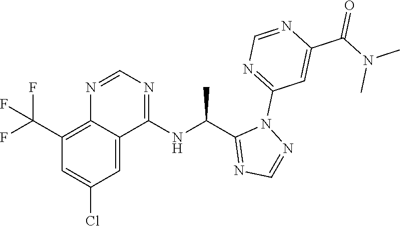 [SMILES: C[C@H](Nc1ncnc2c(C(F)(F)F)cc(Cl)cc12)c1ncnn1-c1cc(C(=O)N(C)C)ncn1] | 1.20 | 492/494 | 3 | 134- 136 |
| P31 | 6-[5-[(1S)-1-[[6-chloro-8- (trifluoromethyl)quina- zolin- 4-yl]amino]ethyl]-1,2,4- triazol-1-yl]-N-ethyl-N- methyl-pyrimidine-4- carboxamide | 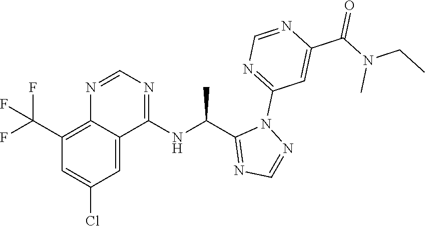 [SMILES: CCN(C)C(=O)c1cc(-n2ncnc2[C@H](C)Nc2ncnc3c(C(F)(F)F)cc(Cl)cc23)ncn1] | 1.23 | 506/508 | 3 | 139- 141 |
| P32 | [6-[5-[(1S)-1-[[6-chloro-8- (trifluoromethyl)quina- zolin- 4-yl]amino]ethyl]-1,2,4- triazol-1-yl]pyrimidin-4- yl]-morpholino- methanone | 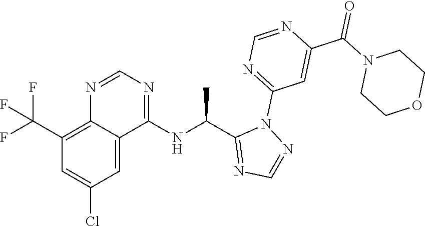 [SMILES: C[C@H](Nc1ncnc2c(C(F)(F)F)cc(Cl)cc12)c1ncnn1-c1cc(C(=O)N2CCOCC2)ncn1] | 1.20 | 534/536 | 3 | 160- 162 |
| P33 | 6-[5-[(1S)-1-[(6-chloro-8- iodo-quinazolin-4-yl)- methyl-amino]ethyl]- 1,2,4-triazol-1-yl]-N- methyl-pyrimidine-4- carboxamide | 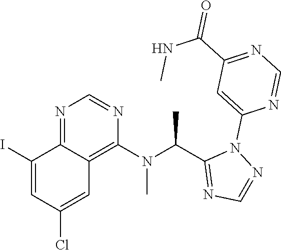 [SMILES: CNC(=O)c1cc(-n2ncnc2[C@H](C)N(C)c2ncnc3c(I)cc(Cl)cc23)ncn1] | 1.15 | 550/552 | 3 | 128- 130 |
| P34 | 6-[5-[(1S)-1-[(8-chloro-6- iodo-quinazolin-4-yl)- methyl-amino]ethyl]- 1,2,4-triazol-1-yl]-N- methyl-pyrimidine-4- carboxamide | 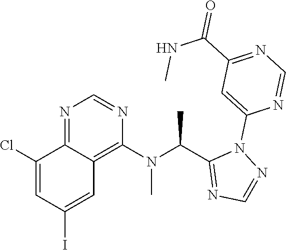 [SMILES: CNC(=O)c1cc(-n2ncnc2[C@H](C)N(C)c2ncnc3c(Cl)cc(I)cc23)ncn1] | 1.12 | 550/552 | 3 | 155- 157 |
| P35 | 2-[5-[(1S)-1-[[8-chloro-6- (trifluoromethyl)quina- zolin- 4-yl]amino]ethyl]-1,2,4- triazol-1-yl]thiazole-5- carboxamide | 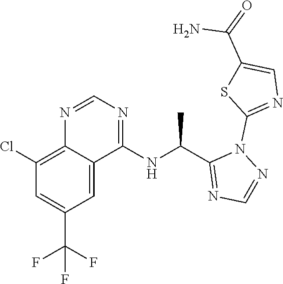 [SMILES: C[C@H](Nc1ncnc2c(Cl)cc(C(F)(F)F)cc12)c1ncnn1-c1ncc(C(N)=O)s1] | 1.76 | 469/471 | 4 | 239- 241 |
| P36 | 2-[5-[(1S)-1-[(6,8- dibromoquinazolin-4- yl)amino]ethyl]-1,2,4- triazol-1-yl]thiazole-5- carboxamide | 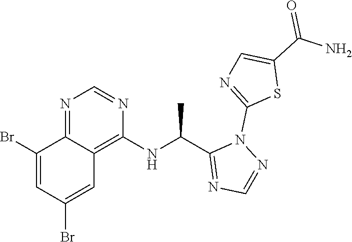 [SMILES: C[C@H](Nc1ncnc2c(Br)cc(Br)cc12)c1ncnn1-c1ncc(C(N)=O)s1] | 1.80 | 523/525/ 527 | 4 | 250- 250 |
| P37 | 2-[5-[(1S)-1-[(8-chloro-6- iodo-quinazolin-4- yl)amino]ethyl]-1,2,4- triazol-1-yl]thiazole-5- carboxamide | 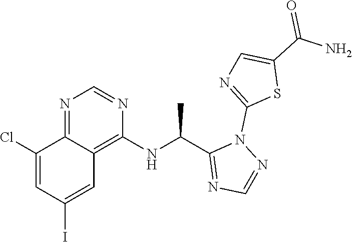 [SMILES: C[C@H](Nc1ncnc2c(Cl)cc(I)cc12)c1ncnn1-c1ncc(C(N)=O)s1] | 1.79 | 527/529 | 4 | 250- 250 |
| P38 | 6-[5-[(1S)-1-[[6-chloro-8- (trifluoromethyl)quina- zolin- 4-yl]amino]ethyl]-1,2,4- triazol-1-yl]-N- (cyanomethyl)pyrimidine- 4-carboxamide | 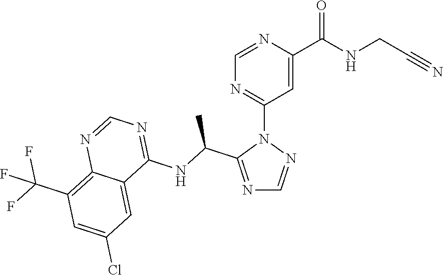 [SMILES: C[C@H](Nc1ncnc2c(C(F)(F)F)cc(Cl)cc12)c1ncnn1-c1cc(C(=O)NCC#N)ncn1] | 1.18 | 503/505 | 3 | 166- 168 |
| P39 | 6-[5-[(1S)-1-[6-chloro-8- (trifluoromethyl)quina- zolin- 4-yl]amino]ethyl]-1,2,4- triazol-1-yl]-N-(2- cyanoethyl)pyrimidine-4- carboxamide | 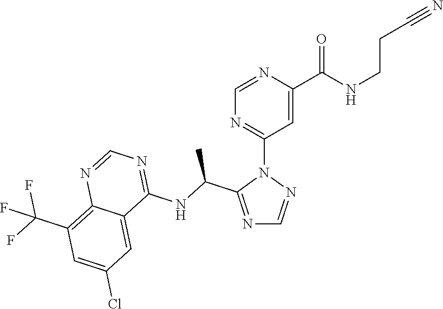 [SMILES: C[C@H](Nc1ncnc2c(C(F)(F)F)cc(Cl)cc12)c1ncnn1-c1cc(C(=O)NCCC#N)ncn1] | 1.19 | 517/519 | 3 | 157- 159 |
| P40 | 6-[5-[(1S)-1-[(6-chloro-8- iodo-quinazolin-4- yl)amino]ethyl]-1,2,4- triazol-1-yl]pyrimidine-4- carbonitrile | 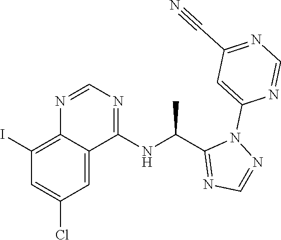 [SMILES: C[C@H](Nc1ncnc2c(I)cc(Cl)cc12)c1ncnn1-c1cc(C#N)ncn1] | 1.16 | 504/506 | 3 | 251- 252 |
| P41 | 6-[5-[(1S)-1-[(6,8- dibromoquinazolin-4- yl)amino]ethyl]-1,2,4- triazol-1-yl]pyrimidine-4- carbonitrile | 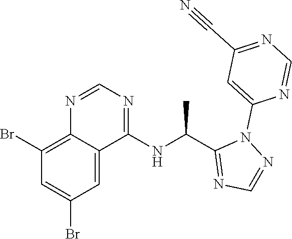 [SMILES: C[C@H](Nc1ncnc2c(Br)cc(Br)cc12)c1ncnn1-c1cc(C#N)ncn1] | 1.17 | 500/502/ 504 | 3 | 247- 249 |
| P42 | 6-[5-[(1S)-1-[[6-chloro-8- (trifluoromethyl)quina- zolin- 4-yl]amino]ethyl]-1,2,4- triazol-1-yl]-N-(1-cyano- 1-cyclopropyl- ethyl)pyrimidine-4- carboxamide | 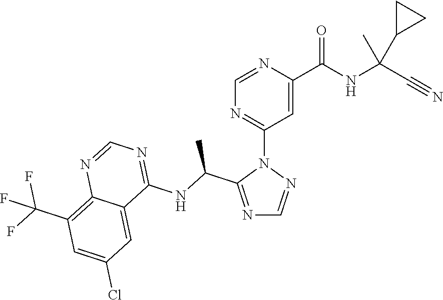 [SMILES: C[C@H](Nc1ncnc2c(C(F)(F)F)cc(Cl)cc12)c1ncnn1-c1cc(C(=O)NC(C)(C#N)C2CC2)ncn1] | 1.20 | 557/559 | 3 | 200- 202 |
| P43 | 6-[5-[(1S)-1-[[6-chloro-8- (trifluoromethyl)quina- zolin- 4-yl]amino]ethyl]-1,2,4- triazol-1-yl]-N-(1-cyano- 1-methyl- ethyl)pyrimidine-4- carboxamide | 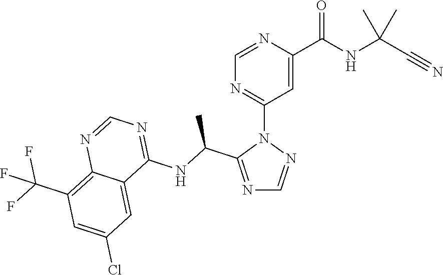 [SMILES: C[C@H](Nc1ncnc2c(C(F)(F)F)cc(Cl)cc12)c1ncnn1-c1cc(C(=O)NC(C)(C)C#N)ncn1] | 1.16 | 531/533 | 3 | 164- 166 |
| P44 | 6-[5-[(1S)-1-[[6-chloro-8- (trifluoromethyl)quina- zolin- 4-yl]amino]ethyl]-1,2,4- triazol-1-yl]-N-(3-methyl- 1,1-dioxo-thietan-3- yl)pyrimidine-4- carboxamide | 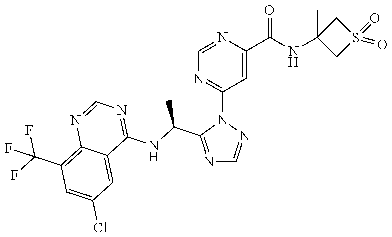 [SMILES: C[C@H](Nc1ncnc2c(C(F)(F)F)cc(Cl)cc12)c1ncnn1-c1cc(C(=O)NC2(C)CS(=O)(=O)C2)ncn1] | 1.13 | 582/584 | 3 | 199- 201 |
| P45 | 6-[5-[(1S)-1-[[6-chloro-8- (trifluoromethyl)quina- zolin- 4-yl]amino]ethyl]-1,2,4- triazol-1-yl]-N-(3- methylthietan-3- yl)pyrimidine-4- carboxamide | 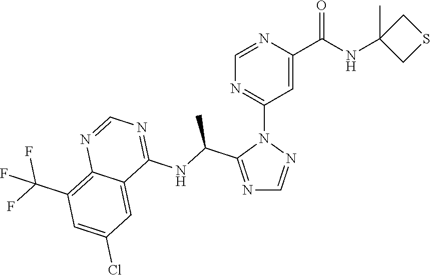 [SMILES: C[C@H](Nc1ncnc2c(C(F)(F)F)cc(Cl)cc12)c1ncnn1-c1cc(C(=O)NC2(C)CSC2)ncn1] | 1.25 | 550/552 | 3 | 162- 164 |
| P46 | 6-[5-[(1S)-1-[[6-chloro-8- (trifluoromethyl)quina- zolin- 4-yl]amino]ethyl]-1,2,4- triazol-1-yl]-N-hydroxy- N-methyl-pyrimidine-4- carboxamide | 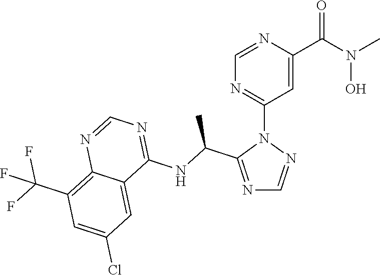 [SMILES: C[C@H](Nc1ncnc2c(C(F)(F)F)cc(Cl)cc12)c1ncnn1-c1cc(C(=O)N(C)O)ncn1] | 1.05 | 494/496 | 3 | 155- 157 |
| P47 | 6-[5-[(1S)-1-[(6-chloro-8- iodo-quinazolin-4-yl)- methyl-amino]ethyl]- 1,2,4-triazol-1- yl]pyrimidine-4- carboxamide | 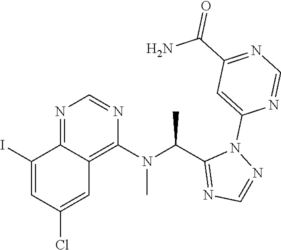 [SMILES: C[C@@H](c1ncnn1-c1cc(C(N)=O)ncn1)N(C)c1ncnc2c(I)cc(Cl)cc12] | 1.11 | 536/538 | 3 | 145- 147 |
| P48 | 6-[5-[(1S)-1-[(6,8- dibromoquinazolin-4-yl)- methyl-amino]ethyl]- 1,2,4-triazol-1- yl]pyrimidine-4- carboxamide | 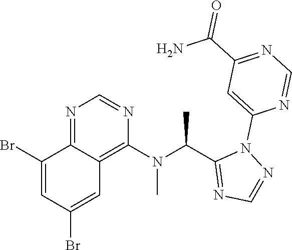 [SMILES: C[C@@H](c1ncnn1-c1cc(C(N)=O)ncn1)N(C)c1ncnc2c(Br)cc(Br)cc12] | 1.08 | 523/534/ 536 | 3 | 178- 180 |
| P49 | 6-[5-[(1S)-1-[(8-chloro-6- iodo-quinazolin-4-yl)- methyl-amino]ethyl]- 1,2,4-triazol-1- yl]pyrimidine-4- carboxamide | 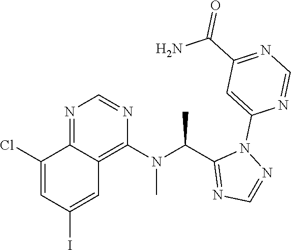 [SMILES: C[C@@H](c1ncnn1-c1cc(C(N)=O)ncn1)N(C)c1ncnc2c(Cl)cc(I)cc12] | 1.05 | 536/538 | 3 | 248- 250 |
| P50 | 6-[5-[(1S)-1-[[6-chloro-8- (trifluoromethyl)quina- zolin-4-yl]-methyl- amino]ethyl]-1,2,4- triazol-1-yl]pyrimidine-4- carboxamide | 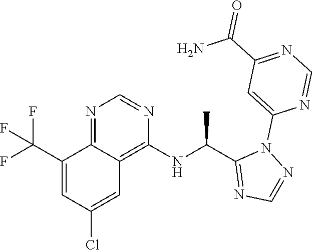 [SMILES: C[C@H](Nc1ncnc2c(C(F)(F)F)cc(Cl)cc12)c1ncnn1-c1cc(C(N)=O)ncn1] | 1.11 | 478/480 | 3 | 126- 128 |
| P51 | 6-[5-[(1S)-1-[[8-chloro-6- (trifluoromethyl)quina- zolin-4-yl]-methyl- amino]ethyl]-1,2,4- triazol-1-yl]pyrimidine-4- carboxamide | 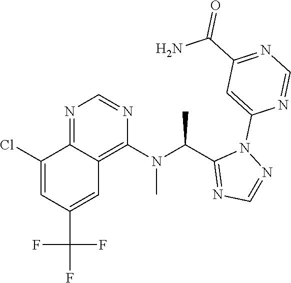 [SMILES: C[C@@H](c1ncnn1-c1cc(C(N)=O)ncn1)N(C)c1ncnc2c(Cl)cc(C(F)(F)F)cc12] | 1.07 | 478/480 | 3 | 237- 239 |
| P52 | 6-[5-[(1S)-1-[[6-chloro-8- (trifluoromethyl)quina- zolin-4-yl]-methyl- amino]ethyl]-1,2,4- triazol-1-yl]-N- (cyanomethyl)pyrimidine- 4-carboxamide | 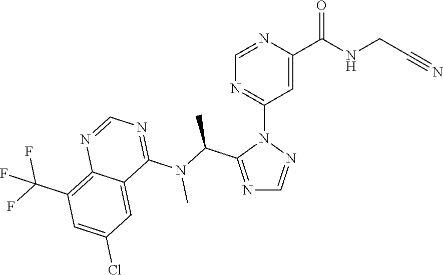 [SMILES: C[C@@H](c1ncnn1-c1cc(C(=O)NCC#N)ncn1)N(C)c1ncnc2c(C(F)(F)F)cc(Cl)cc12] | 1.13 | 517/519 | 3 | 126- 128 |
| P53 | 6-[5-[(1S)-1-[[6-chloro-8- (trifluoromethyl)quina- zolin-4-yl]-methyl- amino]ethyl]-1,2,4- triazol-1-yl]-N-(2- cyanoethyl)pyrimidine-4- carboxamide | 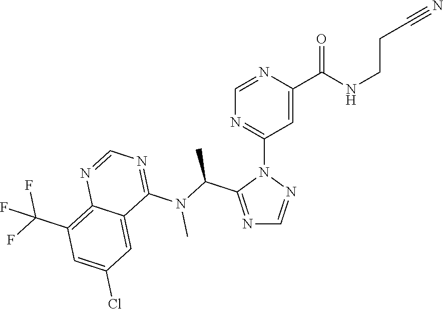 [SMILES: C[C@@H](c1ncnn1-c1cc(C(=O)NCCC#N)ncn1)N(C)c1ncnc2c(C(F)(F)F)cc(Cl)cc12] | 1.13 | 531/533 | 3 | 119- 121 |
| P54 | [6-[5-[(1S)-1-[6-chloro-8- (trifluoromethyl)quina- zolin-4-yl]-methyl- amino]ethyl]-1,2,4- triazol-1-yl]pyrimidin-4- yl]-morpholino- methanone | 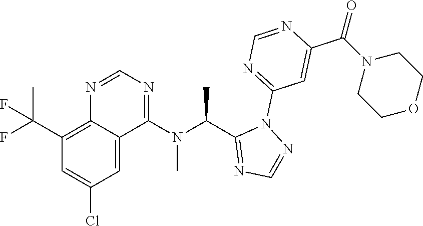 [SMILES: C[C@@H](c1ncnn1-c1cc(C(=O)N2CCOCC2)ncn1)N(C)c1ncnc2c(C(C)(F)F)cc(Cl)cc12] | 1.13 | 548/550 | 3 | 123- 125 |
| P55 | 6-[5-[(1S)-1-[[6-chloro-8- (trifluoromethyl)quina- zolin-4-yl]-methyl- amino]ethyl]-1,2,4- triazol-1-yl]-N-ethyl-N- methyl-pyrimidine-4- carboxamide | 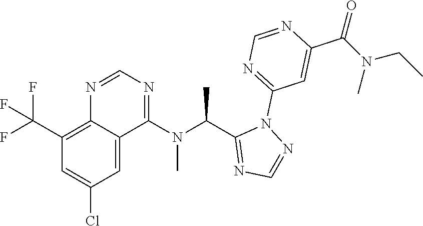 [SMILES: CCN(C)C(=O)c1cc(-n2ncnc2[C@H](C)N(C)c2ncnc3c(C(F)(F)F)cc(Cl)cc23)ncn1] | 1.16 | 520/522 | 3 | 95- 97 |
| P56 | 6-[5-[(1S)-1-[[6-chloro-8- (trifluoromethyl)quina- zolin- 4-yl]amino]ethyl]-1,2,4- triazol-1-yl]pyridazine-4- carboxamide | 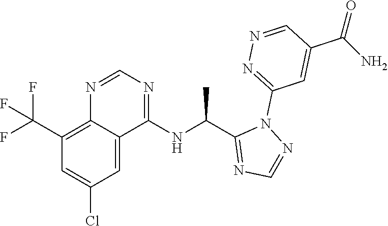 [SMILES: C[C@H](Nc1ncnc2c(C(F)(F)F)cc(Cl)cc12)c1ncnn1-c1cc(C(N)=O)cnn1] | 1.12 | 464/466 | 3 | 192- 194 |
| P57 | 6-[3-[1-[[6-chloro-8- (trifluoromethyl)quina- zolin-4- yl]amino]ethyl]pyrazin-2- yl]pyrimidine-4- carboxamide | 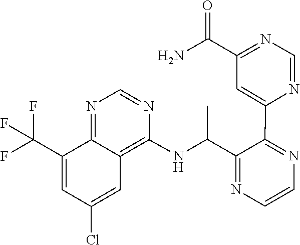 [SMILES: CC(Nc1ncnc2c(C(F)(F)F)cc(Cl)cc12)c1nccnc1-c1cc(C(N)=O)ncn1] | 1.11 | 475/477 | 3 | 260- 262 |
| P58 | 6-[3-[1-[(6-chloro-8-iodo- quinazolin-4- yl)amino]ethyl]pyrazin-2- yl]pyrimidine-4- carboxamide | 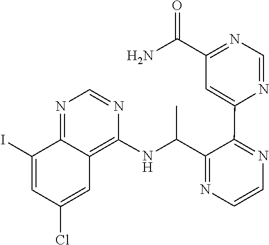 [SMILES: CC(Nc1ncnc2c(I)cc(Cl)cc12)c1nccnc1-c1cc(C(N)=O)ncn1] | 1.09 | 533/535 | 3 | 271- 273 |
| P59 | 6-[5-[(1S)-1-[[6-chloro-8- (trifluoromethylsulfonyl) quinazolin-4- yl]amino]ethyl]-1,2,4- triazol-1-yl]pyrimidine-4- carboxamide | 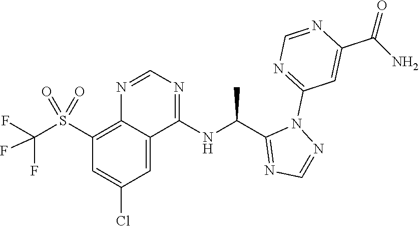 [SMILES: C[C@H](Nc1ncnc2c(S(=O)(=O)C(F)(F)F)cc(Cl)cc12)c1ncnn1-c1cc(C(N)=O)ncn1] | 1.11 | 528/530 | 3 | 178- 180 |
| P60 | 6-[5-[(1S)-1-[[6-chloro-8- (trifluoromethylsulfonyl) quinazolin-4- yl]amino]ethyl]-1,2,4- triazol-1-yl]-N-methyl- pyrimidine-4- carboxamide | 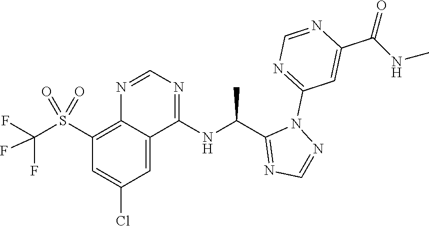 [SMILES: CNC(=O)c1cc(-n2ncnc2[C@H](C)Nc2ncnc3c(S(=O)(=O)C(F)(F)F)cc(Cl)cc23)ncn1] | 1.14 | 542/544 | 3 | 162- 169 |
| P61 | 6-[5-[(1S)-1-[6-chloro-8- (trifluoromethylsulfonyl) quinazolin-4-yl]-methyl- amino]ethyl]-1,2,4- triazol-1-yl]-N-methyl- pyrimidine-4- carboxamide | 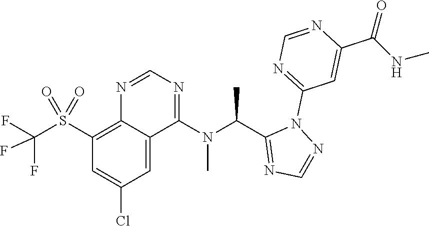 [SMILES: CNC(=O)c1cc(-n2ncnc2[C@H](C)N(C)c2ncnc3c(S(=O)(=O)C(F)(F)F)cc(Cl)cc23)ncn1] | 1.14 | 556/558 | 3 | 119- 121 |
| P62 | 6-[5-[(1S)-1-[[6-chloro-8- (trifluoromethyl)quina- zolin- 4-yl]amino]ethyl]-1,2,4- triazol-1-yl]-N- (cyanomethyl)-N-methyl- pyrimidine-4- carboxamide | 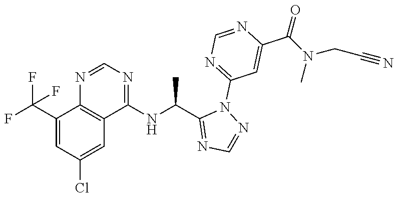 [SMILES: C[C@H](Nc1ncnc2c(C(F)(F)F)cc(Cl)cc12)c1ncnn1-c1cc(C(=O)N(C)CC#N)ncn1] | 1.08 | 517/519 | 3 | 156- 158 |
| P63 | 6-[5-[(1S)-1-[6-chloro-8- (trifluoromethyl)quinazolin- 4-yl]amino]ethyl]-1,2,4- triazol-1-yl]-N- cyclopropyl-N-methyl- pyrimidine-4- carboxamide | 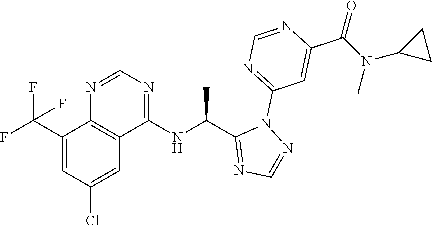 [SMILES: C[C@H](Nc1ncnc2c(C(F)(F)F)cc(Cl)cc12)c1ncnn1-c1cc(C(=O)N(C)C2CC2)ncn1] | 1.11 | 518/520 | 3 | 123- 125 |
| P64 | 6-[5-[(1S)-1-[6-chloro-8- (trifluoromethyl)quina- zolin-4-yl]-methyl- amino]ethyl]-1,2,4- triazol-1-yl]-N- (cyanomethyl)-N-methyl- pyrimidine-4- carboxamide | 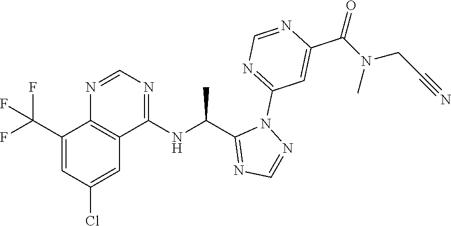 [SMILES: C[C@H](Nc1ncnc2c(C(F)(F)F)cc(Cl)cc12)c1ncnn1-c1cc(C(=O)N(C)CC#N)ncn1] | 1.13 | 531/533 | 3 | 102- 104 |
| P65 | 6-[5-[(1S)-1-[(6-chloro-8- iodo-quinazolin-4- yl)amino]ethyl]-1,2,4- triazol-1-yl]-N,N- dimethyl-pyrimidine-4- carboxamide | 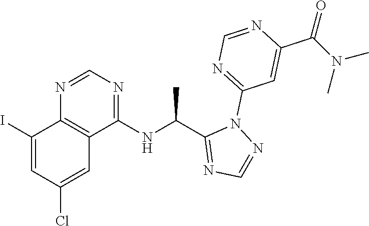 [SMILES: C[C@H](Nc1ncnc2c(I)cc(Cl)cc12)c1ncnn1-c1cc(C(=O)N(C)C)ncn1] | 1.05 | 550/552 | 3 | 139- 142 |
| P66 | 6-[5-[(1S)-1-[(6-chloro-8- iodo-quinazolin-4-yl)- methyl-amino]ethyl]- 1,2,4-triazol-1-yl]-N,N- dimethyl-pyrimidine-4- carboxamide | 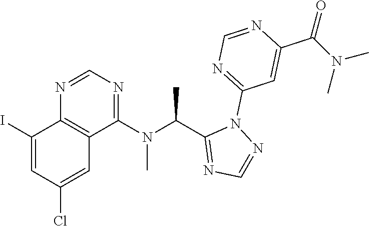 [SMILES: C[C@@H](c1ncnn1-c1cc(C(=O)N(C)C)ncn1)N(C)c1ncnc2c(I)cc(Cl)cc12] | 1.11 | 564/566 | 3 | 141- 144 |
| P67 | 6-[5-[(1S)-1-[[6-chloro-8- (trifluoromethylsulfonyl) quinazolin-4-yl]-methyl- amino]ethyl]-1,2,4- triazol-1-yl]pyrimidine-4- carboxamide | 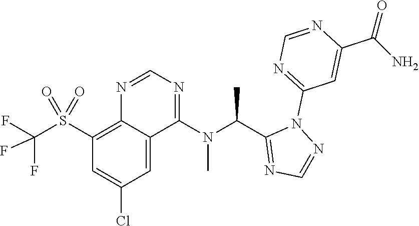 [SMILES: C[C@@H](c1ncnn1-c1cc(C(N)=O)ncn1)N(C)c1ncnc2c(S(=O)(=O)C(F)(F)F)cc(Cl)cc12] | 1.09 | 542/544 | 3 | — |
| P68 | 2-[5-[(1S)-1-[[6-chloro-8- (trifluoromethyl)quina- zolin- 4-yl]amino]ethyl]-1,2,4- triazol-1-yl]pyrimidine-5- carboxamide | 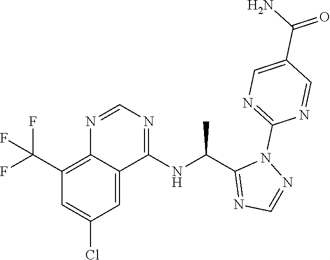 [SMILES: C[C@H](Nc1ncnc2c(C(F)(F)F)cc(Cl)cc12)c1ncnn1-c1ncc(C(N)=O)cn1] | 0.99 | 464/466 | 3 | — |
| P69 | 5-[5-[(1S)-1-[[8-chloro-6- (trifluoromethyl)quinazolin- 4-yl]amino]ethyl]-1,2,4- triazol-1-yl]-N- (cyanomethyl)pyrazine- 2-carboxamide | 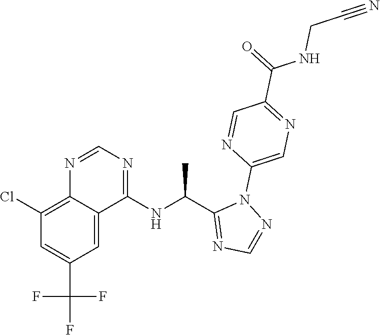 [SMILES: C[C@H](Nc1ncnc2c(Cl)cc(C(F)(F)F)cc12)c1ncnn1-c1cnc(C(=O)NCC#N)cn1] | 1.47 | 503/505 | 5 | — |
| P70 | 5-[5-[(1S)-1-[(8-chloro-6- iodo-quinazolin-4- yl)amino]ethyl]-1,2,4- triazol-1-yl]-N- (cyanomethyl)pyrazine- 2-carboxamide | 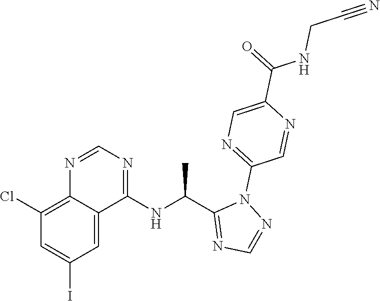 [SMILES: C[C@H](Nc1ncnc2c(Cl)cc(I)cc12)c1ncnn1-c1cnc(C(=O)NCC#N)cn1] | 1.39 | 561/563 | 5 | — |
| P71 | 5-[5-[(1S)-1-[6-chloro-8- (trifluoromethyl)quina- zolin- 4-yl]amino]ethyl]-1,2,4- triazol-1-yl]-N- (cyanomethyl)pyrazine- 2-carboxamide | 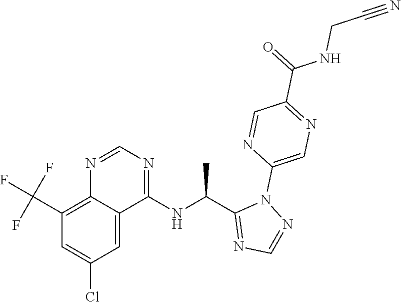 [SMILES: C[C@H](Nc1ncnc2c(C(F)(F)F)cc(Cl)cc12)c1ncnn1-c1cnc(C(=O)NCC#N)cn1] | 0.95 | 503/505 | 1 | — |
| P72 | N-(cyanomethyl)-5-[5- [(1S)-1-[(6,8- dibromoquinazolin-4- yl)amino]ethyl]-1,2,4- triazol-1-yl]pyrazine-2- carboxamide | 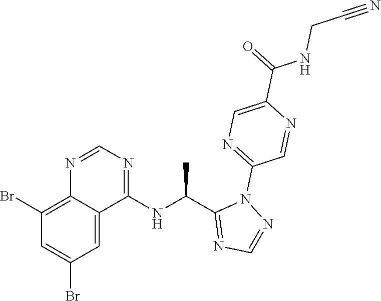 [SMILES: C[C@H](Nc1ncnc2c(Br)cc(Br)cc12)c1ncnn1-c1cnc(C(=O)NCC#N)cn1] | 1.41 | 557/559/ 561 | 5 | — |
| P73 | 5-[5-[(1S)-1-[(6-chloro-8- iodo-quinazolin-4- yl)amino]ethyl]-1,2,4- triazol-1-yl]-N- (cyanomethyl)pyrazine- 2-carboxamide | 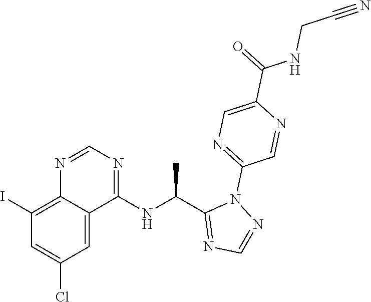 [SMILES: C[C@H](Nc1ncnc2c(I)cc(Cl)cc12)c1ncnn1-c1cnc(C(=O)NCC#N)cn1] | 1.47 | 561/563 | 5 | — |
| P74 | 5-[5-[(1S)-1-[(6,8- dibromoquinazolin-4- yl)amino]ethyl]-1,2,4- triazol-1-yl]pyrazine-2- carboxamide | 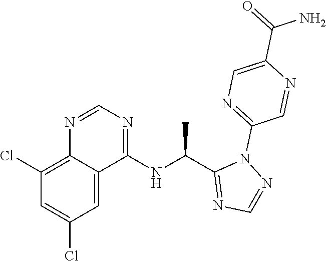 [SMILES: C[C@H](Nc1ncnc2c(Cl)cc(Cl)cc12)c1ncnn1-c1cnc(C(N)=O)cn1] | 0.88 | 518/520/ 522 | 1 | — |
| P75 | 5-[5-[(1S)-1-[(6-chloro-8- iodo-quinazolin-4- yl)amino]ethyl]-1,2,4- triazol-1-yl]pyrazine-2- carboxamide | 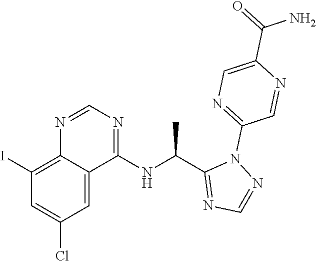 [SMILES: C[C@H](Nc1ncnc2c(I)cc(Cl)cc12)c1ncnn1-c1cnc(C(N)=O)cn1] | 1.38 | 522/524 | 5 | — |
| P76 | 5-[5-[(1S)-1-[[8-chloro-6- (trifluoromethyl)quina- zolin- 4-yl]amino]ethyl]-1,2,4- triazol-1-yl]pyrazine-2- carboxamide | 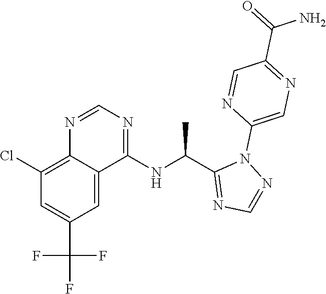 [SMILES: C[C@H](Nc1ncnc2c(Cl)cc(C(F)(F)F)cc12)c1ncnn1-c1cnc(C(N)=O)cn1] | | | | |
| P77 | 5-[5-[(1S)-1-[(8-chloro-6- iodo-quinazolin-4- yl)amino]ethyl]-1,2,4- triazol-1-yl]pyrazine-2- carboxamide | 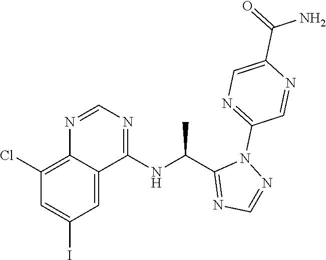 [SMILES: C[C@H](Nc1ncnc2c(Cl)cc(I)cc12)c1ncnn1-c1cnc(C(N)=O)cn1] | 1.28 | 522/524 | 5 | — |
| P78 | 6-[5-[(1S)-1-[[6-chloro-8- (trifluoromethyl)quina- zolin- 4-yl]amino]ethyl]-1,2,4- triazol-1-yl]-N- (cyclopropylmethyl)-N- methyl-pyrimidine-4- carboxamide | 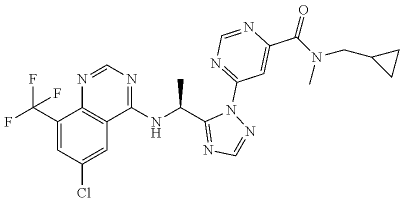 [SMILES: C[C@H](Nc1ncnc2c(C(F)(F)F)cc(Cl)cc12)c1ncnn1-c1cc(C(=O)N(C)CC2CC2)ncn1] | 1.16 | 532/534 | 3 | 130- 132 |
| P79 | 6-[5-[(1S)-1-[(6-chloro-8- iodo-quinazolin-4-yl)- methyl-amino]ethyl]- 1,2,4-triazol-1-yl]-N- (cyanomethyl)pyrimidine- 4-carboxamide | 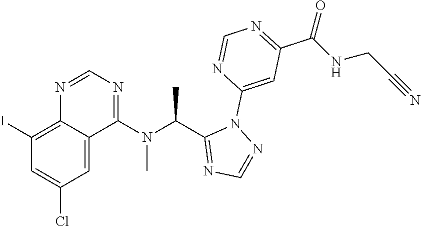 [SMILES: C[C@@H](c1ncnn1-c1cc(C(=O)NCC#N)ncn1)N(C)c1ncnc2c(I)cc(Cl)cc12] | 1.12 | 575/577 | 3 | — |
| P80 | 6-[5-[(1S)-1-[(6-chloro-8- iodo-quinazolin-4- yl)amino]ethyl]-1,2,4- triazol-1-yl]-N- (cyanomethyl)pyrimidine- 4-carboxamide | 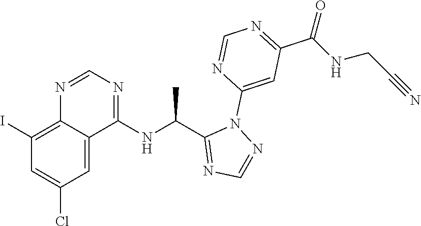 [SMILES: C[C@H](Nc1ncnc2c(I)cc(Cl)cc12)c1ncnn1-c1cc(C(=O)NCC#N)ncn1] | 1.92 | 561/563 | 4 | — |
| P81 | 6-[5-[(1S)-1-[[6-chloro-8- (trifluoromethyl)quina- zolin-4-yl]-methyl- amino]ethyl]-1,2,4- triazol-1-yl]-N- cyclopropyl-N-methyl- pyrimidine-4- carboxamide | 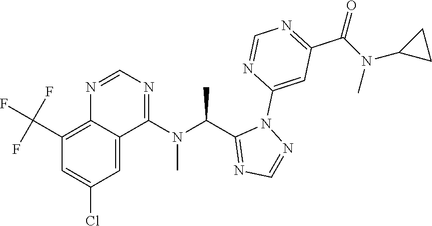 [SMILES: C[C@@H](c1ncnn1-c1cc(C(=O)N(C)C2CC2)ncn1)N(C)c1ncnc2c(C(F)(F)F)cc(Cl)cc12] | 1.17 | 532/534 | 3 | 93- 95 |
| P82 | 6-[5-[(1S)-1-[[6-chloro-8- (trifluoromethyl)quina- zolin-4-yl]-methyl- amino]ethyl]-1,2,4- triazol-1-yl]-N,N- dimethyl-pyrimidine-4- carboxamide | 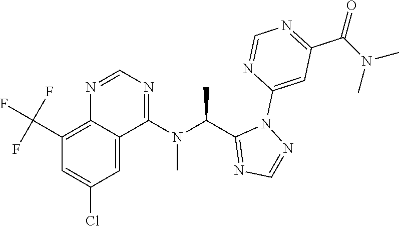 [SMILES: C[C@@H](c1ncnn1-c1cc(C(=O)N(C)C)ncn1)N(C)c1ncnc2c(C(F)(F)F)cc(Cl)cc12] | 1.12 | 506/508 | 3 | 86- 88 |
| P83 | 6-[5-[(1S)-1-[[6-chloro-8- (trifluoromethyl)quina- zolin-4-yl]-methyl- amino]ethyl]-1,2,4- triazol-1-yl]-N- (cyclopropylmethyl)-N- methyl-pyrimidine-4- carboxamide | 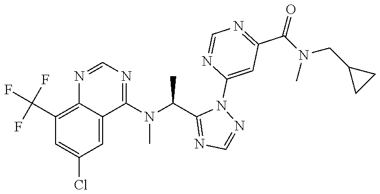 [SMILES: C[C@@H](c1ncnn1-c1cc(C(=O)N(C)CC2CC2)ncn1)N(C)c1ncnc2c(C(F)(F)F)cc(Cl)cc12] | 1.19 | 546/548 | 3 | — |
| P84 | 6-[5-[(1S)-1-[[6-chloro-8- (difluoromethylsulfonyl) quinazolin-4- yl]amino]ethyl]-1,2,4- triazol-1-yl]pyrimidine-4- carboxamide | 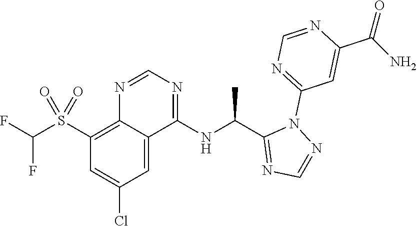 [SMILES: C[C@H](Nc1ncnc2c(S(=O)(=O)C(F)F)cc(Cl)cc12)c1ncnn1-c1cc(C(N)=O)ncn1] | | | | — |
| P85 | 2-[5-[(1S)-1-[[6-chloro-8- (difluoromethylsulfonyl) quinazolin-4- yl]amino]ethyl]-1,2,4- triazol-1-yl]thiazole-5- carboxamide | 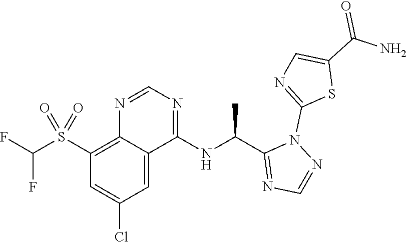 [SMILES: C[C@H](Nc1ncnc2c(S(=O)(=O)C(F)F)cc(Cl)cc12)c1ncnn1-c1ncc(C(N)=O)s1] | | | | — |

Compounds P84 and P85 characterized via ¹H NMR measurements:
P84: ¹H NMR (400 MHz, DMSO-d₆) δ ppm 9.40 (d, 1H), 9.29 (s, 1H), 9.14 (d, 1H), 8.46 (br s, 1H), 8.42 (m, 2H), 8.40 (s, 1H), 8.28 (s, 1H), 8.13 (br s, 1H), 7.67 (t, 1H), 6.57 (quint, 1H), 1.77 (d, 3H).
P85: ¹H NMR (400 MHz, DMSO-d₆) δ ppm 9.40 (d, 1H), 9.15 (d, 1H), 8.47 (s, 1H), 8.44 (d, 1H), 8.28 (s, 1H), 8.26 (br s, 1H), 8.22 (s, 1H), 7.79 (br s, 1H), 7.68 (t, 1H), 6.33 (quint, 1H), 1.74 (d, 3H).

### Preparation of Intermediates

### Example PI-1: Preparation of tert-butyl N-[(1S)-1-[2-(5-carbamoylthiazol-2-yl)-1,2,4-triazol-3-yl]ethyl]carbamate (I-7)

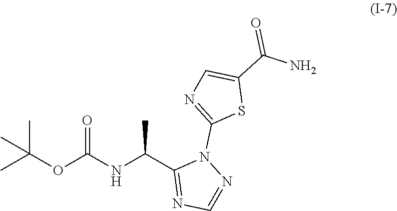 [SMILES: C[C@H](NC(=O)OC(C)(C)C)c1ncnn1-c1ncc(C(N)=O)s1]

### Step 1: Preparation of Silica Gel-Supported Ammonium Chloride (NH₄Cl/SiO₂)

According to Tetrahedron Letters 2005, 46, 6879-6882: Silica gel (5.0 g, Merck Kieselgel 60, particle size 0.063-0.200 mm, 70-230 mesh) was mixed with a solution of ammonium chloride (20 mmol) in water (5.0 mL). Evaporation of water in vacuo gave a white powder which was further dried under reduced pressure. This material was used as an ammonia source in the next step.

### Step 2: Preparation of tert-butyl N-[(1S)-1-[2-(5-carbamoylthiazol-2-yl)-1,2,4-triazol-3-yl]ethyl]carbamate (I-7)

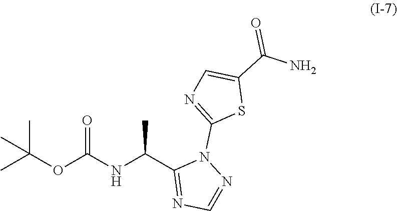 [SMILES: C[C@H](NC(=O)OC(C)(C)C)c1ncnn1-c1ncc(C(N)=O)s1]

To a mixture of 2-[5-[(1S)-1-(tert-butoxycarbonylamino)ethyl]-1,2,4-triazol-1-yl]thiazole-5-carboxylic acid (I-2) (400 mg, 1.18 mmol), silica gel-supported ammonium chloride (NH₄Cl/SiO₂ prepared as described above, 700 mg) and tosyl chloride (227 mg, 1.18 mmol) was added triethylamine (479 mg, 4.71 mmol). The reaction mixture was thoroughly mixed with a spatula. After 2 min the mixture was directly purified by column chromatography without any work up (50% EtOAc in cyclohexane) to afford tert-butyl N-[(1S)-1-[2-(5-carbamoylthiazol-2-yl)-1,2,4-triazol-3-yl]ethyl]carbamate (I-7) as a white solid. LCMS (method 3): retention time 0.44 min, m/z 283 [M+H-tBu]⁺. ¹H NMR (400 MHz, MeOH-d₄) δ ppm 8.21 (s, 1H), 8.05 (s, 1H), 5.77 (m, 1H), 1.54 (d, 1H), 1.40 (br s, 9H).

### Example PI-2: Preparation of 6-chloro-N-[(1S)-1-[2-(6-chloropyrimidin-4-yl)-1,2,4-triazol-3-yl]ethyl]-N-methyl-8-(trifluoromethyl)quinazolin-4-amine (I-21)

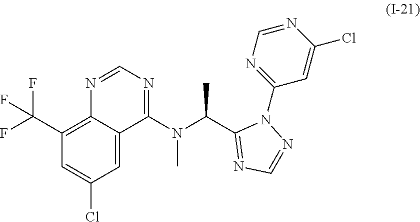 [SMILES: C[C@@H](c1ncnn1-c1cc(Cl)ncn1)N(C)c1ncnc2c(C(F)(F)F)cc(Cl)cc12]

To a solution of 6-chloro-N-[(1S)-1-[2-(6-chloropyrimidin-4-yl)-1,2,4-triazol-3-yl]ethyl]-8-(trifluoromethyl)quinazolin-4-amine (I-18) (4 g, 8.79 mmol) in acetonitrile (40 mL) were added iodomethane (5.50 mL, 87.87 mmol) and cesium carbonate (5.73 g, 17.57 mmol) at room temperature. The reaction mixture was stirred at 80° C. for 6 h. The mixture was cooled to room temperature, diluted with water and the product extracted with ethyl acetate. The organic layer was washed with brine, dried over sodium sulfate and concentrated in vacuo. The crude material was purified by combiflash using ethyl acetate and cyclohexane as eluent to afford 6-chloro-N-[(1S)-1-[2-(6-chloropyrimidin-4-yl)-1,2,4-triazol-3-yl]ethyl]-N-methyl-8-(trifluoromethyl)quinazolin-4-amine (1.7 g) as a pale yellow solid. LCMS (method 3): retention time 1.26 min, m/z 469/471 [M+H]⁺. ¹H NMR (CDCl₃) δ: 8.49-8.55 (m, 2H), 8.01-8.07 (m, 4H), 6.88 (q, 1H), 3.50 (s, 3H), 1.94 (d, 3H).
Similarly, 6-chloro-N-[(1 S)-1-[2-(6-iodopyrimidin-4-yl)-1,2,4-triazol-3-yl]ethyl]-N-methyl-8-(trifluoromethyl)quinazolin-4-amine (compound 1-39) can be obtained from 6-chloro-N-[(1S)-1-[2-(6-iodopyrimidin-4-yl)-1,2,4-triazol-3-yl]ethyl]-8-(trifluoromethyl)quinazolin-4-amine (I-38) with above protocol. LCMS (method 4): retention time 2.32 min, m/z 561/563 [M+H]⁺.

### Example PI-3: Preparation of [(1S)-1-[2-(6-carbamoylpyrimidin-4-yl)-1,2,4-triazol-3-yl]ethyl]-methyl-ammonium chloride (I-29)

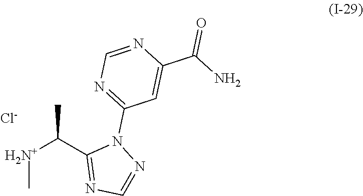 [SMILES: [Cl-] ; C[NH2+][C@@H](C)c1ncnn1-c1cc(C(N)=O)ncn1]

### Step 1: Preparation of tert-butyl N-[(1 S)-1-[2-(6-carbamoylpyrimidin-4-yl)-1,2,4-triazol-3-yl]ethyl]-N-methyl-carbamate (I-28)

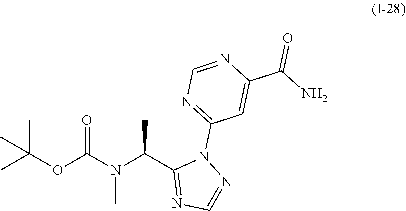 [SMILES: C[C@@H](c1ncnn1-c1cc(C(N)=O)ncn1)N(C)C(=O)OC(C)(C)C]

To a solution of tert-butyl N-[(1S)-2-amino-1-methyl-2-oxo-ethyl]-N-methyl-carbamate (CAS 344609-43-4) (0.454 g, 2.24 mmol) in 2-methyltetrahydrofuran (4.5 mL) was added N,N-dimethylformamide dimethyl acetal (2.69 mmol) at room temperature. The resulting reaction mixture was stirred at 40° C. for 1 hour, then concentrated in vacuo to get the crude intermediate tert-butyl N-[(1 S)-2-[I-dimethylaminomethyleneamino]-1-methyl-2-oxo-ethyl]-N-methyl-carbamate (LCMS (method 3): retention time 0.96 min, m/z 202 [M+H-tBu]⁺).
1,4-Dioxane (2.724 mL), acetic acid (2.724 mL) and 6-hydrazinopyrimidine-4-carboxamide (CAS 2283190-21-4) (0.34 g, 2.22 mmol) were added to this intermediate and the mixture was stirred at 80° C. for 16 hours, then concentrated under reduced pressure. The residue was purified by reversed phase chromatography on a C₁₈ column (acetonitrile in water) to afford tert-butyl N-[(1S)-1-[2-(6-carbamoylpyrimidin-4-yl)-1,2,4-triazol-3-yl]ethyl]-N-methyl-carbamate (I-28).
LCMS (method 3): retention time 1.01 min, m/z 292 [M+H-tBu]⁺.

### Step 2: Preparation of [(1S)-1-[2-(6-carbamoylpyrimidin-4-yl)-1,2,4-triazol-3-yl]ethyl]-methyl-ammonium chloride (I-29)

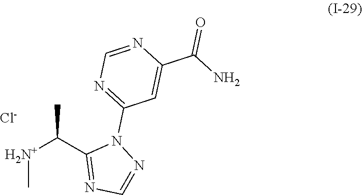 [SMILES: [Cl-] ; C[NH2+][C@@H](C)c1ncnn1-c1cc(C(N)=O)ncn1]

To a solution of tert-butyl N-[(1 S)-1-[2-(6-carbamoylpyrimidin-4-yl)-1,2,4-triazol-3-yl]ethyl]-N-methyl-carbamate (I-28) (0.3 g, 0.86 mmol) in 1,4-dioxane (4.3 mL) was added a 4M hydrochloric acid solution in 1,4-dioxane (2.3 mL) at 0° C. The reaction mixture was stirred at room temperature for 16 hours, then concentrated in vacuo till dryness. The residue was triturated with TBME, the obtained solid was filtered and dried in vacuo to afford [(1S)-1-[2-(6-carbamoylpyrimidin-4-yl)-1,2,4-triazol-3-yl]ethyl]-methyl-ammonium chloride (I-29) as a solid.
LCMS (method 3): retention time 0.16 min, m/z 248 [M+H]⁺ of the corresponding free base.

### Example PI-4: Preparation of [(1S)-1-[2-(5-carbamoylpyrazin-2-yl)-1,2,4-triazol-3-yl]ethyl]ammonium chloride (I-16)

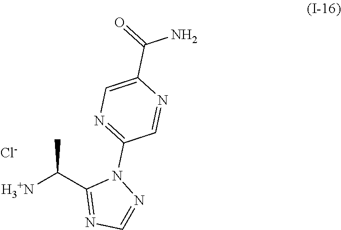

### Step 1: Preparation of tert-butyl N-[(1S)-1-[2-(5-bromopyrazin-2-yl)-1,2,4-triazol-3-yl]ethyl]carbamate

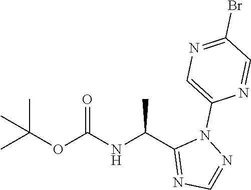 [SMILES: C[C@H](NC(=O)OC(C)(C)C)c1ncnn1-c1cnc(Br)cn1]

To a solution of tert-butyl N-[(1S)-2-amino-1-methyl-2-oxo-ethyl]carbamate (1.8 g, 9.56 mmol) in 2-methyltetrahydrofuran (29 mL) was added N,N-dimethylformamide dimethyl acetal (14.35 mmol) at room temperature. The resulting reaction mixture was stirred at 40° C. for 1.5 hour, then concentrated in vacuo to get the crude intermediate tert-butyl N-[(1 S)-2-[I-dimethylaminomethyleneamino]-1-methyl-2-oxo-ethyl]carbamate.
1,4-Dioxane (9.6 mL), acetic acid (9.6 mL) and 2-bromo-5-hydrazinylpyrazine (CAS 1001050-24-3) (1.6 g, 95%, 8.04 mmol) were added to this intermediate and the mixture was stirred at 80° C. for 2 hours, then cooled to room temperature and poured over water and EtOAc. The phases were separated, the aqueous layer extracted with EtOAc, the combined organic layers washed with water and brine, dried over sodium sulfate, filtered and concentrated in vacuo. The crude material was purified by combiflash (ethyl acetate in cyclohexane) to afford tert-butyl N-[(1S)-1-[2-(5-bromopyrazin-2-yl)-1,2,4-triazol-3-yl]ethyl]carbamate as a gum.
LCMS (method 1): retention time 0.95 min, m/z 369/371 [M+H]⁺.

### Step 2: Preparation of methyl 5-[5-[(1S)-1-(tert-butoxycarbonylamino)ethyl]-1,2,4-triazol-1-yl]pyrazine-2-carboxylate (I-24)

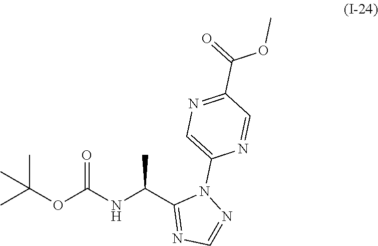 [SMILES: COC(=O)c1cnc(-n2ncnc2[C@H](C)NC(=O)OC(C)(C)C)cn1]

In a pressure vessel under argon atmosphere 1,1′-bis(diphenylphosphino)ferrocene (dppf, 69.9 mg, 0.124 mmol) and bis(benzonitrile)palladium(II) chloride (24.2 mg, 0.0618 mmol) were dissolved in methanol (45.6 mL). N-[(1S)-1-[2-(5-bromopyrazin-2-yl)-1,2,4-triazol-3-yl]ethyl]carbamate (prepared as described above) (1140 mg, 3.09 mmol) and triethylamine (410 mg, 0.565 mL, 4.01 mmol) were added and the mixture was flushed with a stream of argon for 10 minutes, then with carbon monoxide. The reaction mixture was heated to 80° C. under a 10 bar carbon monoxide pressure overnight. After cooling to room temperature, the pressure was carefully released and the vessel flushed with nitrogen. The mixture was evaporated in vacuo, the residue dissolved in ethyl acetate and adsorbed on isolute. The crude material was purified by combiflash (ethyl acetate in cyclohexane) to afford methyl 5-[5-[(1S)-1-(tert-butoxycarbonylamino)ethyl]-1,2,4-triazol-1-yl]pyrazine-2-carboxylate (I-24).
LCMS (method 1): retention time 0.83 min, m/z 349 [M+H]⁺.

### Step 3: Preparation of tert-butyl N-[(1S)-1-[2-(5-carbamoylpyrazin-2-yl)-1,2,4-triazol-3-yl]ethyl]carbamate (I-25)

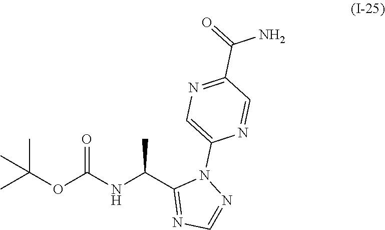 [SMILES: C[C@H](NC(=O)OC(C)(C)C)c1ncnn1-c1cnc(C(N)=O)cn1]

Methyl 5-[5-[(1S)-1-(tert-butoxycarbonylamino)ethyl]-1,2,4-triazol-1-yl]pyrazine-2-carboxylate (I-24) (0.5 g, 1.44 mmol) dissolved in a 7M ammonia solution in methanol (10 mL, 70 mmol) was stirred at 0-5° C. for 4 hours. The reaction mixture was concentrated in vacuo (25° C. bath temperature) to afford crude tert-butyl N-[(1S)-1-[2-(5-carbamoylpyrazin-2-yl)-1,2,4-triazol-3-yl]ethyl]carbamate (I-25), which was used directly into the next step. LCMS (method 1): retention time 0.71 min. m/z 334 [M+H]⁺.

### Step 4: Preparation of [(1S)-1-[2-(5-carbamoylpyrazin-2-yl)-1,2,4-triazol-3-yl]ethyl]ammonium chloride (1-16)

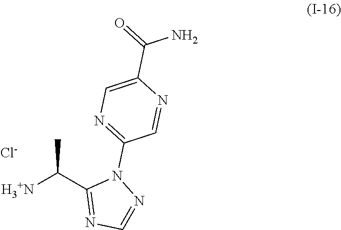

Crude tert-butyl N-[(1S)-1-[2-(5-carbamoylpyrazin-2-yl)-1,2,4-triazol-3-yl]ethyl]carbamate (I-25) (0.504 g, 1.44 mmol) was suspended in a 5-6 M solution of hydrochloric acid in isopropanol (3.59 mL, ca. 20 mmol) and stirred at 0-5° C. for 15 minutes, then at room temperature for 40 minutes. The reaction mixture was concentrated under reduce pressure to afford crude [(1S)-1-[2-(5-carbamoylpyrazin-2-yl)-1,2,4-triazol-3-yl]ethyl]ammonium chloride (I-16) as a solid, which was used directly into the next step. LCMS (method 3): retention time 0.16 min, m/z 234 [M+H]⁺ of the corresponding free base.

### Example PI-5: Preparation of [(1S)-1-[2-[5-(cyanomethylcarbamoyl)pyrazin-2-yl]-1,2,4-triazol-3-yl]ethyl]ammonium chloride (I-27)

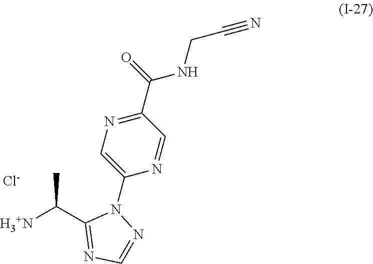

### Step 1: Preparation of 5-[5-[(1 S)-1-(tert-butoxycarbonylamino)ethyl]-1,2,4-triazol-1-yl]pyrazine-2-carboxylic acid

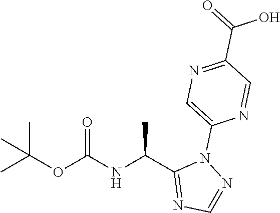 [SMILES: C[C@H](NC(=O)OC(C)(C)C)c1ncnn1-c1cnc(C(=O)O)cn1]

To a solution of methyl 5-[5-[(1S)-1-(tert-butoxycarbonylamino)ethyl]-1,2,4-triazol-1-yl]pyrazine-2-carboxylate (I-24) (0.4 g, 1.09 mmol) in tetrahydrofuran (8.2 mL) and water (2.7 mL) was added an aqueous 1 M solution of sodium hydroxide (1.31 mL, 1.31 mmol). The resulting mixture was stirred at room temperature for 40 minutes. The mixture was acidified carefully by addition of aqueous 1 M hydrochloric acid and the product extracted twice with EtOAc. The combined organic layers were dried over magnesium sulfate, filtered and evaporated in vacuo to afford crude of 5-[5-[(1S)-1-(tert-butoxycarbonylamino)ethyl]-1,2,4-triazol-1-yl]pyrazine-2-carboxylic acid, which was used directly into the next step. LCMS (method 1): retention time 0.69 min, m/z 335 [M+H]⁺ and 333 [M−H]⁻.

### Step 2: Preparation of tert-butyl N-[(1 S)-1-[2-[5-(cyanomethylcarbamoyl)pyrazin-2-yl]-1,2,4-triazol-3-yl]ethyl]carbamate (I-26)

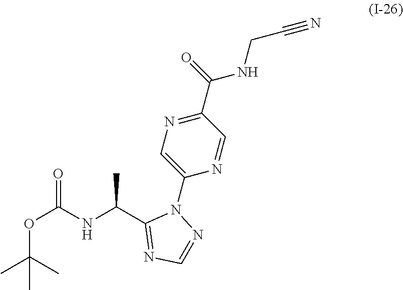 [SMILES: C[C@H](NC(=O)OC(C)(C)C)c1ncnn1-c1cnc(C(=O)NCC#N)cn1]

To a solution of 5-[5-[(1S)-1-(tert-butoxycarbonylamino)ethyl]-1,2,4-triazol-1-yl]pyrazine-2-carboxylic acid (prepared as described above) (500.0 mg, 1.496 mmol), 2-aminoacetonitrile hydrochloride (164.2 mg, 1.795 mmol) and N,N-diisopropylethylamine (585.7 mg, 0.789 mL, 4.487 mmol) in EtOAc (15 mL) was added propanephosphonic acid cyclic anhydride (T3P®, 50 wt. % in ethyl acetate, 2.23 mL, 3.74 mmol). The reaction mixture was stirred at room temperature for 1 hour, then diluted with water and the layers were separated. The aqueous layer was extracted twice with ethylacetate, the combined organic layers washed with brine, dried over magnesium sulfate, filtered and evaporated in vacuo. The residue was purified by combiflash (ethyl acetate in cyclohexane) to afford tert-butyl N-[(1S)-1-[2-[5-(cyanomethylcarbamoyl)pyrazin-2-yl]-1,2,4-triazol-3-yl]ethyl]carbamate (I-26).
LCMS (method 1): retention time 0.78 min, m/z 373 [M+H]⁺.

### Step 3: Preparation of [(1 S)-1-[2-[5-(cyanomethylcarbamoyl)pyrazin-2-yl]-1,2,4-triazol-3-yl]ethyl]ammonium chloride (I-27)

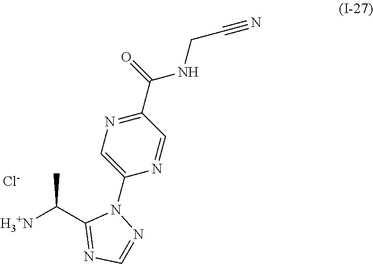

To a suspension of tert-butyl N-[(1 S)-1-[2-[5-(cyanomethylcarbamoyl)pyrazin-2-yl]-1,2,4-triazol-3-yl]ethyl]carbamate (I-26) (0.213 g, 0.572 mmol) in 1 (2.86 mL) at 0-5° C. was added a 4M solution of hydrochloric acid in dioxane (0.286 mL, 1.14 mmol) dropwise with stirring. The reaction mixture was stirred at 0-5° C. for 10 minutes, then at 10-15° C. for 1.5 hours. Stirring was continued for a total of 3.5 hours at 10-15° C. during which three further additions of a 4M solution of hydrochloric acid in dioxane were performed (twice 0.286 mL, and once 0.143 mL). The mixture was concentrated in vacuo to afford crude [(1 S)-1-[2-[5-(cyanomethylcarbamoyl)pyrazin-2-yl]-1,2,4-triazol-3-yl]ethyl]ammonium chloride (I-27), which was used directly into the next step.
LCMS (method 1): retention time 0.15 min, m/z 273 [M+H]⁺ of the corresponding free base.

### Example PI-6: Preparation of 6-chloro-N-[(1 S)-1-[2-(6-iodopyrimidin-4-yl)-1,2,4-triazol-3-yl]ethyl]-8-(trifluoromethyl)quinazolin-4-amine (I-38)

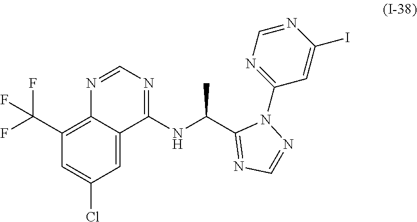 [SMILES: C[C@H](Nc1ncnc2c(C(F)(F)F)cc(Cl)cc12)c1ncnn1-c1cc(I)ncn1]

To a solution of 6-chloro-N-[(1S)-1-[2-(6-chloropyrimidin-4-yl)-1,2,4-triazol-3-yl]ethyl]-8-(trifluoromethyl)quinazolin-4-amine (I-18) (2.0 g, 4.39 mmol) in toluene (20 mL) at 0° C. was added an aqueous solution of hydrogen iodide (˜55% w/w in H₂O, 20 mL, 146.3 mmol) dropwise under nitrogen. After addition, the reaction mixture was stirred at 0° C. for 15 min, then at room temperature for 16 hours. The mixture was neutralized to pH 7 by careful addition of sodium bicarbonate and the product extracted with ethyl acetate (3×10 mL). The combined organic layers were washed with an aqueous sodium metabisulfite solution, dried over sodium sulfate, filtered and concentrated in vacuo. The residue was purified by combiflash (ethyl acetate in cyclohexane) to afford 6-chloro-N-[(1S)-1-[2-(6-iodopyrimidin-4-yl)-1,2,4-triazol-3-yl]ethyl]-8-(trifluoromethyl)quinazolin-4-amine (I-38) as a solid. LCMS (method 3): retention time 1.24 min, m/z 547/549 [M+H]⁺. ¹H NMR (400 MHz, DMSO-d₆) δ ppm 9.11 (d, 1H), 8.95 (d, 1H), 8.91 (d, 1H), 8.41 (d, 1H), 8.39 (s, 1H), 8.23 (s, 1H), 8.22 (d, 1H), 6.46 (quint, 1H), 1.73 (d, 3H).

### Example PI-7: Preparation of 6-chloro-8-(difluoromethylsulfonyl)quinazolin-4-ol (I-44)

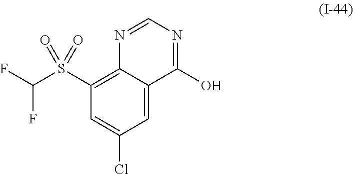 [SMILES: O=S(=O)(c1cc(Cl)cc2c(O)ncnc12)C(F)F]

### Step 1: Preparation of 6-chloro-8-iodo-4-tetrahydropyran-2-yloxy-quinazoline (I-40)

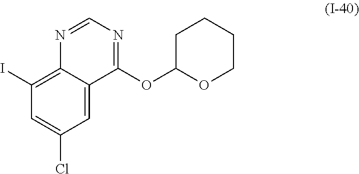 [SMILES: Clc1cc(I)c2ncnc(OC3CCCCO3)c2c1]

To 6-chloro-8-iodo-quinazolin-4-ol (CAS 101581-08-2) (95%, 1.00 g, 3.10 mmol) in toluene (40 mL) under argon were added 3,4-dihydro-2H-pyran (2.60 g, 31.0 mmol) and trifluoroacetic acid (0.25 mL, 3.2 mmol) at room temperature. The reaction mixture was heated to 110° C. for 5 h, then cooled to room temperature, poured in ice cold water and the product extracted with EtOAc (2×50 mL). The combined organic layers were dried over sodium sulfate, filtered and concentrated under reduced pressure. The residue was purified by combiflash (gradient EtOAc in hexane) to afford 6-chloro-8-iodo-4-tetrahydropyran-2-yloxy-quinazoline (I-40) as an off-white solid.

¹H NMR (400 MHz, CDCl₃) δ ppm 8.40 (s, 1H), 8.27 (d, 1H), 8.24 (d, 1H), 5.87 (m, 1H), 4.22 (m, 1H), 3.73 (m, 1H), 2.03 (m, 2H), 1.49-1.88 (br m, 4H).

### Step 2: Preparation of 6-chloro-4-tetrahydropyran-2-yloxy-quinazoline-8-thiol (I-41)

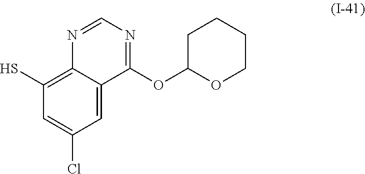 [SMILES: Sc1cc(Cl)cc2c(OC3CCCCO3)ncnc12]

To 6-chloro-8-iodo-4-tetrahydropyran-2-yloxy-quinazoline (I-40) (90%, 500 mg, 1.15 mmol) in N-methyl-2-pyrrolidone (5 mL) under argon was added sodium sulfide hydrate (180 mg, 2.30 mmol). The reaction mixture was heated to 110° C. for 6 h, then cooled to room temperature, poured in ice cold water and the product extracted with EtOAc (2×30 mL). The combined organic layers were dried over sodium sulfate, filtered and concentrated under reduced pressure. The residue was purified by combiflash (gradient EtOAc in hexane) to afford 6-chloro-4-tetrahydropyran-2-yloxy-quinazoline-8-thiol (I-41) as an off-white solid. ¹H NMR (400 MHz, DMSO-d₆) δ ppm 8.52 (s, 1H), 8.01 (d, 1H), 7.79 (d, 1H), 5.81 (m, 1H and s, 1H; overlapping signals), 4.09 (m, 1H), 3.68 (m, 1H), 1.94 (m, 1H), 1.85 (m, 2H), 1.38-1.79 (br m, 3H).

### Step 3: Preparation of 6-chloro-8-(difluoromethylsulfanyl)-4-tetrahydropyran-2-yloxy-quinazoline (I-42)

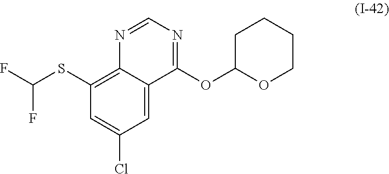 [SMILES: FC(F)Sc1cc(Cl)cc2c(OC3CCCCO3)ncnc12]

To 6-chloro-4-tetrahydropyran-2-yloxy-quinazoline-8-thiol (I-41) (estimated 70.0%, 1.00 g, 2.36 mmol) in N-methyl-2-pyrrolidone (10 mL) under argon were added potassium carbonate (1.30 g, 9.43 mmol) and sodium chlorodifluoroacetate (CAS 1895-39-2) (539 mg, 3.54 mmol) at room temperature. The reaction mixture was heated to 110° C. for 1 h, then cooled to room temperature, poured in ice cold water and the product extracted with EtOAc (2×50 mL). The combined organic layers were dried over sodium sulfate, filtered and concentrated under reduced pressure. The residue was purified by combiflash (gradient EtOAc in hexane) to afford 6-chloro-8-(difluoromethylsulfanyl)-4-tetrahydropyran-2-yloxy-quinazoline (I-42) as an off-white solid. ¹H NMR (400 MHz, DMSO-d₆) δ ppm 8.58 (s, 1H), 8.08 (d, 1H), 7.97 (d, 1H), 7.89 (t, 1H), 5.82 (m, 1H), 4.10 (m, 1H), 3.69 (m, 1H), 1.98-1.81 (br m, 3H), 1.50-1.80 (br m, 3H).

### Step 4: Preparation of 6-chloro-8-(difluoromethylsulfonyl)-4-tetrahydropyran-2-yloxy-quinazoline (I-43)

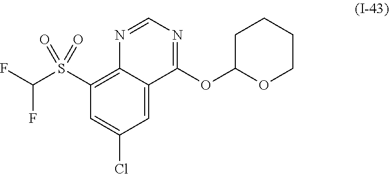 [SMILES: O=S(=O)(c1cc(Cl)cc2c(OC3CCCCO3)ncnc12)C(F)F]

To 6-chloro-8-(difluoromethylsulfanyl)-4-tetrahydropyran-2-yloxy-quinazoline (I-42) (90%, 300 mg, 0.779 mmol) in dichloromethane (6 mL) under argon was added meta-chloroperoxybenzoic acid (mCPBA, 60%, 560 mg, 1.95 mmol) at 0° C. The reaction mixture was stirred at room temperature for 12 h, then diluted with an aqueous saturated sodium hydrogencarbonate solution and stirred for 10 min. The layers were separated, the aqueous phase extracted with dichloromethane (2×30 mL), the combined organic layers washed twice with an aqueous saturated sodium hydrogencarbonate solution, dried over sodium sulfate, filtered and concentrated under reduced pressure. The residue was purified by combiflash (gradient EtOAc in hexane) to afford 6-chloro-8-(difluoromethylsulfonyl)-4-tetrahydropyran-2-yloxy-quinazoline (I-43) as an off-white solid.

¹H NMR (400 MHz, DMSO-d₆) δ ppm 8.67 (s, 1H), 8.55 (d, 1H), 8.44 (d, 1H), 7.60 (t, 1H), 5.83 (m, 1H), 4.11 (m, 1H), 3.72 (m, 1H), 1.99-1.51 (br m, 6H).

### Step 5: Preparation of 6-chloro-8-(difluoromethylsulfonyl)quinazolin-4-ol (I-44)

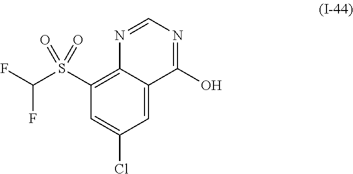 [SMILES: O=S(=O)(c1cc(Cl)cc2c(O)ncnc12)C(F)F]

To 6-chloro-8-(difluoromethylsulfonyl)-4-tetrahydropyran-2-yloxy-quinazoline (I-43) (90%, 200 mg, 0.475 mmol) in a mixture of methanol (5 mL) and tetrahydrofuran (1 mL) under argon was added p-toluenesulfonic acid hydrate (18.1 mg, 0.095 mmol) at room temperature. The reaction mixture was stirred at room temperature for 12 h, then concentrated under reduced pressure. The residue was purified by combiflash (gradient EtOAc in hexane) to afford 6-chloro-8-(difluoromethyl-sulfonyl)quinazolin-4-ol (I-44) as an off-white solid. ¹H NMR (400 MHz, DMSO-d₆) δ ppm 13.00 (br s, 1H), 8.51 (d, 1H), 8.42 (d, 1H), 8.35 (s, 1H), 7.59 (t, 1H).

TABLE PI
Examples of intermediates

| En- | | | RT | [M + H]⁺ | Meth- | mp |
|---|---|---|---|---|---|---|
| try | IUPAC name | Structure | (min) | (measured) | od | (°C.) |
| I-1 | methyl 2-[5-[(1S)-1-(tert- butoxycarbonylamino)ethyl]- 1,2,4-triazol-1-yl]thiazole-5- carboxylate | 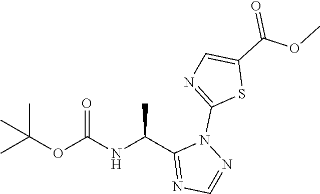 [SMILES: COC(=O)c1cnc(-n2ncnc2[C@H](C)NC(=O)OC(C)(C)C)s1] | 1.11 | 298 [M + H − tBu]⁺ | 2 | — |
| I-2 | 2-[5-[(1S)-1-(tert- butoxycarbonylamino)ethyl]- 1,2,4-triazol-1-yl]thiazole-5- carboxylic acid | 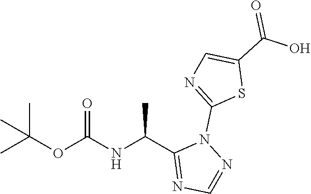 [SMILES: C[C@H](NC(=O)OC(C)(C)C)c1ncnn1-c1ncc(C(=O)O)s1] | 1.03 | 284 [M + H − tBu]⁺ | 2 | — |
| I-3 | tert-butyl N-[(1S)-1-[2-[5- [ethyl(methyl)carbamoyl]thi- azol-2-yl]-1,2,4-triazol-3- yl]ethyl]carbamate | 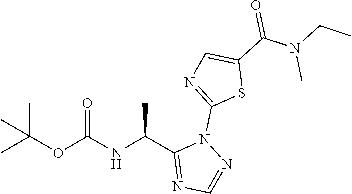 [SMILES: CCN(C)C(=O)c1cnc(-n2ncnc2[C@H](C)NC(=O)OC(C)(C)C)s1] | 1.09 | 381 | 2 | — |
| I-4 | [(1S)-1-[2-[5- [ethyl(methyl)carbamoyl]thi- azol-2-yl]-1,2,4-triazol-3- ylethyl]ammonium chloride | 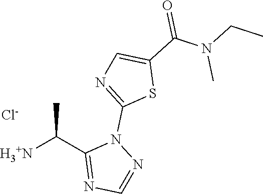 | 0.17 | 281 of the free base | 2 | — |
| I-5 | tert-butyl N-[(1S)-1-[2-[5- (methylcarbamoyl)thiazol-2- yl]-1,2,4-triazol-3- yl]ethyl]carbamate | 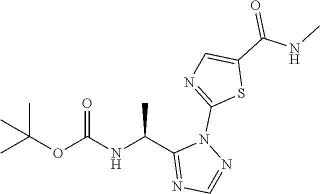 [SMILES: CNC(=O)c1cnc(-n2ncnc2[C@H](C)NC(=O)OC(C)(C)C)s1] | 1.01 | 297 [M + H − tBu]⁺ | 2 | — |
| I-6 | [(1S)-1-[2-[5- (methylcarbamoyl)thiazol-2- yl]-1,2,4-triazol-3- yl]ethyl]ammonium chloride | 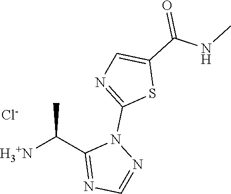 | 0.21 | 253 of the free base | 2 | — |
| I-7 | tert-butyl N-[(1S)-1-[2-(5- carbamoylthiazol-2-yl)- 1,2,4-triazol-3- yl]ethyl]carbamate | 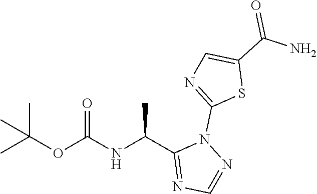 [SMILES: C[C@H](NC(=O)OC(C)(C)C)c1ncnn1-c1ncc(C(N)=O)s1] | 0.44 | 283 [M + H − tBu]⁺ | 3 | — |
| I-8 | [(1S)-1-[2-(5- carbamoylthiazol-2-yl)- 1,2,4-triazol-3- yl]ethyl]ammonium chloride | 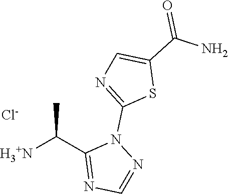 | 0.16 | 239 of the free base | 3 | — |
| I-9 | tert-butyl N-[(1S)-1-[2-[6- (methylcarbamoyl)pyrimidin- 4-yl]-1,2,4-triazol-3- yl]ethyl]carbamate | 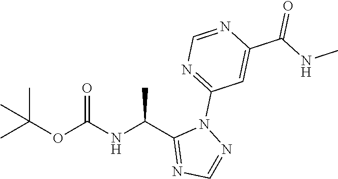 [SMILES: CNC(=O)c1cc(-n2ncnc2[C@H](C)NC(=O)OC(C)(C)C)ncn1] | 0.62 | 292 [M + H − tBu]⁺ | 3 | — |
| I-10 | [(1S)-1-[2-[6- (methylcarbamoyl)pyrimidin- 4-yl]-1,2,4-triazol-3- yl]ethyl]ammonium chloride | 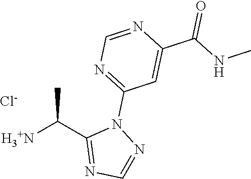 | 0.17 | 248 of the free base | 3 | — |
| I-11 | tert-butyl N-[(1S)-1-[2-(6- carbamoylpyrimidin-4-yl)- 1,2,4-triazol-3- yl]ethyl]carbamate | 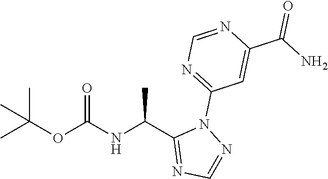 [SMILES: C[C@H](NC(=O)OC(C)(C)C)c1ncnn1-c1cc(C(N)=O)ncn1] | 1.46 | 278 [M + H − tBu]⁺ | 4 | — |
| I-12 | [(1S)-1-[2-(6- carbamoylpyrimidin-4-yl)- 1,2,4-triazol-3- yl]ethyl]ammonium chloride | 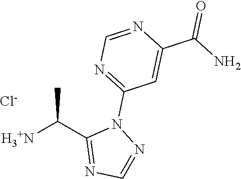 | 0.14 | 234 of the free base | 3 | — |
| I-13 | [(1S)-1-[2-(6- cyanopyrimidin-4-yl)-1,2,4- triazol-3-yl]ethyl]ammonium 2,2,2-trifluoroacetate | 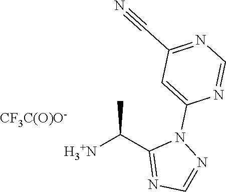 | 0.20 | 216 of the free base | 3 | — |
| I-14 | tert-butyl N-[(1S)-1-[2-(6- carbamoylpyridazin-3-yl)- 1,2,4-triazol-3- yl]ethyl]carbamate | 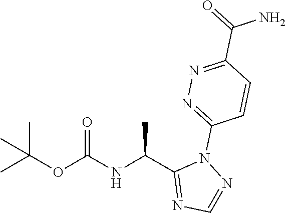 [SMILES: C[C@H](NC(=O)OC(C)(C)C)c1ncnn1-c1ccc(C(N)=O)nn1] | 0.41 | 234 [M + H − Boc]⁺ | 3 | — |
| I-15 | [(1S)-1-[2-(6- carbamoylpyridazin-3-yl)- 1,2,4-triazol-3- yl]ethyl]ammonium chloride | 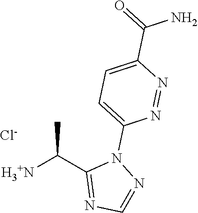 | 0.23 | 234 of the free base | 3 | — |
| I-16 | [(1S)-1-[2-(5- carbamoylpyrazin-2-yl)- 1,2,4-triazol-3- yl]ethyl]ammonium chloride | 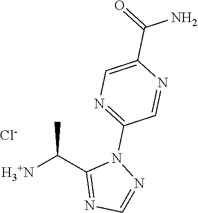 | 0.16 | 234 of the free base | 3 | — |
| I-17 | [(1S)-1-[2-(6- chloropyrimidin-4-yl)-1,2,4- triazol-3-yl]ethyl]ammonium chloride | 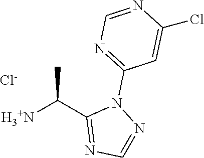 | 0.20 | 225/227 of the free base | 3 | — |
| I-18 | 6-chloro-N-[(1S)-1-[2-(6- chloropyrimidin-4-yl)-1,2,4- triazol-3-yl]ethyl]-8- (trifluoromethyl)quinazolin- 4-amine | 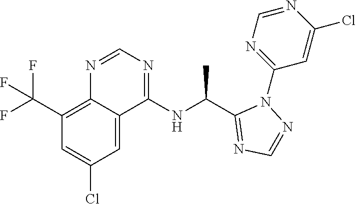 [SMILES: C[C@H](Nc1ncnc2c(C(F)(F)F)cc(Cl)cc12)c1ncnn1-c1cc(Cl)ncn1] | 1.33 | 455/457 | 3 | — |
| I-19 | methyl 6-[5-[(1S)-1-[[6- chloro-8- (trifluoromethyl)quinazolin- 4-yl]amino]ethyl]-1,2,4- triazol-1-yl]pyrimidine-4- carboxylate | 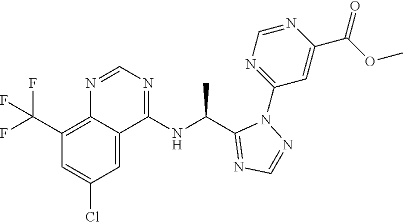 [SMILES: COC(=O)c1cc(-n2ncnc2[C@H](C)Nc2ncnc3c(C(F)(F)F)cc(Cl)cc23)ncn1] | 1.17 | 479/481 | 3 | — |
| I-20 | 6-[5-[(1S)-1-[[6-chloro-8- (trifluoromethyl)quinazolin- 4-yl]amino]ethyl]-1,2,4- triazol-1-yl]pyrimidine-4- carboxylic acid | 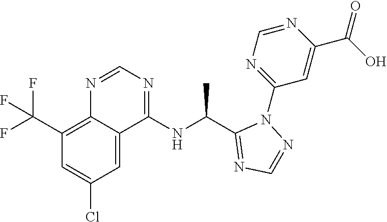 [SMILES: C[C@H](Nc1ncnc2c(C(F)(F)F)cc(Cl)cc12)c1ncnn1-c1cc(C(=O)O)ncn1] | 1.06 | 465/467 | 3 | — |
| I-21 | 6-chloro-N-[(1S)-1-[2-(6- chloropyrimidin-4-yl)-1,2,4- triazol-3-yl]ethyl]-N-methyl- 8- (trifluoromethyl)quinazolin- 4-amine | 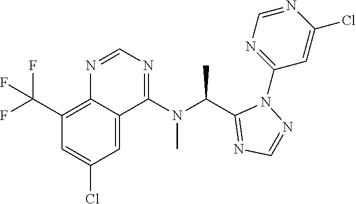 [SMILES: C[C@@H](c1ncnn1-c1cc(Cl)ncn1)N(C)c1ncnc2c(C(F)(F)F)cc(Cl)cc12] | 1.26 | 469/471 | 3 | — |
| I-22 | methyl 6-[5-[(1S)-1-[6- chloro-8- (trifluoromethyl)quinazolin- 4-yl]-methyl-amino]ethyl]- 1,2,4-triazol-1-yl]pyrimidine- 4-carboxylate | 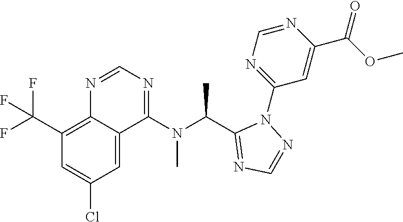 [SMILES: COC(=O)c1cc(-n2ncnc2[C@H](C)N(C)c2ncnc3c(C(F)(F)F)cc(Cl)cc23)ncn1] | 1.21 | 493/495 | 3 | — |
| I-23 | 6-[5-[(1S)-1-[[6-chloro-8- (trifluoromethyl)quinazolin- 4-yl]-methyl-amino]ethyl]- 1,2,4-triazol-1-yl]pyrimidine- 4-carboxylic acid | 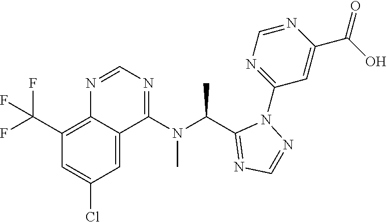 [SMILES: C[C@@H](c1ncnn1-c1cc(C(=O)O)ncn1)N(C)c1ncnc2c(C(F)(F)F)cc(Cl)cc12] | 1.12 | 479/481 | 3 | — |
| I-24 | methyl 5-[5-[(1S)-1-(tert- butoxycarbonylamino)ethyl]- 1,2,4-triazol-1-yl]pyrazine- 2-carboxylate | 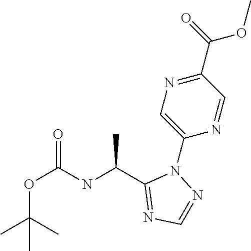 [SMILES: COC(=O)c1cnc(-n2ncnc2[C@H](C)NC(=O)OC(C)(C)C)cn1] | 0.83 | 349 | 1 | — |
| I-25 | tert-butyl N-[(1S)-1-[2-(5- carbamoylpyrazin-2-yl)- 1,2,4-triazol-3- yl]ethyl]carbamate | 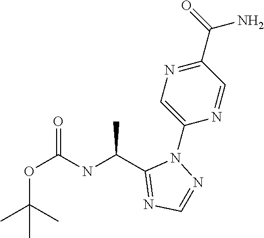 [SMILES: C[C@H](NC(=O)OC(C)(C)C)c1ncnn1-c1cnc(C(N)=O)cn1] | 0.71 | 334 | 1 | — |
| I-26 | tert-butyl N-[(1S)-1-[2-[5- (cyanomethylcarbamoyl)pyra- zin-2-yl]-1,2,4-triazol-3- yl]ethyl]carbamate | 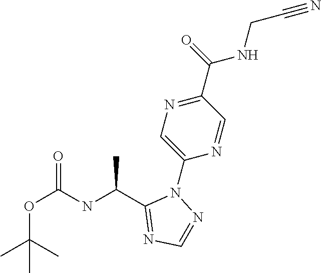 [SMILES: C[C@H](NC(=O)OC(C)(C)C)c1ncnn1-c1cnc(C(=O)NCC#N)cn1] | 0.78 | 373 | 1 | — |
| I-27 | [(1S)-1-[2-[5- (cyanomethylcarbamoyl)pyra- zin-2-yl]-1,2,4-triazol-3- yl]ethyl]ammonium chloride | 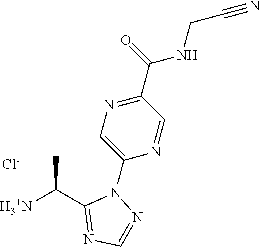 | 0.15 | 273 of the free base | 1 | — |
| I-28 | tert-butyl N-[(1S)-1-[2-(6- carbamoylpyrimidin-4-yl)- 1,2,4-triazol-3-yl]ethyl]-N- methyl-carbamate | 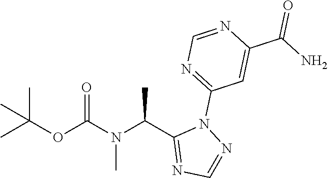 [SMILES: C[C@@H](c1ncnn1-c1cc(C(N)=O)ncn1)N(C)C(=O)OC(C)(C)C] | 1.01 | 292 [M + H − tBu]⁺ | 3 | — |
| I-29 | [(1S)-1-[2-(6- carbamoylpyrimidin-4-yl)- 1,2,4-triazol-3-yl]ethyl]- methyl-ammonium chloride | 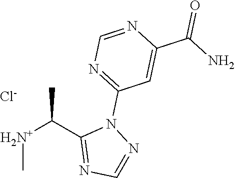 [SMILES: [Cl-] ; C[NH2+][C@@H](C)c1ncnn1-c1cc(C(N)=O)ncn1] | 0.16 | 248 of the free base | 3 | — |
| I-30 | tert-butyl N-[(1S)-1-[2-[6- (dimethylcarbamoyl)pyrimi- din-4-yl]-1,2,4-triazol-3- ylethyl]carbamate | 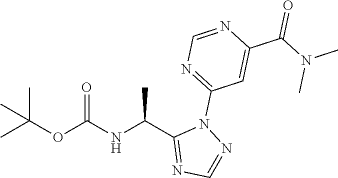 [SMILES: C[C@H](NC(=O)OC(C)(C)C)c1ncnn1-c1cc(C(=O)N(C)C)ncn1] | 0.97 | 306 [M + H − tBu]⁺ | 3 | — |
| I-31 | [(1S)-1-[2-[6- (dimethylcarbamoyl)pyrimi- din-4-yl]-1,2,4-triazol-3- yl]ethyl]ammonium chloride | 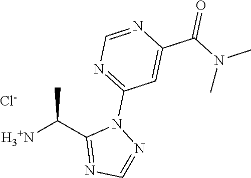 | 0.15 | 262 of the free base | 3 | — |
| I-32 | tert-butyl N-[(1S)-1-[2-[6- [ethyl(methyl)carbamoyl]pyri- midin-4-yl]-1,2,4-triazol-3- yl]ethyl]carbamate | 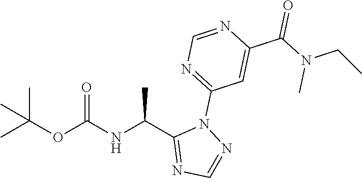 [SMILES: CCN(C)C(=O)c1cc(-n2ncnc2[C@H](C)NC(=O)OC(C)(C)C)ncn1] | 0.98 | 376 | 3 | — |
| I-33 | [(1S)-1-[2-[6- [ethyl(methyl)carbamoyl]pyri- midin-4-yl]-1,2,4-triazol-3- yl]ethyl]ammonium chloride | 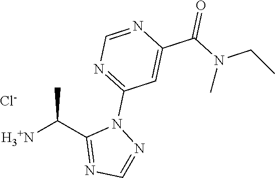 | 0.17 | 276 of the free base | 3 | — |
| I-34 | tert-butyl N-[(1S)-1-[2-[6- (cyanomethylcarbamoyl)pyri- midin-4-yl]-1,2,4-triazol-3- yl]ethyl]carbamate | 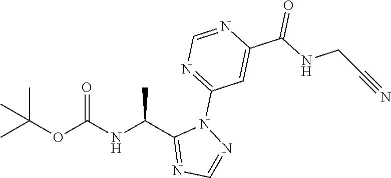 [SMILES: C[C@H](NC(=O)OC(C)(C)C)c1ncnn1-c1cc(C(=O)NCC#N)ncn1] | 0.91 | 273 [M + H − Boc]⁺ | 3 | — |
| I-35 | [(1S)-1-[2-[6- (cyanomethylcarbamoyl)pyri- imidin-4-yl]-1,2,4-triazol-3- yl]ethyl]ammonium 2,2,2- trifluoroacetate | 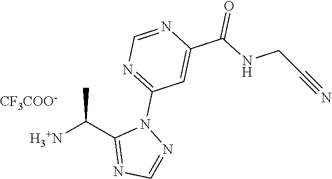 | 0.17 | 273 of the free base | 3 | — |
| I-36 | tert-butyl N-methyl-N-[(1S)- 1-[2-[6- (methylcarbamoyl)pyrimidin- 4-yl]-1,2,4-triazol-3- yl]ethyl]carbamate | 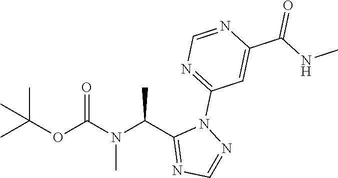 [SMILES: CNC(=O)c1cc(-n2ncnc2[C@H](C)N(C)C(=O)OC(C)(C)C)ncn1] | 0.88 | 362 | 1 | — |
| I-37 | methyl-[(1S)-1-[2-[6- (methylcarbamoyl)pyrimidin- 4-yl]-1,2,4-triazol-3- yl]ethyl]ammonium chloride | 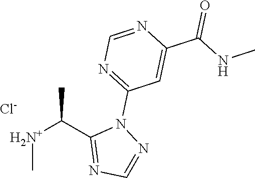 [SMILES: [Cl-] ; CNC(=O)c1cc(-n2ncnc2[C@H](C)[NH2+]C)ncn1] | 0.20 | 262 of the free base | 1 | — |
| I-38 | 6-chloro-N-[(1S)-1-[2-(6- iodopyrimidin-4-yl)-1,2,4- triazol-3-yl]ethyl]-8- (trifluoromethyl)quinazolin- 4-amine | 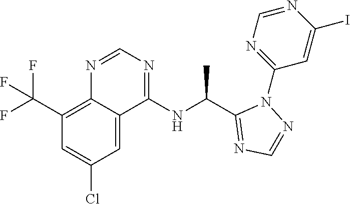 [SMILES: C[C@H](Nc1ncnc2c(C(F)(F)F)cc(Cl)cc12)c1ncnn1-c1cc(I)ncn1] | 1.24 | 547/549 | 3 | 178- 180 |
| I-39 | 6-chloro-N-[(1S)-1-[2-(6- iodopyrimidin-4-yl)-1,2,4- triazol-3-ylethyl]-N-methyl- 8- (trifluoromethyl)quinazolin- 4-amine | 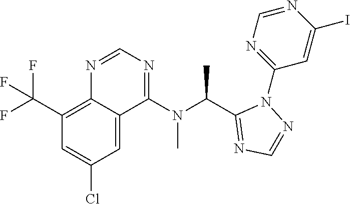 [SMILES: C[C@@H](c1ncnn1-c1cc(I)ncn1)N(C)c1ncnc2c(C(F)(F)F)cc(Cl)cc12] | 2.32 | 561/563 | 4 | — |
| I-40 | 6-chloro-8-iodo-4- tetrahydropyran-2-yloxy- quinazoline | 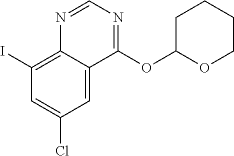 [SMILES: Clc1cc(I)c2ncnc(OC3CCCCO3)c2c1] | | | | — |
| I-41 | 6-chloro-4-tetrahydropyran- 2-yloxy-quinazoline-8-thiol | 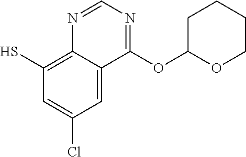 [SMILES: Sc1cc(Cl)cc2c(OC3CCCCO3)ncnc12] | | | | — |
| I-42 | 6-chloro-8- (difluoromethylsulfanyl)-4- tetrahydropyran-2-yloxy- quinazoline | 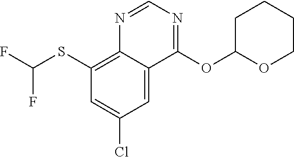 [SMILES: FC(F)Sc1cc(Cl)cc2c(OC3CCCCO3)ncnc12] | | | | — |
| I-43 | 6-chloro-8- (difluoromethylsulfonyl)-4- tetrahydropyran-2-yloxy- quinazoline | 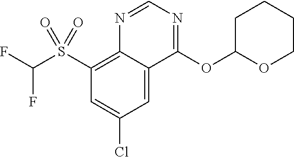 [SMILES: O=S(=O)(c1cc(Cl)cc2c(OC3CCCCO3)ncnc12)C(F)F] | | | | — |
| I-44 | 6-chloro-8- (difluoromethylsulfonyl)quin- azolin-4-ol | 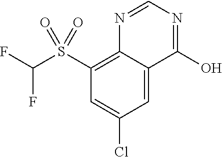 [SMILES: O=S(=O)(c1cc(Cl)cc2c(O)ncnc12)C(F)F] | | | | — |
| I-45 | 6-chloro-8- (trifluoromethylsulfonyl)quin- azolin-4-ol | 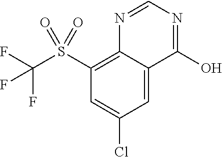 [SMILES: O=S(=O)(c1cc(Cl)cc2c(O)ncnc12)C(F)(F)F] | 1.08 | 313/315 | 3 | — |

Intermediates 1-40 to 1-44 characterized via ¹H NMR measurements:
I-40: ¹H NMR (400 MHz, CDCl₃) δ ppm 8.40 (s, 1H), 8.27 (d, 1H), 8.24 (d, 1H), 5.87 (m, 1H), 4.22 (m, 1H), 3.73 (m, 1H), 2.03 (m, 2H), 1.49-1.88 (br m, 4H).
I-41: ¹H NMR (400 MHz, DMSO-d₆) δ ppm 8.52 (s, 1H), 8.01 (d, 1H), 7.79 (d, 1H), 5.81 (m, 1H and s, 1H; overlapping signals), 4.09 (m, 1H), 3.68 (m, 1H), 1.94 (m, 1H), 1.85 (m, 2H), 1.38-1.79 (br m, 3H).
I-42: ¹H NMR (400 MHz, DMSO-d₆) δ ppm 8.58 (s, 1H), 8.08 (d, 1H), 7.97 (d, 1H), 7.89 (t, 1H), 5.82 (m, 1H), 4.10 (m, 1H), 3.69 (m, 1H), 1.98-1.81 (br m, 3H), 1.50-1.80 (br m, 3H).
I-43: ¹H NMR (400 MHz, DMSO-d₆) δ ppm 8.67 (s, 1H), 8.55 (d, 1H), 8.44 (d, 1H), 7.60 (t, 1H), 5.83 (m, 1H), 4.11 (m, 1H), 3.72 (m, 1H), 1.99-1.51 (br m, 6H).
I-44: ¹H NMR (400 MHz, DMSO-d₆) δ ppm 13.00 (br s, 1H), 8.51 (d, 1H), 8.42 (d, 1H), 8.35 (s, 1H), 7.59 (t, 1H).

### Abbreviations Used in Synthesis Schemes and Preparatory Examples

ACN acetonitrile
CPME cyclopentyl methyl ether (or methoxy cyclopentane)
Boc t-butoxycarbonyl
DBU 1,8-diazabicyclo[5.4.0]undec-7-ene
DCM dichloromethane
DDQ 2,3-dichloro-5,6-dicyano-1,4-benzoquinone
DMF dimethylformamide
DMSO dimethyl sulfoxide
DMSO-d₆ deuterated dimethylsulfoxide
DPEN diphenylethylenediamine
Et₃N triethylamine
EtOAc ethyl acetate
EtOH ethanol
HATU 1-[bis(dimethylamino)methylene]-1H-1,2,3-triazolo[4,5-b]pyridinium 3-oxide hexafluorophosphate, also known as hexafluorophosphate azabenzo-triazole tetramethyl uronium
HCl hydrochloric acid
MeCN acetonitrile
MeOH methanol
Ms methanesulfonyl (mesyl)
n-Bu n-butyl
n-BuLi n-butyllithium
NaHCO₃ sodium hydrogen carbonate
NHC N-heterocyclic carbene
NPhth phthalimide-1-yl
OMs mesylate group
OTf triflate group
OTs tosylate group
PdCl2dppf 1,1′-bis(diphenylphosphino)ferrocene]palladium(II) dichloride
TBME tert-butyl methyl ether
TEA triethylamine
TEMPO (2,2,6,6-tetramethylpiperidin-1-yl)oxidanyl
Tf trifluoromethanesulfonyl (triflyl)
TFA trifluoroacetic acid
THE tetrahydrofuran
Ts p-toluenesulfonyl (tosyl)
XPhos 2-dicyclohexylphosphino-2′,4′,6′-triisopropylbiphenyl
aq. aqueous
° C. degrees Celsius
equiv. equivalent
h hour(s)
LC/MS or LCMS liquid chromatography mass spectrometry
M molar
MHz megahertz
min minutes
mp or M.P. melting point
NMR nuclear magnetic resonance
ppm parts per million
RT room temperature
Rt retention time
RBF round-bottom flask
TLC thin layer chromatography
   The following combinations of a compound of formula (I) with another active substance in a weight ratio of 1:1 are preferred (the abbreviation “TX” means “one compound selected from the compounds defined in Tables A-1 to A-71, and compounds P1 to P85 (i.e. compounds P1 to P5, P6 to P17, P18 to P41, and P42 to P85):
an adjuvant selected from the group of substances consisting of petroleum oils (alternative name) (628)+TX;
abamectin+TX, acequinocyl+TX, acetamiprid+TX, acetoprole+TX, acrinathrin+TX, acynonapyr+TX, afidopyropen+TX, afoxolaner+TX, alanycarb+TX, allethrin+TX, alpha-cypermethrin+TX, alphamethrin+TX, amidoflumet+TX, aminocarb+TX, azocyclotin+TX, bensultap+TX, benzoximate+TX, benzpyrimoxan+TX, betacyfluthrin+TX, beta-cypermethrin+TX, bifenazate+TX, bifenthrin+TX, binapacryl+TX, bioallethrin+TX, S-bioallethrin+TX, bioresmethrin+TX, bistrifluron+TX, broflanilide+TX, brofluthrinate+TX, bromophos-ethyl+TX, buprofezine+TX, butocarboxim+TX, cadusafos+TX, carbaryl+TX, carbosulfan+TX, cartap+TX, CAS number: 1632218-00-8+TX, CAS number: 1808115-49-2+TX, CAS number: 2032403-97-5+TX, CAS number: 2044701-44-0+TX, CAS number: 2128706-05-6+TX, CAS number: 2095470-94-1+TX, CAS number: 2377084-09-6+TX, CAS number: 1445683-71-5+TX, CAS number: 2408220-94-8+TX, CAS number: 2408220-91-5+TX, CAS number: 1365070-72-9+TX, CAS number: 2171099-09-3+TX, CAS number: 2396747-83-2+TX, CAS number: 2133042-31-4+TX, CAS number: 2133042-44-9+TX, CAS number: 1445684-82-1+TX, CAS number: 1445684-82-1+TX, CAS number: 1922957-45-6+TX, CAS number: 1922957-46-7+TX, CAS number: 1922957-47-8+TX, CAS number: 1922957-48-9+TX, CAS number: 2415706-16-8+TX, CAS number: 1594624-87-9+TX, CAS number: 1594637-65-6+TX, CAS number: 1594626-19-3+TX, CAS number: 1990457-52-7+TX, CAS number: 1990457-55-0+TX, CAS number: 1990457-57-2+TX, CAS number: 1990457-77-6+TX, CAS number: 1990457-66-3+TX, CAS number: 1990457-85-6+TX, CAS number: 2220132-55-6+TX, CAS number: 1255091-74-7+TX, CAS number: 2719848-60-7+TX, CAS number: 1956329-03-5+TX, chlorantraniliprole+TX, chlordane+TX, chlorfenapyr+TX, chloroprallethrin+TX, chromafenozide+TX, clenpirin+TX, cloethocarb+TX, clothianidin+TX, 2-chlorophenyl N-methylcarbamate (CPMC)+TX, cyanofenphos+TX, cyantraniliprole+TX, cyclaniliprole+TX, cyclobutrifluram+TX, cycloprothrin+TX, cycloxaprid+TX, cyenopyrafen+TX, cyetpyrafen (or etpyrafen)+TX, cyflumetofen+TX, cyfluthrin+TX, cyhalodiamide+TX, cyhalothrin+TX, cypermethrin+TX, cyphenothrin+TX, cyproflanilide+TX, cyromazine+TX, deltamethrin+TX, diafenthiuron+TX, dialifos+TX, dibrom+TX, dicloromezotiaz+TX, diflovidazine+TX, diflubenzuron+TX, dimpropyridaz+TX, dinactin+TX, dinocap+TX, dinotefuran+TX, dioxabenzofos+TX, emamectin (or emamectin benzoate)+TX, empenthrin+TX, epsilon-momfluorothrin+TX, epsilon-metofluthrin+TX, esfenvalerate+TX, ethion+TX, ethiprole+TX, etofenprox+TX, etoxazole+TX, famphur+TX, fenazaquin+TX, fenfluthrin+TX, fenmezoditiaz+TX, fenitrothion+TX, fenobucarb+TX, fenothiocarb+TX, fenoxycarb+TX, fenpropathrin+TX, fenpyroximate+TX, fensulfothion+TX, fenthion+TX, fentinacetate+TX, fenvalerate+TX, fipronil+TX, flometoquin+TX, flonicamid+TX, fluacrypyrim+TX, fluazaindolizine+TX, fluazuron+TX, flubendiamide+TX, flubenzimine+TX, fluchlordiniliprole+TX, flucitrinate+TX, flucycloxuron+TX, flucythrinate+TX, fluensulfone+TX, flufenerim+TX, flufenprox+TX, flufiprole+TX, fluhexafon+TX, flumethrin+TX, fluopyram+TX, flupentiofenox+TX, flupyradifurone+TX, flupyroxystrobin+TX, flupyrimin+TX, fluralaner+TX, fluvalinate+TX, fluxametamide+TX, fosthiazate+TX, gamma-cyhalothrin+TX, guadipyr+TX, halofenozide+TX, halfenprox+TX, heptafluthrin+TX, hexythiazox+TX, hydramethylnon+TX, imicyafos+TX, imidacloprid+TX, imiprothrin+TX, indazapyroxamet+TX, indoxacarb+TX, iodomethane+TX, iprodione+TX, isocycloseram+TX, isothioate+TX, ivermectin+TX, kappa-bifenthrin+TX, kappa-tefluthrin+TX, lambda-Cyhalothrin+TX, ledprona+TX, lepimectin+TX, lotilaner+TX, lufenuron+TX, metaflumizone+TX, metaldehyde+TX, metam+TX, methomyl+TX, methoxyfenozide+TX, metofluthrin+TX, metolcarb+TX, mexacarbate+TX, milbemectin+TX, momfluorothrin+TX, niclosamide+TX, nicofluprole+TX; nitenpyram+TX, nithiazine+TX, omethoate+TX, oxamyl+TX, oxazosulfyl+TX, parathion-ethyl+TX, permethrin+TX, phenothrin+TX, phosphocarb+TX, piperonylbutoxide+TX, pirimicarb+TX, pirimiphos-ethyl+TX, pirimiphos-methyl+TX, Polyhedrosis virus+TX, prallethrin+TX, profenofos+TX, profluthrin+TX, propargite+TX, propetamphos+TX, propoxur+TX, prothiophos+TX, protrifenbute+TX, pyflubumide+TX, pymetrozine+TX, pyraclofos+TX, pyrafluprole+TX, pyridaben+TX, pyridalyl+TX, pyrifluquinazon+TX, pyrimidifen+TX, pyriminostrobin+TX, pyriprole+TX, pyriproxyfen+TX, resmethrin+TX, sarolaner+TX, selamectin+TX, silafluofen+TX, spinetoram+TX, spinosad+TX, spirobudifen+TX; spirodiclofen+TX, spiromesifen+TX, spiropidion+TX, spirotetramat+TX, spidoxamat+TX, sulfoxaflor+TX, tebufenozide+TX, tebufenpyrad+TX, tebupirimiphos+TX, tefluthrin+TX, temephos+TX, tetrachlorantraniliprole+TX, tetradiphon+TX, tetramethrin+TX, tetramethylfluthrin+TX, tetranactin+TX, tetraniliprole+TX, theta-cypermethrin+TX, thiacloprid+TX, thiamethoxam+TX, thiocyclam+TX, thiodicarb+TX, thiofanox+TX, thiometon+TX, thiosultap+TX, tigolaner+TX, tiorantraniliprole+TX; tioxazafen+TX, tolfenpyrad+TX, toxaphene+TX, tralomethrin+TX, transfluthrin+TX, triazamate+TX, triazophos+TX, trichlorfon+TX, trichloronate+TX, trichlorphon+TX, trifluenfuronate+TX, triflumezopyrim+TX, tyclopyrazoflor+TX, zeta-cypermethrin+TX, Extract of seaweed and fermentation product derived from melasse+TX, Extract of seaweed and fermentation product derived from melasse comprising urea+TX, amino acids+TX, potassium and molybdenum and EDTA-chelated manganese+TX, Extract of seaweed and fermented plant products+TX, Extract of seaweed and fermented plant products comprising phytohormones+TX, vitamins+TX, EDTA-chelated copper+TX, zinc+TX, and iron+TX, azadirachtin+TX, Bacillus aizawai+TX, Bacillus chitinosporus AQ746 (NRRL Accession No B-21 618)+TX, Bacillus firmus+TX, Bacillus kurstaki+TX, Bacillus mycoides AQ726 (NRRL Accession No. B-21664)+TX, Bacillus pumilus (NRRL Accession No B-30087)+TX, Bacillus pumilus AQ717 (NRRL Accession No. B-21662)+TX, Bacillus sp. AQ178 (ATCC Accession No. 53522)+TX, Bacillus sp. AQ175 (ATCC Accession No. 55608)+TX, Bacillus sp. AQ177 (ATCC Accession No. 55609)+TX, Bacillus subtilis unspecified+TX, Bacillus subtilis AQ153 (ATCC Accession No. 55614)+TX, Bacillus subtilis AQ30002 (NRRL Accession No. B-50421)+TX, Bacillus subtilis AQ30004 (NRRL Accession No. B—50455)+TX, Bacillus subtilis AQ713 (NRRL Accession No. B-21661)+TX, Bacillus subtilis AQ743 (NRRL Accession No. B-21665)+TX, Bacillus thuringiensis AQ52 (NRRL Accession No. B-21619)+TX, Bacillus thuringiensis BD #32 (NRRL Accession No B-21530)+TX, Bacillus thuringiensis subspec. kurstaki BMP 123+TX, Beauveria bassiana+TX, D-limonene+TX, Granulovirus+TX, Harpin+TX, Helicoverpa armigera Nucleopolyhedrovirus+TX, Helicoverpa zea Nucleopolyhedrovirus+TX, Heliothis virescens Nucleopolyhedrovirus+TX, Heliothis punctigera Nucleopolyhedrovirus+TX, Metarhizium spp.+TX, Muscodor albus 620 (NRRL Accession No. 30547)+TX, Muscodor roseus A3-5 (NRRL Accession No. 30548)+TX, Neem tree based products+TX, Paecilomyces fumosoroseus+TX, Paecilomyces lilacinus+TX, Pasteuria nishizawae+TX, Pasteuria penetrans+TX, Pasteuria ramosa+TX, Pasteuria thornei+TX, Pasteuria usgae+TX, P-cymene+TX, Plutella xylostella Granulosis virus+TX, Plutella xylostella Nucleopolyhedrovirus+TX, Polyhedrosis virus+TX, pyrethrum+TX, QRD 420 (a terpenoid blend)+TX, QRD 452 (a terpenoid blend)+TX, QRD 460 (a terpenoid blend)+TX, Quillaja saponaria+TX, Rhodococcus globerulus AQ719 (NRRL Accession No B-21663)+TX, Spodoptera frugiperda Nucleopolyhedrovirus+TX, Streptomyces galbus (NRRL Accession No. 30232)+TX, Streptomyces sp. (NRRL Accession No. B-30145)+TX, Terpenoid blend+TX, and Verticillium spp.+TX;
an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347)+TX, and triphenyltin hydroxide (IUPAC name) (347)+TX; an anthelmintic selected from the group of substances consisting of abamectin (1)+TX, crufomate (1011)+TX, cyclobutrifluram+TX, doramectin (alternative name) [CCN]+TX, emamectin (291)+TX, emamectin benzoate (291)+TX, eprinomectin (alternative name) [CCN]+TX, ivermectin (alternative name) [CCN]+TX, milbemycin oxime (alternative name) [CCN]+TX, moxidectin (alternative name) [CCN]+TX, piperazine [CCN]+TX, selamectin (alternative name) [CCN]+TX, spinosad (737)+TX, and thiophanate (1435)+TX;
an avicide selected from the group of substances consisting of chloralose (127)+TX, endrin (1122)+TX, fenthion (346)+TX, pyridin-4-amine (IUPAC name) (23)+TX, and strychnine (745)+TX; a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen (alternative name) [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal (alternative name) [CCN]+TX; a biological agent selected from the group of substances consisting of Adoxophyes orana GV (alternative name) (12)+TX, Agrobacterium radiobacter (alternative name) (13)+TX, Amblyseius spp. (alternative name) (19)+TX, Anagrapha falcifera NPV (alternative name) (28)+TX, Anagrus atomus (alternative name) (29)+TX, Aphelinus abdominalis (alternative name) (33)+TX, Aphidius colemani (alternative name) (34)+TX, Aphidoletes aphidimyza (alternative name) (35)+TX, Autographa californica NPV (alternative name) (38)+TX, Bacillus firmus (alternative name) (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (alternative name) (53)+TX, Beauveria brongniartii (alternative name) (54)+TX, Chrysoperla carnea (alternative name) (151)+TX, Cryptolaemus montrouzieri (alternative name) (178)+TX, Cydia pomonella GV (alternative name) (191)+TX, Dacnusa sibirica (alternative name) (212)+TX, Diglyphus isaea (alternative name) (254)+TX, Encarsia formosa (scientific name) (293)+TX, Eretmocerus eremicus (alternative name) (300)+TX, Helicoverpa zea NPV (alternative name) (431)+TX, Heterorhabditis bacteriophora and H. megidis (alternative name) (433)+TX, Hippodamia convergens (alternative name) (442)+TX, Leptomastix dactylopii (alternative name) (488)+TX, Macrolophus caliginosus (alternative name) (491)+TX, Mamestra brassicae NPV (alternative name) (494)+TX, Metaphycus helvolus (alternative name) (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (alternative name) (575)+TX, Orius spp. (alternative name) (596)+TX, Paecilomyces fumosoroseus (alternative name) (613)+TX, Phytoseiulus persimilis (alternative name) (644)+TX, Spodoptera exigua multicapsid nuclear polyhedrosis virus (scientific name) (741)+TX, Steinernema bibionis (alternative name) (742)+TX, Steinernema carpocapsae (alternative name) (742)+TX, Steinernema feltiae (alternative name) (742)+TX, Steinernema glaseri (alternative name) (742)+TX, Steinernema riobrave (alternative name) (742)+TX, Steinernema riobravis (alternative name) (742)+TX, Steinernema scapterisci (alternative name) (742)+TX, Steinernema spp. (alternative name) (742)+TX, Trichogramma spp. (alternative name) (826)+TX, Typhlodromus occidentalis (alternative name) (844)+TX, and Verticillium lecanii (alternative name) (848)+TX;
a soil sterilant selected from the group of substances consisting of iodomethane (IUPAC name) (542)+TX, and methyl bromide (537)+TX;
a chemosterilant selected from the group of substances consisting of apholate [CCN]+TX, bisazir (alternative name) [CCN]+TX, busulfan (alternative name) [CCN]+TX, diflubenzuron (250)+TX, dimatif (alternative name) [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron (alternative name) [CCN]+TX, tepa [CCN]+TX, thiohempa (alternative name) [CCN]+TX, thiotepa (alternative name) [CCN]+TX, tretamine (alternative name) [CCN]+TX, and uredepa (alternative name) [CCN]+TX;
an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin (alternative name) [CCN]+TX, brevicomin (alternative name) [CCN]+TX, codlelure (alternative name) [CCN]+TX, codlemone (alternative name) (167)+TX, cuelure (alternative name) (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure (alternative name) [CCN]+TX, ethyl 4-methyloctanoate (IUPAC name) (317)+TX, eugenol (alternative name) [CCN]+TX, frontalin (alternative name) [CCN]+TX, Gossyplure® (alternative name; 1:1 mixture of the (Z,E) and (Z,Z) isomers of hexadeca-7,11-dien-1-yl-acetate) (420)+TX, grandlure (421)+TX, grandlure I (alternative name) (421)+TX, grandlure II (alternative name) (421)+TX, grandlure III (alternative name) (421)+TX, grandlure IV (alternative name) (421)+TX, hexalure [CCN]+TX, ipsdienol (alternative name) [CCN]+TX, ipsenol (alternative name) [CCN]+TX, japonilure (alternative name) (481)+TX, lineatin (alternative name) [CCN]+TX, litlure (alternative name) [CCN]+TX, looplure (alternative name) [CCN]+TX, medlure [CCN]+TX, megatomoic acid (alternative name) [CCN]+TX, methyl eugenol (alternative name) (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure (alternative name) [CCN]+TX, oryctalure (alternative name) (317)+TX, ostramone (alternative name) [CCN]+TX, siglure [CCN]+TX, sordidin (alternative name) (736)+TX, sulcatol (alternative name) [CCN]+TX, tetradec-11-en-1-yl acetate (IUPAC name) (785)+TX, trimedlure (839)+TX, trimedlure A (alternative name) (839)+TX, trimedlure B₁ (alternative name) (839)+TX, trimedlure B₂ (alternative name) (839)+TX, trimedlure C (alternative name) (839) TX, and trunc-call (alternative name) [CCN]+TX;
an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN]+TX, and picaridin [CCN]+TX;
a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347)+TX, triphenyltin hydroxide (IUPAC name) (347)+TX, and pyriprole [394730-71-3]+TX;
a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (alternative name) (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben (alternative name)+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cyclobutrifluram+TX, cytokinins (alternative name) (210)+TX, dazomet (216)+TX, DBCP (1045)+TX, DCIP (218)+TX, diamidafos (1044)+TX, dichlofenthion (1051)+TX, dicliphos (alternative name)+TX, dimethoate (262)+TX, doramectin (alternative name) [CCN]+TX, emamectin (291)+TX, emamectin benzoate (291)+TX, eprinomectin (alternative name) [CCN]+TX, ethoprophos (312)+TX, ethylene dibromide (316)+TX, fenamiphos (326)+TX, fenpyrad (alternative name)+TX, fensulfothion (1158)+TX, fosthiazate (408)+TX, fosthietan (1196)+TX, furfural (alternative name) [CCN]+TX, GY-81 (development code) (423)+TX, heterophos [CCN]+TX, iodomethane (IUPAC name) (542)+TX, isamidofos (1230)+TX, isazofos (1231)+TX, ivermectin (alternative name) [CCN]+TX, kinetin (alternative name) (210)+TX, mecarphon (1258)+TX, metam (519)+TX, metam-potassium (alternative name) (519)+TX, metam-sodium (519)+TX, methyl bromide (537)+TX, methyl isothiocyanate (543)+TX, milbemycin oxime (alternative name) [CCN]+TX, moxidectin (alternative name) [CCN]+TX, Myrothecium verrucaria composition (alternative name) (565)+TX, NC-184 (compound code)+TX, oxamyl (602)+TX, phorate (636)+TX, phosphamidon (639)+TX, phosphocarb [CCN]+TX, sebufos (alternative name)+TX, selamectin (alternative name) [CCN]+TX, spinosad (737)+TX, terbam (alternative name)+TX, terbufos (773)+TX, tetrachlorothiophene (IUPAC/Chemical Abstracts name) (1422)+TX, thiafenox (alternative name)+TX, thionazin (1434)+TX, triazophos (820)+TX, triazuron (alternative name)+TX, xylenols [CCN]+TX, YI-5302 (compound code)+TX, zeatin (alternative name) (210)+TX, fluensulfone [318290-98-1]+TX, and fluopyram+TX;
a nitrification inhibitor selected from the group of substances consisting of potassium ethylxanthate [CCN]+TX, and nitrapyrin (580)+TX;
a plant activator selected from the group of substances consisting of acibenzolar (6)+TX, acibenzolar-S-methyl (6)+TX, probenazole (658)+TX, and Reynoutria sachalinensis extract (alternative name) (720)+TX;
a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (including alpha-bromadiolone)+TX, bromethalin (92)+TX, calcium cyanide (444)+TX, chloralose (127)+TX, chlorophacinone (140)+TX, cholecalciferol (alternative name) (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851)+TX, and zinc phosphide (640)+TX;
a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (alternative name) (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394)+TX, and sulfoxide (1406)+TX;
an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN]+TX, and ziram (856)+TX;
a virucide selected from the group of substances consisting of imanin (alternative name) [CCN] and ribavirin (alternative name) [CCN]+TX;
a wound protectant selected from the group of substances consisting of mercuric oxide (512)+TX, octhilinone (590)+TX, and thiophanate-methyl (802)+TX;
a biologically active substance selected from 1,1-bis(4-chloro-phenyl)-2-ethoxyethanol+TX, 2,4-dichlorophenyl benzenesulfonate+TX, 2-fluoro-N-methyl-N-1-naphthylacetamide+TX, 4-chlorophenyl phenyl sulfone+TX, acetoprole+TX, aldoxycarb+TX, amidithion+TX, amidothioate+TX, amiton+TX, amiton hydrogen oxalate+TX, amitraz+TX, aramite+TX, arsenous oxide+TX, azobenzene+TX, azothoate+TX, benomyl+TX, benoxa-fos+TX, benzyl benzoate+TX, bixafen+TX, brofenvalerate+TX, bromo-cyclen+TX, bromophos+TX, bromopropylate+TX, buprofezin+TX, butocarboxim+TX, butoxycarboxim+TX, butylpyridaben+TX, calcium polysulfide+TX, camphechlor+TX, carbanolate+TX, carbophenothion+TX, cymiazole+TX, chino-methionat+TX, chlorbenside+TX, chlordimeform+TX, chlordimeform hydrochloride+TX, chlorfenethol+TX, chlorfenson+TX, chlorfensulfide+TX, chlorobenzilate+TX, chloromebuform+TX, chloromethiuron+TX, chloropropylate+TX, chlorthiophos+TX, cinerin I+TX, cinerin II+TX, cinerins+TX, closantel+TX, coumaphos+TX, crotamiton+TX, crotoxyphos+TX, cufraneb+TX, cyanthoate+TX, DCPM+TX, DDT+TX, demephion+TX, demephion-O+TX, demephion-S+TX, demeton-methyl+TX, demeton-O+TX, demeton-O-methyl+TX, demeton-S+TX, demeton-S-methyl+TX, demeton-S-methylsulfon+TX, dichlofluanid+TX, dichlorvos+TX, dicliphos+TX, dienochlor+TX, dimefox+TX, dinex+TX, dinex-diclexine+TX, dinocap-4+TX, dinocap-6+TX, dinocton+TX, dino-penton+TX, dinosulfon+TX, dinoterbon+TX, dioxathion+TX, diphenyl sulfone+TX, disulfiram+TX, DNOC+TX, dofenapyn+TX, doramectin+TX, endothion+TX, eprinomectin+TX, ethoate-methyl+TX, etrimfos+TX, fenazaflor+TX, fenbutatin oxide+TX, fenothiocarb+TX, fenpyrad+TX, fen-pyroximate+TX, fenpyrazamine+TX, fenson+TX, fentrifanil+TX, flubenzimine+TX, flucycloxuron+TX, fluenetil+TX, fluorbenside+TX, FMC 1137+TX, formetanate+TX, formetanate hydrochloride+TX, formparanate+TX, gamma-HCH+TX, glyodin+TX, halfenprox+TX, hexadecyl cyclopropanecarboxylate+TX, isocarbophos+TX, jasmolin I+TX, jasmolin II+TX, jodfenphos+TX, lindane+TX, malonoben+TX, mecarbam+TX, mephosfolan+TX, mesulfen+TX, methacrifos+TX, methyl bromide+TX, metolcarb+TX, mexacarbate+TX, milbemycin oxime+TX, mipafox+TX, monocrotophos+TX, morphothion+TX, moxidectin+TX, naled+TX, 4-chloro-2-(2-chloro-2-methyl-propyl)-5-[(6-iodo-3-pyridyl)methoxy]pyridazin-3-one+TX, nifluridide+TX, nikkomycins+TX, nitrilacarb+TX, nitrilacarb 1:1 zinc chloride complex+TX, omethoate+TX, oxydeprofos+TX, oxydisulfoton+TX, pp′-DDT+TX, parathion+TX, permethrin+TX, phenkapton+TX, phosalone+TX, phosfolan+TX, phosphamidon+TX, polychloroterpenes+TX, polynactins+TX, proclonol+TX, promacyl+TX, propoxur+TX, prothidathion+TX, prothoate+TX, pyrethrin I+TX, pyrethrin II+TX, pyrethrins+TX, pyridaphenthion+TX, pyrimitate+TX, quinalphos+TX, quintiofos+TX, R-1492+TX, phosglycin+TX, rotenone+TX, schradan+TX, sebufos+TX, selamectin+TX, sophamide+TX, SSI-121+TX, sulfiram+TX, sulfluramid+TX, sulfotep+TX, sulfur+TX, diflovidazin+TX, tau-fluvalinate+TX, TEPP+TX, terbam+TX, tetradifon+TX, tetrasul+TX, thiafenox+TX, thiocarboxime+TX, thiofanox+TX, thiometon+TX, thioquinox+TX, thuringiensin+TX, triamiphos+TX, triarathene+TX, triazophos+TX, triazuron+TX, trifenofos+TX, trinactin+TX, vamidothion+TX, vaniliprole+TX, bethoxazin+TX, copper dioctanoate+TX, copper sulfate+TX, cybutryne+TX, dichlone+TX, dichlorophen+TX, endothal+TX, fentin+TX, hydrated lime+TX, nabam+TX, quinoclamine+TX, quinonamid+TX, simazine+TX, triphenyltin acetate+TX, triphenyltin hydroxide+TX, crufomate+TX, piperazine+TX, thiophanate+TX, chloralose+TX, fenthion+TX, pyridin-4-amine+TX, strychnine+TX, 1-hydroxy-1H-pyridine-2-thione+TX, 4-(quinoxalin-2-ylamino)benzenesulfonamide+TX, 8-hydroxyquinoline sulfate+TX, bronopol+TX, copper hydroxide+TX, cresol+TX, dipyrithione+TX, dodicin+TX, fenaminosulf+TX, formaldehyde+TX, hydrargaphen+TX, kasugamycin+TX, kasugamycin hydrochloride hydrate+TX, nickel bis(dimethyldithiocarbamate)+TX, nitrapyrin+TX, octhilinone+TX, oxolinic acid+TX, oxytetracycline+TX, potassium hydroxyquinoline sulfate+TX, probenazole+TX, streptomycin+TX, streptomycin sesquisulfate+TX, tecloftalam+TX, thiomersal+TX, Adoxophyes orana GV+TX, Agrobacterium radiobacter+TX, Amblyseius spp.+TX, Anagrapha falcifera NPV+TX, Anagrus atomus+TX, Aphelinus abdominalis+TX, Aphidius colemani+TX, Aphidoletes aphidimyza+TX, Autographa californica NPV+TX, Bacillus sphaericus Neide+TX, Beauveria brongniartii+TX, Chrysoperla carnea+TX, Cryptolaemus montrouzieri+TX, Cydia pomonella GV+TX, Dacnusa sibirica+TX, Diglyphus isaea+TX, Encarsia formosa+TX, Eretmocerus eremicus+TX, Heterorhabditis bacteriophora and H. megidis+TX, Hippodamia convergens+TX, Leptomastix dactylopii+TX, Macrolophus caliginosus+TX, Mamestra brassicae NPV+TX, Metaphycus helvolus+TX, Metarhizium anisopliae var. acridum+TX, Metarhizium anisopliae var. anisopliae+TX, Neodiprion sertifer NPV and N. lecontei NPV+TX, Orius spp.+TX, Paecilomyces fumosoroseus+TX, Phytoseiulus persimilis+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, Trichogramma spp.+TX, Typhlodromus occidentalis+TX, Verticillium lecanii+TX, apholate+TX, bisazir+TX, busulfan+TX, dimatif+TX, hemel+TX, hempa+TX, metepa+TX, methiotepa+TX, methyl apholate+TX, morzid+TX, penfluron+TX, tepa+TX, thiohempa+TX, thiotepa+TX, tretamine+TX, uredepa+TX, (E)-dec-5-en-1-yl acetate with (E)-dec-5-en-1-ol+TX, (E)-tridec-4-en-1-yl acetate+TX, (E)-6-methylhept-2-en-4-ol+TX, (E,Z)-tetradeca-4,10-dien-1-yl acetate+TX, (Z)-dodec-7-en-1-yl acetate+TX, (Z)-hexadec-11-enal+TX, (Z)-hexadec-11-en-1-yl acetate+TX, (Z)-hexadec-13-en-11-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, (7E,9Z)-dodeca-7,9-dien-1-yl acetate+TX, (9Z,11E)-tetradeca-9,11-dien-1-yl acetate+TX, (9Z,12E)-tetradeca-9,12-dien-1-yl acetate+TX, 14-methyloctadec-1-ene+TX, 4-methylnonan-5-ol with 4-methylnonan-5-one+TX, alpha-multistriatin+TX, brevicomin+TX, codlelure+TX, codlemone+TX, cuelure+TX, disparlure+TX, dodec-8-en-1-yl acetate+TX, dodec-9-en-1-yl acetate+TX, dodeca-8+TX, 10-dien-1-yl acetate+TX, dominicalure+TX, ethyl 4-methyloctanoate+TX, eugenol+TX, frontalin+TX, grandlure+TX, grandlure I+TX, grandlure II+TX, grandlure III+TX, grandlure IV+TX, hexalure+TX, ipsdienol+TX, ipsenol+TX, japonilure+TX, lineatin+TX, litlure+TX, looplure+TX, medlure+TX, megatomoic acid+TX, methyl eugenol+TX, muscalure+TX, octadeca-2,13-dien-1-yl acetate+TX, octadeca-3,13-dien-1-yl acetate+TX, orfralure+TX, oryctalure+TX, ostramone+TX, siglure+TX, sordidin+TX, sulcatol+TX, tetradec-11-en-1-yl acetate+TX, trimedlure+TX, trimedlure A+TX, trimedlure Bi+TX, trimedlure B₂+TX, trimedlure C+TX, trunc-call+TX, 2-(octylthio)-ethanol+TX, butopyronoxyl+TX, butoxy(polypropylene glycol)+TX, dibutyl adipate+TX, dibutyl phthalate+TX, dibutyl succinate+TX, diethyltoluamide+TX, dimethyl carbate+TX, dimethyl phthalate+TX, ethyl hexanediol+TX, hexamide+TX, methoquin-butyl+TX, methylneodecanamide+TX, oxamate+TX, picaridin+TX, 1-dichloro-1-nitroethane+TX, 1,1-dichloro-2,2-bis(4-ethylphenyl)-ethane+TX, 1,2-dichloropropane with 1,3-dichloropropene+TX, 1-bromo-2-chloroethane+TX, 2,2,2-trichloro-1-(3,4-dichloro-phenyl)ethyl acetate+TX, 2,2-dichlorovinyl 2-ethylsulfinylethyl methyl phosphate+TX, 2-(1,3-dithiolan-2-yl)phenyl dimethylcarbamate+TX, 2-(2-butoxyethoxy)ethyl thiocyanate+TX, 2-(4,5-dimethyl-1,3-dioxolan-2-yl)phenyl methylcarbamate+TX, 2-(4-chloro-3,5-xylyloxy)ethanol+TX, 2-chlorovinyl diethyl phosphate+TX, 2-imidazolidone+TX, 2-isovalerylindan-1,3-dione+TX, 2-methyl(prop-2-ynyl)aminophenyl methylcarbamate+TX, 2-thiocyanatoethyl laurate+TX, 3-bromo-1-chloroprop-1-ene+TX, 3-methyl-1-phenylpyrazol-5-yl dimethyl-carbamate+TX, 4-methyl(prop-2-ynyl)amino-3,5-xylyl methylcarbamate+TX, 5,5-dimethyl-3-oxocyclohex-1-enyl dimethylcarbamate+TX, acethion+TX, acrylonitrile+TX, aldrin+TX, allosamidin+TX, allyxycarb+TX, alpha-ecdysone+TX, aluminium phosphide+TX, aminocarb+TX, anabasine+TX, athidathion+TX, azamethiphos+TX, Bacillus thuringiensis delta endotoxins+TX, barium hexafluorosilicate+TX, barium polysulfide+TX, barthrin+TX, Bayer 22/190+TX, Bayer 22408+TX, beta-cyfluthrin+TX, beta-cypermethrin+TX, bioethanomethrin+TX, biopermethrin+TX, bis(2-chloroethyl) ether+TX, borax+TX, bromfenvinfos+TX, bromo-DDT+TX, bufencarb+TX, butacarb+TX, butathiofos+TX, butonate+TX, calcium arsenate+TX, calcium cyanide+TX, carbon disulfide+TX, carbon tetrachloride+TX, cartap hydrochloride+TX, cevadine+TX, chlorbicyclen+TX, chlordane+TX, chlordecone+TX, chloroform+TX, chloropicrin+TX, chlorphoxim+TX, chlorprazophos+TX, cis-resmethrin+TX, cismethrin+TX, clocythrin+TX, copper acetoarsenite+TX, copper arsenate+TX, copper oleate+TX, coumithoate+TX, cryolite+TX, CS 708+TX, cyanofenphos+TX, cyanophos+TX, cyclethrin+TX, cythioate+TX, d-tetramethrin+TX, DAEP+TX, dazomet+TX, decarbofuran+TX, diamidafos+TX, dicapthon+TX, dichlofenthion+TX, dicresyl+TX, dicyclanil+TX, dieldrin+TX, diethyl 5-methylpyrazol-3-yl phosphate+TX, dilor+TX, dimefluthrin+TX, dimetan+TX, dimethrin+TX, dimethylvinphos+TX, dimetilan+TX, dinoprop+TX, dinosam+TX, dinoseb+TX, diofenolan+TX, dioxabenzofos+TX, dithicrofos+TX, DSP+TX, ecdysterone+TX, EI 1642+TX, EMPC+TX, EPBP+TX, etaphos+TX, ethiofencarb+TX, ethyl formate+TX, ethylene dibromide+TX, ethylene dichloride+TX, ethylene oxide+TX, EXD+TX, fenchlorphos+TX, fenethacarb+TX, fenitrothion+TX, fenoxacrim+TX, fenpirithrin+TX, fensulfothion+TX, fenthion-ethyl+TX, flucofuron+TX, fosmethilan+TX, fospirate+TX, fosthietan+TX, furathiocarb+TX, furethrin+TX, guazatine+TX, guazatine acetates+TX, sodium tetrathiocarbonate+TX, halfenprox+TX, HCH+TX, HEOD+TX, heptachlor+TX, heterophos+TX, HHDN+TX, hydrogen cyanide+TX, hyquincarb+TX, IPSP+TX, isazofos+TX, isobenzan+TX, isodrin+TX, isofenphos+TX, isolane+TX, isoprothiolane+TX, isoxathion+TX, juvenile hormone I+TX, juvenile hormone II+TX, juvenile hormone 11+TX, kelevan+TX, kinoprene+TX, lead arsenate+TX, leptophos+TX, lirimfos+TX, lythidathion+TX, m-cumenyl methylcarbamate+TX, magnesium phosphide+TX, mazidox+TX, mecarphon+TX, menazon+TX, mercurous chloride+TX, mesulfenfos+TX, metam+TX, metam-potassium+TX, metam-sodium+TX, methanesulfonyl fluoride+TX, methocrotophos+TX, methoprene+TX, methothrin+TX, methoxychlor+TX, methyl isothiocyanate+TX, methylchloroform+TX, methylene chloride+TX, metoxadiazone+TX, mirex+TX, naftalofos+TX, naphthalene+TX, NC-170+TX, nicotine+TX, nicotine sulfate+TX, nithiazine+TX, nornicotine+TX, O-5-dichloro-4-iodophenyl O-ethyl ethylphosphonothioate+TX, O,O-diethyl O-4-methyl-2-oxo-2H-chromen-7-yl phosphorothioate+TX, O,O-diethyl O-6-methyl-2-propylpyrimidin-4-yl phosphorothioate+TX, O,O,O′,O′-tetrapropyl dithiopyrophosphate+TX, oleic acid+TX, para-dichlorobenzene+TX, parathion-methyl+TX, pentachlorophenol+TX, pentachlorophenyl laurate+TX, PH 60-38+TX, phenkapton+TX, phosnichlor+TX, phosphine+TX, phoxim-methyl+TX, pirimetaphos+TX, polychlorodicyclopentadiene isomers+TX, potassium arsenite+TX, potassium thiocyanate+TX, precocene I+TX, precocene II+TX, precocene III+TX, primidophos+TX, profluthrin+TX, promecarb+TX, prothiofos+TX, pyrazophos+TX, pyresmethrin+TX, quassia+TX, quinalphos-methyl+TX, quinothion+TX, rafoxanide+TX, resmethrin+TX, rotenone+TX, kadethrin+TX, ryania+TX, ryanodine+TX, sabadilla+TX, schradan+TX, sebufos+TX, SI-0009+TX, thiapronil+TX, sodium arsenite+TX, sodium cyanide+TX, sodium fluoride+TX, sodium hexafluorosilicate+TX, sodium pentachlorophenoxide+TX, sodium selenate+TX, sodium thiocyanate+TX, sulcofuron+TX, sulcofuron-sodium+TX, sulfuryl fluoride+TX, sulprofos+TX, tar oils+TX, tazimcarb+TX, TDE+TX, tebupirimfos+TX, temephos+TX, terallethrin+TX, tetrachloroethane+TX, thicrofos+TX, thiocyclam+TX, thiocyclam hydrogen oxalate+TX, thionazin+TX, thiosultap+TX, thiosultap-sodium+TX, tralomethrin+TX, transpermethrin+TX, triazamate+TX, trichlormetaphos-3+TX, trichloronat+TX, trimethacarb+TX, tolprocarb+TX, triclopyricarb+TX, triprene+TX, veratridine+TX, veratrine+TX, XMC+TX, zetamethrin+TX, zinc phosphide+TX, zolaprofos+TX, meperfluthrin+TX, tetramethylfluthrin+TX, bis(tributyltin) oxide+TX, bromoacetamide+TX, ferric phosphate+TX, niclosamide-olamine+TX, tributyltin oxide+TX, pyrimorph+TX, trifenmorph+TX, 1,2-dibromo-3-chloropropane+TX, 1,3-dichloropropene+TX, 3,4-dichlorotetrahydrothio-phene 1,1-dioxide+TX, 3-(4-chlorophenyl)-5-methylrhodanine+TX, 5-methyl-6-thioxo-1,3,5-thiadiazinan-3-ylacetic acid+TX, 6-isopentenylaminopurine+TX, anisiflupurin+TX, benclothiaz+TX, cytokinins+TX, DCIP+TX, furfural+TX, isamidofos+TX, kinetin+TX, Myrothecium verrucaria composition+TX, tetrachlorothiophene+TX, xylenols+TX, zeatin+TX, potassium ethylxanthate+TX,acibenzolar+TX, acibenzolar-S-methyl+TX, Reynoutria sachalinensis extract+TX, alpha-chlorohydrin+TX, antu+TX, barium carbonate+TX, bisthiosemi+TX, brodifacoum+TX, bromadiolone+TX, bromethalin+TX, chlorophacinone+TX, cholecalciferol+TX, coumachlor+TX, coumafuryl+TX, coumatetralyl+TX, crimidine+TX, difenacoum+TX, difethialone+TX, diphacinone+TX, ergocalciferol+TX, flocoumafen+TX, fluoroacetamide+TX, flupropadine+TX, flupropadine hydrochloride+TX, norbormide+TX, phosacetim+TX, phosphorus+TX, pindone+TX, pyrinuron+TX, scilliroside+TX, -sodium fluoroacetate+TX, thallium sulfate+TX, warfarin+TX, -2-(2-butoxyethoxy)ethyl piperonylate+TX, 5-(1,3-benzodioxol-5-yl)-3-hexylcyclohex-2-enone+TX, farnesol with nerolidol+TX, verbutin+TX, MGK 264+TX, piperonyl butoxide+TX, piprotal+TX, propyl isomer+TX, S421+TX, sesamex+TX, sesasmolin+TX, sulfoxide+TX, anthraquinone+TX, copper naphthenate+TX, copper oxychloride+TX, dicyclopentadiene+TX, thiram+TX, zinc naphthenate+TX, ziram+TX, imanin+TX, ribavirin+TX, chloroinconazide+TX, mercuric oxide+TX, thiophanate-methyl+TX, azaconazole+TX, bitertanol+TX, bromuconazole+TX, cyproconazole+TX, difenoconazole+TX, diniconazole-+TX, epoxiconazole+TX, fenbuconazole+TX, fluquinconazole+TX, flusilazole+TX, flutriafol+TX, furametpyr+TX, hexaconazole+TX, imazalil-+TX, imiben-conazole+TX, ipconazole+TX, metconazole+TX, myclobutanil+TX, paclobutrazole+TX, pefurazoate+TX, penconazole+TX, prothioconazole+TX, pyrifenox+TX, prochloraz+TX, propiconazole+TX, pyrisoxazole+TX, -simeconazole+TX, tebucon-azole+TX, tetraconazole+TX, triadimefon+TX, triadimenol+TX, triflumizole+TX, triticonazole+TX, ancymidol+TX, fenarimol+TX, nuarimol+TX, bupirimate+TX, dimethirimol+TX, ethirimol+TX, dodemorph+TX, fenpropidin+TX, fenpropimorph+TX, spiroxamine+TX, tridemorph+TX, cyprodinil+TX, mepanipyrim+TX, pyrimethanil+TX, fenpiclonil+TX, fludioxonil+TX, benalaxyl+TX, furalaxyl+TX, metalaxyl+TX, R-metalaxyl+TX, ofurace+TX, oxadixyl+TX, carbendazim+TX, debacarb+TX, fuberidazole-+TX, thiabendazole+TX, chlozolinate+TX, dichlozoline+TX, myclozoline-+TX, procymidone+TX, vinclozoline+TX, boscalid+TX, carboxin+TX, fenfuram+TX, flutolanil+TX, mepronil+TX, oxycarboxin+TX, penthiopyrad+TX, thifluzamide+TX, dodine+TX, iminoctadine+TX, azoxystrobin+TX, dimoxystrobin+TX, enestroburin+TX, fenaminstrobin+TX, flufenoxystrobin+TX, fluoxastrobin+TX, kresoxim-methyl+TX, metominostrobin+TX, trifloxystrobin+TX, orysastrobin+TX, picoxystrobin+TX, pyraclostrobin+TX, pyrametostrobin+TX, pyraoxystrobin+TX, ferbam+TX, mancozeb+TX, maneb+TX, metiram+TX, propineb+TX, zineb+TX, captafol+TX, captan+TX, fluoroimide+TX, folpet+TX, tolylfluanid+TX, bordeaux mixture+TX, copper oxide+TX, mancopper+TX, oxine-copper+TX, nitrothal-isopropyl+TX, edifenphos+TX, iprobenphos+TX, phosdiphen+TX, tolclofos-methyl+TX, anilazine+TX, benthiavalicarb+TX, blasticidin-S+TX, chloroneb-+TX, chloro-tha-lonil+TX, cyflufenamid+TX, cymoxanil+TX, cyclobutrifluram+TX, diclocymet+TX, diclomezine+TX, dicloran+TX, diethofencarb+TX, dimethomorph-+TX, flumorph+TX, dithianon+TX, ethaboxam+TX, etridiazole+TX, famoxadone+TX, fenamidone+TX, fenoxanil+TX, ferimzone+TX, fluazinam+TX, flumetylsulforim+TX, fluopicolide+TX, fluoxytioconazole+TX, flusulfamide+TX, fluxapyroxad+TX, fenhexamid+TX, fosetyl-aluminium-+TX, hymexazol+TX, iprovalicarb+TX, cyazofamid+TX, methasulfocarb+TX, metrafenone+TX, pencycuron+TX, phthalide+TX, polyoxins+TX, propamocarb+TX, pyribencarb+TX, proquinazid+TX, pyroquilon+TX, pyriofenone+TX, quinoxyfen+TX, quintozene+TX, tiadinil+TX, triazoxide+TX, tricyclazole+TX, triforine+TX, validamycin+TX, valifenalate+TX, zoxamide+TX, mandipropamid+TX, flubeneteram+TX, isopyrazam+TX, sedaxane+TX, benzovindiflupyr+TX, pydiflumetofen+TX, 3-difluoromethyl-1-methyl-1H-pyrazole-4-carboxylic acid (3′,4′,5′-trifluoro-biphenyl-2-yl)-amide+TX, isoflucypram+TX, isotianil+TX, dipymetitrone+TX, 6-ethyl-5,7-dioxo-pyrrolo[4,5][1,4]dithiino[1,2-c]isothiazole-3-carbonitrile+TX, 2-(difluoromethyl)-N-[3-ethyl-1,1-dimethyl-indan-4-yl]pyridine-3-carboxamide+TX, 4-(2,6-difluorophenyl)-6-methyl-5-phenyl-pyridazine-3-carbonitrile+TX, (R)-3-(difluoromethyl)-1-methyl-N-[1,1,3-trimethylindan-4-yl]pyrazole-4-carboxamide+TX, 4-(2-bromo-4-fluoro-phenyl)-N-(2-chloro-6-fluoro-phenyl)-2,5-dimethyl-pyrazol-3-amine+TX, 4-(2-bromo- 4-fluorophenyl)-N-(2-chloro- 6-fluorophenyl)-1, 3-dimethyl- 1H—pyrazol- 5-amine+TX, fluindapyr+TX, coumethoxystrobin (jiaxiangjunzhi)+TX, Ivbenmixianan+TX, dichlobentiazox+TX, mandestrobin+TX, 3-(4,4-difluoro-3,4-dihydro-3,3-dimethylisoquinolin-1-yl)quinolone+TX, 2-[2-fluoro-6-[(8-fluoro-2-methyl-3-quinolyl)oxy]phenyl]propan-2-ol+TX, oxathiapiprolin+TX, tert-butyl N-[6-[[[(1-methyltetrazol-5-yl)-phenyl-methylene]amino]oxymethyl]-2-pyridyl]carbamate+TX, pyraziflumid+TX, inpyrfluxam+TX, trolprocarb+TX, mefentrifluconazole+TX, ipfentrifluconazole+TX, 2-(difluoromethyl)-N-[(3R)-3-ethyl-1,1-dimethyl-indan-4-yl]pyridine-3-carboxamide+TX, N′-(2,5-dimethyl-4-phenoxy-phenyl)-N-ethyl-N-methyl-formamidine+TX, N′-[4-(4,5-dichlorothiazol-2-yl)oxy-2,5-dimethyl-phenyl]-N-ethyl-N-methyl-formamidine+TX, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl]methanesulfonate+TX, but-3-ynyl N-[6-[[(Z)-[(1-methyltetrazol-5-yl)-phenyl-methylene]amino]oxymethyl]-2-pyridyl]carbamate+TX, methyl N-[[5-[4-(2,4-dimethylphenyl)triazol-2-yl]-2-methyl-phenyl]methyl]carbamate+TX, 3-chloro-6-methyl-5-phenyl-4-(2,4,6-trifluorophenyl)pyridazine+TX, pyridachlometyl+TX, 3-(difluoromethyl)-1-methyl-N-[1,1,3-trimethylindan-4-yl]pyrazole-4-carboxamide+TX, 1-[2-[[1-(4-chlorophenyl)pyrazol-3-yl]oxymethyl]-3-methyl-phenyl]-4-methyl-tetrazol-5-one+TX, 1-methyl-4-[3-methyl-2-[[2-methyl-4-(3,4,5-trimethylpyrazol-1-yl)phenoxy]methyl]phenyl]tetrazol-5-one+TX, aminopyrifen+TX, ametoctradin+TX, amisulbrom+TX, penflufen+TX, (Z,2E)-5-[1-(4-chlorophenyl)pyrazol-3-yl]oxy-2-methoxyimino-N,3-dimethyl-pent-3-enamide+TX, florylpicoxamid+TX, fenpicoxamid+TX, metarylpicoxamid+TX, tebufloquin+TX, ipflufenoquin+TX, quinofumelin+TX, isofetamid+TX, ethyl 1-[[4-[[2-(trifluoromethyl)-1,3-dioxolan-2-yl]methoxy]phenyl]methyl]pyrazole-3-carboxylate+TX (may be prepared from the methods described in WO 2020/056090), ethyl 1-[[4-[(Z)-2-ethoxy-3,3,3-trifluoro-prop-1-enoxy]phenyl]methyl]pyrazole-3-carboxylate+TX (may be prepared from the methods described in WO 2020/056090), methyl N-[[4-[1-(4-cyclopropyl-2,6-difluoro-phenyl)pyrazol-4-yl]-2-methyl-phenyl]methyl]carbamate+TX (may be prepared from the methods described in WO 2020/097012), methyl N-[[4-[1-(2,6-difluoro-4-isopropyl-phenyl)pyrazol-4-yl]-2-methyl-phenyl]methyl]carbamate+TX (may be prepared from the methods described in WO 2020/097012), 6-chloro-3-(3-cyclopropyl-2-fluoro-phenoxy)-N-[2-(2,4-dimethylphenyl)-2,2-difluoro-ethyl]-5-methyl-pyridazine-4-carboxamide+TX (may be prepared from the methods described in WO 2020/109391), 6-chloro-N-[2-(2-chloro-4-methyl-phenyl)-2,2-difluoro-ethyl]-3-(3-cyclopropyl-2-fluoro-phenoxy)-5-methyl-pyridazine-4-carboxamide+TX (may be prepared from the methods described in WO 2020/109391), 6-chloro-3-(3-cyclopropyl-2-fluoro-phenoxy)-N-[2-(3,4-dimethylphenyl)-2,2-difluoro-ethyl]-5-methyl-pyridazine-4-carboxamide+TX (may be prepared from the methods described in WO 2020/109391), N-[2-[2,4-dichloro-phenoxy]phenyl]-3-(difluoromethyl)-1-methyl-pyrazole-4-carboxamide+TX, N-[2-[2-chloro-4-(trifluoromethyl)phenoxy]phenyl]-3-(difluoromethyl)-1-methyl-pyrazole-4-carboxamide+TX, benzothiostrobin+TX, phenamacril+TX, 5-amino-1,3,4-thiadiazole-2-thiol zinc salt (2:1)+TX, fluopyram+TX, flufenoxadiazam+TX, flutianil+TX, fluopimomide+TX, pyrapropoyne+TX, picarbutrazox+TX, 2-(difluoromethyl)-N-(3-ethyl-1, 1-dimethyl-indan-4-yl)pyridine-3-carboxamide+TX, 2-(difluoromethyl)-N—((3R)-1, 1, 3-trimethylindan- 4-yl) pyridine- 3-carboxamide+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, metyltetraprole+TX, 2-(difluoromethyl)-N—((3R)-1, 1, 3-trimethylindan- 4-yl) pyridine- 3-carboxamide+TX, a- (1, 1-dimethylethyl)-a- [4′-(trifluoromethoxy) [1, 1′-biphenyl]-4-yl]-5-pyrimidinemethanol+TX, fluoxapiprolin+TX, enoxastrobin+TX, methyl (Z)-3-methoxy-2-[2-methyl-5-[4-(trifluoromethyl)triazol-2-yl]phenoxy]prop-2-enoate+TX, methyl (Z)-3-methoxy-2-[2-methyl-5-(4-propyltriazol-2-yl)phenoxy]prop-2-enoate+TX, methyl (Z)-2-[5-(3-isopropylpyrazol-1-yl)-2-methyl-phenoxy]-3-methoxy-prop-2-enoate+TX, methyl (Z)-3-methoxy-2-[2-methyl-5-(3-propylpyrazol-1-yl)phenoxy]prop-2-enoate+TX, methyl (Z)-3-methoxy-2-[2-methyl-5-[3-(trifluoromethyl)pyrazol-1-yl]phenoxy]prop-2-enoate+TX (these compounds may be prepared from the methods described in WO2020/079111), methyl (Z)-2-(5-cyclohexyl-2-methyl-phenoxy)-3-methoxy-prop-2-enoate+TX, methyl (Z)-2-(5-cyclopentyl-2-methyl-phenoxy)-3-methoxy-prop-2-enoate+TX (these compounds may be prepared from the methods described in WO2020/193387), 4-[[6-[2-(2,4-difluorophenyl)-1,1-difluoro-2-hydroxy-3-(1,2,4-triazol-1-yl)propyl]-3-pyridyl]oxy]benzonitrile+TX, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile+TX, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+TX, trinexapac+TX, coumoxystrobin+TX, zhongshengmycin+TX, thiodiazole copper+TX, zinc thiazole+TX, amectotractin+TX, iprodione+TX, seboctylamine+TX; N′-[5-bromo-2-methyl-6-[(1S)-1-methyl-2-propoxy-ethoxy]-3-pyridyl]-N-ethyl-N-methyl-formamidine+TX, N′-[5-bromo-2-methyl-6-[(1R)-1-methyl-2-propoxy-ethoxy]-3-pyridyl]-N-ethyl-N-methyl-formamidine+TX, N′-[5-bromo-2-methyl-6-(1-methyl-2-propoxy-ethoxy)-3-pyridyl]-N-ethyl-N-methyl-formamidine+TX, N′-[5-chloro-2-methyl-6-(1-methyl-2-propoxy-ethoxy)-3-pyridyl]-N-ethyl-N-methyl-formamidine+TX, N′-[5-bromo-2-methyl-6-(1-methyl-2-propoxy-ethoxy)-3-pyridyl]-N-isopropyl-N-methyl-formamidine+TX (these compounds may be prepared from the methods described in WO2015/155075); N′-[5-bromo-2-methyl-6-(2-propoxypropoxy)-3-pyridyl]-N-ethyl-N-methyl-formamidine+TX (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine+TX, N′-[4-(1-cyclopropyl-2,2,2-trifluoro-1-hydroxy-ethyl)-5-methoxy-2-methyl-phenyl]-N-isopropyl-N-methyl-formamidine+TX (these compounds may be prepared from the methods described in WO2018/228896); N-ethyl-N′-[5-methoxy-2-methyl-4-[(2-trifluoromethyl)oxetan-2-yl]phenyl]-N-methyl-formamidine+TX, N-ethyl-N′-[5-methoxy-2-methyl-4-[(2-trifuoromethyl)tetrahydrofuran-2-yl]phenyl]-N-methyl-formamidine+TX (these compounds may be prepared from the methods described in WO2019/110427); N-[(1R)-1-benzyl-3-chloro-1-methyl-but-3-enyl]-8-fluoro-quinoline-3-carboxamide+TX, N-[(1S)-1-benzyl-3-chloro-1-methyl-but-3-enyl]-8-fluoro-quinoline-3-carboxamide+TX, N-[(1R)-1-benzyl-3,3,3-trifluoro-1-methyl-propyl]-8-fluoro-quinoline-3-carboxamide+TX, N-[(1S)-1-benzyl-3,3,3-trifluoro-1-methyl-propyl]-8-fluoro-quinoline-3-carboxamide+TX, N-[(1R)-1-benzyl-1,3-dimethyl-butyl]-7,8-difluoro-quinoline-3-carboxamide+TX, N-[(1S)-1-benzyl-1,3-dimethyl-butyl]-7,8-difluoro-quinoline-3-carboxamide+TX, 8-fluoro-N-[(1R)-1-[(3-fluorophenyl)methyl]-1,3-dimethyl-butyl]quinoline-3-carboxamide+TX, 8-fluoro-N-[(1S)-1-[(3-fluorophenyl)methyl]-1,3-dimethyl-butyl]quinoline-3-carboxamide+TX, N-[(1R)-1-benzyl-1,3-dimethyl-butyl]-8-fluoro-quinoline-3-carboxamide+TX, N-[(1S)-1-benzyl-1,3-dimethyl-butyl]-8-fluoro-quinoline-3-carboxamide+TX, N-((1R)-1-benzyl-3-chloro-1-methyl-but-3-enyl)-8-fluoro-quinoline-3-carboxamide+TX, N-((1S)-1-benzyl-3-chloro-1-methyl-but-3-enyl)-8-fluoro-quinoline-3-carboxamide+TX (these compounds may be prepared from the methods described in WO2017/153380); 1-(6,7-dimethylpyrazolo[1,5-a]pyridin-3-yl)-4,4,5-trifluoro-3,3-dimethyl-isoquinoline+TX, 1-(6,7-dimethylpyrazolo[1,5-a]pyridin-3-yl)-4,4,6-trifluoro-3,3-dimethyl-isoquinoline+TX, 4,4-difluoro-3,3-dimethyl-1-(6-methylpyrazolo[1,5-a]pyridin-3-yl)isoquinoline+TX, 4,4-difluoro-3,3-dimethyl-1-(7-methylpyrazolo[1,5-a]pyridin-3-yl)isoquinoline+TX, 1-(6-chloro-7-methyl-pyrazolo[1,5-a]pyridin-3-yl)-4,4-difluoro-3,3-dimethyl-isoquinoline+TX (these compounds may be prepared from the methods described in WO2017/025510); 1-(4,5-dimethylbenzimidazol-1-yl)-4,4,5-trifluoro-3,3-dimethyl-isoquinoline+TX, 1-(4,5-dimethylbenzimidazol-1-yl)-4,4-difluoro-3,3-dimethyl-isoquinoline+TX, 6-chloro-4,4-difluoro-3,3-dimethyl-1-(4-methylbenzimidazol-1-yl)isoquinoline+TX, 4,4-difluoro-1-(5-fluoro-4-methyl-benzimidazol-1-yl)-3,3-dimethyl-isoquinoline+TX, 3-(4,4-difluoro-3,3-dimethyl-1-isoquinolyl)-7,8-dihydro-6H-cyclopenta[e]benzimidazole+TX (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide+TX, N,2-dimethoxy-N-[[4-[5-(trifluoromethyl)-1,2,4-oxadiazol-3-yl]phenyl]methyl]propanamide+TX, N-ethyl-2-methyl-N-[[4-[5-(trifluoromethyl)-1,2,4-oxadiazol-3-yl]phenyl]methyl]propanamide+TX, 1-methoxy-3-methyl-1-[[4-[5-(trifluoromethyl)-1,2,4-oxadiazol-3-yl]phenyl]methyl]urea+TX, 1,3-dimethoxy-1-[[4-[5-(trifluoromethyl)-1,2,4-oxadiazol-3-yl]phenyl]methyl]urea+TX, 3-ethyl-1-methoxy-1-[[4-[5-(trifluoromethyl)-1,2,4-oxadiazol-3-yl]phenyl]methyl]urea+TX, N-[[4-[5-(trifluoromethyl)-1,2,4-oxadiazol-3-yl]phenyl]methyl]propanamide+TX, 4,4-dimethyl-2-[[4-[5-(trifluoromethyl)-1,2,4-oxadiazol-3-yl]phenyl]methyl]isoxazolidin-3-one+TX, 5,5-dimethyl-2-[[4-[5-(trifluoromethyl)-1,2,4-oxadiazol-3-yl]phenyl]methyl]isoxazolidin-3-one+TX, ethyl 1-[[4-[5-(trifluoromethyl)-1,2,4-oxadiazol-3-yl]phenyl]methyl]pyrazole-4-carboxylate+TX, N,N-dimethyl-1-[[4-[5-(trifluoromethyl)-1,2,4-oxadiazol-3-yl]phenyl]methyl]-1,2,4-triazol-3-amine+TX. The compounds in this paragraph may be prepared from the methods described in WO 2017/055473, WO 2017/055469, WO 2017/093348 and WO 2017/118689; 2-[6-(4-chlorophenoxy)-2-(trifluoromethyl)-3-pyridyl]-1-(1,2,4-triazol-1-yl)propan-2-ol+TX (this compound may be prepared from the methods described in WO 2017/029179); 2-[6-(4-bromophenoxy)-2-(trifluoromethyl)-3-pyridyl]-1-(1,2,4-triazol-1-yl)propan-2-ol+TX (this compound may be prepared from the methods described in WO 2017/029179); 3-[2-(1-chlorocyclopropyl)-3-(2-fluorophenyl)-2-hydroxy-propyl]imidazole-4-carbonitrile+TX (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile+TX (this compound may be prepared from the methods described in WO 2016/156290); (4-phenoxyphenyl)methyl 2-amino-6-methyl-pyridine-3-carboxylate+TX (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone+TX (this compound may be prepared from the methods described in WO 2011/138281); N-methyl-4-[5-(trifluoromethyl)-1,2,4-oxadiazol-3-yl]benzenecarbothioamide+TX; N-methyl-4-[5-(trifluoromethyl)-1,2,4-oxadiazol-3-yl]benzamide+TX; (Z,2E)-5-[1-(2,4-dichlorophenyl)pyrazol-3-yl]oxy-2-methoxyimino-N,3-dimethyl-pent-3-enamide+TX (this compound may be prepared from the methods described in WO 2018/153707); N′-(2-chloro-5-methyl-4-phenoxy-phenyl)-N-ethyl-N-methyl-formamidine+TX; N′-[2-chloro-4-(2-fluorophenoxy)-5-methyl-phenyl]-N-ethyl-N-methyl-formamidine+TX (this compound may be prepared from the methods described in WO 2016/202742); 2-(difluoromethyl)-N-[(3S)-3-ethyl-1,1-dimethyl-indan-4-yl]pyridine-3-carboxamide+TX (this compound may be prepared from the methods described in WO 2014/095675); (5-methyl-2-pyridyl)-[4-[5-(trifluoromethyl)-1,2,4-oxadiazol-3-yl]phenyl]methanone+TX, (3-methylisoxazol-5-yl)-[4-[5-(trifluoromethyl)-1,2,4-oxadiazol-3-yl]phenyl]methanone+TX (these compounds may be prepared from the methods described in WO 2017/220485); 2-oxo-N-propyl-2-[4-[5-(trifluoromethyl)-1,2,4-oxadiazol-3-yl]phenyl]acetamide+TX (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate+TX (this compound may be prepared from the methods described in WO 2018/158365); 2,2-difluoro-N-methyl-2-[4-[5-(trifluoromethyl)-1,2,4-oxadiazol-3-yl]phenyl]acetamide+TX, N—[(E)-methoxyiminomethyl]-4-[5-(trifluoromethyl)-1,2,4-oxadiazol-3-yl]benzamide+TX, N—[(Z)-methoxyiminomethyl]-4-[5-(trifluoromethyl)-1,2,4-oxadiazol-3-yl]benzamide+TX, N—[N-methoxy-C-methyl-carbonimidoyl]-4-[5-(trifluoromethyl)-1,2,4-oxadiazol-3-yl]benzamide+TX (these compounds may be prepared from the methods described in WO 2018/202428); microbials including: Acinetobacter lwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum (MicroAZ®, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (e.g, Biostart™, formerly Rhizoboost®)+TX, Bacillus licheniformis strain 3086 (EcoGuard®, Green Releaf®)+TX, Bacillus circulans+TX, Bacillus firmus (BioSafe®, BioNem-WP®, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®, Serenade®, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®, Rhizopro®)+TX, Bacillus thuringiensis Cry 2Ae+TX, Bacillus thuringiensis CrylAb+TX, Bacillus thuringiensis aizawai GC 91 (Agree®)+TX, Bacillus thuringiensis israelensis (BMP123®, Aquabac®, VectoBac®)+TX, Bacillus thuringiensis kurstaki (Javelin®, Deliver®, CryMax®, Bonide®, Scutella VP®, Turilav VP®, Astuto®, Dipel WP®, Biobit®, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD #32+TX, Bacillus thuringiensis strain AQ52+TX, Bacillus thuringiensis var. aizawai (XenTari®, DiPel®)+TX, bacteria spp. (GROWMEND®, GROWSWEET®X, Shootup®)+TX, bacteriophage of Clavipacter michiganensis (AgriPhage®)+TX, Bakflor®+TX, Beauveria bassiana (Beaugenic®, Brocaril WP®)+TX, Beauveria bassiana GHA (Mycotrol ES®, Mycotrol O®, BotaniGuard®)+TX, Beauveria brongniartii (Engerlingspilz®, Schweizer Beauveria®, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®, Intercept®, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmo-miniatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®, Madex® Plus, Madex® Max, Carpovirusine Evo2®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®, Biofox C®)+TX, Fusarium proliferatum+TX, Fusarium spp.+TX, Galactomyces geotrichum+TX, Gliocladium catenulatum (Primastop®, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone-formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria Dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®, Root Maximizer®)+TX, Myrothecium verrucaria strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97®, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C9-1®)+TX, Pantoea spp.+TX, Pasteuria spp. (Econem®)+TX, Pasteuria nishizawae+TX, Penicillium aurantiogriseum+TX, Penicillium billai (Jumpstart®, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomonas fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®, Polyversum®)+TX, Pythium periplocum+TX, Rhanella aquatilis+TX, Rhanella spp.+TX, Rhizobia (Dormal®, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®, PlantShield HCO, RootShield®, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Verticillium lecanii (Mycotal®, Vertalec®)+TX, Vip3Aa20 (VIPtera®)+TX, Virgibaclillus marismortui+TX, Xanthomonas campestris pv. Poae (Camperico®)+TX, Xenorhabdus bovienii+TX, and Xenorhabdus nematophilus+TX;
Plant extracts including: pine oil (Retenol®)+TX, azadirachtin (Plasma Neem Oil®, AzaGuard®, MeemAzal®, Molt-X®)+TX, Botanical IGR (Neemazad®, Neemix®)+TX, canola oil (Lilly Miller Vegol®)+TX, Chenopodium ambrosioides near ambrosioides (Requiem®)+TX, Chrysanthemum extract (Crisant®)+TX, extract of neem oil (Trilogy®)+TX, essentials oils of Labiatae (Botania®)+TX, extracts of clove rosemary peppermint and thyme oil (Garden insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®)+TX, Reynoutria ®(Regalia®, Sakalia®)+TX, rotenone (Eco Roten®)+TX, Rutaceae plant extract (Soleo®)+TX, soybean oil (Ortho Ecosense®)+TX, Melaleuca alternifolia extract (also called tea tree oil) (Timorex Gold®)+TX, thymus oil+TX, AGNIQUE® MMF+TX, BugOil®+TX, mixture of rosemary sesame pepermint thyme and cinnamon extracts (EF 300®)+TX, mixture of clove rosemary and peppermint extract (EF 400®)+TX, mixture of clove pepermint garlic oil and mint (Soil Shot®)+TX, kaolin (Screen®)+TX, storage glucam of brown algae (Laminarin®)+TX;
pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC-LR Sprayable Pheromone®)+TX, Muscamone (Snip7 Fly Bait®, Starbar Premium Fly Bait®)+TX, Oriental Fruit Moth Pheromone (3M oriental fruit moth sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CM®)+TX, (3E,8Z,11Z)-3,8,11-Tetradecatrienyl acetate+TX, (7Z,11Z,13E)-7,11,13-Hexadecatrienal+TX, (E,Z)-7,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate+TX;
Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®, Andersoni-System®)+TX, Amblyseius californicus (Amblyline®, Spical®)+TX, Amblyseius cucumeris (Thripex®, Bugline cucumeris®)+TX, Amblyseius fallacis (Fallacis®)+TX, Amblyseius swirskii (Bugline Swirskii®, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®, Tripol®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®, Digline®)+TX, Dacnusa sibirica (DacDigline®, Minex®)+TX, Diversinervus spp.+TX, Encarsia citrina+TX, Encarsia formosa (Encarsia Max®, Encarline®, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Ercal®, Eretline E®)+TX, Eretmocerus eremicus (Bemimix®)+TX, Eretmocerus hayati+TX, Eretmocerus mundus (Bemipar®, Eretline M®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®, Nemaseek®, Terranem-Nam®, Terranem®, Larvanem®, B-Green®, NemAttack®, Nematop®)+TX, Heterorhabditis megidis (Nemasys H®, BioNem H®, Exhibitline Hm®, Larvanem-M®)+TX, Hippodamia convergens+TX, Hypoaspis aculeifer (Aculeifer-System®, Entomite-A®)+TX, Hypoaspis miles (Hypoline M®, Entomite-M®)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®, Macroline C®, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®, Nesibug®)+TX, Ophyra aenescens (Biofly®)+TX, Orius insidiosus (Thripor-I®, Oriline I®)+TX, Orius laevigatus (Thripor-L®, Oriline I®)+TX, Orius majusculus (Oriline M®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®, Phytoline P®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®, Millenium®, BioNem C®, NemAttack®, Nemastar®, Capsanem®)+TX, Steinernema feltiae (NemaShield®, Nemasys F®, BioNem F, Steinernema-System®, NemAttack®, Nemaplus®, Exhibitline Sf®, Scia-Rid®, Entonem®)+TX, Steinernema kraussei (Nemasys L®, BioNem L®, Exhibitline Srb®)+TX, Steinernema riobrave (BioVector®, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine B®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator+TX; other biologicals including: abscisic acid+TX, bioSea®+TX, Chondrostereum purpureum (Chontrol Paste®)+TX, Colletotrichum gloeosporioides (Collego®)+TX, Copper Octanoate (Cueva®)+TX, Delta traps (Trapline D®)+TX, Erwinia amylovora (Harpin) (ProAct®, Ni-HIBIT Gold CST®)+TX, fatty acids derived from a natural by-product of extra virgin olive oil (FLIPPER®), Ferri-phosphate (Ferramol®)+TX, Funnel traps (Trapline Y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline F®)+TX, Microctonus hyperodae+TX, Mycoleptodiscus terrestris (Des-X®)+TX, BioGain®+TX, Aminomite®+TX, Zenox®+TX, Pheromone trap (Thripline Ams®)+TX, potassium bicarbonate (MilStop®)+TX, potassium salts of fatty acids (Sanova®)+TX, potassium silicate solution (Sil-Matrix®)+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®, Rebell Amarillo®)+TX and Traps (Takitrapline y+B®)+TX;
(1) antibacterial agents selected from the group of:
(1.1) bacteria, examples of which are Bacillus mojavensis strain R3B (Accession No. NCAIM (P) B001389) (WO 2013/034938) from Certis USA LLC+TX; Bacillus pumilus, in particular strain BU F-33, having NRRL Accession No. 50185 (CARTISSA® from BASF, EPA Reg. No. 71840-19)+TX; Bacillus subtilis, in particular strain QST713/AQ713 (SERENADE OPTI or SERENADE ASO from Bayer CropScience LP, US, having NRRL Accession No. B21661, U.S. Pat. No. 6,060,051)+TX; Bacillus subtilis strain BU1814, (VELONDIS® PLUS, VELONDIS® FLEX and VELONDIS® EXTRA from BASF SE)+TX; Bacillus subtilis var. amyloliquefaciens strain FZB24 having Accession No. DSM 10271 (available from Novozymes as TAEGRO® or TAEGRO®ECO (EPA Registration No. 70127-5))+TX; Bacillus subtilis CX-9060 from Certis USA LLC+TX; Bacillus sp., in particular strain D747 (available as DOUBLE NICKEL® from Kumiai Chemical Industry Co., Ltd.), having Accession No. FERM BP-8234, U.S. Pat. No. 7,094,592+TX; Paenibacillus sp. strain having Accession No. NRRL B-50972 or Accession No. NRRL B-67129, WO 2016/154297+TX; Paenibacillus polymyxa, in particular strain AC-1 (e.g. TOPSEED® from Green Biotech Company Ltd.)+TX; Pantoea agglomerans, in particular strain E325 (Accession No. NRRL B-21856) (available as BLOOMTIME BIOLOGICAL™ FD BIOPESTICIDE from Northwest Agri Products)+TX; Pseudomonas proradix (e.g. PRORADIX® from Sourcon Padena)+TX; and
(1.2) fungi, examples of which are Aureobasidium pullulans, in particular blastospores of strain DSM14940, blastospores of strain DSM 14941 or mixtures of blastospores of strains DSM14940 and DSM14941 (e.g., BOTECTOR® and BLOSSOM PROTECT® from bio-ferm, CH)+TX; Pseudozyma aphidis (as disclosed in WO2011/151819 by Yissum Research Development Company of the Hebrew University of Jerusalem)+TX; Saccharomyces cerevisiae, in particular strains CNCM No. 1-3936, CNCM No. 1-3937, CNCM No. 1-3938 or CNCM No. 1-3939 (as disclosed in WO 2010/086790 from Lesaffre et Compagnie, FR)+TX;
(2) biological fungicides selected from the group of:
(2.1) bacteria, examples of which are Agrobacterium radiobacter strain K84 (e.g. GALLTROL-A® from AgBioChem, CA)+TX; Agrobacterium radiobacter strain K1026 (e.g. NOGALL™ from BASF SE)+TX; Bacillus subtilis var. amyloliquefaciens strain FZB24 having Accession No. DSM 10271 (available from Novozymes as TAEGRO® or TAEGRO®ECO (EPA Registration No. 70127-5))+TX; Bacillus amyloliquefaciens, in particular strain D747 (available as Double Nickel™ from Kumiai Chemical Industry Co., Ltd., having accession number FERM BP-8234, U.S. Pat. No. 7,094,592)+TX; Bacillus amyloliquefaciens strain F727 (also known as strain MB1110) (NRRL Accession No. B-50768, WO 2014/028521) (STARGUS® from Marrone Bio Innovations)+TX; Bacillus amyloliquefaciens strain FZB42, Accession No. DSM 23117 (available as RHIZOVITAL® from ABiTEP, DE)+TX; Bacillus amyloliquefaciens isolate B246 (e.g. AVOGREEN™ from University of Pretoria)+TX; Bacillus licheniformis, in particular strain SB3086, having Accession No. ATCC 55406, WO 2003/000051 (available as ECOGUARD® Biofungicide and GREEN RELEAF™ from Novozymes)+TX; Bacillus licheniformis FMCH001 and Bacillus subtilis FMCH002 (QUARTZO®(WG) and PRESENCE®(WP) from FMC Corporation)+TX; Bacillus methylotrophicus strain BAC-9912 (from Chinese Academy of Sciences' Institute of Applied Ecology)+TX; Bacillus mojavensis strain R3B (Accession No. NCAIM (P) B001389) (WO 2013/034938) from Certis USA LLC+TX; Bacillus mycoides, isolate, having Accession No. B-30890 (available as BMJ TGAI® or WG and LifeGard™ from Certis USA LLC)+TX; Bacillus pumilus, in particular strain QST2808 (available as SONATA® from Bayer CropScience LP, US, having Accession No. NRRL B-30087 and described in U.S. Pat. No. 6,245,551)+TX; Bacillus pumilus, in particular strain GB34 (available as Yield Shield® from Bayer AG, DE)+TX; Bacillus pumilus, in particular strain BU F-33, having NRRL Accession No. 50185 (available as part of the CARTISSA product from BASF, EPA Reg. No. 71840-19)+TX; Bacillus subtilis, in particular strain QST713/AQ713 (available as SERENADE OPTI or SERENADE ASO from Bayer CropScience LP, US, having NRRL Accession No. B21661 and described in U.S. Pat. No. 6,060,051)+TX; Bacillus subtilis Y1336 (available as BIOBAC®WP from Bion-Tech, Taiwan, registered as a biological fungicide in Taiwan under Registration Nos. 4764, 5454, 5096 and 5277)+TX; Bacillus subtilis strain MBI 600 (available as SUBTILEX from BASF SE), having Accession Number NRRL B-50595, U.S. Pat. No. 5,061,495+TX; Bacillus subtilis strain GB03 (available as Kodiak® from Bayer AG, DE)+TX; Bacillus subtilis strain BU1814, (available as VELONDIS®PLUS, VELONDIS®FLEX and VELONDIS® EXTRA from BASF SE)+TX; Bacillus subtilis CX-9060 from Certis USA LLC+TX; Bacillus subtilis KTSB strain (FOLIACTIVE® from Donaghys)+TX; Bacillus subtilis IAB/BS03 (AVIV™ from STK Bio-Ag Technologies, PORTENTO® from Idai Nature)+TX; Bacillus subtilis strain Y1336 (available as BIOBAC® WP from Bion-Tech, Taiwan, registered as a biological fungicide in Taiwan under Registration Nos. 4764, 5454, 5096 and 5277)+TX; Paenibacillus epiphyticus (WO 2016/020371) from BASF SE+TX; Paenibacillus polymyxa ssp. plantarum (WO 2016/020371) from BASF SE+TX; Paenibacillus sp. strain having Accession No. NRRL B-50972 or Accession No. NRRL B-67129, WO 2016/154297+TX; Pseudomonas chlororaphis strain AFS009, having Accession No. NRRL B-50897, WO 2017/019448 (e.g., HOWLER™ and ZIO® from AgBiome Innovations, US)+TX; Pseudomonas chlororaphis, in particular strain MA342 (e.g. CEDOMON®, CERALL®, and CEDRESS® by Bioagri and Koppert)+TX; Pseudomonas fluorescens strain A506 (e.g. BLIGHTBAN®A506 by NuFarm)+TX; Pseudomonas proradix (e.g. PRORADIX® from Sourcon Padena)+TX; Streptomyces griseoviridis strain K61 (also known as Streptomyces galbus strain K61) (Accession No. DSM 7206) (MYCOSTOP® from Verdera, PREFENCE® from BioWorks, cf. Crop Protection 2006, 25, 468-475)+TX; Streptomyces lydicus strain WYEC108 (also known as Streptomyces lydicus strain WYCD108US) (ACTINO-IRON® and ACTINOVATE® from Novozymes)+TX; and
(2.2) fungi, examples of which are Ampelomyces quisqualis, in particular strain AQ 10 (e.g. AQ 10® by IntrachemBio Italia)+TX; Ampelomyces quisqualis strain AQ10, having Accession No. CNCM 1-807 (e.g., AQ 10® by IntrachemBio Italia)+TX; Aspergillus flavus strain NRRL 21882 (products known as AFLA-GUARD® from Syngenta/ChemChina)+TX; Aureobasidium pullulans, in particular blastospores of strain DSM14940+TX; Aureobasidium pullulans, in particular blastospores of strain DSM 14941+TX; Aureobasidium pullulans, in particular mixtures of blastospores of strains DSM14940 and DSM 14941 (e.g. Botector® by bio-ferm, CH)+TX; Chaetomium cupreum (Accession No. CABI 353812) (e.g. BIOKUPRUM™ by AgriLife)+TX; Chaetomium globosum (available as RIVADIOM® by Rivale)+TX; Cladosporium cladosporioides, strain H39, having Accession No. CBS122244, US 2010/0291039 (by Stichting Dienst Landbouwkundig Onderzoek)+TX; Coniothyrium minitans, in particular strain CON/M/91-8 (Accession No. DSM9660, e.g. Contans® from Bayer CropScience Biologics GmbH)+TX; Cryptococcus flavescens, strain 3C (NRRL Y-50378), (B2.2.99)+TX; Dactylaria candida+TX; Dilophosphora alopecuri (available as TWIST FUNGUS®)+TX; Fusarium oxysporum, strain Fo47 (available as FUSACLEAN® by Natural Plant Protection)+TX; Gliocladium catenulatum (Synonym: Clonostachys rosea f. catenulate) strain J1446 (e.g. Prestop® by Lallemand)+TX; Gliocladium roseum (also known as Clonostachys rosea f rosea), in particular strain 321U from Adjuvants Plus, strain ACM941 as disclosed in Xue A.G (Efficacy of Clonostachys rosea strain ACM941 and fungicide seed treatments for controlling the root tot complex of field pea, Can. J. Plant Sci. 2003, 83(3): 519-524), or strain IK726 (Jensen D F, et al. Development of a biocontrol agent for plant disease control with special emphasis on the near commercial fungal antagonist Clonostachys rosea strain ‘IK726’, Australasian Plant Pathol. 2007,36(2), 95-101)+TX; Lecanicillium lecanii (formerly known as Verticillium lecanii) conidia of strain KV01 (e.g. Vertalec® by Koppert/Arysta)+TX; Metschnikowia fructicola, in particular strain NRRL Y-30752, (B2.2.3)+TX; Microsphaeropsis ochracea+TX; Muscodor roseus, in particular strain A3-5 (Accession No. NRRL 30548)+TX; Penicillium steckii (DSM 27859, WO 2015/067800) from BASF SE+TX; Penicillium vermiculatum+TX; Phlebiopsis gigantea strain VRA 1992 (ROTSTOP®C from Danstar Ferment)+TX; Pichia anomala, strain WRL-076 (NRRL Y-30842), U.S. Pat. No. 7,579,183+TX; Pseudozyma flocculosa, strain PF-A22 UL (available as SPORODEX® L by Plant Products Co., CA)+TX; Saccharomyces cerevisiae, in particular strain LASO2 (from Agro-Levures et Derives), strain LAS117 cell walls (CEREVISANE® from Lesaffre, ROMEO® from BASF SE), strains CNCM No. 1-3936, CNCM No. 1-3937, CNCM No. 1-3938, CNCM No. 1-3939 (WO 2010/086790) from Lesaffre et Compagnie, FR+TX; Simplicillium lanosoniveum+TX; Talaromyces flavus, strain V117b+TX; Trichoderma asperelloides JM41R (Accession No. NRRL B-50759) (TRICHO PLUS® from BASF SE)+TX; Trichoderma asperellum, in particular, strain kd (e.g. T-Gro from Andermatt Biocontrol)+TX; Trichoderma asperellum, in particular strain SKT-1, having Accession No. FERM P-16510 (e.g. ECO-HOPE® from Kumiai Chemical Industry), strain T34 (e.g. T34 Biocontrol by Biocontrol Technologies S.L., ES) or strain ICC 012 from Isagro+TX; Trichoderma atroviride, in particular strain SC1 (Accession No. CBS 122089, WO 2009/116106 and U.S. Pat. No. 8,431,120 (from Bi-PA)), strain 77B (T77 from Andermatt Biocontrol) or strain LU132 (e.g. Sentinel from Agrimm Technologies Limited)+TX; Trichoderma atroviride, strain CNCM 1-1237 (e.g. Esquive® WP from Agrauxine, FR)+TX; Trichoderma atroviride, strain no. V08/002387+TX; Trichoderma atroviride, strain NMI no. V08/002388+TX; Trichoderma atroviride, strain NMI no. V08/002389+TX; Trichoderma atroviride, strain NMI no. V08/002390+TX; Trichoderma atroviride, strain LC52 (e.g. Tenet by Agrimm Technologies Limited)+TX; Trichoderma atroviride, strain ATCC 20476 (IMI 206040)+TX; Trichoderma atroviride, strain T11 (IM1352941/CECT20498)+TX; Trichoderma atroviride, strain SKT-1 (FERM P-16510), JP Patent Publication (Kokai) 11-253151 A+TX; Trichoderma atroviride, strain SKT-2 (FERM P-16511), JP Patent Publication (Kokai) 11-253151 A+TX; Trichoderma atroviride, strain SKT-3 (FERM P-17021), JP Patent Publication (Kokai) 11-253151 A+TX; Trichoderma fertile (e.g. product TrichoPlus from BASF)+TX; Trichoderma gamsii (formerly T. viride), strain ICCO80 (IMI CC 392151 CABI, e.g. BioDerma by AGROBIOSOL DE MEXICO, S.A. DE C.V.)+TX; Trichoderma gamsii (formerly T. viride), strain ICC 080 (IMI CC 392151 CABI) (available as BIODERMA® by AGROBIOSOL DE MEXICO, S.A. DE C.V.)+TX; Trichoderma harmatum+TX; Trichoderma harmatum, having Accession No. ATCC 28012+TX; Trichoderma harzianum strain T-22 (e.g. Trianum-P from Andermatt Biocontrol or Koppert) or strain Cepa SimbT5 (from Simbiose Agro)+TX; Trichoderma harzianum+TX; Trichoderma harzianum rifai T39 (e.g. Trichodex® from Makhteshim, US)+TX; Trichoderma harzianum, strain ITEM 908 (e.g. Trianum-P from Koppert)+TX; Trichoderma harzianum, strain TH35 (e.g. Root-Pro by Mycontrol)+TX; Trichoderma harzianum, strain DB 103 (available as T-GRO® 7456 by Dagutat Biolab)+TX; Trichoderma polysporum, strain IMI 206039 (e.g. Binab TF WP by BINAB Bio-Innovation AB, Sweden)+TX; Trichoderma stromaticum, having Accession No. Ts3550 (e.g. Tricovab by CEPLAC, Brazil)+TX; Trichoderma virens (also known as Gliocladium virens), in particular strain GL-21 (e.g. SoilGard by Certis, US)+TX; Trichoderma virens strain G-41, formerly known as Gliocladium virens (Accession No. ATCC 20906) (e.g., ROOTSHIELD® PLUS WP and TURFSHIELD® PLUS WP from BioWorks, US)+TX; Trichoderma viride, strain TV1(e.g. Trianum-P by Koppert)+TX; Trichoderma viride, in particular strain B35 (Pietr et al., 1993, Zesz. Nauk. A R w Szczecinie 161: 125-137)+TX; mixtures of Trichoderma asperellum strain ICC 012 (also known as Trichoderma harzianum I00012), having Accession No. CABI CC IMI 392716 and Trichoderma gamsii (formerly T. viride) strain ICC 080, having Accession No. IMI 392151 (e.g., BIO-TAM™ from Isagro USA, Inc. or BIODERMA® by Agrobiosol de Mexico, S.A. de C.V.)+TX; Ulocladium oudemansii strain U3, having Accession No. NM 99/06216 (e.g., BOTRY-ZEN® by Botry-Zen Ltd, New Zealand and BOTRYSTOP® from BioWorks, Inc.)+TX; Verticillium alboatrum (formerly V. dahliae), strain WCS850 having Accession No. WCS850, deposited at the Central Bureau for Fungi Cultures (e.g., DUTCH TRIG® by Tree Care Innovations)+TX; Verticillium chlamydosporium+TX;
(3) biological control agents having an effect for improving plant growth and/or plant health selected from the group of:
(3.1) bacteria, examples of which are Azospirillum brasilense (e.g., VIGOR® from KALO, Inc.)+TX; Azospirillum lipoferum (e.g., VERTEX-IF™ from TerraMax, Inc.)+TX; Azorhizobium caulinodans, in particular strain ZB—SK-5+TX; Azotobacter chroococcum, in particular strain H23+TX; Azotobacter vinelandii, in particular strain ATCC 12837+TX; a mixture of Azotobacter vinelandii and Clostridium pasteurianum (available as INVIGORATE® from Agrinos)+TX; Bacillus amyloliquefaciens μm414 (LOLI-PEPTA® from Biofilm Crop Protection)+TX; Bacillus amyloliquefaciens SB3281 (ATCC #PTA-7542, WO 2017/205258)+TX; Bacillus amyloliquefaciens TJ1000 (available as QUIKROOTS® from Novozymes)+TX; Bacillus amyloliquefaciens, in particular strain IN937a+TX; Bacillus amyloliquefaciens, in particular strain FZB42 (e.g. RHIZOVITAL® from ABiTEP, DE)+TX; Bacillus amyloliquefaciens BS27 (Accession No. NRRL B-5015)+TX; Bacillus cereus family member EE128 (NRRL No. B-50917)+TX; Bacillus cereus family member EE349 (NRRL No. B-50928)+TX; Bacillus cereus, in particular strain BP01 (ATCC 55675, e.g. MEPICHLOR® from Arysta Lifescience, US)+TX; Bacillus firmus, in particular strain CNMC 1-1582 (e.g. VOTIVO® from BASF SE)+TX; Bacillus mycoides BT155 (NRRL No. B-50921)+TX; Bacillus mycoides EE118 (NRRL No. B-50918)+TX; Bacillus mycoides EE141 (NRRL No. B-50916)+TX; Bacillus mycoides BT46-3 (NRRL No. B-50922)+TX; Bacillus pumilus, in particular strain QST2808 (Accession No. NRRL No. B-30087)+TX; Bacillus pumilus, in particular strain GB34 (e.g. YIELD SHIELD® from Bayer Crop Science, DE)+TX; Bacillus siamensis, in particular strain KCTC 13613T+TX; Bacillus subtilis, in particular strain QST713/AQ713 (having NRRL Accession No. B-21661 and described in U.S. Pat. No. 6,060,051, available as SERENADE® OPTI or SERENADE® ASO from Bayer CropScience LP, US)+TX; Bacillus subtilis, in particular strain AQ30002 (Accession No. NRRL No. B-50421 and described in U.S. patent application Ser. No. 13/330,576)+TX; Bacillus subtilis, in particular strain AQ30004 (NRRL No. B-50455 and described in U.S. patent application Ser. No. 13/330,576)+TX; Bacillus subtilis strain BU1814, (available as TEQUALIS® from BASF SE), Bacillus subtilis rm303 (RHIZOMAX® from Biofilm Crop Protection)+TX; Bacillus thuringiensis BT013A (NRRL No. B-50924) also known as Bacillus thuringiensis 4Q7+TX; a mixture of Bacillus licheniformis FMCH001 and Bacillus subtilis FMCH002 (available as QUARTZO®(WG), PRESENCE®(WP) from FMC Corporation)+TX; Bacillus subtilis, in particular strain MBI 600 (e.g. SUBTILEX® from BASF SE)+TX; Bacillus tequilensis, in particular strain NII-0943+TX; Bradyrhizobium japonicum (e.g. OPTIMIZE® from Novozymes)+TX; Delftia acidovorans, in particular strain RAY209 (e.g. BIOBOOST® from Brett Young Seeds)+TX; Mesorhizobium cicer (e.g., NODULATOR from BASF SE)+TX; Lactobacillus sp. (e.g. LACTOPLANT® from LactoPAFI)+TX; Rhizobium leguminosarium biovar viciae (e.g., NODULATOR from BASF SE)+TX; Pseudomonas proradix (e.g. PRORADIX® from Sourcon Padena)+TX; Pseudomonas aeruginosa, in particular strain PN1+TX; Rhizobium leguminosarum, in particular bv. viceae strain Z25 (Accession No. CECT 4585)+TX; Paenibacillus polymyxa, in particular strain AC-1 (e.g. TOPSEED® from Green Biotech Company Ltd.)+TX; Serratia marcescens, in particular strain SRM (Accession No. MTCC 8708)+TX; Sinorhizobium meliloti strain NRG-185-1 (NITRAGIN® GOLD from Bayer CropScience)+TX; Thiobacillus sp. (e.g. CROPAID® from Cropaid Ltd UK)+TX; and (3.2) fungi, examples of which are Purpureocillium lilacinum (previously known as Paecilomyces lilacinus) strain 251 (AGAL 89/030550, e.g. BioAct from Bayer CropScience Biologics GmbH)+TX; Penicillium bilaii, strain ATCC 22348 (e.g. JumpStart® from Acceleron BioAg), Talaromyces flavus, strain V117b+TX; Trichoderma atroviride strain CNCM 1-1237 (e.g. Esquive® WP from Agrauxine, FR), Trichoderma viride, e.g. strain B35 (Pietr et al., 1993, Zesz. Nauk. A R w Szczecinie 161: 125-137)+TX; Trichoderma atroviride strain LC52 (also known as Trichoderma atroviride strain LU132, e.g. Sentinel® from Agrimm Technologies Limited)+TX; Trichoderma atroviride strain SC1 described in WO2009/116106)+TX; Trichoderma asperellum strain kd (e.g. T-Gro from Andermatt Biocontrol)+TX; Trichoderma asperellum strain (Eco-T from Plant Health Products, ZA)+TX, Trichoderma harzianum strain T-22 (e.g. Trianum-P from Andermatt Biocontrol or Koppert)+TX; Myrothecium verrucaria strain AARC-0255 (e.g. DiTera™ from Valent Biosciences)+TX; Penicillium bilaii strain ATCC ATCC20851+TX; Pythium oligandrum strain M1 (ATCC 38472, e.g. Polyversum from Bioprepraty, CZ)+TX; Trichoderma virens strain GL-21 (e.g. SoilGard® from Certis, USA)+TX; Verticillium alboatrum (formerly V. dahliae) strain WCS850 (CBS 276.92, e.g. Dutch Trig from Tree Care Innovations)+TX; Trichoderma atroviride, in particular strain no. V08/002387, strain no. NMI No. V08/002388, strain no. NMI No. V08/002389, strain no. NMI No. V08/002390+TX; Trichoderma harzianum strain ITEM 908, Trichoderma harzianum, strain TSTh20+TX; Trichoderma harzianum strain 1295-22+TX; Pythium oligandrum strain DV74+TX; Rhizopogon amylopogon (e.g. Myco-Sol from Agri-Enterprise, LLC, formerly Helena Chemical Company)+TX; Rhizopogon fulvigleba (e.g. Myco-Sol from Agri-Enterprise, LLC, formerly Helena Chemical Company)+TX; Trichoderma virens strain GI-3+TX;
(4) insecticidally active biological control agents selected from
(4.1) bacteria, examples of which are Agrobacterium radiobacter strain K84 (Galltrol from AgBiochem Inc.)+TX; Bacillus amyloliquefaciens, in particular strain PTS-4838 (e.g. AVEO from Valent Biosciences, US)+TX; Bacillus firmus, in particular strain CNMC 1-1582 (e.g. VOTIVO® from BASF SE)+TX; Bacillus mycoides, isolate J. (e.g. BmJ from Certis USA LLC.)+TX; Bacillus sphaericus, in particular Serotype H5a5b strain 2362 (strain ABTS-1743) (e.g. VECTOLEX® from Valent BioSciences, US)+TX; Bacillus thuringiensis subsp. aizawai, in particular strain ABTS-1857 (SD-1372, e.g. XENTARI® from Valent BioSciences)+TX; Bacillus thuringiensis subsp. aizawai, in particular serotype H-7 (e.g. FLORBAC® WG from Valent BioSciences, US)+TX; Bacillus thuringiensis israelensis strain BMP 144 (e.g. AQUABAC® by Becker Microbial Products IL)+TX; Bacillus thuringiensis subsp. israelensis (serotype H-14) strain AM65-52 (Accession No. ATCC 1276) (e.g. VECTOBAC® by Valent BioSciences, US)+TX; Bacillus thuringiensis subsp. aizawai strain GC-91+TX; Bacillus thuringiensis var. Colmeri (e.g. TIANBAOBTC by Changzhou Jianghai Chemical Factory)+TX; Bacillus thuringiensis var. japonensis strain Buibui+TX; Bacillus thuringiensis subsp. kurstaki strain BMP 123 (from Becker Microbial Products, IL, BARITONE from Bayer CropScience)+TX; Bacillus thuringiensis subsp. kurstaki strain HD-1 (e.g. DIPEL® ES from Valent BioSciences, US)+TX; Bacillus thuringiensis var. kurstaki strain EVB-113-19 (e.g., BIOPROTEC® from AEF Global)+TX; Bacillus thuringiensis subsp. kurstaki strain ABTS 351+TX; Bacillus thuringiensis subsp. kurstaki strain PB 54+TX; Bacillus thuringiensis subsp. kurstaki strain SA 11 (JAVELIN from Certis, US)+TX; Bacillus thuringiensis subsp. kurstaki strain SA 12 (THURICIDE from Certis, US)+TX; Bacillus thuringiensis subsp. kurstaki strain EG 2348 (LEPINOX® from Certis, US)+TX; Bacillus thuringiensis subsp. kurstaki strain EG 7841 (CRYMAX® from Certis, US)+TX; Bacillus thuringiensis subsp. tenebrionis strain NB 176 (SD-5428, e.g. NOVODOR® FC from BioFa DE)+TX; Brevibacillus laterosporus (LATERAL® from Ecolibrium Biologicals)+TX; Burkholderia spp., in particular Burkholderia rinojensis strain A396 (also known as Burkholderia rinojensis strain MBI 305) (Accession No. NRRL B-50319); WO 2011/106491 and WO 2013/032693; e.g. MB1206 TGAI and ZELTO® from Marrone Bio Innovations)+TX; Chromobacterium subtsugae, in particular strain PRAA4-1T (e.g. MBI-203; e.g. GRANDEVO® from Marrone Bio Innovations)+TX; Lecanicillium muscarium Ve6 (MYCOTAL from Koppert)+TX; Paenibacillus popilliae (formerly Bacillus popilliae+TX; e.g. MILKY SPORE POWDER™ or MILKY SPORE GRANULAR™ from St. Gabriel Laboratories)+TX; Pasteuria nishizawae strain Pn1 (CLARIVA from Syngenta/ChemChina)+TX; Serratia entomophila (e.g. INVADE® by Wrightson Seeds)+TX; Serratia marcescens, in particular strain SRM (Accession No. MTCC 8708)+TX; Trichoderma asperellum (TRICHODERMAX from Novozymes)+TX; Wolbachia pipientis ZAP strain (e.g., ZAP MALES® from MosquitoMate)+TX; and (4.2) fungi, examples of which are Beauveria bassiana strain ATCC 74040 (e.g. NATURALIS® from Intrachem Bio Italia)+TX; Beauveria bassiana strain GHA (Accession No. ATCC74250, e.g. BOTANIGUARD® ES and MYCONTROL-O® from Laverlam International Corporation)+TX; Beauveria bassiana strain ATPO2 (Accession No. DSM 24665)+TX; Isaria fumosorosea (previously known as Paecilomyces fumosoroseus) strain Apopka 97 (PREFERAL® from SePRO)+TX; Metarhizium anisopliae 3213-1 (deposited under NRRL accession number 67074 disclosed in WO 2017/066094; Pioneer Hi-Bred International)+TX; Metarhizium robertsii 15013-1 (deposited under NRRL accession number 67073)+TX; Metarhizium robertsii 23013-3 (deposited under NRRL accession number 67075)+TX; Paecilomyces lilacinus strain 251 (MELOCON® from Certis, US)+TX; Zoophtora radicans+TX;
(5) Viruses selected from the group consisting of Adoxophyes orana (summer fruit tortrix) granulosis virus (GV)+TX; Cydia pomonella (codling moth) granulosis virus (GV)+TX; Helicoverpa armigera (cotton bollworm) nuclear polyhedrosis virus (NPV)+TX; Spodoptera exigua (beet armyworm) mNPV+TX; Spodoptera frugiperda (fall armyworm) mNPV+TX; Spodoptera littoralis (African cotton leafworm) NPV+TX;
(6) Bacteria and fungi which can be added as ‘inoculant’ to plants or plant parts or plant organs and which, by virtue of their particular properties, promote plant growth and plant health selected from Agrobacterium spp.+TX; Azorhizobium caulinodans+TX; Azospirillum spp.+TX; Azotobacter spp.+TX; Bradyrhizobium spp.+TX; Burkholderia spp., in particular Burkholderia cepacia (formerly known as Pseudomonas cepacia)+TX; Gigaspora spp., or Gigaspora monosporum+TX; Glomus spp.+TX; Laccaria spp.+TX; LactoBacillus buchneri+TX; Paraglomus spp.+TX; Pisolithus tinctorus+TX; Pseudomonas spp.+TX; Rhizobium spp., in particular Rhizobium trifolii+TX; Rhizopogon spp.+TX; Scleroderma spp.+TX; Suillus spp.+TX; Streptomyces spp.+TX;
(7) Plant extracts and products formed by microorganisms including proteins and secondary metabolites which can be used as biological control agents, selected from Allium sativum (NEMGUARD from Eco-Spray; BRALIC from ADAMA)+TX; Armour-Zen+TX; Artemisia absinthium+TX; Azadirachtin (e.g. AZATIN XL from Certis, US)+TX; Biokeeper WP+TX; Brassicaceae extract, in particular oilseed rape powder or mustard powder+TX; Cassia nigricans+TX; Celastrus angulatus+TX; Chenopodium anthelminticum+TX; Chitin+TX; Dryopteris filix-mas+TX; Equisetum arvense+TX; Fortune Aza+TX; Fungastop+TX; Chenopodium quinoa saponin extract from quinoa seeds (e.g. Heads Up® (Saponins of Quinoa) from Heads Up plant Protectants, CA)+TX; naturally occurring Blad polypeptide extracted from Lupin seeds (PROBLAD® from Certis EU)+TX; naturally occurring Blad polypeptide extracted from Lupin seeds (FRACTURE® from FMC)+TX; Pyrethrum/Pyrethrins+TX; Quassia amara+TX; Quercus+TX; Quillaja extract (QL AGRI 35 from BASF)+TX; Reynoutria sachalinensis extract (REGALLIA®, REGALIA®MAXX from Marrone Bio)+TX; “Requiem™ Insecticide”+TX; Rotenone+TX; ryania/ryanodine+TX; Symphytum officinale+TX; Tanacetum vulgare+TX; Thymol+TX; Thymol mixed with Geraniol (CEDROZ from Eden Research)+TX;
Thymol mixed with Geraniol and Eugenol (MEVALONE® from Eden Research)+TX; Triact 70+TX; TriCon+TX; Tropaeulum majus+TX; Melaleuca alternifolia extract (TIMOREX GOLD from STK)+TX; Urtica dioica+TX; Veratrin+TX; and Viscum album+TX; and a safener, such as benoxacor+TX, cloquintocet (including cloquintocet-mexyl)+TX, cyprosulfamide+TX, dichlormid+TX, fenchlorazole (including fenchlorazole-ethyl)+TX, fenclorim+TX, fluxofenim+TX, furilazole+TX, isoxadifen (including isoxadifen-ethyl)+TX, mefenpyr (including mefenpyr-diethyl)+TX, metcamifen+TX and oxabetrinil+TX.
   The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticides/acetoprole.html.
   Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.
   The active ingredient mixture of the compounds of formula (I) selected from the compounds defined in the Tables A-1 to A-71, and Table P, with active ingredients described above comprises a compound selected from one compound defined in the Tables A-1 to A-71, and Table P, and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 to 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.
   The compounds and mixtures as described above can be used in a method for controlling pests, which comprises applying a composition comprising a compound or mixture respectively as described above to the pests or their environment, with the exception of a method for treatment of the human or animal body by surgery or therapy and diagnostic methods practiced on the human or animal body.
   The mixtures comprising a compound of formula (I) selected from the compounds defined in the Tables A-1 to A-71, and Table P, and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula (I) and the active ingredients as described above is not essential for working the present invention.
   The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.
   The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.
   The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.
   A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.
   The compounds of formula (I) of the invention and compositions thereof are also suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.
   The term seed embraces seeds and plant propagules of all kinds including but not limited to true seeds, seed pieces, suckers, corns, bulbs, fruit, tubers, grains, rhizomes, cuttings, cut shoots and the like and means, in a preferred embodiment, true seeds.
   The present invention also comprises seeds coated or treated with or containing a compound of formula (I). The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula (I). Further, it is hereby made available, a composition comprising a plant propagation material treated with a compound of formula (I).
   Seed treatment comprises all suitable seed treatment techniques known in the art, such as seed dressing, seed coating, seed dusting, seed soaking and seed pelleting. The seed treatment application of the compound formula (I) can be carried out by any known methods, such as spraying or by dusting the seeds before sowing or during the sowing/planting of the seeds.
   The compounds of the invention can be distinguished from other similar compounds by virtue of greater efficacy at low application rates and/or different pest control, which can be verified by the person skilled in the art using the experimental procedures, using lower concentrations if necessary, for example 10 ppm, 5 ppm, 2 ppm, 1 ppm or 0.2 ppm; or lower application rates, such as 300, 200 or 100, mg of Al per m². The greater efficacy can be observed by an increased safety profile (against non-target organisms above and below ground (such as fish, birds and bees), improved physico-chemical properties, or increased biodegradability).
   In each aspect and embodiment of the invention, “consisting essentially” and inflections thereof are a preferred embodiment of “comprising” and its inflections, and “consisting of and inflections thereof are a preferred embodiment of “consisting essentially of” and its inflections.
   The disclosure in the present application makes available each and every combination of embodiments disclosed herein.
   It should be noted that the disclosure herein in respect of a compound of formula (I) applies equally in respect of a compound of each of formulae I*, I′a, Iaa, Iab, Iac, Iad, Iae, I′aa, I′ab, I′ac, I′ad, ′Iae, and Tables A-1 to A-71.

### Biological Examples

The Examples which follow serve to illustrate the invention. Certain compounds of the invention can be distinguished from known compounds by virtue of greater efficacy at low application rates, which can be verified by the person skilled in the art using the experimental procedures outlined in the Examples, using lower application rates if necessary, for example 50 ppm, 24 ppm, 12.5 ppm, δ ppm, 3 ppm, 1.5 ppm, 0.8 ppm or 0.2 ppm.

### Example B1: Activity Against Chilo suppressalis (Striped Rice Stemborer)

24-well microtiter plates with artificial diet were treated with aqueous test solutions prepared from 10′000 ppm DMSO stock solutions by pipetting. After drying, the plates were infested with L2 larvae (6-8 per well). The samples were assessed for mortality, anti-feeding effect, and growth inhibition in comparison to untreated samples 6 days after infestation. Control of Chilo suppressalis by a test sample is given when at least one of the categories mortality, anti-feedant effect, and growth inhibition is higher than the untreated sample.
The following compounds resulted in at least 80% control at an application rate of 200 ppm:
P1, P2, P3, P4, P5, P6, P7, P8, P9, P10, P11, P12, P13, P14, P15, P16, P17, P18, P19, P20, P21, P22, P23, P24, P25, P26, P28, P29, P30, P31, P32, P33, P34, P35, P36, P37, P38, P39, P40, P41, P42, P43, P46, P47, P48, P49, P50, P51, P52, P53, P54, P55, P57, P58, P59, P60, P61, P62, P63, P64, P65, P66, P67, P69, P70, P71, P72, P73, P75, P77, P81, P83.

### Example B2: Activity Against Diabrotica Balteata (Corn Root Worm)

Maize sprouts placed onto an agar layer in 24-well microtiter plates were treated with aqueous test solutions prepared from 10′000 ppm DMSO stock solutions by spraying. After drying, the plates were infested with L2 larvae (6 to 10 per well). The samples were assessed for mortality and growth inhibition in comparison to untreated samples 4 days after infestation.
The following compounds gave an effect of at least 80% in at least one of the two categories (mortality or growth inhibition) at an application rate of 200 ppm:
P1, P2, P3, P4, P5, P7, P8, P9, P10, P11, P12, P13, P14, P15, P16, P17, P18, P19, P20, P21, P22, P23, P24, P25, P26, P27, P28, P29, P30, P31, P33, P34, P35, P36, P37, P38, P39, P40, P41, P42, P43, P45, P46, P47, P48, P49, P50, P51, P52, P53, P55, P57, P58, P59, P60, P61, P62, P63, P64, P65, P66, P67, P69, P71, P75, P81, P82, P83.

### Example B3: Activity Against Euschistus heros (Neotropical Brown Stink Bug)

Soybean leaves on agar in 24-well microtiter plates were sprayed with aqueous test solutions prepared from 10′000 ppm DMSO stock solutions. After drying the leaves were infested with N2 nymphs. The samples were assessed for mortality and growth inhibition in comparison to untreated samples 5 days after infestation.
The following compounds gave an effect of at least 80% in at least one of the two categories (mortality or growth inhibition) at an application rate of 200 ppm:

### P1, P3, P5, P7, P9, P15, P17, P35, P38, P47, P48, P50, P51, P52, P55, P59, P60, P61, P63, P64, P67, P68, P74, P75.

### Example B4: Activity Against Myzus persicae (Green Peach Aphid). Intrinsic Activity

Test compounds prepared from 10′000 ppm DMSO stock solutions were applied by pipette into 24-well microtiter plates and mixed with sucrose solution. The plates were closed with a stretched Parafilm. A plastic stencil with 24 holes was placed onto the plate and infested pea seedlings were placed directly on the Parafilm. The infested plate was closed with a gel blotting paper and another plastic stencil and then turned upside down. The samples were assessed for mortality 5 days after infestation.
The following compounds resulted in at least 80% mortality at a test rate of 12 ppm:
P1, P2, P3, P4, P5, P8, P9, P10, P11, P12, P13, P14, P15, P16, P17, P18, P19, P21, P23, P24, P25, P26, P29, P30, P31, P33, P34, P35, P36, P37, P38, P39, P40, P43, P46, P47, P48, P49, P50, P51, P52, P53, P55, P59, P60, P61, P62, P63, P64, P65, P66, P67, P69, P71, P72, P73, P75, P81, P82, P83.

### Example B5: Activity Against Plutella xylostella (Diamond Back Moth)

24-well microtiter plates with artificial diet were treated with aqueous test solutions prepared from 10′000 ppm DMSO stock solutions by pipetting. After drying, Plutella eggs were pipetted through a plastic stencil onto a gel blotting paper and the plate was closed with it. The samples were assessed for mortality and growth inhibition in comparison to untreated samples 8 days after infestation.
The following compounds gave an effect of at least 80% in at least one of the two categories (mortality or growth inhibition) at an application rate of 200 ppm:
P1, P2, P3, P4, P5, P6, P7, P8, P9, P10, P11, P12, P13, P14, P15, P16, P17, P18, P19, P20, P21, P22, P23, P24, P25, P26, P27, P28, P29, P30, P31, P32, P33, P34, P35, P36, P37, P38, P39, P40, P41, P42, P43, P44, P45, P46, P47, P48, P49, P50, P51, P52, P53, P54, P55, P57, P58, P59, P60, P61, P62, P63, P64, P65, P66, P67, P69, P70, P71, P72, P73, P74, P75, P77, P78, P81, P82, P83.

### Example B6: Activity Against Spodoptera littoralis (Egyptian Cotton Leaf Worm)

Cotton leaf discs were placed onto agar in 24-well microtiter plates and sprayed with aqueous test solutions prepared from 10′000 ppm DMSO stock solutions. After drying the leaf discs were infested with five L1 larvae. The samples were assessed for mortality, anti-feeding effect, and growth inhibition in comparison to untreated samples 3 days after infestation. Control of Spodoptera littoralis by a test sample is given when at least one of the categories mortality, anti-feedant effect, and growth inhibition is higher than the untreated sample.
The following compounds resulted in at least 80% control at an application rate of 200 ppm:
P1, P2, P3, P4, P5, P6, P7, P8, P9, P10, P11, P12, P13, P14, P15, P16, P17, P18, P19, P21, P22, P23, P24, P25, P26, P27, P28, P29, P30, P31, P32, P33, P34, P35, P36, P37, P38, P39, P40, P41, P42, P43, P45, P46, P47, P48, P49, P50, P51, P52, P53, P54, P55, P57, P58, P59, P60, P61, P62, P63, P64, P65, P66, P67, P69, P70, P71, P72, P73, P74, P75, P81, P82, P83.

### Example B7a: Activity Against Myzus persicae (Green Peach Aphid). Feeding/Contact Activity

Sunflower leaf discs were placed onto agar in a 24-well microtiter plate and sprayed with aqueous test solutions prepared from 10′000 ppm DMSO stock solutions. After drying, the leaf discs were infested with an aphid population of mixed ages. The samples were assessed for mortality 6 days after infestation.
The following compounds resulted in at least 80% mortality at an application rate of 200 ppm:

### P2, P9, P13, P14, P29.

### Example B7b: Activity Against Myzus persicae (Green Peach Aphid). Feeding/Contact Activity

Eggplant leaf discs were placed onto agar in a 24-well microtiter plate and sprayed with aqueous test solutions prepared from 10′000 ppm DMSO stock solutions. After drying, the leaf discs were infested with an aphid population of mixed ages. The samples were assessed for mortality 6 days after infestation.
The following compounds resulted in at least 80% mortality at an application rate of 200 ppm:

### P21, P30, P31, P38, P41, P50, P52, P55, P59, P61, P63, P67, P82.

### Example B8: Activity Against Tetranychus urticae (Two-Spotted Spider Mite)

Bean leaf discs on agar in 24-well microtiter plates were sprayed with aqueous test solutions prepared from 10′000 ppm DMSO stock solutions. After drying the leaf discs were infested with a mite population of mixed ages. The samples were assessed for mortality on mixed population (mobile stages) 8 days after infestation.
The following compounds resulted in at least 80% mortality at an application rate of 200 ppm:

### P13, P17, P50, P71.

### Example B9: Activity Against Pseudoplusia includens (Soybean Looper). Larvicide, Feeding/Contact

Soybean plants were treated in a spray chamber, cut off and placed into petri dishes containing wet filter paper. 1d after application leaves were infested with 5 L2 larvae and covered with a fabric filter and plastic lids. 5 days after infestation the samples were assessed for mortality and growth inhibition. The following compounds gave an effect of at least 80% in at least one of the two categories (mortality or growth inhibition) at an application rate of 50 ppm:

### P1, P2, P3, P5, P8, P9, P10, P13, P14, P15, P16, P17, P21, P30, P31, P33, P38, P47, P50, P52, P53, P55, P60, P61, P62.

### Example B10: Activity Against Carpocapsa (Cydia) Pomonella (Codling Moth). Larvicide, Feeding/Contact

Diet cubes coated with paraffin were sprayed with diluted test solutions in an application chamber. After drying off the treated cubes (10 replicates) were infested with 1 L1 larvae. Samples were incubated at 26-27° C. and checked 14 days after infestation for mortality and growth inhibition.
The following compounds gave an effect of at least 80% in at least one of the two categories (mortality or growth inhibition) at an application rate of 50 ppm:

### P1, P2, P3, P9, P10, P13, P17, P53.

### Example B11: Comparison of the Insecticidal Activity of Compound P37 According to the Invention with a Structurally Most Closely Comparable Compound from the State of the Art

Activity of compound P37 according to the preparatory examples and of a compound from WO 22/268648 against Spodoptera littoralis (Example B6 above) is summarized in Table B11.

TABLE B11

| | Concentration | | Mortality |
|---|---|---|---|
| Compound | (ppm) | Insect | (%) |
| Compound P37 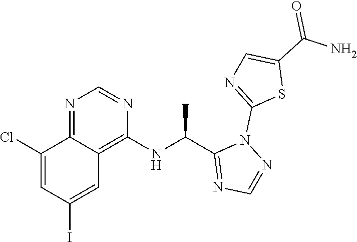 [SMILES: C[C@H](Nc1ncnc2c(Cl)cc(I)cc12)c1ncnn1-c1ncc(C(N)=O)s1] Present invention | 50 | Spodoptera littoralis | 100 |
| Described in WO 22/268648 as | 50 | Spodoptera | 0 |
| compound P20 (page 160) | | littorallis | |
| 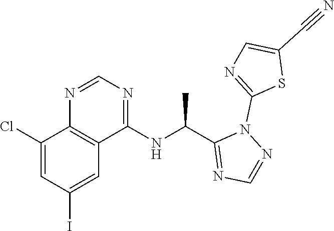 [SMILES: C[C@H](Nc1ncnc2c(Cl)cc(I)cc12)c1ncnn1-c1ncc(C#N)s1] | | | |
| State of the art | | | |

Table B11 shows that compound P37 according to the invention exert a substantially better insecticidal action on Spodoptera littoralis than the compound from the state of the art. This enhanced effect was not to be expected on the basis of the structural similarity of these compounds.

### Example B12: Comparison of the Insecticidal Activity of Compound P57 According to the Invention with a Structurally Most Closely Comparable Compound from the State of the Art

Activity of compound P57 according to the preparatory examples and of a compound from WO 21/083936 against Spodoptera littoralis (Example B6 above) and Chilo suppressalis (Example B1 above) is summarized in Table B12.

TABLE B12

| | Concentration | | Mortality |
|---|---|---|---|
| Compound | (ppm) | Insect | (%) |
| Compound P57 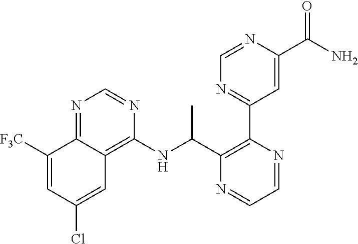 [SMILES: CC(Nc1ncnc2c(C(F)(F)F)cc(Cl)cc12)c1nccnc1-c1cc(C(N)=O)ncn1] Present invention | 3.12 50 | Spodoptera littoralis Chilo suppressalis | 80 100 |
| Described in WO 21/083936 as compound P.130 (page 153) 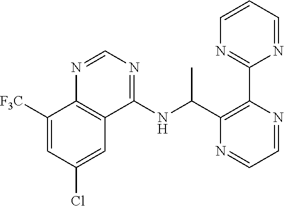 [SMILES: CC(Nc1ncnc2c(C(F)(F)F)cc(Cl)cc12)c1nccnc1-c1ncccn1] State of the art | 3.12 50 | Spodoptera littoralis Chilo suppressalis | 0 0 |

Table B12 shows that compounds P57 according to the invention exert a substantially better insecticidal action on Spodoptera littoralis and Chilo suppressalis than the compound from the state of the art. This enhanced effect was not to be expected on the basis of the structural similarity of these compounds.

### Example B13: Comparison of the Insecticidal Activity of Compound P19 According to the Invention with a Structurally Most Closely Comparable Compound from the State of the Art

Activity of compounds P19 according to the preparatory examples and of a compound from WO 22/268648 against Chilo suppressalis (Example B1 above) is summarized in Table B13.

TABLE B13

| | Concentration | | Mortality |
|---|---|---|---|
| Compound | (ppm) | Insect | (%) |
| Compound P19 | 12.5 | Chilo suppressalis | 80 |
| 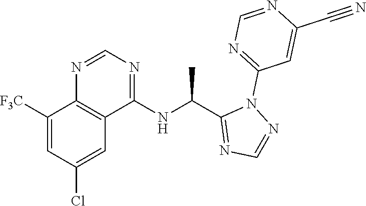 [SMILES: C[C@H](Nc1ncnc2c(C(F)(F)F)cc(Cl)cc12)c1ncnn1-c1cc(C#N)ncn1] | | | |
| Present Invention | | | |
| Described in WO 22/268648 as | 12.5 | Chilo suppressalis | 0 |
| compound P10 (page 158) | | | |
| 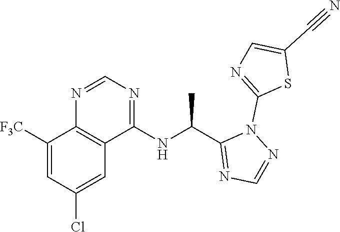 [SMILES: C[C@H](Nc1ncnc2c(C(F)(F)F)cc(Cl)cc12)c1ncnn1-c1ncc(C#N)s1] | | | |
| State of the art | | | |

Table B13 shows that compounds P19 according to the invention exert a substantially better insecticidal action on Chilo suppressalis than the compound from the state of the art. This enhanced effect was not to be expected on the basis of the structural similarity of these compounds.

### Example B14: Comparison of the Insecticidal Activity of Compounds P5, P17 and P21 According to the Invention with a Structurally Most Closely Comparable Compound from the State of the Art

Activity of compounds P5, P17 and P21 according to the preparatory examples and of a compound from WO 21/083936 against Spodoptera littoralis (Example B6 above), Chilo suppressalis (Example B1 above), Diabrotica balteata (Example B14a below), Plutella xylostella (Example B14b below) and Pseudoplusia includens (Example B9 above) is summarized in Table B14.
B14a: Diabrotica balteata (Corn root worm). Larvicide, feeding/contact
3 Corn seedlings were placed on wetted filter paper in plastic cups, and 3 ml of diluted test solutions were pipetted onto them. The cups were infested with 10 L2 larvae and checked for mortality and growth regulation 5 days after treatment.
B14b: Plutella xylostella (Diamond back moth). Larvicide L3, feeding/contact
Chinese cabbage plants were sprayed with diluted test solutions in an application chamber. Cut off leaves were placed into petri dishes with wetted filter paper and infested 1 day after application with 10 L3 larvae. Samples were assessed 4 days after infestation for mortality and growth regulation.

TABLE B14

| | Concentration | | Mortality |
|---|---|---|---|
| Compound | (ppm) | Insect | (%) |
| Compound P5 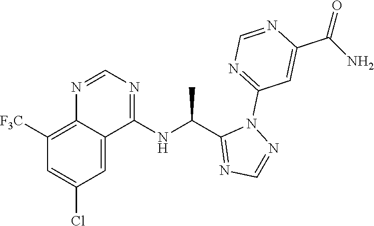 [SMILES: C[C@H](Nc1ncnc2c(C(F)(F)F)cc(Cl)cc12)c1ncnn1-c1cc(C(N)=O)ncn1] Present invention | 3.12 3.12 0.8 3 12.5 | Spodoptera littoralis Chilo suppressalis Diabrotica balteata Plutella xylostella Pseudoplusia includens | 100 80 100 100 100 |
| Coompound P21 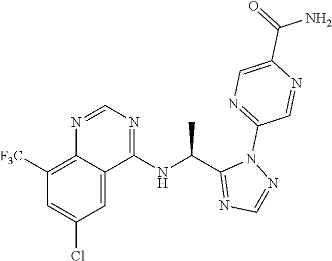 [SMILES: C[C@H](Nc1ncnc2c(C(F)(F)F)cc(Cl)cc12)c1ncnn1-c1cnc(C(N)=O)cn1] Present invention | 3.12 3.12 0.8 3 12.5 | Spodoptera littoralis Chilo suppressalis Diabrotica balteata Plutella xylostella Pseudoplusia includens | 100 0 65 100 40 |
| Compound P17 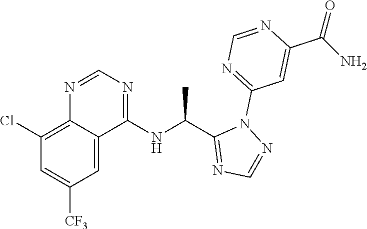 [SMILES: C[C@H](Nc1ncnc2c(Cl)cc(C(F)(F)F)cc12)c1ncnn1-c1cc(C(N)=O)ncn1] Present invention | 3.12 3.12 0.8 3 12.5 | Spodoptera littoralis Chilo suppressalis Diabrotica balteata Plutella xylostella Pseudoplusia includens | 100 40 100 100 100 |
| Described in WO 21/083936 as compound P.157 (page 159) 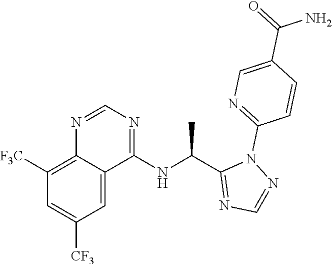 [SMILES: C[C@H](Nc1ncnc2c(C(F)(F)F)cc(C(F)(F)F)cc12)c1ncnn1-c1ccc(C(N)=O)cn1] State of the art | 3.12 3.12 0.8 3 12.5 | Spodoptera littoralis Chilo suppressalis Diabrotica balteata Plutella xylostella Pseudoplusia includens | 0 0 35 65 10 |

Table B14 shows that compounds P5, P17 and P21 according to the invention exert a substantially better insecticidal action on Spodoptera littoralis, Chilo suppressalis, Diabrotica balteata, Plutella xylostella and Pseudoplusia includens than the compound from the state of the art. This enhanced effect was not to be expected on the basis of the structural similarity of these compounds.

### Example B15: Comparison of the Insecticidal Activity of Compounds P5. P10 and P17 According to the Invention with Structurally Most Closely Comparable Compounds from the State of the Art

Activity of compounds P5, P10 and P17 according to the preparatory examples and of compounds from WO 21/083936 against Euschistus heros (Example B3 above), Plutella xylostella (Example B4b above) and Pseudoplusia includens (Example B9 above) is summarized in Table B05.

TABLE B15

| | Concentration | | Mortality |
|---|---|---|---|
| Compound | (ppm) | Insect | (%) |
| Compound P17 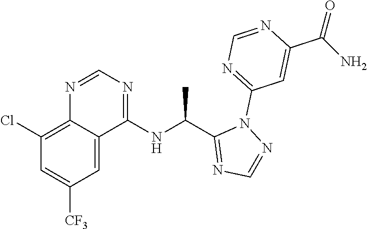 [SMILES: C[C@H](Nc1ncnc2c(Cl)cc(C(F)(F)F)cc12)c1ncnn1-c1cc(C(N)=O)ncn1] Present invention | 200 0.8 3 | Euschistus heros Plutella xylostella Pseudoplusia includens | 80 100 80 |
| Described in WO 21/083936 as compound P.18 (page 129) 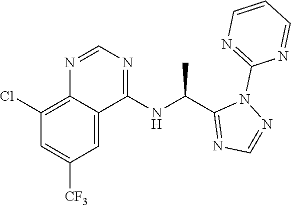 [SMILES: C[C@H](Nc1ncnc2c(Cl)cc(C(F)(F)F)cc12)c1ncnn1-c1ncccn1] State of the art | 200 0.8 3 | Euschistus heros Plutella xylostella Pseudoplusia includens | 0 65 40 |
| Compound P5 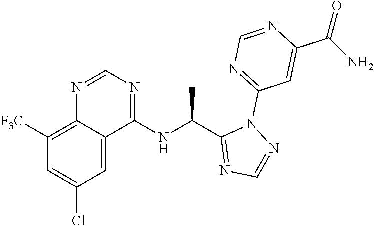 [SMILES: C[C@H](Nc1ncnc2c(C(F)(F)F)cc(Cl)cc12)c1ncnn1-c1cc(C(N)=O)ncn1] Present invention | 200 0.8 0.8 | Euschistus heros Plutella xylostella Pseudoplusia includens | 100 95 70 |
| Described in WO 21/083936 as compound P.24 (page 131) 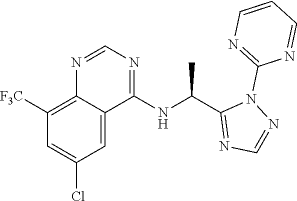 [SMILES: C[C@H](Nc1ncnc2c(C(F)(F)F)cc(Cl)cc12)c1ncnn1-c1ncccn1] State of the art | 200 0.8 0.8 | Euschistus heros Plutella xylostella Pseudoplusia includens | 0 5 0 |
| Compound P10 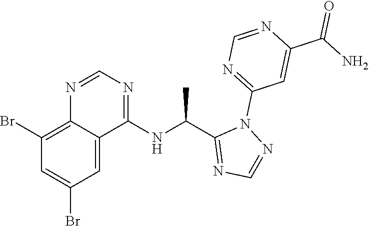 [SMILES: C[C@H](Nc1ncnc2c(Br)cc(Br)cc12)c1ncnn1-c1cc(C(N)=O)ncn1] Present invention | 200 3 3 | Euschistus heros Plutella xylostella Pseudoplusia includens | 0 100 80 |
| Described in WO 21/083936 as compound P.29 (page 132) 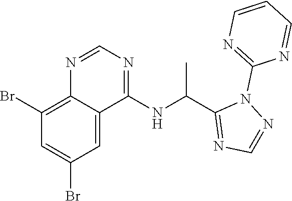 [SMILES: CC(Nc1ncnc2c(Br)cc(Br)cc12)c1ncnn1-c1ncccn1] State of the art | 200 3 3 | Euschistus heros Plutella xylostella Pseudoplusia includens | 0 60 30 |

Table B15 shows that compounds P5, P10 and P17 according to the invention exert a substantially better insecticidal action on Euschistus heros, Plutella xylostella and Pseudoplusia includens than the compounds from the state of the art. This enhanced effect was not to be expected on the basis of the structural similarity of these compounds.

1. A compound of the formula (I) 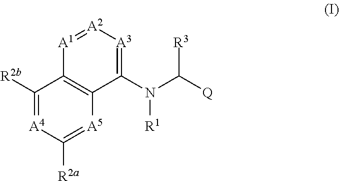
wherein:
Ar¹, A² and A³ are, independently from each other, N or CR^{Y}; or
A¹=A²-A³, taken together, are NR—C(═O)—N;
A⁴ and A⁵ are, independently from each other, N or CR^{Y};
Q is 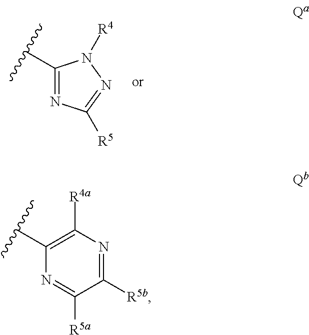
where the staggered line represents the connection of Q to the rest of compound of the formula (I);
R is hydrogen, C₁-C₃alkyl, C₁-C₃haloalkyl, C₁-C₃alkoxy, or C₁-C₃haloalkoxy;
R¹ is hydrogen, C₁-C₆alkyl, C₁-C₆cyanoalkyl, aminocarbonylC₁-C₆alkyl, hydroxycarbonylC₁-C₆alkyl, C₁-C₆nitroalkyl, trimethylsilaneC₁-C₆alkyl, C₁-C₃alkoxy-C₁-C₆alkyl, C₁-C₆haloalkyl, C₂-C₆alkenyl, C₂-C₆haloalkenyl, C₂-C₆alkynyl, C₂-C₆haloalkynyl, C₃-C₄cycloalkylC₁-C₂alkyl-, C₃-C₄cycloalkylC₁-C₂alkyl- wherein the C₃-C₄cycloalkyl group is substituted with 1 or 2 halogen atoms, oxetan-3-yl-CH₂—, C₁-C₆alkylcarbonyl, C₁-C₆alkoxycarbonyl, phenyloxycarbonyl, benzyloxycarbonyl, benzyl or benzyl substituted with 1 to 3 substituents independently selected from halogen, C₁-C₆alkoxy and C₁-C₆haloalkyl;
R^{2a} and R^{2b} are each independently selected from hydrogen, C₁-C₃alkyl, C₁-C₃haloalkyl, C₁-C₃haloalkylsulfanyl, C₁-C₃alkoxy, C₁-C₃haloalkoxy, halogen, NO₂, SF₅, CN, C(O)NH₂, C(O)OH, C(S)NH₂, C₃-C₆cycloalkyl, C₃-C₆cycloalkyl substituted with one to three substituents independently selected from R^{X}, C₃-C₆cycloalkylcarbonyl, phenyl, phenyl substituted with one to three substituents independently selected from R^{X}, heteroaryl, heteroaryl substituted with one to three substituents independently selected from R^{X}; OR⁶, piperidin-2-one-1-yl, piperidin-2-one-1-yl substituted with one to two substituents independently selected from R^{X}, pyridin-2-one-1-yl, pyridin-2-one-1-yl substituted with one to two substituents independently selected from R^{X}, azetidin-1-yl, azetidin-1-yl substituted with one to two substituents independently selected from R^{X}, pyrrolidin-1-yl, pyrrolidin-1-yl substituted with one to two substituents independently selected from R^{X}, C₃-C₆cycloalkylC₁-C₄alkyl, C₃-C₆cycloalkylC₁-C₄alkyl substituted with one to two substituents independently selected from R^{Z}; C₃-C₆cycloalkylC₁-C₃alkoxy, C₃-C₆cycloalkylC₁-C₃alkoxy substituted with one to two substituents independently selected from R^{x}, C₁-C₅cyanoalkyl, C₁-C₅cyanoalkoxy, C₁-C₄alkylsulfanyl, C₁-C₄alkylsulfanyl substituted with one to three substituents independently selected from R^{X}, C₁-C₄alkylsulfonyl, C₁-C₄alkylsulfonyl substituted with one to three substituents independently selected from R^{X}, C₁-C₄alkylsulfinyl, and C₁-C₄alkylsulfinyl substituted with one to three substituents independently selected from R^{X};
R³ is C₁-C₃alkyl or C₁-C₃haloalkyl;
R⁴ and R^{4a} are independently pyrimidinyl, pyrazinyl, pyridazinyl or thiazolyl, each of which, independently of each other, is substituted with a single —C(O)NR¹⁰R¹¹; or
R^{4a} is N-linked pyrazolyl or N-linked triazolyl, each of which, independently of each other, is substituted with a single —C(O)NR¹⁰R¹¹; or
R⁴ and R^{4a} are 4-cyano-pyrimidin-6-yl;
R¹⁰ is hydrogen, hydroxy, C₁-C₃alkyl, C₁-C₃cyanoalkyl, C₁-C₃haloalkyl, C₁-C₃alkoxy, C₃-C₄cycloalkyl, C₃-C₄halocycloalkyl, cyanoC₃-C₄cycloalkyl, C₃-C₄cycloalkylC₁-C₃cyanoalkyl, oxetan-3-yl, thietan-3-yl, 3-methylthietan-3-yl, 1,1-dioxothietan-3-yl, or 3-methyl-1,1-dioxothietan-3-yl; and
R¹¹ is hydrogen, C₁-C₃alkyl or C₁-C₃haloalkyl; or
R¹⁰ and R¹¹, together with the nitrogen they are attached to, form a pyrrolidin-1-yl, piperidin-1-yl or 4-morpholinyl group;
R⁵ is hydrogen, C₁-C₃alkyl, C₁-C₃haloalkyl, C₃-C₄cycloalkyl, C₁-C₃alkoxy, C₃-C₄alkoxyC(O)—, (C₁-C₃alkoxy)₂CH—, halogen, CN, NH₂C(O), amino (i.e. NH₂), (C₁-C₃alkyl)amino, di(C₁-C₃alkyl)amino, hydroxy, C₃-C₄halocycloalkyl, C₃-C₄cyanocycloalkyl, C₂-C₆alkenyl, C₂-C₆haloalkenyl, C₂-C₆alkynyl, C₂-C₆haloalkynyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl, C₁-C₄alkylsulfonyl, C₁-C₃alkoxy-C₁-C₃alkyl, C₁-C₃alkoxy-C₁-C₃alkoxy-C₁-C₃alkyl, (C₁-C₃alkyl)sulfonylamino, (C₁-C₃alkyl)sulfonyl(C₁-C₃alkyl)amino, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), (C₁-C₃alkyl)C(O)(C₁-C₃alkyl)N, (C₁-C₃alkyl)C(O)NH, (C₁-C₃alkyl)C(O), (C₁-C₃alkoxy)C(O), HC(O), diphenylmethanimine, C₁-C₃haloalkoxy, phenyl, or a 5-membered heteroaromatic ring; or
R⁵ is phenyl substituted with one to three substituents selected from C₁-C₃alkyl, C₁-C₃haloalkyl, C₁-C₃alkoxy, C₃-C₄cycloalkyl, halogen, CN and hydroxyl; or
R⁵ is a 5-membered heteroaromatic ring substituted with one to three substituents selected from C₁-C₃alkyl, C₁-C₃haloalkyl, C₁-C₃alkoxy, C₃-C₄cycloalkyl, halogen, CN and hydroxyl;
R^{5a} and R^{5b} are, independently of each other, selected from hydrogen, halogen, CN, C₁-C₃alkyl, C₁-C₃haloalkyl, C₃-C₄cycloalkyl, C₁-C₃alkoxy, and C₁-C₃haloalkoxy;
R⁶ is phenyl, benzyl, heteroaryl, or C₃-C₆ cycloalkyl; or
R⁶ is phenyl, benzyl, heteroaryl, or C₃-C₆ cycloalkyl, each of which, independent of each other, is substituted with one to three substituents independently selected from R^{X};
R^{x} is independently selected from halogen, C₁-C₃alkyl, C₁-C₃haloalkyl, C₁-C₃alkoxy, C₁-C₃haloalkoxy, NO₂, SF₅, CN, C(O)NH₂, C(S)NH₂, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl and C₁-C₄alkylsulfonyl;
R^{Y} is selected from hydrogen, C₁-C₃ alkyl, C₁-C₃ haloalkyl, hydroxy, C₁-C₃ alkoxy, C₁-C₃ haloalkoxy, halogen, CN and cyclopropyl;
R^{YY} is selected from hydrogen, C₁-C₃ alkyl, C₁-C₃ haloalkyl, hydroxy, C₁-C₃ alkoxy, C₁-C₃ haloalkoxy, halogen, CN and cyclopropyl; and
R^{Z} is selected from oxo, halogen, C₁-C₃ alkyl, C₁-C₃haloalkyl, C₁-C₃alkoxy, C₁-C₃haloalkoxy and CN;
or an agrochemically acceptable salt, stereoisomer, enantiomer, tautomer or N-oxide of the compound of formula (I).

2. The compound according to claim 1 wherein A¹ and, A³ are N and A² is CH, and A⁴ is CR^{Y} and A⁵ is CH; and R^{Y} is selected from hydrogen, methyl, trifluoromethyl, and methoxy.

3. The compound according to claim 1 wherein R¹ is hydrogen, methyl, ethyl, cyanomethyl, methoxymethyl, cyclopropyl-methyl, allyl, propargyl, benzyloxycarbonyl, or benzyl.

4. The compound according to claim 1 wherein R^{2a} is halogen, C₁-C₃haloalkyl, C₁-C₃haloalkylsulfanyl, C₁-C₃haloalkoxy, C₃-C₆cycloalkyl, C₃-C₆cycloalkyl substituted with one or two substituents independently selected from C₁-C₃haloalkyl, cyano, and halogen, C₃-C₆cycloalkylC₁-C₄alkyl, C₃-C₆cycloalkylC₁-C₄alkyl substituted with one to three substituents independently selected from C₁-C₃haloalkyl, cyano, and halogen, C₁-C₅cyanoalkyl, C₁-C₄alkylsulfonyl, C₁-C₄haloalkylsulfonyl, C₁-C₄alkylsulfinyl, C₁-C₄haloalkylsulfinyl, C₃-C₆cycloalkylsulfanyl, C₃-C₆cycloalkylsulfinyl, or C₃-C₆cycloalkylsulfonyl.

5. The compound according to claim 1 wherein R^{2b} is halogen, C₁-C₃haloalkyl, C₁-C₃haloalkylsulfanyl, C₁-C₃haloalkylsulfonyl, C₁-C₃alkoxy, C₁-C₃haloalkoxy, or CN.

6. The compound according to claim 1 wherein R³ is C₁-C₃alkyl or C₁-C₃haloalkyl.

7. The compound according to claim 1 wherein Q is selected from Q^{a}-1 to Q^{a}-16 and R⁴ is thiazol-2-yl, thiazol-4-yl, thiazol-5-yl, 1,2,4-thiadiazol-3-yl, 1,3,4-thiadiazol-2-yl or 1,2,4-thiadiazol-5-yl, each of which, independently of each other, is unsubstituted or substituted with one to two substituents independently selected from C₁-C₃alkyl, C₁-C₃haloalkyl, C₁-C₃alkoxy, C₃-C₄cycloalkyl, halo, hydroxyl, CN, C₁-C₆haloalkoxy, C₂-C₆haloalkenyloxy, C₂-C₆haloalkynyloxy, C₃-C₄halocycloalkoxy, NH₂C(O)—, NH₂C(S)—, (OH)N=C(NH₂)—and a 5-membered heteroaryl ring optionally substituted by 1 to 3 substituents independently selected from halogen, C₁-C₃alkyl, C₁-C₃haloalkyl, C₁-C₃alkoxy and C₁-C₃haloalkoxy.

8. The compound according to claim 1 wherein Q is selected from Q^{b}-1 to Q^{b}-13 and R^{4a} is thiazol-2-yl, thiazol-4-yl, thiazol-5-yl, 1,2,4-thiadiazol-3-yl, 1,3,4-thiadiazol-2-yl or 1,2,4-thiadiazol-5-yl, each of which, independently of each other, is unsubstituted or substituted with one to two substituents independently selected from C₁-C₃alkyl, C₁-C₃haloalkyl, C₁-C₃alkoxy, C₃-C₄cycloalkyl, halogen, hydroxyl, cyano, and C₁-C₃haloakoxy.

9. The compound according to claim 1, wherein R⁴ is selected from Q^{c}-1, Q^{c}-2, Q^{c}-3, Q^{c}-4, Q^{c}-5, Q^{c}-6, and Q^{c}-10; or R^{4a} is selected from Q^{c}-1, Q^{c}-2, Q^{c}-3, Q^{c}-4, Q^{c}-5, Q^{c}-6, Q^{c}-7, Q^{c}-8, and Q^{c}-10: 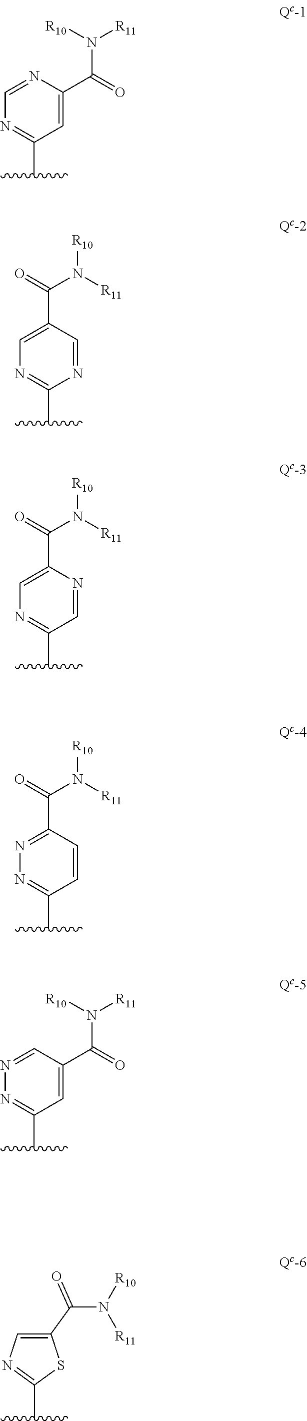 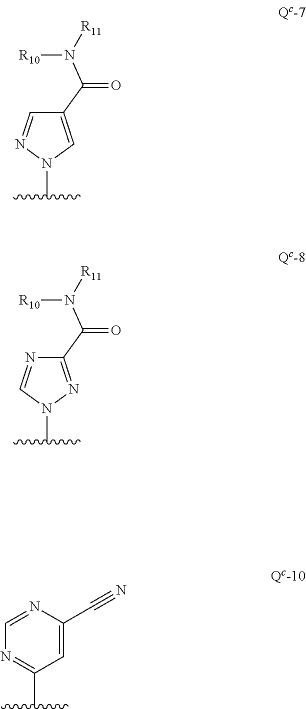
wherein R¹⁰ is hydrogen, methyl, ethyl, cyanomethyl, difluoromethyl, trifluoromethyl, cyclopropyl, or 1-cyanocyclopropyl; or
wherein R¹⁰ is hydrogen, hydroxy, methyl, ethyl, trifluoromethyl, cyanoethyl, methoxy, ethoxy, cyclopropyl, 1-cyanocyclopropyl, 1-cyano-1-cyclopropyl-ethyl, oxetan-3-yl, thietan-3-yl, 3-methylthietan-3-yl, 1,1-dioxothietan-3-yl, or 3-methyl-1,1-dioxothietan-3-yl;
and R¹¹ is hydrogen, methyl, ethyl, difluoromethyl, or trifluoromethyl.

10. The compound according to claim 1 wherein the formula (I) is represented by formulae Iaa, Iab, Iac, lad, or Iae, with an asterisk indicating a stereogenic centre:

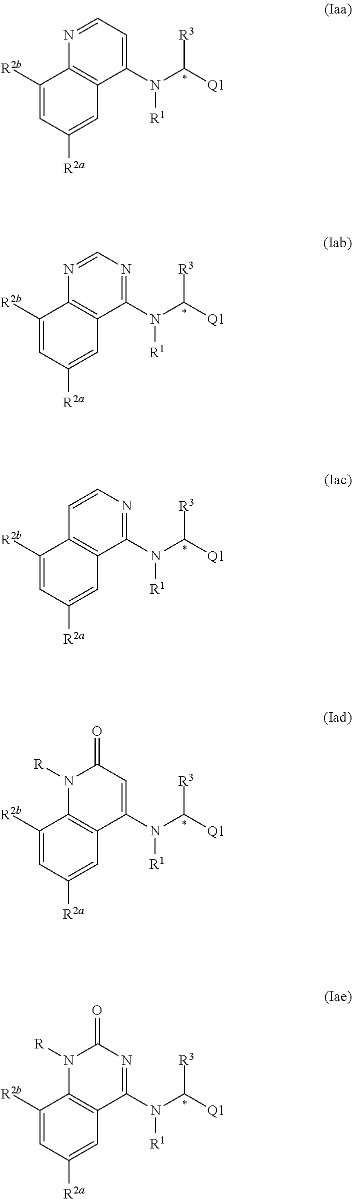

11. The compound according to claim 10 wherein Q₁ is selected from Q^{a}-1 and Q^{b}-1: 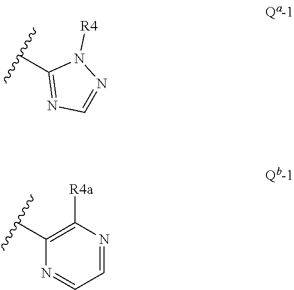
and wherein R⁴ and R^{4a} are independently selected from 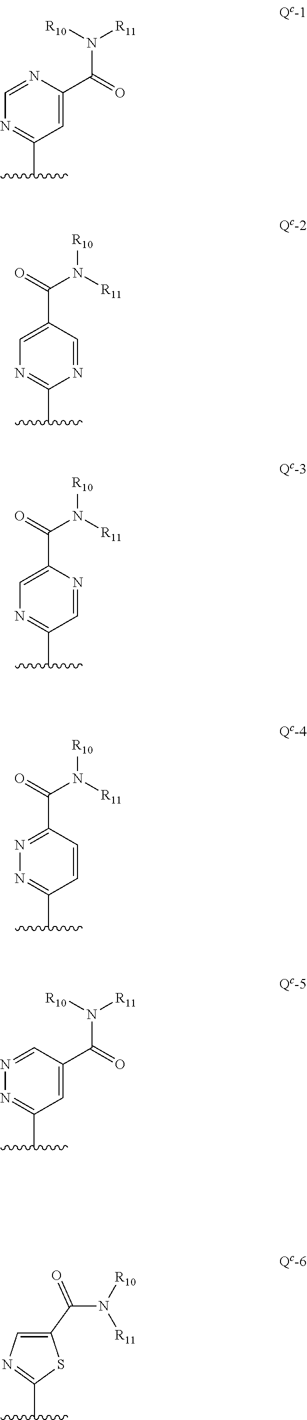
wherein R¹⁰ is hydrogen, methyl, ethyl, cyanomethyl, difluoromethyl, trifluoromethyl, cyclopropyl, or 1-cyanocyclopropyl; and R¹¹ is hydrogen, methyl, ethyl, difluoromethyl, or trifluoromethyl.

12. The compound according to claim 10 wherein Q₁ is selected from Q^{a}-1 and Q^{b}-1: 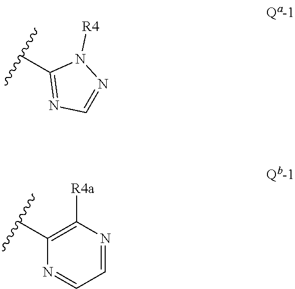
and wherein R⁴ and R^{4a} are Q^{c}-10.

13. A composition comprising a compound as defined in claim 1, one or more auxiliaries and diluent, and optionally one or more other active ingredient.

14. A method
(i) of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound as defined in claim 1, or a composition thereof; or
(ii) for the protection of plant propagation material from the attack by insects, acarines, nematodes or molluscs, which comprises treating the propagation material or the site, where the propagation material is planted, with an effective amount of a compound as defined in claim 1, or a composition thereof; or
(iii) of controlling parasites in or on an animal in need thereof comprising administering an effective amount of a compound as defined in claim 1, or a composition thereof.

15. A plant propagation material, such as a seed, comprising, or treated with or adhered thereto, a compound as defined in claim 1, or a composition thereof.

16. A compound of formula XL-Q^{ac}-a, XL-Q^{ac}-b, or XL-Q^{ac}-c: 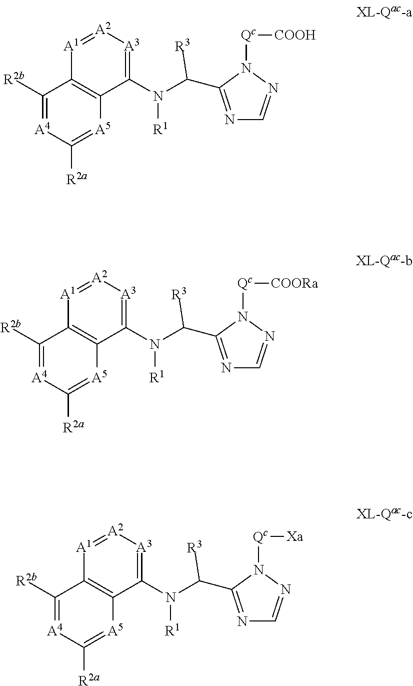
wherein in each case, as applicable:
A¹, A², A³, A⁴, A⁵, R^{2a}, and R^{2b} are as defined in claim 1,
R¹ is hydrogen or methyl,
R³ is methyl,
Q^{c}-COOH, Q^{c}-COORa and Q^{c}-Xa are: 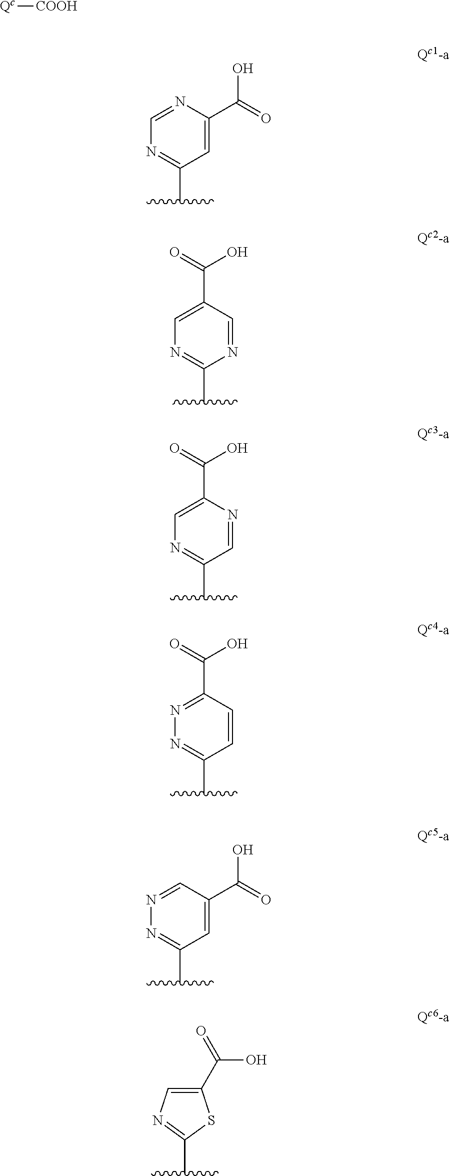 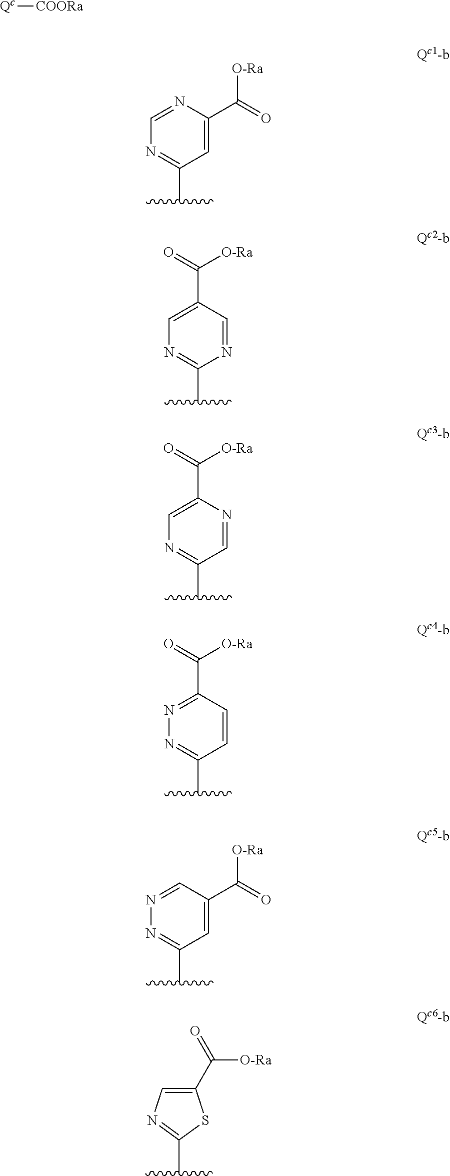 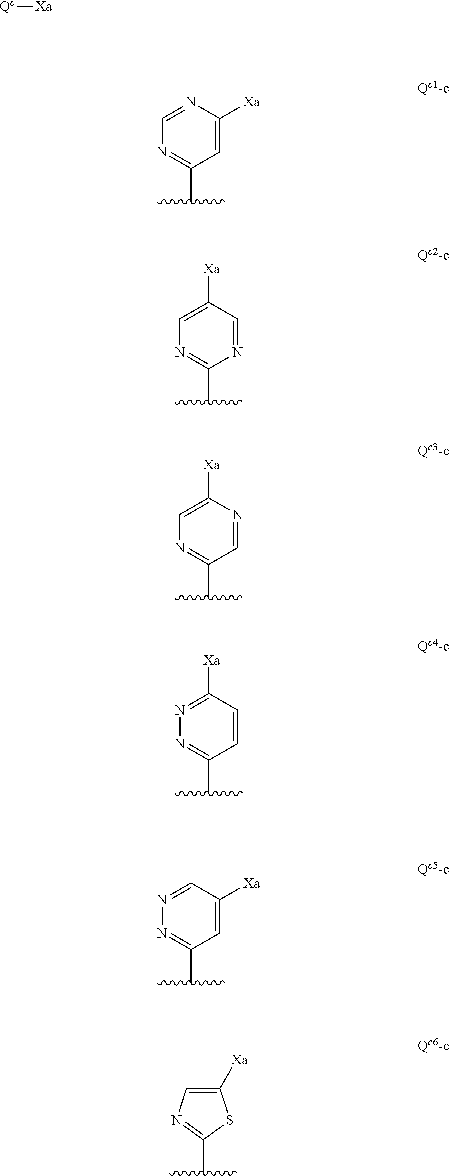
Ra is benzyl or C₁₋₆alkyl, preferably Ra is methyl, an
Xa is a halogen, such as Br, Cl or I, preferably Cl.

17. A compound of formula XL-Q^{bc}-a XL-Q^{bc}-b, or XL-Q^{bc}-c: 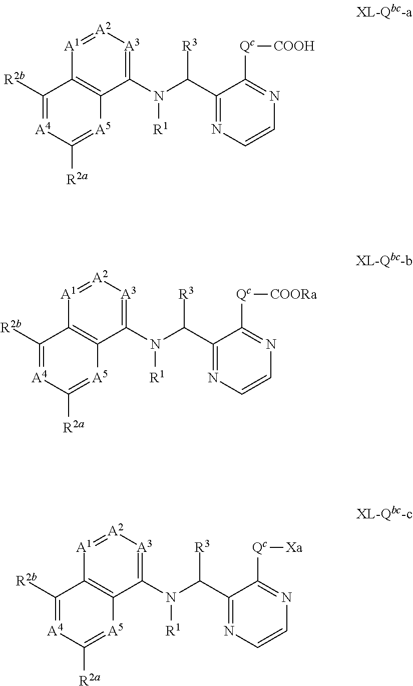
wherein in each case, as applicable:
A¹, A², A³, A⁴, A⁵, R^{2a}, and R^{2b} are as defined in claim 1,
R¹ is hydrogen or methyl,
R³ is methyl,
Q^{c}-COOH, Q^{c}-COORa and Q^{c}-Xa are Q^{c1}-a, Q^{c1}-b and Q^{c1}-c respectively, as defined thereof,
Ra is benzyl or C₁-C₆alkyl, preferably Ra is methyl, and
Xa is a halogen, such as Br, Cl or I, preferably Cl.

18. A compound of formula XLIIIa(i), XLIIIb(i), XLIIIb-1(i), XLIIId(i), or XLIIIe(i): 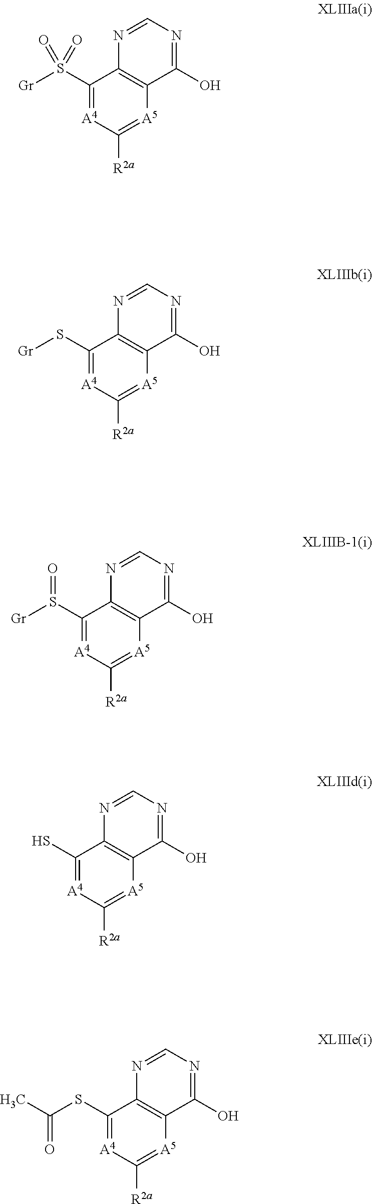
wherein A⁴, A⁵, and R^{2a} are as defined in claim 1 and wherein Gr is difluoromethyl or trifluoromethyl.
